(19) United States
(12) Patent Application Publication
(10) Pub. No.: US 2025/0302938 A1
(43) Pub. Date: Oct. 2, 2025

(54) RECOMBINANT SUBUNIT BASED UNIVERSAL INFLUENZA AND RESPIRATORY VIRUS VACCINES

(71) Applicant: GEORGIA STATE UNIVERSITY RESEARCH FOUNDATION, INC., Atlanta, GA (US)

(72) Inventors: Sang-Moo KANG, Lilburn, GA (US); Jeeva SUBBIAH, Duluth, GA (US); Ki-Hye KIM, Atlanta, GA (US)

(21) Appl. No.: 18/863,015

(22) PCT Filed: May 10, 2023
(86) PCT No.: PCT/US2023/066804

§ 371 Date: Nov. 5, 2024

Related U.S. Application Data
(60) Provisional application No. 63/364,502, filed on May 11, 2022.

(51) Int. Cl. A61K 39/145; A61K 39/00; A61P 31/16; C12N 7/00; C12N 15/62

(52) U.S. Cl. CPC A61K 39/145; A61P 31/16; C12N 7/00; C12N 15/62; A61K 2039/5258; A61K 2039/53; A61K 2039/54; A61K 2039/70; C12N 2760/16123; C12N 2760/16134; C12N 2760/16234

(57) ABSTRACT

In accordance with the purpose(s) of the present disclosure, as embodied and broadly described herein, the disclosure relates to universal influenza vaccines and methods of making the same. Also disclosed is method for vaccinating a subject for influenza A that involves administering a cross-protective influenza vaccine disclosed herein to a subject in need thereof by intranasal, intramuscular, subcutaneous, transdermal, or sublingual administration.

### CROSS-REFERENCE TO RELATED APPLICATIONS

This application claims the benefit of U.S. Provisional Application No. 63/364,502, filed on May 11, 2022, which is incorporated herein by reference in its entirety.

### STATEMENT REGARDING FEDERALLY SPONSORED RESEARCH OR DEVELOPMENT

This invention was made with government support under grant numbers AI093772 and AI152800 awarded by the National Institutes of Health. The government has certain rights in the invention.

### SEQUENCE LISTING

This application contains a sequence listing filed in ST.26 format entitled “220702_2470_Sequence_Listing” created on May 9, 2023. The content of the sequence listing is incorporated herein in its entirety.

### BACKGROUND

Hemagglutinin (HA) stem-based vaccines have limitations in providing broad and effective protection against cross-group influenza viruses despite being a promising universal vaccine target. To overcome the limited cross protection and low efficacy by HA stem vaccination, we genetically engineered a chimeric conjugate of thermostable H1 HA stem and highly conserved M2e repeat (M2e-H1stem), which was expressed at high yields in Escherichia coli. M2e-H1stem protein presented native-like epitopes reactive to antisera of live virus infection. M2e-H1stem protein vaccination of mice induced strong M2e- and HA stem-specific immune responses, conferring broadly effective cross-protection against both antigenically distinct group 1 (H1N1, H5N1, H9N2 subtypes) and group 2 (H3N2, H7N9 subtypes) seasonal and pandemic potential influenza viruses. M2e-H1stem vaccination generated CD4+ and CD8+ T cell responses and antibody-dependent cytotoxic cellular and humoral immunity, which contributed to enhancing cross-protection. Furthermore, comparable broad cross-group protection was observed in older aged mice after M2e-H1stem vaccination. This study provides evidence warranting further development of chimeric M2e-stem proteins as a promising universal influenza vaccine candidate in adult and aged populations.

A new chimeric M2e and H3 hemagglutinin (HA) stalk protein vaccine (M2e-H3 stalk) was generated by genetic engineering of modified H3 stalk domain conjugated with conserved M2e epitopes to overcome the drawbacks of low efficacy by monomeric domain-based universal vaccines. M2e-H3 stalk protein expressed and purified from Escherichia coli was thermostable, displaying native-like antigenic epitopes recognized by antisera of different HA subtype proteins and influenza A virus infections. Adjuvanted M2e-H3 stalk vaccination induced M2e and stalk-specific IgG antibodies recognizing viral antigens on virus particles and on the infected cell surface, CD4+ and CD8+ T cell responses, and antibody-dependent cytotoxic cell surrogate activity in mice. M2e-H3 stalk was found to confer protection against heterologous and heterosubtypic cross-group subtype viruses (H1N1, H5N1, H9N2, H3N2, H7N9) at similar levels in adult and aged mice. These results provide evidence that M2e-H3 stalk chimeric proteins can be developed as a universal influenza A virus vaccine candidate for young and aged populations.

Annual influenza vaccination is recommended to update the variable hemagglutinin antigens. Here, a virus-like particle (VLP) was designed displaying consensus multi-neuraminidase (NA) subtypes (cN1, cN2, B cNA) and M2 ectodomain (M2e) tandem repeat (m-cNA-M2e VLP). Vaccination of mice with m-cNA-M2e VLP induced broad NA inhibition (NAI), M2e antibodies as well as interferon-gamma secreting T cell responses. Mice vaccinated with m-cNA-M2e VLP were protected against influenza A (H1N1, H5N1, H3N2, H9N2, H7N9) and influenza B (Yamagata and Victoria lineage) viruses containing substantial antigenic variations. Protective immune contributors include cellular and humoral immunity as well as antibody-dependent cellular cytotoxicity. Furthermore, comparable cross protection by m-cNA-M2e VLP vaccination was induced in aged mice.

### SUMMARY

In accordance with the purpose(s) of the present disclosure, as embodied and broadly described herein, the disclosure, in one aspect, relates to universal influenza vaccines and methods of making the same.

For example, disclosed herein is a cross-protective influenza vaccine involving a virus-like particle (VLP) comprising an influenza matrix protein 1 (M1) and displaying on its surface consensus N1 neuraminidase (cN1), consensus N2 neuraminidase (cN2), consensus influenza B neuraminidase (B-cNA), and a tandem repeat of two influenza virus matrix protein 2 extracellular (M2e) domains derived from a human influenza A subtype (hM2e), one M2e domain derived from a swine influenza A subtype (sM2e), one avian M2e domain derived an avian type I influenza A subtype (a1M2e), and one avian M2e domain derived an avian type II influenza A subtype (a2M2e).

Therefore, also disclosed is an expression vector construct encoding the proteins to produce the disclosed VLP. In some embodiments, the expression vector has the formula:

M1-cN1-cN2-B-cNA-5xM2e-X₂-X₃,

M1-cN2-cN1-B-cNA-5xM2e-X₂-X₃,

M1-B-cNA-cN1-cN2-5xM2e-X₂-X₃,

M1-B-cNA-cN2-cN1-5xM2e-X₂-X₃,

M1-cN1-B-cNA-cN2-5xM2e-X₂-X₃, or

M1-cN2-B-cNA-cN1-5xM2e-X₂-X₃,
wherein “cN1” consists of a gene encoding the consensus N1 neuraminidase,
wherein “cN2” consists of a gene encoding the consensus N2 neuraminidase,
wherein “B-cNA” consists of a gene encoding the consensus influenza B neuraminidase,
wherein “5xM2e” consists of a gene encoding the two hM2e domains, one sM2e domains, one a1M2e domain and one a2M2e domain in any order, and
wherein “-” consists of a nucleic acid linker.

For example, in some embodiments, the expression vector construct has the formula:

M1-cN1-cN2-B-cNA-hM2e-hM2e-sM2e-a1M2e-a3M2e.

In some embodiments, the vaccine is produced by coinfecting insect cells with one or more expression vectors disclosed herein, culturing the insect cells under physiological conditions, and purifying the VLPs from insect cell culture supernatants.

In some embodiments, the vaccine further contains an influenza virus-like particle (VLP) vaccine, an mRNA vaccine, a whole inactivated virus, split viral vaccine, or live attenuated influenza vaccine. In some embodiments, the vaccine is formulated for delivery via intranasal, intramuscular, subcutaneous, transdermal or sublingual administration.

Also disclosed herein is a cross-protective influenza vaccine that involves a fusion protein having two influenza virus matrix protein 2 extracellular (M2e) domains, amino acids 37-61 of an influenza virus hemagglutinin (HA) H3 stalk head domain (HA1), amino acids 305-338 of the HA1, amino acids 1-117 of an HA H3 stalk stem domain (HA2), and a trimeric foldon domain.

In some embodiments, the fusion protein has an amino acid sequence having a formula:

M2e-M2e-HA1a-HA1b-HA2-FO,
wherein “M2e” consists of an hM2e domain,
wherein “HA1a” consists of amino acids 37-61 of an HA1 domain,
wherein “HA1b” consists of amino acids 305-338 of an HA1 domain,
wherein “HA2” consists of amino acids 1-117 of an HA2 domain,
wherein “FO” consists of a trimeric foldon, and
wherein “-” consists of a flexible peptide linker or a peptide bond.

In some embodiments, the fusion protein is expressed by an E. coli bacteria. In some embodiments, the M2e domain is derived from a human (hM2e) or swine (sM2e) influenza virus.

Also disclosed herein is a cross-protective influenza vaccine involving a fusion protein containing two influenza virus matrix protein 2 extracellular (M2e) domains, amino acids 31-54 of an influenza virus hemagglutinin (HA) H1 stalk head domain (HA1), amino acids 304-337 of the HA1, amino acids 1-117 of an HA H3 stalk stem domain (HA2), and a trimeric foldon domain.

In some embodiments, the fusion protein has an amino acid sequence having a formula:

M2e-M2e-HA1a-HA1b-HA2-FO,
wherein “M2e” consists of an hM2e domain,
wherein “HA1a” consists of amino acids 37-61 of an HA1 domain,
wherein “HA1b” consists of amino acids 305-338 of an HA1 domain,
wherein “HA2” consists of amino acids 1-117 of an HA2 domain,
wherein “FO” consists of a trimeric foldon, and
wherein “-” consists of a flexible peptide linker or a peptide bond.

In some embodiments, the fusion protein is expressed by an E. Coli bacteria.

Also disclosed herein are polynucleotides comprising mRNA or cDNA that encode a fusion protein disclosed herein.

Also disclosed herein is a composition comprising a cross-protective influenza vaccine disclosed herein in a pharmaceutically acceptable excipient.

Also disclosed herein is a method for vaccinating a subject for influenza A that involves administering a cross-protective influenza vaccine disclosed herein to a subject in need thereof by intranasal, intramuscular, subcutaneous, transdermal, or sublingual administration. In some embodiments, the method further involves administering to the subject a composition comprising a VLP vaccine, a mRNA vaccine, a whole inactivated virus, split viral vaccine, or live attenuated influenza vaccine. For example, in some embodiments, the cross-protective influenza vaccine and the influenza virus-like particle (VLP) vaccine, the mRNA vaccine, the whole inactivated virus, the split viral vaccine, or the live attenuated influenza vaccine are in the same composition. In other embodiments, the composition comprising influenza virus-like particle (VLP) vaccine, a whole inactivated virus, split viral vaccine, or live attenuated influenza vaccine is administered before or after the cross-protective influenza vaccine. In some embodiments, the cross-protective influenza vaccine is administered prior to influenza seasonal vaccination or after influenza seasonal vaccination. In some embodiments, the period between cross-protective influenza vaccine and seasonal vaccination administration is one day to 10 years.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1A shows a schematic diagram of full-length HA (A/PR8). The regions that were selected as a vaccine target are numbered in amino acid (aa 31-54, 304-337, 1-117) residues. FIG. 1B shows chimeric M2e-H1stem construct with linker sequences (AAAGGAA (SEQ ID NO:39); GGGGS (SEQ ID NO:40); GSA; GSAGSA (SEQ ID NO:41); PG; QGTGG (SEQ ID NO:42)). M2e sequence: MSLLTEVETPIRNEWGSRSNDSSD (SEQ ID NO: 43) Cys to Ser changes underlined). FIGS. 1C-1D show structural modeling of HA and M2e-H1stem construct. A/PR8/1934 HA (ID: YP_163736) was used as a template to predict the structure of HA and M2e-H1stem by SWISS-Model (Expasy web server); the 3D cartoon structure was generated by PyMol. The structure of M2e and foldon was derived from the PDB ID code 4N8C and 1 RFO, respectively. In FIG. 1D, the locations of the point mutations are marked in pink, whereas the HA1 region is colored in blue. FIG. 1E shows Coomassie Blue staining of M2e-H1stem protein. M: protein size marker (kDa), TP: Total cell lysates (30 μg); M2e-H1stem: purified M2e-H1stem protein (20 μg). FIG. 1F shows Western blot of M2e-H1stem protein. 14C2: M2e-specific mAb; Stem: anti-stem polyclonal antibodies (pAbs) recognizing HA2 aa103-116 peptide. i:protein ladder kDa, ii: purified M2e-H1stem protein, iii: empty vector transformed E. coli cell lysate.

FIGS. 2A-2K show antigenic characterization of M2e-H1 stem protein. The antigenicity of M2e and stem epitopes in the M2e-H1stem protein was determined by ELISA using epitope-specific antibodies. FIG. 2A shows M2e-specific monoclonal antibody (mAb 14C2). FIG. 2B shows unit-1 mAb: rabbit mAb specific for fusion domain (GLFGAIAGFIEGGW, SEQ ID NO:44). FIG. 2C shows H3-FP polyclonal antibody (pAb) (HA2 aa14-27): purified rabbit pAbs specific for HA2 aa14-27 (WEGMVDGWYGFRHQ, SEQ ID NO:45). FIG. 2D shows H1stem pAb: purified rabbit pAbs specific for HA2 aa103-116 (ENERTLDYHDSNVK, SEQ ID NO:46). FIGS. 2E-2H: Antigenicity of M2e-H1stem to pAbs specific for recombinant HA. FIG. 2E shows pAb to H1 HA: antisera for H1 HA (A/California/04/2009 H1N1), FIG. 2F shows pAb to H5 HA: antisera for H5 HA (A/Vietnam/1203/04/H5N1), FIG. 2G shows pAb to H3 HA: antisera for H3 HA (A/Hong Kong/1/1968/H3N2), FIG. 2H shows pAb to H7 HA: antisera for H7 HA (A/Anhui/1/2013/H7N9). FIGS. 2I-2K shows Antigenicity of M2e-H1stem to antisera from live virus infection. FIG. 2I shows H5N1 antisera (A/Viet/H5N1), FIG. 2J shows H3N2 antisera (A/Phil/1982 H3N2), FIG. 2K shows H7N9 antisera (A/Sha/2013 H7N9). Control(−ve): BSA, HA Full(+ve): Full HA protein of A/Cal/H1N1 virus.

FIGS. 3A-3I show vaccination with adjuvanted M2e-H1stem induces antibodies recognizing M2e, stem, and group 1 and 2 viral antigens. BALB/c mice (n=10) were intramuscular prime-boost immunized with M2e-H1stem (20 μg) with or without adjuvants (QS21-MPL), and sera were collected 2 weeks after vaccination. FIGS. 3A-3C show IgG antibodies specific for M2e (FIG. 3A), stem protein (FIG. 3B), and H1 stem peptide HA2 aa74-98 (FIG. 3C). FIGS. 3D-3E show IgG antibodies specific for group 1 and 2 influenza A viruses. group 1 (G1) HA viruses. FIG. 3D shows A/Cal/09/H1N1, A/Viet/rgH5N1, A/HK/rgH9N2); group 2 (G2) HA viruses (FIG. 3E: A/HK/H3N2, A/Phil/H3N2, A/Sha/H7N9). Mock Ctrl: adjuvanted naïve sera. FIGS. 3F-3G show M2e-H1stem vaccination induced IgG antibodies recognizing both group 1 (FIG. 3F) and 2 (FIG. 3G) viral antigens on the surface of virus-infected MDCK cells. FIGS. 3H-3I show antibodies induced by M2e-H1stem vaccine engage in Fc-mediated activation of Jurkat effector cells, mimicking a surrogate ADCC activation pathway. The statistical significance was determined by using two way ANOVA; error bars indicate mean±SEM; *, P<0.05; **, P<0.01; ***, P<0.001.

FIGS. 4A-4J show adjuvanted M2e-H1stem vaccination provides cross protection against both group 1 and 2 viruses. M2e-H1stem (20 μg+adjuvant) prime-boost vaccinated BALB/c mice (n=5 per group) were intranasally challenged with influenza A viruses. Body weight changes and survival rates were monitored for 14 days. Group1 viruses; (FIG. 4A) A/WSN/1933 H1N1 (2×LD₅₀, 1.5×10² EID₅₀), (B) A/Viet/2004 rgH5N1 (3×LD₅₀, 2.6×10⁴ EID₅₀), (C) A/HK/1999 H9N2 (4×LD₅₀, 7.8×10¹ EID₅₀), (D) A/PR8/1934 H1N1 (4×LD₅₀, 1.2×10³ EID₅₀), (E) A/Cal/2009 H1N1 (3×LD₅₀, 2×10³ EID₅₀), (F) A/FM/1947 H1N1 (3×LD₅₀, 8×10³ EID₅₀). Group 2 viruses; (G) A/Nanchang/1995 H3N2 (2×LD₅₀, 3×10⁶ EID₅₀), (H) A/Sha/2013 H7N9 (3×LD₅₀, 1.1×10⁴ EID₅₀), (I) A/Phil/1982 H3N2 (3×LD₅₀, 2.3×10² EID₅₀) and (J) A/HK/1968 H3N2 (3×LD₅₀, 4×10¹ EID₅₀). The statistical significance was determined by using two-way ANOVA; error bars indicate mean±SEM; *, P<0.05; **, P<0.01; ***, P<0.001.

FIGS. 5A-5N show adjuvanted M2e-H1stem vaccination bestows cross protection by lowering lung viral loads and inducing protective humoral and cellular immune responses. FIG. 5A shows body weight changes in M2e-H1stem (20 μg) prime-boost vaccinated BALB/c mice for 6 days after challenge with A/rgH7N9 virus. FIG. 5B shows lung viral titers at 6 days post-infection as in FIG. 5A. Mock inf: mock group with virus infection; non-infected: naïve group with no virus infection. FIGS. 5C-5D show body weight changes in naïve mice after intranasal inoculation with a mixture of M2e-H1stem immune or naïve sera and virus (4.2×LD₅₀). A/WSN (H1N1, in FIG. 5C) and A/rgH7N9 virus (A/Sha/2013, in FIG. 5D). FIG. 5E shows lung viral titers in naïve mice at 6 days post infection with a mixture of sera and A/rgH7N9 virus as in FIG. 5D. FIGS. 5F-5M show IFN-γ+ T cell responses analyzed by flow cytometry of intracellular cytokine staining; lung and spleen cells were collected at 5 days-post-infection with rgH7N9 virus. FIGS. 5F-5I show IFN-γ+ CD4+ T cells responses. Lung (FIG. 5F) and spleen (FIG. 5G) IFN-γ+ CD4+ T cells upon M2e stimulation. Lung (FIG. 5H) and spleen (FIG. 5I) IFN-γ+ CD4+ T cells upon stem protein stimulation. (FIGS. 5J-5M) IFN-γ+ CD8+ T cell responses. Lung (FIG. 5J) and spleen (FIG. 5K) IFN-γ+ CD8 T cells upon M2e stimulation. Lung (FIG. 5L) and spleen (FIG. 5M) IFN-γ+ CD8+ T cells upon stem protein stimulation. +/−: with/without CD4/CD8 depletion. (FIG. 5N) Impact of CD4+ or CD8+ T cell depletion on protection in adjuvanted M2e-H1stem (20 μg) vaccinated mice before challenge with A/rgH7N9 virus. Statistical significance was determined using the one- or two-way ANOVA followed by Tukey's multiple comparison or Bonferroni post-test. Error bars indicate means±SEM; *, P<0.05; **, P<0.01; ***, P<0.001.

FIGS. 6A-6J show evaluating the thermostability of adjuvanted M2e-H1stem vaccine and protective immunity in aged mice. FIGS. 6A-6B show the impact of high temperature storage of M2e-H1stem vaccine on inducing protective immunity. FIG. 6A shows M2e-H1 stem specific serum IgG antibody responses to vaccination of 8-weeks old mice (n=5) with adjuvanted M2e-H1stem pre-stored at 50° C. for 10 days. Mock inf: mock (no vaccine) group with virus infection FIG. 6B shows body weight changes in vaccinated mice with 50° C. pre-stored M2e-H1 stem after challenge with A/WSN/1933 H1N1 (2×LD₅₀, 1.5×10² EID₅₀). (FIGS. 6C-6J) Determination of immunogenicity and protection efficacy of M2e-H1stem vaccine in 14 month-old mice (n=6). (FIG. 6C) M2e- and stem-specific IgG antibodies. FIGS. 6D-6E show body weight changes in vaccinated mice after challenge with H1N1 virus (FIG. 6D, A/WSN/1933, 2×LD₅₀, 1.5×10² EID₅₀) or rgH7N9 (FIG. 6E, A/Sha/2013, 3×LD₅₀, 1.1×10⁴ EID₅₀). (FIG. 6F) Lung viral titers of FIG. 6E. FIGS. 6G-6H show in vitro IgG production from spleen cells. IgG antibodies specific for M2e (FIG. 6G) and stem (FIG. 6H) in spleen cells. FIGS. 6I-6J show IFN-γ-secreting splenocytes stimulated with M2e peptide (FIG. 6I) or stem (FIG. 6J). Statistical significance was determined using the one- or two-way ANOVA followed by Tukey's multiple comparison or Bonferroni post-test. Error bars indicate means±SEM; *, P<0.05; **, P<0.01; ***, P<0.001; ns, no significance between two compared groups.

FIGS. 7A-7B show a phylogenetic tree of influenza A viruses and sequence homology of amino acids between M2e-H1stem protein and its corresponding domain of challenge viruses. FIG. 7A shows a phylogenetic tree was constructed based on the amino acid (aa) sequences of the HA genes in H1-H18 influenza viruses obtained from GenBank. FIG. 7B shows the aa sequence homology between the M2e-H1stem protein and its corresponding HA domain of the challenge viruses. Influenza A (H1-H18) HA sequences were obtained from GenBank: GQ214335 for H1 HA of A/California/2009 (protein id: ACR47014), L11125 for H2 HA of A/Berkeley/1968 (AAA43089), M55059 for H3 HA of A/Aichi/1968 (AAA43239), MT421019 for H4 HA (QJI55045), EU122404 for H5 HA of A/Viet Nam/2004 (ABW90135), CY166897 for H6 HA (AHL82551), KC853228 for H7 HA of A/Shanghai/2013 (AG160292), CY097534 for H8 HA (AEM75966), AJ404627 for H9 HA (CAB95857), MF613851 for H10 HA (ASV60666), CY191275 for H11 HA (AKF35393), CY133357 for H12 HA (AGE03167), CY054300 for H13 HA (ADB46159), MK327694 for H14 HA (AZQ09016), KP087869 for H15 HA (AIY68624), CY177441 for H16 HA (AHM98288), CY103892 for H17 HA (AFC35438), and CY125945 for H18 HA (AGX84934). The complete HA and HA2 sequences were used to construct phylogenetic tree by using Clone Manager program and online tools. The HA sequences of the challenging viruses used in this study were obtained from GenBank to analyze the identity of aa sequences: GenBank accession No: NC_002017 H1 HA (YP_163736) for A/Puerto Rico/8/1934 (H1N1); CY010788 H1 HA (ABF47955) for A/WSN/1933 (H1N1); IVU02464 H1 HA (AAC53844) for A/Fort Monmouth/1/1947 (H1N1); NC_026433A H1 HA (YP_009118626) for A/California/07/2009 (H1N1); EU122404 H5 HA (ABW90135) for A/Vietnam/1203/2004 (H5N1); KF188366 H9 HA (AGO17823) for A/chicken/Hong Kong/G9/1997 (H9N2); IVU08858 H3 HA (AAA18781) for A/Philippines/2/82 (H3N2); KC853228 H7 HA (AG160292) for A/Shanghai/2013 (H7N9); CY080523 H3 HA (ADV76673) for A/Hong Kong/1-10-MA21-1/1968 (H3N2); KM821307 H3 HA (AIU46048) for A/Nanchang/933/1995 (H3N2)). The percentages of the aa homology of the vaccine construct M2e-H1stem HA1 and HA2 domains with HA sequences of challenge viruses were calculated using Needle (EMBOSS; EMBLEBI).

FIGS. 8A-8C show M2e-H1 stem prime vaccination induces higher levels of IgG antibodies specific for M2e than those for stem epitopes. The levels of IgG antibodies specific for M2e (FIG. 8A), stem (FIG. 8B), and H1 stem peptide (FIG. 8C) in immune sera after prime vaccination of mice with M2e-H1 stem in the presence or absence adjuvants (QS-21+MPL), detected by ELISA. Mock: adjuvanted naïve sera; Adj: Adjuvant. Statistical significance was determined using the two-way ANOVA. Error bars indicate means±SEM, *, P<0.05; **, P<0.01; ***, P<0.001.

FIGS. 9A-9H show adjuvant effects on IgG and IgG isotype antibodies induced by M2e-H1stem vaccination. The levels of total IgG, IgG1 and IgG2a antibodies specific for M2e-H1 stem were measured from prime (FIGS. 9A-9C) and boost (FIGS. 9D-9F) immune sera collected at two weeks after prime and boost vaccination of mice (n=10) with M2e-H1 stem protein with or without adjuvants (QS-21+MPL). FIG. 9G shows IgG reactivity to inactivated influenza A viruses. FIG. 9H shows IgG to M2e-H1stem foldon versus spike-foldon in M2e-H1stem boost sera. The IgG and IgG isotypes were determined using horseradish peroxidase-conjugated goat anti-mouse IgG, IgG1, and IgG2a secondary antibodies. Mock: adjuvanted naïve sera. Statistical significance was determined using the two-way ANOVA. Error bars indicate means±SEM, *, P<0.05; **, P<0.01; ***, P<0.001.

FIGS. 10A-10B show reactivity of adjuvanted M2e-H1stem antisera to viral antigens on the rgH5N1-infected MDCK cell surface by immunofluorescence and ELISA. Adjuvanted M2e-H1stem vaccination in mice induced IgG antibodies that recognize viral antigens on the surface of rgH5N1 virus-infected (100×TCID₅₀) MDCK cells, as evaluated by immunofluorescence assay (FIG. 10A) and ELISA (FIG. 10B). The assays were repeated in two independent experiments. Mock sera: adjuvanted boost naïve sera.

FIG. 11 shows adjuvant effects on enhancing the protective efficacy induced by M2e-H1 stem vaccination. M2e-H1 stem protein (20 μg) with or without adjuvants was used to prime-boost immunize 6-week-old BALB/c mice (n=3 per group). Body weight changes and survival rates were monitored for 14 days after infection with a lethal dose (8×LD₅₀, 3×10⁴ EID₅₀) of A/PR8/1934 H1N1 influenza virus. Mock inf: mock group with virus infection; Adj: Adjuvant. The statistical significance was determined by using two-way ANOVA; error bars indicate mean±SEM; *, P<0.05; **, P<0.01; ***, P<0.001.

FIGS. 12A-12B show antisera of adjuvanted M2e and stem vaccination provide protection against group 1 and group 2 influenza virus infection. M2e only antisera were obtained from the mice that received adjuvanted vaccination with M2e conjugated to the receptor binding domain (M2e-RBD) of SARS-CoV-2 spike protein. H1stem antisera were from the mice that received adjuvanted vaccination with HA2 stem protein. A mixture of virus (group1 A/WSN/H1N1 or group2 A/Phil/H3N2) and boost sera (mock control sera or M2e specific sera or stem specific sera or combination of M2e and stem sera) was used to intranasally infect naïve mice. Bodyweight changes and survival rates were monitored for 14 days and presented.

FIGS. 13A-13F show adjuvanted M2e-H1stem vaccination induces M2e- and stem-specific IgG antibody-secreting plasma cells and cytokine-secreting T cell responses upon in vitro antigen stimulation. Humoral and cytokine-secreting T cell immune responses were evaluated in immunized mice at 6 days-postinfection with rgH7N9 virus. FIGS. 13A-13B show in vitro production of IgG antibodies specific for M2e (FIG. 13A) and M2e-H1stem (FIG. 13B) in mediastinal lymph node (MLN) and spleen cultures, determined by ELISA. The cells were cultured on the plates pre-coated with antigens (M2e peptide, M2e-H1 stem, or stem protein) for 5 days at 37° C. FIGS. 13C-13F show IFN-γ-secreting ELISpot assays of spleen and lung cells from M2e-H1stem vaccinated mice after in vitro antigen stimulation with M2e—(FIG. 13C), M2e-H1 stem—(FIG. 13D), stem—(FIG. 13E), and H7N9 virus—(FIG. 13F). Mock inf: mock group with virus infection. Statistical significance was determined using the two-way ANOVA. Error bars indicate means±SEM; **, P<0.01; ***, P<0.001.

FIGS. 14A-14C show a gating strategy and representative flow cytometry profiles of IFN-γ+ CD4 and CD8 T cells. Spleen cells from adjuvanted M2e-stem or mock control immunized mice were isolated at day 6 upon infection with rgH7N9 virus. The cells were stimulated with 5 μg/mL of stem protein or M2e peptide with 20 μg/mL of Brefeldin A for 5 hours. T cells were stained with surface T cell marker and IFN-γ cytokine antibodies. FIG. 14A shows a gating strategy for identifying CD4 and CD8 subsets secreting IFN-γ. FIGS. 14B-14C show representative dot plots of CD4 and CD8 T cells secreting IFN-γ in response to stimulation with stem protein and M2e peptide. +: CD4 and CD8 T cells were depleted by treating T cell-depleting antibodies in vaccinated mice prior to challenge. −: intact vaccinated mice (No T cell depletion).

FIGS. 15A-15D show M2e-H1stem protein displays antigenic integrity and retention of epitopes after storage at high temperatures. The antigenicity of the M2e-H1 stem protein stored at different temperatures (4° C., 20° C., 37° C., or 50° C.) for 10 days was analyzed using epitope-specific antibodies, including anti-M2e (14C2) (FIG. 15A), anti-H3-FP (fusion peptide) (FIG. 15B), and anti-H1 stem (FIG. 15C), as well as immune sera from the mice infected with A/Viet/2004 H5N1 virus (FIG. 15D).

FIGS. 16A-16F show dosage effects of adjuvanted M2e-H1stem vaccine on inducing IgG antibodies and conferring protection. Different doses (5 μg, 10 μg, and 20 μg) of M2e-H1stem vaccine with adjuvants (QS-21+MPL) were used to prime-boost immunize 6-week-old BALB/c mice (n=3). The levels of vaccine antigen-specific IgG antibodies in immune sera were determined by ELISA and protection efficacy was assessed after lethal challenge with influenza A virus. FIGS. 16A and 16C show M2e- and M2e-H1 stem-specific IgG antibodies in prime vaccinated sera. FIGS. 16B and 16D show the levels of M2e- and M2e-H1 stem-specific IgG antibodies in boost vaccinated sera. Mock: adjuvant only naïve sera; mock inf: mock group with virus infection. (FIGS. 16E-16F) Body weight changes and survival rates in vaccinated mice with adjuvanted M2e-H1 stem and challenged with rgH7N9 (A/Sha/2013, (3×LD₅₀, 1.1×10⁴ EID₅₀). The statistical significance was determined by using two-way ANOVA; error bars indicate mean±SEM; *, P<0.05; **, P<0.01; ***, P<0.001.

FIGS. 17A-17F show rational design of chimeric M2e-H3 stalk protein, purification, and confirmation. FIG. 17A is a schematic of full-length HA gene of influenza A virus (A/Aichi/H3N2), and the selective domains as a vaccine target are numbered in amino acid (aa 37-61, 305-338, 1-117) residues. FIG. 17B shows M2e-H3 stalk vaccine construct with flexible and soluble linker sequences (AAAGGAA (SEQ ID NO:39); GGGGS (SEQ ID NO:40); GSA; GSAGSA; QGTGG (SEQ ID NO:42)). FIG. 17C shows the monomeric H3 HA 3D cartoon structure as predicted by the SWISS model and visualized in PyMol. FIG. 17D is an illustration of monomeric cartoon structure of M2e-H3 stalk domain marking the positions of point mutations. M2e and foldon structures were modeled using PDB ID codes 4N8C and IRFO respectively. FIG. 17E shows Coomassie Blue staining of M2e-H3 stalk protein. Marker: protein size marker (kDa), Crude TP: Total cell lysates (25 μg); M2e H3 stalk: purified M2e-H3 stalk protein (15 μg). (FIG. 17F) Western blot of M2e-H3 stalk protein. 14C2: M2e-specific mAb; stalk: anti-fusion peptide (FP) polyclonal antibody (pAb) recognizing HA2 aal-14 epitope.

FIGS. 18A-18F show characterization of M2e-H3 stalk protein antigenicity, stability, and its cross-reactivity. FIGS. 18A, 18E-18F show the antigenicity of purified M2e-H3 stalk protein was determined by standard ELISA using antibodies specific for M2e (14C2), HA2 domain (rabbit poly IgO Abs purified against HA2 epitopes including aal-13 (poly HA2 #1-13) or aa14-27 (poly HA2 #14-27), and polyclonal antibodies (pAbs) against recombinant HA proteins from different subtypes (H1N1, H5NI, H3N2, H7N9); antisera of mice infected with influenza A live viruses (rgH5NI, H3N2, rgH7N9) were used. FIGS. 18B-18D show thermostability of M2e-H3 stalk protein was evaluated after storage at different temperatures (4, 20, 37, 50° C.) for 11 days by determination of retaining antigenicity. FIG. 18A shows antigen reactivity specific to conserved HA2 FP and M2e antibodies. FIG. 18B shows thermostable M2e-H3 stalk protein reactivity to 14C2 mAb. FIG. 18C shows thermostable M2e-H3 stalk protein reactivity to FP pAb. FIG. 18D shows thermostable M2e-H3 stalk protein reactivity to H5NI virus antisera. FIG. 18E shows M2e-H3 stalk protein reactivity to pAbs against HA proteins from group 1 (G1) viruses (H1N1, H5NI) and group 2 (G2) viruses (H3N2, H7N9). FIG. 18F shows M2e-H3 stalk protein reactivity against the antisera of live G1 and G2 influenza A viruses. Ctrl: BSA control. Mock: Naive mice sera. Statistical significance was determined by using one-way ANOVA followed by Tukey's Multiple Comparison Test or two-way ANO VA followed by Bonferroni post-test; error bars indicate mean±SEM; *, P<0.05; **, P<0.01; ***, P<0.001.

FIGS. 19A-19J show adjuvanted M2e-H3 stalk vaccination induces antibodies recognizing M2e, FP, stalk, and group 1 and 2 viral antigens and virus-infected cell surface. BALB/c mice (n=10) were intramuscular (i.m.) prime-boost immunized with adjuvanted M2e-H3 stalk (20 μg) protein. IgG antibodies and ADCC were determined in sera collected 2 weeks after vaccination. FIGS. 19A-19C show IgG and IgG antibody subtypes specific for M2e-H3 Stalk protein. IgG (FIG. 19A), IgG I (FIG. 19B) and IgG2a (FIG. 18C) antibodies specific for M2e-H3 stalk protein. (FIGS. 19D-19F) stalk specific IgG antibodies in boost sera. IgG antibodies specific for HA2 FP epitope region aa14-27 of HA2 (FIG. 19D), stalk epitope residues aa74-98 of HA2 (FIG. 19E), and full-length stalk protein (aa 1-185 of HA2) (FIG. 19F). FIGS. 19G-19H shows M2e-H3 stalk vaccination induced IgG antibodies recognizing both group 2 (FIG. 19G) and group 1 (FIG. 19H) viral antigens on the surface of virus-infected MDCK cells. FIGS. 19I-19J show antibodies induced by M2e-H3 stalk vaccine engage in Fe-mediated activation of Jurkat effector cells, mimicking a surrogate ADCC activation pathway. Group 2 virus (G2) A/Phil/H3N2(H) and group 1 virus (G1) NWSN/H1N1 (FIG. 19J). Mock: adjuvanted naive sera, M2e-depleted: M2e specific IgG depleted M2e-H3 stalk sera. Statistical significance was determined by using two-way ANOVA; error bars indicate mean±SEM; *, P<0.05; **, P<0.01; ***, P<0.001.

FIGS. 20A-20I show adjuvanted M2e-H3 stalk vaccination induces heterologous cross-protection against group 2 influenza A viruses. FIG. 20A shows M2e-specific serum IgG antibody levels in the different vaccine groups. FIG. 20B shows serum IgG antibodies specific for M2e-H3 stalk antigen in the M2e-H3 stalk groups with (M2e-H3 stalk) and without adjuvant (M2e-H3 stalk/No adj). FIGS. 20C-20H show the groups of mice (n=5, 6-8 weeks old) vaccinated (prime or prime-boost) with M2e-H3 stalk protein (20 μg+/−adjuvant) or M2e-RBD (20 μg plus adjuvant) were intranasally challenged with group 2 influenza A viruses (H3N2, H7N9). Body weight changes and survival rates were daily monitored. FIGS. 20C-20D show A/Phil/1982 H3N2 (3×LD₅₀, 2.3×10² EID₅₀), (FIG. 20E) A/Nanchang/1995 H3N2 (2×LD₅₀, 3×10⁶ EID₅₀), (FIG. 20F) A/Sha/2013 H7N9 (3×LD₅₀, 1.1×10⁴ EID₅₀), (FIG. 20G) A/HK/1968 H3N2 (3×LD₅₀, 4×10¹ EID₅₀). FIG. 20H shows efficacy of thermostable M2e-H3 stalk protein. Mice vaccinated with M2e-H3 stalk protein pre-incubated at 50° C. for 11 days prior to prime-boost vaccination were challenged with A/Nanchang/1995 H3N2 (2×LD₅₀, 3×10⁶ EID₅₀). FIG. 20I shows efficacy of single dose M2e-H3 stalk vaccination. Mice with M2e-H3 stalk prime dose only were challenged with A/Nanchang/1995 H3N2 (2×LD₅₀, 3×10⁶ EID₅₀) at 4 weeks after vaccination. Mock inf: mock group (adjuvant only) with virus infection, No Adj: M2e-H3 stalk vaccinated group without adjuvant. Statistical significance was determined using the two-way AN OVA followed by Bonferroni post-test. Error bars indicate means±SEM; * P<0.05; **, P<0.01; ***, P<0.001.

FIGS. 21A-21F show adjuvanted M2e-H3 stalk protein provides protection against heterologous cross-group 1 influenza A viruses. The groups of mice (n=5, 6-8 weeks old) vaccinated with adjuvanted M2e-H3 stalk (prime and prime-boost for WSN/H1N1 virus challenge group or prime-boost for the rest of virus challenge) were intranasally challenged with group I influenza A viruses (H1N1, H5N1, H9N2). Body weight changes and survival rates were monitored for 14 days. FIGS. 21A to 21F show A/WSN/1933 HINI (2×LD₅₀, 1.5×10² EID₅₀) (FIG. 21A), AIPR8/1934 H1N1 (4×LD₅₀, 1.2×10³ EID₅₀) (FIG. 21B), A/Viet/2004 rgH5N1 (3×LD₅₀, 2.6×10⁴ EID₅₀) (FIG. 21C), A/HK/1999 H9N2 (4×LD₅₀, 7.8×10¹ EID₅₀) (FIG. 21D), A/Cal/2009 HIN1(3×LD₅₀, 2×10³ EID₅₀) (FIG. 21E), and A/FM/1947 H1N1 (3×LD₅₀, 8×10³ EID₅₀) (FIG. 21F). Mock inf: mock group (adjuvant only) with virus infection. Statistical significance was determined using the two-way ANOVA followed by Bonferroni post-test. Error bars indicate means±SEM; *, P<0.05; **, P<0.01; ***, P<0.001.

FIGS. 22A-22I show adjuvanted M2e-H3 stalk vaccination bestows cross protection by lowering lung viral loads and inducing protective humoral immune responses. FIG. 22A shows body weight changes in M2e-H3 stalk (20 μg) vaccinated young BALB/c mice (n=5) for 6 days after challenge with lethal dose (2×LD₅₀, 3×10⁶ EID₅₀) of A/Nanchang/H3N2 virus. FIG. 22B shows lung viral titers at 6 days post-infection. FIGS. 22C-22G show in vitro production of IgG antibodies specific for M2e (FIG. 22C) and stalk (FIG. 22D) in mediastinal lymph node (MLN) and spleen cultures IgG antibodies specific for M2e (FIG. 22E), stalk (FIG. 22F) or M2e-H3 stalk (FIG. 22G), determined by ELISA. FIG. 22H-22I show protective efficacy of vaccine immune sera. Body weight changes in naive mice after intranasal inoculation with a mixture of M2e-H3 stalk vaccine immune sera or naive sera and group 1 influenza A virus (A/WSN H1N1, 4.2×LD₅₀) (FIG. 22H) and group 2 influenza A virus (A/rgH7N9, 5×LD₅₀) (FIG. 22I). Mock: mock group (adjuvant only no vaccine group (FIGS. 22A-22G)) with virus infection. Naive: mice group with no immunization and no virus infection. Mock: adjuvanted naive sera (FIGS. 22H-22I). Statistical significance was determined by using one-way ANOV A followed by Tukey's Multiple Comparison Test or two-way ANOVA followed by Bonferroni post-test; error bars indicate mean±SEM; * P<0.05; **, P<0.01; ***, P<0.001.

FIGS. 23A-23I show adjuvanted M2e-H3 stalk vaccine induces protective T cell immunity. Cytokine-secreting T cell immune responses were evaluated in M2e-H3 stalk immunized mice at 6 days-post-infection with A/Nanchang H3N2 vims. FIGS. 23A-23D show IFN-γ+ secreting ELISpot assays of spleen cells after in vitro antigen stimulation with M2e- (FIG. 23A) or stalk (FIG. 23B), and in Lung cells stimulated with M2e (FIG. 23C) or Stalk (FIG. 23D). FIGS. 23E-23H show IFN-·t CD4+ or CD8+ T cells responses in BALF and Lung cells were determined by intracellular cytokine staining and flow cytometry analysis. IFN-γ+ CD4+ T cells response in BALF (FIG. 23E) and Lung (FIG. 23F). IFN-γ+ CD8+ T cells response in BALF (FIG. 23G) and Lung (FIG. 23H). Mock: mock group (adjuvant only) with virus infection. Naive: mice group with no immunization and no virus infection. Impact of CD4+ and CD8+ T cell depletion on protection in M2e-H3 stalk vaccinated mice before challenging with A/rgH7N9 virus (FIG. 23I). Statistical significance was determined by using one-way ANOV A followed by Tukey's Multiple Comparison Test or two-way ANOV A followed by Bonferroni post-test; error bars indicate mean±SEM; *, P<0.05; **, P<0.01; ***, P<0.001.

FIGS. 24A-24J show aged mice with adjuvanted M2e-H3 stalk vaccination induces cross-group virus protection. Aged mice (16 months old, n=5) were i.m. prime-boost immunized with adjuvanted M2e-H3 stalk protein. FIGS. 24A-24F show M2e-H3 stalk vaccination of aged mice induced IgG, IgG1 and IgG2a antibodies against M2e and stalk protein. IgG to M2e (FIG. 24A), IgG1 to M2e (FIG. 24B), IgG2a to M2e (FIG. 24C). IgG to stalk (FIG. 24D), IgG1 to stalk (FIG. 24E), IgG 2a to stalk (FIG>24F). FIGS. 24G-24J show efficacy of cross-group virus protection in aged mice as measured by body weight changes and survival rates. Aged mice (n=5) at 3 weeks after boost vaccination were intranasally challenged with a lethal dose of either group 1 (A/WSN H1N1, A/Cal H1N1) or group 2 (A/Phil H3N2, A/Sha/H7N9) influenza A viruses. A/Phil H3N2 (FIG. 24G), A/Sha/H7N9 (FIG. 24H), A/WSN H1N1 (FIG. 24I), A/Cal H1N1 (FIG. 24J). Statistical significance was determined using the two-way ANOV A followed by Bonferroni post-test. Error bars indicate means±SEM; *, P<0.05; **, P<0.01; ***, P<0.001.

FIGS. 25A-25E show adjuvanted M2e-H3 stalk vaccine dosage effects and protective advantages over M2e only vaccines. Young adult mice (6-8 weeks old, n=5) were vaccinated with 5xM2e-VLP (10 μg) or M2e-RBD (20 μg) or M2-H3 stalk protein at different doses (5 μg, 10 μg, 20 μg). FIG. 25A shows IgG antibodies specific for A/Sha H7N9 virus. FIG. 25B shows IgG antibodies specific for A/HK H3N2 virus. FIG. 25C shows vaccine dosage effects of M2e-H3 stalk on body weight changes and survival rates after A/Sha/2013 H7N9 ((3×LD₅₀, 1.1×10⁴ EID₅₀) virus challenge. FIG. 25D shows protective efficacy of M2e only vaccines after A/Sha/2013 H7N9 (3×LDso, 1.1×10⁴ EID₅₀) virus challenge. FIG. 25E shows protective efficacy comparison of M2e-H3 stalk and M2e only vaccines after A/HK/1968 H3N2 (12×LD₅₀, 1.4×10² EID₅₀) virus challenge. Statistical significance was determined using the two-way ANOVA followed by Bonfenoni post-test. Error bars indicate means±SEM; *, P<0.05; **, P<0.01; ***, P <0.001.

FIGS. 26A-26G show adjuvant and M2e-H3 stalk vaccine dosage effects on inducing IgG antibodies specific for vaccine antigen and M2e. The levels of IgG, IgG I and IgG2a antibodies specific for M2e-H3 stalk vaccine antigen (FIGS. 26A-26C) or M2e (FIGS. 26D-26G) were measured from prime or boost immune sera collected at two weeks after prime and boost vaccination of young adult mice (n=10) with M2e-H3 stalk protein (5, 10, 20 μg) with adjuvant (QS-21+MPL) or M2e-H3 stalk protein (20 μg) without adjuvant. Mock: adjuvant only naive sera, No Adj: M2e-H3 stalk vaccinated group without adjuvants. Statistical significance was determined using the two-way ANOV A followed by Bonferroni post-test. Error bars indicate means±SEM, *, P<0.05; **, P<0.01; ***, P<0.00 1.

FIGS. 27A-27D show adjuvanted M2e-H3 stalk vaccination induces IgG antibodies recognizing group 1 and group 2 viruses. Boost sera of M2e-H3 stalk vaccination were used to determine IgG antibodies specific for group 1 viruses AIHK/H9N2 (FIG. 27A) A/Cal/H1N1 (FIG. 27B) and group 2 viruses, A/Hong Kong (HK)/H3N2 (FIG. 27C), A/Phil/H3 2 (FIG. 27D). Statistical significance was determined using the two-way ANOVA followed by Bonferroni post-test. Error bars indicate means±SEM, * P<0.05; **, P<0.01; *** P<0.001.

FIGS. 28A-28C show aged mice with adjuvanted M2e-H3 talk vaccination induce high levels of IgG antibodies specific for vaccine antigen. Aged BALB/c mice (16 months old) were i.rn. prime-boost vaccinated with adjuvanted M2e-H3 stalk and vaccine antigen specific IgG and isotype antibody level were analyzed in prime and boost sera. IgG (FIG. 28A) IgG1 (FIG. 28B) and IgG 2a (FIG. 28C) antibody responses to M2e-H3 stalk protein. Statistical significance was determined between prime and boost groups using the two-way ANOVA. Error bars indicate means±SEM *, P<0.05; ** P<0.01 *** P<0.001.

FIGS. 29A-29D show characterization of M2e-RBD (M2e only) vaccine. FIG. 29A is a schematic diagram of M2e-RBD (receptor binding domain of SARS-CoV-2 spike protein) vaccine construct. FIG. 29B shows SDS-PAGE gel analysis of the purified M2e-RBD protein. FIG. 29C shows M2e-RBD antigenic reactivity to M2e specific mAb (14C2). FIG. 29D shows IgG antibody of M2e-RBD reactivity to M2e epitope. Statistical significance was determined between prime and boost groups using the two-way ANOVA followed by Bonferroni post-test. Error bars indicate means: SEM, *, P<0.05; **, P<0.01; *** P<0.001.

FIGS. 30A-30F show adjuvanted M2e-H3 stalk vaccination induced higher levels of IgG antibodies binding to viral antigens on the influenza A virus infected MDCK cell surface than M2e only vaccines. Boost antisera of M2e-H3 stalk and M2e only vaccination was compared in the levels of IgG antibodies specific for 5xM2e repeat protein and virus infected cell surface viral antigens. FIG. 30A shows IgG antibodies for 5xM2e. FIG. 30B shows A/Nanchang H3N2 virus infected MDCK cell surface ELISA. (FIG. 30C) A/Hong Kong H3 2 virus infected MDCK cell surface ELISA. FIG. 30D shows M2e specific IgG quantification between the groups from panel A. FIG. 30E shows A/WS H1N1 virus infected MDCK cell surface ELISA. FIG. 30F shows A/HK 119N2 virus infected MDCK cell surface ELISA. Statistical significance was determined using the one or two-way ANOVA. Error bars indicate means±SEM *, P<0.05; ** P<0.01·***, P<0.001.

FIGS. 31A-31B show adjuvanted M2e-H3 stalk vaccination induces the generation of stalk-specific IgG antibody-secreting plasma cell but not M2e-RBD. Mediastinal lymph nodes (MLN) were collected at 6 days-post-infection with A/Nanchang H3N2 virus from adjuvanted M2e-RBD (20 μg) or M2e-H3 stalk (20 μg) vaccinated young adult mice. In vitro production of IgG antibodies specific for M2e (FIG. 31A) and stalk (FIG. 31B) was assessed in MLN cultures. Mock: adjuvant only naive sera; mock inf: mock group with virus infection. Statistical significance was determined using the one-way ANOVA. Error bars indicate means±SEM * P<0.05; ** P<0.01·*** P<0.001.

FIG. 32 shows analysis result of amino acid sequence homology among M2e-H3 stalk and its corresponding domain of group 1 and 2 challenge viruses. The HA2 domain sequences of the challenging viruses used in this study were obtained from GenBank to analyze the identity of amino acid (aa) sequences: GenBank accession: NC_002017 HI HA (YP_163736) for A/Puerto Rico/8/1934 (HI 1/PR8)·CY010788 HI HA (ABF47955) for A/WS/1933 (H1N1/WSN); IVU02464 H1 HA (AAC53844) for Fort Monmouth/1/1947 (H1N1/FM); C_026433A H1 HA (YP_009118626) for A/California/07/2009 (H1N1/Cal)·EU122404 H5 HA (ABW90135) for Vietnam/1203/2004 (H5N1Net); KF188366 H9 HA (AGO17823) for A/chicken/Hong Kong/G9/1997 (H9M2/HK); IVU08858 H3 HA (AAA18781) for A/Philippines/2/82 (H3N2/Phil) KC853228 H7 HA (AG160292) for A/Shanghai/2013 (H7N9/Sh); CY080523 H3 HA (ADV76673) for A/Hong Kong/1-10-MA21-1/1968 (H3N2/HK); KM821307 H3 HA (ATU46048) for Nanchang/933/1995 (H3N2/Nanchang). The percentage of the aa homology of the vaccine construct M2e-H3 talk HA1 and HA2 domains with HA sequence of challenge viruses were calculated using Needle program (EMBOSS; EMBL-EBI).

FIG. 33 shows ADCC reporter assay activity on H9N2 virus infected MOCK cells. The boost era of adjuvanted M2e-H3 stalk were used to determine the ADCC reporter as ay activity using A/HK/H9N2 infected MDCK cells. Statistical significance was determined using the two-way ANOVA followed by Bonferroni post-test. Error bars indicate means±SEM * P<0.05·**, P<0.01·*** P<0.001.

FIGS. 34A-34E show adjuvanted M2e-H3 stalk vaccinated mice survived 100% against group 2 influenza A virus infection. The survival rates of adjuvanted M2e-H3 stalk group were determined against group 2 influenza A virus infection from FIGS. 20C-20H.

FIGS. 35A-35B show flow cytometry gating strategy to identify CD4 and CDS T cells secreting IFN-γ. The effector T cells were analyzed in BALF (FIG. 35A) and lung cells (FIG. 35B) obtained from mice after intracellular cytokine staining and acquisition by flow cytometry. G1: Lymphocyte gated from total cell G2: CD3+ T cells gated from the gated lymphocytes (G1) G3 and G4: CD4+ CD3+ T cells or CD8+ CD3+ T cell gated from CD3+ T cells (G2) respectively G5: IFN-γ+ CD4+ T cells gated from CD4+ CD3+ T cells (G3) G6: IFN-γ+ CD8+ T cell gated from CD8+ CD3+ T cells (G4).

FIGS. 36A-36J show design and characterization of m-cNA-M2e VLPs containing consensus multi-subtype cNA and tandem repeat 5xM2e. FIG. 36A is a scheme diagram of multi-component m-cNA-M2e VLP vaccine expressing M1, multi subtype cNA (cN1, cN2, and B-cNA), and 5xM2e genes under each polyhedrin promoter. FIG. 36B shows PCR analysis for confirmation of five genes cloned into the rBV transfer plasmid pFastBac using gene specific primers. FIGS. 36C and 36D show size distribution of m-cNA-M2e, mono cNA, and 5xM2e VLP. d.nm: diameter. FIGS. 36E and 36F show Western blot analysis of m-cNA-M2e and mono cNA VLPs using HCA2 mAb specific for pan NA and 14C2 mAb specific for M2e. M: size marker; kDa: kilodalton. FIG. 36E shows VLPs in each line loaded with 20-30 μg. lane 1: M1 VLP (left, right), lane 2: m-cNA-M2e VLP (left, right), lane 3: 5xM2e VLP. FIG. 36F show VLP and protein samples loaded with 30 μg and 5 μg respectively. cN1: consensus NA1 VLP, N1: NA protein from A/California/04/2009 H1 N1 (BEi, NR-19234), cN2: consensus NA2 VLP, N2: NA protein from A/Brisbane/10/2007 H3N2 (BEi, NR-43784), B-cN: influenza B consensus NA VLP. FIGS. 36G and 36H show the reactivity of m-cNA-M2e VLP, mono cNA VLPs and 5xM2e VLP to HCA2 (FIG. 36G) or 14C2 (FIG. 36H) mAbs by ELISA. FIG. 36I shows functional NA activity of m-cNA-M2e VLP and mono cNA VLPs by ELLA. FIG. 36J shows schematic overview of vaccination and bleed schedule of mice, prior to influenza virus challenge. BALB/c mice (n=10 per group) were intramuscularly immunized twice with each VLP vaccine as indicated, with a 3-week interval. Boost sera (W5) used for measuring immunogenicity, NAI, and ADCC assay, followed by intranasal (IN) influenza virus challenge at W7 and tissue samples were collected at day 5 or 6 post infection for lung viral titters and analysis. Infected mice were monitored daily for weight loss and survival in the 2 weeks following challenge.

FIGS. 37A-37I show vaccination with m-cNA-M2e VLP induces IgG antibodies specific for M2e and multi subtype NA as well as broad NA inhibition activity. BALB/c mice (n=8 per group) were intramuscularly immunized twice with each VLP vaccine as indicated, with a 3-week interval as shown in FIG. 36J. cN1: monomeric consensus cN1 NA VLP (3 μg), cN2: monomeric consensus cN2 NA VLP (3 μg), 5xM2e: monomeric 5xM2e VLP (3 μg), m-cNA-M2e VLP: multi-subtype consensus NA (cN1-cN2-B cNA) plus 5xM2e VLP (10 μg), B cNA: consensus influenza B NA VLP (3 μg). Na'ive mice (PBS) were used as a mock control. (A-E and H) Antibody response specific for M2e (FIG. 37A), N1 (A/Cal/2009 H1 N1) (FIG. 37B), N2 (A/Brisbane/2007 H3N2) (FIG. 37C), inactivated influenza viruses (FIGS. 37D and 37E), and flu B NA protein (B/Florida/4/2006, Yamagata) (FIG. 37H) were determined in boost immune sera by ELISA. (FIGS. 37F-37G and 37I) Neuraminidase (NA) inhibition activity. NA inhibition activity were measured from 40-fold diluted boost immune sera by ELLA. rgA/PR8-Swz (rgA/H1N2): reassortant containing N2 of A/Switzerland/2013 (H3N2) and A/PR8 (H1N1) backbone. All viruses are as described in Materials and Methods. Data represented as mean±SEM; statistical significances were performed by two-way ANOVA with Bonferroni posttest and indicated as**, P <0.01; ***, P<0.001.

FIGS. 38A-38H show broad cross protection against N2 and N9 NA influenza A viruses after m-cNA-M2e VLP vaccination. Immunized mice (n=6 per group) were infected with lethal dose of N2 (A/Phil H3N2, rgA/H1N2, rgA/HK H9N2) (FIGS. 38A-38C) or N9 (rgA/SH H7N9) (FIG. 38D), and N1 (rgA/VNH5N1, A/Cal H1N1) (FIGS. 38E and 38F) influenza A, as well as influenza B (B/Florida, B/Malaysia) viruses (FIGS. 38G and 38H). (FIG. 38A) A/Phil/1982 (H3N2) (3×LD₅₀, 2.3×10² EID₅₀), (FIG. 38B) rgA/H1N2 (3×LD₅₀, 1.3×103 EID₅₀), (FIG. 38C) rgA/HK/1999 (H9N2) (5×LD₅₀, 1.4×10² EID₅₀), (FIG. 38D) rgA/SH/2013 (H7N9) (3×LD₅₀, 1.1×10⁴ EID₅₀), (FIG. 38E) rgA/VN/2004 H5N1 (3×LD₅₀, 2.6×10⁴ EID₅₀), (FIG. 38F) A/Cal/2009 H1N1 (3×LD₅₀, 2×10³ EIDso). (FIGS. 38G and 38H) BALB/c mice (n=10 per group) were intramuscularly prime-boost immunized with m-cNA-M2e VLP (10 μg) or B-cNA VLP (3 μg), were infected with B/Florida/4/2006 (2×LD₅₀) (FIG. 38G) and B/Malaysia/2056/2004 (3×LD₅₀) (FIG. 38H). Morbidity and mortality were monitored daily for 14 days post infection. The VLP vaccine dose and groups are the same as in the FIG. 37 legend except for the cN2 (cN2 VLP) group in FIG. 38B, which received 10 μg. The statistical significances were performed with one-way ANOVA with Tukey's Multiple Comparison test and indicated as *, #P<0.05; **, ##, P<0.01; P<0.001; ns, no significant difference between two compared groups.

FIGS. 39A-39J show CD4+ and CD8+ T cells responses and diminished lung viral loads were elicited by m-cNA-M2e VLP vaccination. Immunized mice (n=4 per group) were challenged with a lethal dose of rgA/VN/2004 H5N1 (3×LD₅₀, 2.6×10⁴ EID₅₀) (A-D) and A/Phil/1982 (H3N2) virus (3×LD₅₀, 2.3×10² EID₅₀) (E-G). (A-G) Effector CD4 or CD8 T cells secreting IFN-γ were analyzed in lung cells harvested on day 6 post infection after in vitro stimulation with 5 μg/ml of M2e, N1 (A/Vietnam/1203/2004 H5N1) or N2 (A/Brisbane/10/2007) NA pooled peptides. (A and B) IFN-γ+ CD4+ T cells specific for M2e peptide (upper panel or red bar) or N1 NA peptide pools (lower panel). (C and D) IFN-γ+ CD8+ T cells to M2e or N1 NA peptide pools. Following A/Phil/19S2 (H3N2) challenge, (E and F) IFN-γ+ CD4+ T cells specific for M2e (upper panel) or N2 NA (A/Brisbane/10/2007) peptide pools (lower panel). (G) IFN-γ + CD8+ T cells to M2e (red bar) or N2 NA (green bar) peptide pools. (H-J) Lung viral titers at day 6 post lethal dose infection with rgA/VN/2004 H5N1 (H), A/Phil (H3N2) (1), and rgA/SH (H7N9) (J) by an egg inoculation assay in 10-day embryonated chicken eggs. EID₅₀: 50% egg infectious dose. The statistical significances were performed with one-way ANOVA with Tukey's Multiple Comparison test and indicated as*, P<0.05; **, P<0.01; ***, P<0.001; ns, no significant difference between two compared groups.

FIGS. 40A-40K show m-cNA-M2e VLP vaccination induces humoral and cellular immune responses and confers cross protection against influenza A viruses in aged mice. BALB/c mice (14-months old, n=10 per group) were intramuscularly immunized with m-cNA-M2e VLP (10 μg) prime-boost at a 3-week interval. (A-C) ELISA IgG and isotype antibodies specific for M2e, N2 NA (A/Brisbane/10/2007 H3N2), and N1 NA (A/California/4/2009 H1 N1) protein in boost immune sera from aged mice. (D-F) Body weight changes and survival rates were monitored daily for 14 days after challenge with influenza A viruses. (D) A/Phil/1982 H3N2 (3×LD₅₀, 7×10 EID₅₀), (E) A/Cal/2009 H1 N1 (3×LD₅₀, 2×10⁴ EID₅₀), (F) rgA/SH/2013 H7N9 (H7 HA, N9 NA from A/Shanghai/2013) (3×LD₅₀, 5.6×10³ EID₅₀). (G) NA inhibition activity in 40-fold diluted boost immune sera against A/Phil (H3N2) virus by ELLA. (H) Lung viral titers at day 6 post infection with A/Phil (H3N2). (1) Antigen-specific IgG levels in mLN cells collected at day 6 post challenge with A/Phil (H3N2) after in vitro culture with M2e (4 μg/ml) or N2 NA protein (A/Brisbane/10/2007 H3N2, 200 ng/ml) for 1 (D1) or 5 (D5) days. (J) IFN-γ cytokine secreting spots in lung cells after stimulation with 5 μg/ml of M2e or N2 NA (A/Brisbane/10/2007, H3N2) pooled peptide. (K) IFN-γ secreting CD4 T cells in lungs after in vitro stimulation with 5 μg/ml of M2e or N2 NA pooled peptide by intracellular cytokine staining and flow cytometry. The statistical significances were performed with one-way ANOVA with Tukey's Multiple comparison test or two-way ANOVA with Bonferroni posttest and indicated as*, P<0.05; **, P<0.01; ***, P <0.001.

FIGS. 41A-41H show ADCC function and T cell response contribute to protection by m-cNA-M2e VLP-vaccination. (A-C) MDCKs were infected with 100xTCID₅₀ influenza A viruses (A/Nanchang/933/1995 H3N2, A/California/04/2009 H1N1, and rgA/Vietnam/1203/2004 H5N1) in DMEM with 1 μg/ml TPCK-treated trypsin for 24 hours. The cells were fixed with 10% natural buffered formalin prior to adding diluted immune sera pre-inactivated (56° C., 30 min). The binding reactivities to virus antigens expressed on MDCKs were determined by ELISA. Binding reactivity of immune sera to H3N2-(A), H1N1-(B), H5N1-(C) infected MDCKs. (D-F) ADCC reporter assays of antisera from immunized mice against to target MOCK cells infected with A/Nanchang/933/1995 (H3N2) (D), A/California/04/2009 (H1N1) (E), and rgA/VN/1203/2004 H5N1 (F). Subsequently, the ADCC reporter assay was performed using Jurkat effector cells expressing mouse FcrRIII, and the relative luminescence unit (RLU) was measured. (G) Roles of immune sera from each vaccine group in conferring protection in na·1ve mice. No immunized naive mice (n=3 per group) were intranasally infected with a mixture of influenza virus A/Phil H3N2 (4×LD₅₀, 3.7×102 EID₅₀) and immune sera from the m-cNA-M2e VLP, 5xM2e VLP, cN2 VLP, or naive control (PBS) groups. Survival rates were monitored daily for 14 days. (H) Contribution of T cells to induce protection in vaccinated mice (n=4 per group) with m-cNA-M2e VLP. CD4 T cells and CD8 T cells were depleted by intraperitoneal injection with anti-CD4 (clone GK1.5) or anti-CD8 (clone 53.6.7) twice, prior to and after infection with A/Phil H3N2 virus (4×LD₅₀, 3.7×10² EID₅₀). Survival rates were monitored daily for 14 days. The statistical significances were performed with two-way ANOVA with Bonferroni posttest and indicated as **, P<0.01; ***, ###, P<0.001; ns, no significant difference between two compared groups.

FIGS. 42A-42B show Neuraminidase (NA) inhibition function by immune sera. NA inhibition activity were measured from serially diluted boost immune sera by ELLA.

FIGS. 43A-43C show Cross protection against N2 and N1 NA influenza A viruses in mice of m-cNA-M2e VLP vaccination. Immunized mice (n=6 per group) were infected with lethal dose of N2 [A/NC (NNanchang/1995, 2×LD₅₀, 3×106 EID₅₀) (A), NHK H3N2 (3×LD₅₀, 4×10 EID₅₀) (B)] and N1 [NFM/1947 H1N1 (3×LD₅₀, 8×103 EID₅₀)](C). Morbidity and mortality were monitored daily for 14 days post infection. The statistical significances were performed with one-way ANOVA with Tukey's Multiple Comparison test and indicated as**, P<0.01; ***, P<0.001.

FIGS. 44A-44D show Protective cellular and humoral immunity in mice of m-cNA-M2e VLP vaccination against N2 NA influenza A virus. Immunized mice (n=4 per group) were infected with lethal dose of A/Phil/1982 (H3N2) virus. (A and B) IFN-γ-secreting cell spots. Splenocytes and lung cells were cultured on the ELISpot plate pre-coated with cytokine capture antibody in the presence of 5 μg/ml of M2e or N2 NA (A/Brisbane/10/2007 H3N2) peptide pools. (C and D) Antigen-specific IgG antibodies were determined from mediastinal lymph node (mLN) and spleen harvested on day 6 post infection and subsequent in vitro culture for 5 days (D5) on the plate precoated with 2 μg/mL of M2e peptide or 200 ng/ml of N2 NA protein (A/Brisbane/10/2007 H3N2). The statistical significances were performed with one-way ANOVA with Tukey's Multiple Comparison test and indicated as*, P<0.05; **, P<0.01; ***, P<0.001 between indicated groups.

FIGS. 45A-45H show Enhanced humoral immunity and reduced lung inflammation by m-cNA-M2e vaccinated young and aged mice upon influenza A virus infection. (A-D) Immunized young mice (6-8-week-old, n=4 per group) were infected with a lethal dose of A/Phil H3N2 virus. (A and B) IgG levels specific for M2e peptide or NA2 protein (A/Brisbane/10/2007 H3N2) were determined in the bronchoalveolar lavage fluid (BALF) and lung lysates harvested on day 6 post infection by ELISA. (C and D) The levels of IFN-γ and IL-6 in BALF and lungs by ELISA. (E-H) Immunized aged BALB/c mice (14-month-old, n=4 per group) were infected with a lethal dose of A/Phil H3N2 virus. (E and F) IgG levels specific for M2e peptide or N2 NA protein (A/Brisbane/10/2007 H3N2) were determined in the BALF and lung lysates harvested on day 6 post infection by ELISA. (G and H) The levels of IFNY and IL-6 in BALF and lungs of aged BALB/c mice. Data represented as mean±SEM; statistical significances were performed by one-way ANOVA with Tukey's multiple comparison test and indicated as*, P<0.05; **, P<0.01; ***, P<0.001; ns, no significant difference between compared groups.

FIGS. 46A-46B show The roles of immune sera and T cell responses on conferring protection in naive mice. (A) No immunized naive mice (n=3 per group) were intranasally infected with a mixture of influenza virus A/Phil H3N2 (4×LD₅₀, 3.7×10² EID₅₀) and immune sera from the mcNA-M2e VLP, 5xM2e VLP, cN2 VLP, or naive control groups. Weight changes were daily monitored for 14 days. (B) Contribution of T cells to induce protection in vaccinated mice (n=4 per group) with m-cNA-M2e VLP. CD4 T cells and CD8 T cells were depleted by intraperitoneal injection with anti-CD4 (clone GK1.5) or anti-CD8 (clone 53.6.7) twice, prior to and after infection with A/Phil H3N2 virus (4×LD₅₀, 3.7×10² EID₅₀). Weight changes were monitored daily for 14 days. The statistical significances were performed with two-way ANOVA with Bonferroni posttest and indicated as*, #, +, P<0.05; **. ##, P<0.01; ***, ###, +++, P<0.001.

FIGS. 47A-47C show Sequence homology between the consensus NA vaccines and influenza viruses used for challenge. The sequence similarity was identified using basic local alignment search tool (BLAST) with protein BLAST. (A) Sequence homology between the consensus N1 (cN1) and N2 (cN2) NA vaccines and influenza viruses containing N2 NA. (B) Sequence homology between the consensus N1 (cN1) and N2 (cN2) NA vaccines and influenza viruses containing N1 or N9 NA. (C) Sequence homology between the consensus influenza B NA (B cNA) vaccine and influenza B viruses.

FIG. 48 is a schematic diagram for universal flu mRNA and HA mRNA vaccine constructs. m1Ψ: N1-methylpseudouridine. See the detail descriptions (Cap, UTR, poly-A, SP, tPA SP, GCN4, TM) in the text. Cap: 5′Capping of mRNA, UTR: untranslated regions, poly A: poly A addition, tPA: Tissue plasminogen activator, SP: signal peptide, Foldon: tetramer stabilizing domain, TM: transmembrane domain.

FIG. 49 shows expression of mRNA constructs by fluorescent microscope and confocal microscope of cells after mRNA transfection. H1 HA, 5xM2e, influenza B NA-M2e, and M2e-H3 stalk mRNA vaccines express protein antigens in HEK293T cells after each corresponding mRNA transfection. (A) H1 HA (full length) mRNA expression as a proof-of-concept of mRNA vaccine: Fluorescent microscope with A/Cal/09 H1N1 polyclonal antisera, (B) Flu B NA (consensus NA)—M2e mRNA: The expression of Flu B NA (consensus NA)—M2e mRNA was confirmed by M2e monoclonal antibody (mAb) 14C2. (C-D) Confocal microscope with M2e 14C2 mAb probe and nuclei stained with DAPI dye. (C) 5xM2e mRNA expression by 14C2 mAb. (D) M2e-H3 stalk mRNA expression by 14C2 mAb. (A-D) Alexa Fluor 488 (displaying green color of fluorescence) conjugate 2nd Abs to visualize mRNA expression.

FIGS. 50A to 50D show expression of mRNA constructs by cell surface ELISA (Enzyme linked immunosorbent assay). HEK293T cell surfaces after each corresponding mRNA transfection were probed by ELISA using antigen specific monoclonal antibodies (mAb). FIG. 50A shows cell surface expression of 5xM2e mRNA by 14C2 mAb. FIG. 50B shows cell surface expression of influenza B consensus NA-M2e mRNA by 14C2 mAb. FIG. 50C shows cell surface expression of M2e-H3 stalk mRNA by stalk specific HCA-2 mAb. FIG. 50D shows cell surface expression of M2e-H3 stalk mRNA by M2e specific 14C2 mAb.

FIGS. 51A to 51D show IgG antibody immune responses to mRNA-LNP prime vaccination in mice. Optimized lipid nanoparticle (LNP) of lipid mixtures (GenVoy-ILM, Precision Nanosystems) was used to encapsulate mRNA vaccines (mRNA-LNP) in the NanoAssemblr Benchtop Instrument (Precision Nanosystems). FIG. 51A is a schematic diagram for mouse vaccination protocol: The groups of BALB/c mice (n=5) were immunized with mRNA vaccines (mRNA-LNP) at a dose of mRNA indicated by prime (week 0) and boost (week 4) intramuscular (IM) injection immunization strategies. FIG. 51B shows A/California/2009 (H1N1) virus specific IgG antibody immune responses in bloods collected after prime vaccination with H1 HA mRNA-LNP. Positive control of H1 HA mRNA vaccine at 0.5 ug or 4 ug mRNA dose. FIG. 51C shows human influenza M2e (hM2e) specific IgG antibody immune responses in bloods collected after prime vaccination with 5xM2e mRNA-LNP. FIG. 51D shows M2e-H3 stalk protein specific IgG antibody immune responses in bloods collected after prime vaccination with M2e-H3 stalk mRNA-LNP.

FIGS. 52A to 52D show IgG antibody immune responses to mRNA-LNP boost vaccination in mice. FIG. 52A shows the groups of BALB/c mice (n=5) were immunized with mRNA vaccines (mRNA-LNP) at a dose of mRNA indicated by prime (week 0) and boost (week 4) IM injection immunization strategies. FIG. 52B shows human influenza M2e (hM2e) specific IgG antibody immune responses in bloods collected after boost vaccination with 5xM2e mRNA-LNP. FIG. 52C shows H3 stalk protein specific IgG antibody immune responses in bloods collected after prime (0.5 ug mRNA) and then boost vaccination with 0.25 ug mRNA of M2e-H3 stalk or 5xM2e mRNA-LNP. FIG. 52D shows IgG antibody responses for hM2e in bloods after prime (0.5 ug mRNA) and boost (0.25 ug mRNA) immunization with bivalent M2e-H3stalk mRNA+5xM2e mRNA or M2e-H3stalk only mRNA.

FIGS. 53A to 53B show protection against influenza virus at a lethal dose in vaccinated mice with 5xM2e mRNA-LNP or M2e-H3 Stalk mRNA-LNP by prime boost immunization regimen. FIG. 53A shows body weight changes after challenge with A/Nanchang/1995 H3N2 as a measure of protection. The groups of BALB/c mice (n=5) were immunized with 5xM2e mRNA-LNP or 5xM2e mRNA-LNP at a dose of mRNA indicated by prime and boost IM immunization, and then challenged with A/Nanchang H3N2 virus at 8 weeks after boost. FIG. 53B shows body weight changes after challenge with A/California/2009 as a measure of protection in mice (BALB/c, n=5) with 5xM2e mRNA (4 ug) at 8 weeks after boost. FIG. 53C shows body weight changes after challenge with reassortant rgH5N1 virus (containing H5 HA and N1 NA from A/Vietnam/2004 H5N1 and internal backbone genes of A/PR8 virus) as a measure of protection in mice (BALB/c, n=5) with 5xM2e mRNA (4 ug) at 8 weeks after boost.

FIGS. 54A to 54D show IgG antibody immune responses after influenza A NA mRNA-LNP or influenza B NA mRNA-LNP boost immunization in mice. FIG. 54A shows IgG antibody specific for N2 NA (A/Brisbane H3N2) in bloods from BALB/c mice (n=5) at 2 weeks after boost immunization with N2 NA mRNA-LNP (1 ug mRNA). FIGS. 54B to 54D show IgG antibody specific for B NA protein (B/Florida/2006) (FIG. 54B) or for inactivated B/Florida/2006 (iB/FL, Yamagata lineage) (FIG. 54C), or for inactivated B/Malaysia/2004 (iB/ML, Victoria lineage) (FIG. 54D) in bloods from BALB/c mice (n=5) at 2 weeks after boost immunization with influenza B NA mRNA-LNP (3 ug mRNA).

FIGS. 55A to 556 show protection against influenza virus at a lethal dose in vaccinated mice with N1 NA mRNA-LNP (FIG. 55A), N2 NA mRNA-LNP (FIG. 55B), or B cNA-M2e mRNA-LNP (FIG. 55C) by prime boost immunization regimen. FIG. 55A shows body weight changes after challenge with A/California/2009 as a measure of protection in mice (BALB/c, n=5) with N1 NA mRNA (4 ug) at 4 weeks after boost. FIG. 55B shows body weight changes after challenge with A/Nanchang/1995 H3N2 as a measure of protection in BALB/c mice (n=5) which were prime and boost IM immunized with N2 NA mRNA-LNP (4 ug) at 4 weeks after boost. FIG. 55C shows body weight changes after challenge with B/Florida/2006 (Yamagata lineage) as a measure of protection in mice (BALB/c, n=5) that were IM prime boost vaccinated with B cNA-M2e mRNA-LNP (3 ug) at 4 weeks after boost.

FIGS. 56A and 56B show adjuvant effects of 5xM2e mRNA-LNP and M2e-H3 stalk mRNA-LNP on enhancing immune responses to inactivated split influenza or NA protein vaccines when co-immunized in mice. The groups of BALB/c mice (n=5) were co-immunized with multi-valent mRNA (5xM2e mRNA-LNP 0.5 μg+M2e-H3 stalk mRNA-LNP 0.5 μg) and inactivated split influenza vaccine (sCal, 0.8 μg) or N2 NA protein (1 μg), or each vaccine standalone. FIG. 56A shows IgG antibodies specific for inactivated influenza virus (iA/Cal/H1N1, A/California/2009 H1N1) at 2 weeks after prime dose. FIG. 56B shows IgG antibodies specific for N2 NA protein (N2 NA of A/Brisbane, H3N2) at 2 weeks after prime dose.

FIGS. 57A and 57B show adjuvant effects of 5xM2e mRNA-LNP on enhancing protection against influenza virus when co-immunized with inactivated influenza vaccines in mice after prime dose. The groups of BALB/c mice (n=5) were co-immunized with 5xM2e mRNA-LNP (1 μg) and inactivated split influenza vaccine (sCal, 0.8 μg) or inactivated A/Philippines/82 H3N2 virus vaccine (iPhil/H3N2, 0.3 μg), or each vaccine standalone. FIG. 57A shows body weight changes after challenge with A/California/2009 as a measure of protection in mice (BALB/c, n=5) with a prime dose of 5xM2e mRNA-LNP (1 μg) and inactivated split influenza vaccine (sCal, 0.8 μg) or each vaccine standalone. FIG. 57B shows body weight changes after challenge with A/Nanchang/1995 H3N2 as a measure of protection in BALB/c mice (n=5) which were prime IM co-immunized with 5xM2e mRNA-LNP (1 μg) and inactivated A/Philippines/82 H3N2 virus vaccine (iPhil/H3N2, 0.3 μg), or immunized with each vaccine standalone.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a vaccine,” “a protein,” or “an influenza virus,” includes, but is not limited to, mixtures or combinations of two or more such vaccines, proteins, or influenza viruses, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not.

Unless otherwise specified, pressures referred to herein are based on atmospheric pressure (i.e. one atmosphere).

“Optional” or “optionally” means that the subsequently described event or circumstance can or cannot occur, and that the description includes instances where the event or circumstance occurs and instances where it does not. For example, the phrase “optionally a signal peptide” means that the signal peptide may or may not be included.

The term “universal influenza A vaccine” refers to vaccine capable of providing cross-protection against at least two, including three, four, five or more, subtypes of influenza A.

The term “individual,” “host,” “subject,” and “patient” are used interchangeably to refer to any individual who is the target of administration, treatment, or vaccination. The subject can be a vertebrate, for example, a mammal. Thus, the subject can be a human or veterinary patient.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

The terms “peptide,” “protein,” and “polypeptide” are used interchangeably to refer to a natural or synthetic molecule comprising two or more amino acids linked by the carboxyl group of one amino acid to the alpha amino group of another.

The term “protein domain” refers to a portion of a protein, portions of a protein, or an entire protein showing structural integrity; this determination may be based on amino acid composition of a portion of a protein, portions of a protein, or the entire protein.

The term “nucleic acid” refers to a natural or synthetic molecule comprising a single nucleotide or two or more nucleotides linked by a phosphate group at the 3′ position of one nucleotide to the 5′ end of another nucleotide. The nucleic acid is not limited by length, and thus the nucleic acid can include deoxyribonucleic acid (DNA) or ribonucleic acid (RNA).

The term “variant” refers to an amino acid sequence having conservative amino acid substitutions, non-conservative amino acid substitutions (i.e. a degenerate variant), or a peptide having 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the recited sequence.

The term “percent (%) sequence identity” or “homology” is defined as the percentage of amino acids in a candidate sequence that are identical with the nucleotides or amino acids in a reference nucleic acid sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods. For purposes herein, the % sequence identity of a given nucleotides or amino acids sequence C to, with, or against a given nucleic acid sequence D (which can alternatively be phrased as a given sequence C that has or comprises a certain % sequence identity to, with, or against a given sequence D) is calculated as follows:
100 times the fraction W/Z,
where W is the number of nucleotides or amino acids scored as identical matches by the sequence alignment program in that program's alignment of C and D, and where Z is the total number of nucleotides or amino acids in D. It will be appreciated that where the length of sequence C is not equal to the length of sequence D, the % sequence identity of C to D will not equal the % sequence identity of D to C.

A “fusion protein” refers to a polypeptide formed by the joining of two or more polypeptides through a peptide bond formed between the amino terminus of one polypeptide and the carboxyl terminus of another polypeptide. The fusion protein can be formed by the chemical coupling of the constituent polypeptides or it can be expressed as a single polypeptide from nucleic acid sequence encoding the single contiguous fusion protein. A single chain fusion protein is a fusion protein having a single contiguous polypeptide backbone. Fusion proteins can be prepared using conventional techniques in molecular biology to join the two genes in frame into a single nucleic acid, and then expressing the nucleic acid in an appropriate host cell under conditions in which the fusion protein is produced.

A “spacer” as used herein refers to a peptide that joins the proteins of a fusion protein. Generally a spacer has no specific biological activity other than to join the proteins or to preserve some minimum distance or other spatial relationship between them. However, the constituent amino acids of a spacer may be selected to influence some property of the molecule, such as the folding, net charge, or hydrophobicity of the molecule.

### Sequences

In some embodiments, the consensus N1 NA (cN1)
has the amino acid sequence
(SEQ ID NO: 1)
MNPNQKIITIGSVCMTIGMANLILQIGNIISIWVSHSIQIGNQSQ
IETCNQSVITYENNTWVNQTYVNISNTNFAAGQSWSVKLAGNSSL
CPVSGWAIYSKDNSVRIGSKGDVFVIREPFISCSPLECRTFFLTQ
GALLNDKHSNGTIKDRSPHRTLMSCPIGEAPSPYNSRFESVAWSA
SACHDGTSWLTIGISGPDSGAVAVLKYNGIITDTIKSWRNNILRT
QESECACVNGSCFTIMTDGPSDGQASYKIFKMEKGKIVKSVEMDA
PNYHYEECSCYPDSSEITCVCRDNWHGSNRPWVSFNQNLEYQIGY
ICSGVFGDNPRPNDKTGSCGPVSSNGANGVKGFSFKYGNGVWIGR
TKSTSSRKGFEMIWDPNGWTGTDNKFSIKQDIVGINEWSGYSGSF
VQHPELTGLDCIRPCFWVELIRGRPEENTIWTSGSSISFCGVNSD
TVGWSWPDGAELPFTIDK,
or a conservative variant thereof having at
least 70%, 71%, 72%, 73%, 74%, 75%, 76%, 77%,
78%, 79%, 80%, 81%, 82%, 83%, 84%, 85%, 86%,
87%, 88%, 89%, 90%, 91%, 92%,
93%, 94%, 95%, 96%, 97%, 98%, or 99%
sequence identity to SEQ ID NO: 1.
In some embodiments, the consensus N2 NA (cN2)
has the amino acid sequence
(SEQ ID NO: 2)
MNPNQKIITIGSVSLTISTICFFMQIAILITTVTLHFKQYEFNSP
PNNQVMLCEPTIIERNITEIVYLTNTTIEKEICPKPAEYRNWSKP
QCGITGFAPFSKDNSIRLSAGGDIWVTREPYVSCDPDKCYQFALG
QGTTLNNVHSNDTVRDRTPYRTLLMNELGVPFHLGTKQVCIAWSS
SSCHDGKAWLHVCITGDDKNATASFIYNGRLVDSWSWSKDILRTQ
ESECVCINGTCTWMTDGSASGKADTKILFIEEGKIVHTSKLSGSA
QHVEECSCYPRYPGVRCVCRDNWKGSNRPIVDINIKDHSIVSSYV
CSGLVGDTPRKNDSSSSSHCLDPNNEEGGHGVKGWAFDDGNDVWM
GRTISEKSRSGYETFKWEGWSNPKSKLQINRQVIVDRGDRSGYSG
IFSVEGKSCINRCFYVELIRGRKEETEVLWTSNSIWFCGTSG
TYGTGSWPDGADLNLMPI,
or a conservative variant thereof having
at least
70%, 71%, 72%, 73%, 74%, 75%, 76%, 77%, 78%,
79%, 80%, 81%, 82%, 83%, 84%, 85%, 86%, 87%,
88%, 89%, 90%, 91%, 92%, 93%, 94%, 95%, 96%,
97%, 98%, or 99% sequence identity to
SEQ ID NO: 2.
In some embodiments, the consensus influenza
B NA (B-cNA) has the amino acid sequence
(SEQ ID NO: 3)
MLPSTIQTLTLFLTSGGVLLSLYVSASLSYLLYSDILLKFSRTEI
TAPIMPLDCANASNVQAVNRSATKGVTPLLPEPEWTYPRLSCPGS
TFQKALLISPHRFGETKGNSAPLIIREPFIACGPKECKHFALTHY
AAQPGGYYNGTREDRNKLRHLISVKLGKIPTVENSIFHMAAWSGS
ACHDGKEWTYIGVDGPDSNALLKIKYGEAYTDTYHSYAKNILRTQ
ESACNCIGGDCYLMITDGPASGVSECRFLKIREGRIIKEIFPTGR
VKHTEECTCGFASNKTIECACRDNSYTAKRPFVKLNVETDTAEIR
LMCTKTYLDTPRPNDGSITGPCESDGDKGSGGIKGGFVHQRMASK
IGRWYSRTMSKTKRMGMGLYVKYDGDPWTDSEALALSGVMVSMEE
PGWYSFGFEIKDKKCDVPCIGIEMVHDGGKTTWHSAATAIYCLM
GSGQLLWDTVTGVNMTL, or a conservative variant
thereof having at least 70%, 71%, 72%, 73%,
74%, 75%, 76%, 77%, 78%, 79%, 80%, 81%, 82%,
83%, 84%, 85%, 86%, 87%, 88%, 89%, 90%, 91%,
92%, 93%, 94%, 95%, 96%, 97%, 98%, or 99%
sequence identity to SEQ ID NO: 3.

In some embodiments, the human M2e sequence comprises the amino acid sequence PIRNEWGSRSN (SEQ ID NO:4), or a conservative variant thereof having at least about 70%, 80%, or 90% sequence identity to SEQ ID NO:4 (i.e., one, two, or three conservative amino acid substitutions). For example, human M2e isolates H1N1 (A/PR8, A/NC/99) and H3N2 (A/Phil/82) have the amino acid sequence SLLTEVETPIRNEWGSRSNDSSD (SEQ ID NO:5).

In some embodiments, amino acids that are conserved across species are maintained, e.g., Arg at position three and nine, Trp at position six, and Cys at position eight of SEQ ID NO:4. In other embodiments, conserved residues are conservatively substituted, e.g., Arg to Lys. In some embodiments, amino acids that are unique to a given species are conserved to increase heterogeneity and cross-protection, e.g., Ile at position two and Asp at position eleven of SEQ ID NO:4. Candidate sequence variants containing conserved substitutions may be tested using antibodies against the reference protein. In some embodiments, immune sera against M2e may be tested against the M2e variants for the cross-reactivity.

In some embodiments, the swine M2e sequence comprises the amino acid sequence PTRSEWESRSS (SEQ ID NO:6), or a conservative variant thereof having at least 70%, 80%, or 90% sequence identity to SEQ ID NO:6. For example, swine M2e isolates from the 2009 H1N1 pandemic (A/California/4/2009) have the amino acid sequence

(SEQ ID NO: 7)
SLLTEVETPTRSEWESRSSDSSD.

In some embodiments, the avian M2e sequence (referred to herein as “avian type I”) comprises the amino acid sequence PTRX₁X₂WESRSS (SEQ ID NO:47), wherein X₁ is N, H, or K, wherein X₂ is E or G, or a conservative variant thereof having at least 70%, 80%, or 90% sequence identity to SEQ ID NO:47. For example, avian type I M2e isolates from H5N1 (A/Vietnam/1203/04, A/Indonesia/05, A/mandarin/kr/2010, A/ck/kr/2006) have the amino acid sequence SLLTEVETPTRNEWESRSSDSSD (SEQ ID NO:8). Avian type I M2e isolates from H7N3 (A/dk/Kr/2007), H9N2 (A/ck/Kr/2012) have the amino acid sequence SLLTEVEPTRNGWECRCSDSSD (SEQ ID NO:9). Avian type I M2e isolates from H5N1 (A/ck/Kr/Gimje/2008) have the amino acid sequence SLLTEVETPTRHEWECRCSDSSD (SEQ ID NO:10). Avian type I M2e isolates from H5N1 (A/ck/Vietnam/2011) have the amino acid sequence SLLTEVETPTRKEWECRCSDSSD (SEQ ID NO:11).

In some embodiments, the avian M2e sequence (referred to herein as “avian type 1l”) comprises the amino acid sequence LTRNGWGCRCS (SEQ ID NO:12), or a conservative variant thereof having at least 70%, 80%, or 90% sequence identity to SEQ ID NO:12. For example, avian type II M2e isolates from H5N1 (A/HK/156/97), H9N2 (A/HK/1073/99) have the amino acid sequence SLLTEVETLTRNGWGCRCSDSSD (SEQ ID NO:13).

In some embodiments, the 5xM2e (Human (x2)-
Swine-Avian I/II) has the
amino acid sequence
(SEQ ID NO: 14)
MKFLVNVALVFMWYISYIYADPINMTTSINNNLQRVRELAVQSAN
SAAAPGAAVDGTSLLTEVETPIRNEWGSRSNDSSDAAAGGAASLL
TEVETPIRNEWGSRSNDSSDAAAPGAASLLTEVETPTRSEWESRS
SDSSDAAAGGAASLLTEVETPTRNEWESRSSDSSDAAAPGAASLL
TEVETLTRNGWGCRCSDSSDGGLKQIEDKLEEILSKLYHIENELA
RIKKLLGELEILAIYSTVASSLVLLVSLGAISFWMCSNGSLQCRI
CI,
or a conservative variant thereof having at
least 70%, 71%, 72%, 73%, 74%, 75%, 76%, 77%,
78%, 79%, 80%, 81%, 82%, 83%, 84%, 85%, 86%,
87%, 88%, 89%, 90%, 91%, 92%, 93%, 94%, 95%,
96%, 97%, 98%, or 99% sequence identity
to SEQ ID NO: 14.
In some embodiments, the 5xM2e has the amino
acid sequence
(SEQ ID NO: 15)
MDAMKRGLCCVLLLCGAVFVSASQESLLTEVETPIRNEWGSRSND
SSDAAAGGASLLTEVETPTRSEWESRSSDSSDAAAPGAASLLTEV
ETPTRNEWESRSSDSSDAAAGGASLLTEVETPTRTGWESNSNGSS
DAAAPGAASLLTEVETPIRNEWGSRSNDSSDAAAGGGQIEDKLEE
ILSKLYHIENELARIKKLLGEYQILAIYSTVASSLVLLVSLGAIS
FWMCSNGSLQCRICI,
or a conservative variant thereof having at
least 70%, 71%, 72%, 73%, 74%, 75%, 76%, 77%,
78%, 79%, 80%, 81%, 82%, 83%, 84%, 85%, 86%,
87%, 88%, 89%, 90%, 91%, 92%, 93%, 94%, 95%,
96%, 97%, 98%, or 99% sequence identity to
SEQ ID NO:15.
In some embodiments, the M2e-H3 stalk has the
amino acid sequence
(SEQ ID NO: 16)
MDAMKRGLCCVLLLCGAVFVSASQESLLTEVETPIRNEWGSRSND
SSDAAAGGAASLLTEVETPTRSEWESRSSDSSDGGGGSPNGTLVK
TITDDQIEVTNATELVQSSGSAPNDKPFQNTNKNTTGASPKYVKQ
NTLKLATGMRNGSAGSAGLFGAIAGFIENGWEGMIDGWYGFRHQN
SEGTGQAADLKSTQAAIDQINGKLNRVIEKTNEKDHQDEKEFSED
EGRIQDLEKYVEDTKIDLWSYNAELLVALENQHTIDATDSEMNKQ
GTGGGYIPEAPRDGQAYVRKDGEWVLLSTFL,
or a conservative variant thereof having at
least 70%, 71%, 72%, 73%, 74%, 75%, 76%, 77%,
78%, 79%, 80%, 81%, 82%, 83%, 84%, 85%, 86%,
87%, 88%, 89%, 90%, 91%, 92%, 93%, 94%, 95%,
96%, 97%, 98%, or 99% sequence identity to
SEQ ID NO: 16.
In some embodiments, the N1 NA (consensus) has
the amino acid sequence
(SEQ ID NO: 17)
MDAMKRGLCCVLLLCGAVFVSASQESLLTEVETPIRNEWGSRSND
SSDGGSGGGSLLTEVETPTRSEWESRSSDSSDAAAPGAASHSIQI
GNQSQIETCNQSVITYENNTWVNQTYVNISNTNFAAGQSVVSVKL
AGNSSLCPVSGWAIYSKDNSVRIGSKGDVFVIREPFISCSPLECR
TFFLTQGALLNDKHSNGTIKDRSPYRTLMSCPIGEVPSPYNSRFE
SVAWSASACHDGINWLTIGISGPDSGAVAVLKYNGIITDTIKSWR
NNILRTQESECACVNGSCFTIMTDGPSDGQASYKIFRIEKGKIIK
SVEMKAPNYHYEECSCYPDSSEITCVCRDNWHGSNRPWVSFNQNL
EYQMGYICSGVFGDNPRPNDKTGSCGPVSSNGANGVKGFSFKYGN
GVWIGRTKSISSRKGFEMIWDPNGWTGTDNKFSIKQDIVGINEWS
GYSGSFVQHPELTGLDCIRPCFWVELIRGRPEENTIWTSGSSISF
CGVNSDTVGWSWPDGAELPFTIDK,
or a conservative variant thereof having at
least 70%, 71%, 72%, 73%, 74%, 75%, 76%, 77%,
78%, 79%, 80%, 81%, 82%, 83%, 84%, 85%, 86%,
87%, 88%, 89%, 90%, 91%, 92%, 93%, 94%, 95%,
96%, 97%, 98%, or 99% sequence identity
to SEQ ID NO: 17.
In some embodiments, the N2 NA (consensus)
mRNA encoding protein amino acid sequence
(SEQ ID NO: 18)
MNPNQKIITIGSVSLTISTICFFMQIAILITTVTLHFKQYEFNSP
PNNQVMLCEPTIIERNITEIVYLTNTTIEKEICPKPAEYRNWSKP
QCGITGFAPFSKDNSIRLSAGGDIWVTREPYVSCDPDKCYQFALG
QGTTLNNVHSNDTVRDRTPYRTLLMNELGVPFHLGTKQVCIAWSS
SSCHDGKAWLHVCITGDDKNATASFIYNGRLVDSVVSWSKDILRT
QESECVCINGTCTVVMTDGSASGKADTKILFIEEGKIVHTSTLSG
SAQHVEECSCYPRYPGVRCVCRDNWKGSNRPIVDINIKDHSIVSS
YVCSGLVGDTPRKNDSSSSSHCLDPNNEEGGHGVKGWAFDDGNDV
WMGRTISEKSRSGYETFKVVEGWSNPKSKLQINRQVIVDRGDRSG
YSGIFSVEGKSCINRCFYVELIRGRKEETEVLWTSNSIVVFCGTS
GTYGTGSWPDGADLNLMPI,
or a conservative variant thereof having at
least 70%, 71%, 72%, 73%, 74%, 75%, 76%, 77%,
78%, 79%, 80%, 81%, 82%, 83%, 84%, 85%, 86%,
87%, 88%, 89%, 90%, 91%, 92%, 93%, 94%, 95%,
96%, 97%, 98%, or 99% sequence identity to
SEQ ID NO: 18.
In some embodiments, the Flu B NA-M2e has the
amino acid sequence
(SEQ ID NO: 19)
MDAMKRGLCCVLLLCGAVFVSASQESLLTEVETPIRNEWGSRSND
SSDGGSGGGSLLTEVETPTRSEWESRSSDSSDAAAPGAASLLTEV
ETPTRNEWESRSSDSSDAAGGGASLLTEVETPTRTGWESNSNGSS
DAAPGGSGIINETADDIVYRLTVIIDDRYESLKNLITLRADRLEM
IINDNVSTILASGGSGGPEWTYPRLSCPGSTFQKALLISPHRFGE
TKGNSAPLIIREPFIACGPKECKHFALTHYAAQPGGYYNGTREDR
NKLRHLISVKLGKIPTVENSIFHMAAWSGSACHDGKEWTYIGVDG
PDSNALLKIKYGEAYTDTYHSYAKNILRTQESACNCIGGDCYLMI
TDGPASGVSECRFLKIREGRIIKEIFPTGRVKHTEECTCGFASNK
TIECACRDNSYTAKRPFVKLNVETDTAEIRLMCTKTYLDTPRPND
GSITGPCESDGDKGSGGIKGGFVHQRMASKIGRWYSRTMSKTKRM
GMGLYVKYDGDPWTDSEALALSGVMVSMEEPGWYSFGFEIKDKKC
DVPCIGIEMVHDGGKTTWHSAATAIYCLMGSGQLLWDTVTGVNMT
L,
or a conservative variant thereof having
at least 70%, 71%, 72%, 73%, 74%, 75%, 76%,
77%, 78%, 79%, 80%, 81%, 82%, 83%, 84%, 85%,
86%, 87%, 88%, 89%, 90%, 91%, 92%, 93%, 94%,
95%, 96%, 97%, 98%, or 99% sequence identity
to SEQ ID NO: 19.

The fusion protein may further comprise a signal peptide at the N-terminus to facilitate secretion. For example, the fusion protein may contain a mellitin signal peptide. In some embodiments, the melittin signal peptide has the amino acid sequence MKFLVNVALVFMVVYISYIYADPINMT (SEQ ID NO:20), or a conservative variant thereof having at least 72%, 76%, 80%, 84%, 88%, 92%, or 96% sequence identity to SEQ ID NO:20. Alternatively, the fusion protein may contain a baculovirus gp64 signal peptide (MVSAIVLYVLLAAAAHSAFA, SEQ ID NO:21) (Wang, B., et al. J Virol 2007 81: 10869-10878), or a modified signal peptide of Tissue plasminogen activator (MDAMKRGLCCVLLLCGAVFVSASQE, SEQ ID NO: 22) or a conservative variant thereof having at least 70%, 75%, 80%, 85%, 90%, or 95% sequence identity to SEQ ID NO:21 or SEQ ID NO:22.

Influenza M2 is naturally a homotetramer. Therefore, in some embodiments, the fusion protein also contains an oligomer stabilization domain. In some embodiments, the disclosed vaccine contains a tetramer stabilizing domain called GCN4 (leucine zipper tetramerization motif) (De Filette, M., et al. J Biol Chem 2008 283:11382-11387). For example, the GCN4 domain can have the amino acid sequence GGLKQIEDKLEEILSKLYHIENELARIKKLLGE (SEQ ID NO:23), or a conservative variant thereof having at least 70%, 73%, 76%, 79%, 82%, 85%, 88%, 91%, 94%, or 97% sequence identity to SEQ ID NO:23. In some embodiments, the disclosed vaccine contains a NSP498-135 fragment of rotavirus (QMDRVVKEMRRQLEMIDKLTTREIEQVELLKRIYDKL, SEQ ID NO:24) (Andersson, A. M., K. 0. Hakansson, et al. (2012). PLoS One 7(10): e46395.), or a conservative variant thereof having at least 70%, 73%, 76%, 79%, 82%, 85%, 88%, 91%, 94%, or 97% sequence identity to SEQ ID NO:24, as the parallel tetrameric coiled-coil stabilizing domain.

To anchor multiple copies of heterologous tandem repeat M2e on the surface of a particle, the fusion protein may be expressed in a membrane-anchored form and incorporated in virus-like particles (VLPs). Therefore, in some embodiments, the fusion protein further comprises a membrane anchor domain, such as a transmembrane domain and optional cytoplasmic domain of a viral envelope protein. For example, fusion proteins containing M2e domains with the transmembrane domain and cytoplasmic domain of influenza A hemagglutinin (HA) have been shown to incorporate into VLPs at a higher rate than wild type M2 protein. In some embodiments, the membrane anchor domain comprises the full HA protein sequence. The transmembrane-cytoplasmic domain from hemagglutinin of A/PR/8/34 virus can have the amino acid sequence ILAIYSTVASSLVLLVSLGAISFWMCSNGSLQCRICI (SEQ ID NO:25), or a conservative variant thereof having at least 76%, 79%, 82%, 85%, 88%, 91%, 94%, or 97% sequence identity to SEQ ID NO:25. The disclosed fusion protein may also comprise a HA stalk domain. The HA stalk domain can have the following amino acid sequence TKCQTPLGAINSSLPYQNIHPVTIGECPKYVRSAKLRMVTGLRNNPSIQSRGLFGAIAGFIEGGW TGMIDGWYGYHHQNEQGSGYAADQKSTQNAINGITNKVNTVIEKMNIQFTAVGKEFNKLEKRM ENLNKKVDDGFLDIWTYNAELLVLLENERTLDFHDSNVKNLYEKVKSQLKNNAKEIGNGCFEFY HKCDNECMESVRNGTYDYPKYSEESKLNREKVDGVKLESMGIYQILAIYSTVASSLVLLVSLGAI SFWMCSNGSLQCRICI (SEQ ID NO:26), or a conservative variant thereof having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:26.

The HA stalk domain can be a mini-stem (aa 1-40-PG-117 without a membrane anchor) of H1 HA for effective expression) having the following amino acid sequence: GLFGAIAGFIEGGWTGMIDGWYGYHHQNEQGSGYAADQKSPGTQNAINGITNKVNTVIEKMNI QDTATGKEFNKDEKRMENLNKKVDDGFLDIWTYNAELLVLLENERTLDAHDSNVKN (SEQ ID NO:27), or a conservative variant thereof having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:27.

The full length H1 HA stalk domain has the amino acid sequence

(SEQ ID NO: 28)
MKAILVVLLYTFATANADTLCIGYHANNSTDTVDTVLEKNVTVTH
SVNLLEDKHNGKLCKLRGVAPLHLGKCNIAGWILGNPECESLSTA
SSWSYIVETSSSDNGTCYPGDFINYEELREQLSSVSSFERFEIFP
KTSSWPNHESNKGVTAACPHAGAKSFYKNLIWLVKKGNSYPKLSQ
SYINDKGKEVLVLWGIHHPPTTADQQSLYQNADAYVFVGTSRYSK
KFKPEIAIRPKVRDQEGRMNYYWTLVEPGDKITFEATGNLVVPRY
AFTMERNAGSGIIISDTPVHDCNTTCQTPEGAINTSLPFQNIHPI
TIGKCPKYVKSTKLRLATGLRNVPSIQSRGLFGAIAGFIEGGWTG
MVDGWYGYHHQNEQGSGYAADLKSTQNAIDKITNKVNSVIEKMNT
QFTAVGKEFNHLEKRIENLNKKVDDGFLDIWTYNAELLVLLENER
TLDYHDSNVKNLYEKVRNQLKNNAKEIGNGCFEFYHKCDNTCMES
VKNGTYDYPKYSEEAKLNREKIDGVKLESTRIYQILAIYSTVASS
VVLVVSLGAISFWMCSNGSLQCRICI

Influenza A virus HA subtypes are phylogenetically divided into group 1 (H1, H2, H5, H6, H8, H9, H11, H12, H13, H16, H17, H18) and group 2 (H3, H4, H7, H10, H14, H15). The HA on the virion is in the prefusion state and cleaved by host proteases into HA1 (the head domain) and HA2 (the stem domain).

The following is the amino acid sequence for the influenza A H3 stalk ectodomain: MKTIIALSYIFCLPLGQDLPGNDNSTATLCLGHHAVPNGTLVKTITDDQIEVTNATELVQSSSTGK ICNNPHRILDGIDCTLIDALLGDPHCDVFQNETWDLFVERSKAFSNCYPYDVPDYASLRSLVASS GTLEFITEGFTWTGVTQNGGSNACKRGPGSGFFSRLNWLTKSGSTYPVLNVTMPNNDNFDKL YIWGIHHPSTNQEQTSLYVQASGRVTVSTRRSQQTIIPNIGSRPWVRGLSSRISIYWTIVKPGDV LVINSNGNLIAPRGYFKMRTGKSSIMRSDAPIDTCISECITPNGSIPNDKPFQNVNKITYGACPKY VKQNTLKLATGMRNVPEKQTRGLFGAIAGFIENGWEGMIDGWYGFRHQNSEGTGQAADLKST QAAIDQINGKLNRVIEKTNEKFHQIEKEFSEVEGRIQDLEKYVEDTKIDLWSYNAELLVALENQHT IDLTDSEMNKLFEKTRRQLRENAEEMGNGCFKIYHKCDNACIESIRNGTYDHDVYRDEALNNRF QIKGVELKSGYK (SEQ ID NO:29). The corresponding amino acid sequences for other HA group 2 subtypes are known. Therefore, reference to specific amino acids within SEQ ID NO:29 is also a reference to the corresponding amino acids in the known amino acid sequences for the other group 2 subtypes.

In some embodiments, the HA1a domain in the construct of M2e-H3(Aichi)-stalk fusion protein has the amino acid sequence PNGTLVKTITDDQIEVTNATELVQSS (SEQ ID NO:30), or a conservative variant thereof having at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:30.

In some embodiments, the HA1b domain in the construct of M2e-H3(Aichi)-stalk fusion protein has the amino acid sequence PNDKPFQNVNKITYGACPKYVKQNTLKLATGMRN (SEQ ID NO:31), or a conservative variant thereof having at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:31.

In some embodiments, the HA2 domain in the
construct of M2e-H3(Aichi)-stalk fusion
protein has the amino acid sequence
(SEQ ID NO: 32)
GLFGAIAGFIENGWEGMIDGWYGFRHQNSEGTGQAADLKSTQAAI
DQINGKLNRVIEKTNEKFHQIEKEFSEVEGRIQDLEKYVEDTKID
LWSYNAELLVALENQHTIDLTDSEMNK,
or a conservative variant thereof having
at least 80%, 81%, 82%, 83%, 84%, 85%, 86%,
87%, 88%, 89%, 90%, 91%, 92%, 93%, 94%, 95%,
96%, 97%, 98%, or 99% sequence
identity to SEQ ID NO: 32.

The following is the amino acid sequence for the influenza A H1 stalk ectodomain: GLFGAIAGFIEGGWTGMIDGWYGYHHQNEQGSGYAADQKSTQNAINGITNKVNSVIEKMNIQF TAVGKEFNKLEKRMENLNKKVDDGFLDIWTYNAELLVLLENERTLDFHDSNVKNLYEKVKSQLK NNAKEIGNGCFEFYHKCDNECMESVRNGTYDYPKYSEESKLNREKVDGVKLESMG (SEQ ID NO:33). The corresponding amino acid sequences for other group 1 HA subtypes are known. Therefore, reference to specific amino acids within SEQ ID NO:33 is also a reference to the corresponding amino acids in the known amino acid sequences for the other group 1 subtypes.

In some embodiments, the HA1a domain has the
amino acid sequence
(SEQ ID NO: 34)
DTVDTVLEKNVTVTHSVNLLEDSH,
or a conservative variant thereof having at
least 80%, 81%, 82%, 83%, 84%, 85%, 86%, 87%, 88%,
89%, 90%, 91%, 92%, 93%, 94%, 95%, 96%, 97%, 98%,
or 99% sequence identity to SEQ ID NO: 34.
In some embodiments, the HA1b domain has the
amino acid sequence
(SEQ ID NO: 35)
NSSLPYQNTHPTTNGESPKYVRSAKLRMVTGLRN,
or a conservative variant
thereof having at least 80%, 81%, 82%, 83%, 84%,
85%, 86%, 87%, 88%, 89%, 90%, 91%, 92%, 93%, 94%,
95%, 96%, 97%, 98%, or 99% sequence identity
to SEQ ID NO: 35.
In some embodiments, the HA2 domain has the
amino acid sequence
(SEQ ID NO: 36)
GLFGAIAGFIEGGWTGMIDGWYGYHHQNEQGSGYAADQKSPGTQN
AINGITNKVNTVIEKMNIQDTATGKEFNKDEKRMENLNKKVDDGF
LDIWTYNAELLVLLENERTLDAHDSNVKN,
or a conservative variant thereof having at least
80%, 81%, 82%, 83%, 84%, 85%, 86%, 87%, 88%, 89%,
90%, 91%, 92%, 93%, 94%, 95%, 96%, 97%, 98%, or
99% sequence identity to SEQ ID NO: 36.
In some embodiments, the M2e-H1 stalk mRNA has
the amino acid sequence
(SEQ ID NO: 37)
MDAMKRGLCCVLLLCGAVFVSASQESLLTEVETPIRNEWGSRSND
SSDAAAGGAASLLTEVETPTRSEWESRSSDSSDGGGGSDTVDTVL
EKNVTVTHSVNLLEDSHGSANSSLPYQNTHPTTNGESPKYVRSAK
LRMVTGLRNGSAGSAPGGLFGAIAGFIEGGWTGMIDGWYGYHHQN
EQGSGYAADQKSPGTQNAINGITNKVNTVIEKMNIQDTATGKEFN
KDEKRMENLNKKVDDGFLDIWTYNAELLVLLENERTLDAHDSNVK
NQGTGGGYIPEAPRDGQAYVRKDGEWVLLSTFLKL,
or a conservative variant thereof having at
least 70%, 71%, 72%, 73%, 74%, 75%, 76%, 77%,
78%, 79%, 80%, 81%, 82%, 83%, 84%, 85%, 86%,
87%, 88%, 89%, 90%, 91%, 92%, 93%, 94%, 95%,
96%, 97%, 98%, or 99% sequence identity to
SEQ ID NO: 37.
In some embodiments, the M2e-H1 stalk mRNA has
the amino acid sequence
(SEQ ID NO: 54)
MDAMKRGLCCVLLLCGAVFVSASQESLLTEVETPIRNEWGSRSND
SSDAAAGGAASLLTEVETPTRSEWESRSSDSSDGGGGSPNGTLVK
TITDDQIEVTNATELVQSSGSAPNDKPFQNTNKNTTGASPKYVKQ
NTLKLATGMRNGSAGSAGLFGAIAGFIENGWEGMIDGWYGFRHQN
SEGTGQAADLKSTQAAIDQINGKLNRVIEKTNEKDHQDEKEFSED
EGRIQDLEKYVEDTKIDLWSYNAELLVALENQHTIDATDSEMNKQ
GTGGGYIPEAPRDGQAYVRKDGEWVLLSTFL,
or a conservative variant thereof having at
least 80%, 81%, 82%, 83%, 84%, 85%, 86%, 87%,
88%, 89%, 90%, 91%, 92%, 93%, 94%, 95%, 96%,
97%, 98%, or 99% sequence identity to
SEQ ID NO: 54.
In some embodiments, the M2e-H3 stalk mRNA has
the amino acid sequence
(SEQ ID NO: 55)
MDAMKRGLCCVLLLCGAVFVSASQESLLTEVETPIRNEWGSRSND
SSDAAAGGAASLLTEVETPTRSEWESRSSDSSDGGGGSPNGTLVK
TITDDQIEVTNATELVQSSGSAPNDKPFQNTNKNTTGASPKYVKQ
NTLKLATGMRNGSAGSAGLFGAIAGFIENGWEGMIDGWYGFRHQN
SEGTGQAADLKSTQAAIDQINGKLNRVIEKTNEKDHQDEKEFSED
EGRIQDLEKYVEDTKIDLWSYNAELLVALENQHTIDATDSEMNKQ
GTGGGYIPEAPRDGQAYVRKDGEWVLLSTFL,
or a conservative variant thereof having at least
80%, 81%, 82%, 83%, 84%, 85%, 86%, 87%, 88%, 89%,
90%, 91%, 92%, 93%, 94%, 95%, 96%, 97%, 98%, or
99% sequence identity to SEQ ID NO: 55.
In some embodiments, the timeric Foldon has the
amino acid sequence
(SEQ ID NO: 38)
GYIPEAPRDGQAYVRKDGEWVLLSTFL,
or a conservative variant thereof having at least
80%, 81%, 82%, 83%, 84%, 85%, 86%, 87%, 88%, 89%,
90%, 91%, 92%, 93%, 94%, 95%, 96%, 97%, 98%, or
99% sequence identity to SEQ ID NO: 38.

mRNA Vaccines

Also disclosed herein are cross-protective mRNA influenza vaccines.

In some embodiments, an mRNA 5xM2e vaccine
has the nucleic acid sequence
(SEQ ID NO: 48)
ATGGACGCCATGAAAAGAGGCCTGTGCTGCGTGCTGCTGCTCTGT
GGCGCCGTGTTCGTGTCCGCCTCTCAGGAGAGCCTGCTGACCGAG
GTTGAGACACCAATTCGGAACGAATGGGGATCTAGATCTAATGAC
AGCAGCGATGCCGCAGCTGGAGGAGCCAGCCTGCTGACCGAGGTG
GAAACCCCTACCAGAAGCGAGTGGGAGTCCAGATCCTCCGACTCT
AGCGACGCTGCAGCCCCCGGCGCCGCTTCTCTGCTGACAGAAGTG
GAAACCCCTACAAGAAACGAGTGGGAGAGCAGATCCAGCGACAGC
TCCGATGCAGCCGCTGGAGGTGCCAGCCTGCTGACAGAGGTGGAA
ACACCCACCCGGACCGGCTGGGAAAGCAACAGCAATGGCAGCAGC
GACGCTGCCGCTCCTGGCGCTGCCAGCCTGCTGACCGAGGTGGAA
ACCCCTATCAGAAACGAGTGGGGCAGCCGGAGCAACGACAGCTCT
GATGCTGCCGCTGGAGGTGGCCAGATCGAGGACAAGCTGGAAGAG
ATCCTGAGCAAGCTGTACCACATCGAGAACGAACTGGCCAGAATC
AAGAAGCTGCTGGGCGAGTACCAGATTCTGGCTATCTACAGCACC
GTGGCCTCTTCCCTGGTGCTGCTGGTCAGCCTCGGCGCCATCAGC
TTCTGGATGTGCAGCAACGGCTCCCTGCAATGTAGAATCTGCATC
TAATAA.
In some embodiments, an mRNA M2e-H3 stalk vaccine
has the nucleic acid sequence
(SEQ ID NO: 49)
ATGGACGCCATGAAGAGAGGACTGTGCTGCGTGCTGCTGCTGTGT
GGAGCTGTGTTCGTGTCCGCCTCCCAGGAGTCCCTGCTGACCGAG
GTGGAAACCCCTATCCGGAACGAGTGGGGCAGCAGAAGCAACGAC
AGCTCTGATGCAGCTGCTGGAGGAGCAGCCAGCCTGCTGACAGAG
GTCGAGACACCCACCCGGAGCGAATGGGAGAGCAGAAGCAGCGAC
AGCAGCGATGGCGGCGGCGGAAGCCCTAATGGCACACTGGTGAAA
ACCATCACCGACGACCAAATCGAGGTGACCAATGCCACAGAACTG
GTGCAGAGCAGCGGCAGCGCTCCTAACGACAAGCCTTTCCAGAAC
ACCAACAAGAACACAACCGGCGCCTCCCCAAAGTACGTGAAGCAG
AATACCCTGAAACTGGCCACCGGCATGAGAAATGGCTCAGCTGGC
TCTGCCGGACTGTTCGGCGCCATCGCCGGCTTCATCGAGAACGGA
TGGGAAGGCATGATCGACGGCTGGTACGGCTTTAGACACCAGAAC
AGCGAGGGCACAGGCCAGGCCGCCGACCTCAAGAGCACCCAGGCT
GCCATCGACCAGATCAACGGCAAGCTGAACCGGGTGATCGAGAAG
ACCAACGAGAAAGACCACCAAGACGAAAAGGAATTCAGCGAAGAT
GAGGGCAGAATCCAGGACCTGGAAAAGTACGTGGAAGATACCAAG
ATCGACCTGTGGTCCTACAACGCCGAGCTGCTGGTCGCCCTGGAA
AACCAGCACACCATCGACGCCACCGATAGCGAGATGAACAAGCAG
GGAACAGGCGGCGGCTACATCCCCGAGGCCCCTAGAGATGGCCAG
GCCTACGTGAGAAAGGACGGCGAGTGGGTGCTGCTCAGCACCTTC
CTGTAATAA.
In some embodiments, an mRNA N1 NA vaccine has
the nucleic acid sequence
(SEQ ID NO: 50)
ATGAACCCCAACCAGAAGATCATCACCATCGGCAGCGTGTGTATG
ACAATCGGAATGGCCAACCTCATCCTGCAGATCGGCAACATCATC
AGCATCTGGGTCAGCCACTCTATCCAGATCGGCAACCAGTCCCAG
ATTGAGACATGCAACCAAAGCGTGATCACCTACGAGAACAACACC
TGGGTCAACCAGACCTACGTGAACATCAGCAATACCAACTTCGCC
GCTGGCCAGAGCGTGGTGTCCGTGAAGCTGGCCGGCAACTCTAGC
CTGTGCCCCGTGAGCGGCTGGGCCATCTACTCTAAGGACAACAGC
GTGCGGATCGGCTCCAAGGGAGATGTGTTCGTGATCAGGGAACCC
TTCATCAGCTGTTCTCCCCTGGAATGCAGGACCTTCTTCCTGACC
CAGGGCGCTCTGCTGAACGACAAGCATTCCAATGGCACAATCAAG
GATAGGTCTCCATATCGGACCCTGATGTCCTGTCCCATCGGCGAG
GTGCCATCTCCCTACAACTCCAGGTTCGAGTCTGTGGCATGGAGC
GCCTCCGCCTGCCACGACGGAATCAACTGGCTGACCATCGGCATC
AGCGGCCCTGACAGCGGAGCCGTGGCTGTGCTGAAGTACAACGGA
ATCATCACAGATACCATCAAGAGCTGGCGGAACAACATCCTGCGG
ACACAGGAGAGCGAGTGCGCCTGTGTGAACGGCTCCTGTTTTACC
ATCATGACCGACGGCCCTAGCGATGGACAGGCCAGCTACAAGATC
TTCAGAATCGAAAAGGGCAAGATCGTGAAGAGCGTGGAAATGAAG
GCCCCTAACTACCACTACGAGGAATGCAGCTGCTACCCCGACAGC
AGCGAGATCACATGCGTGTGCCGGGACAACTGGCACGGCTCCAAT
AGACCCTGGGTCAGCTTCAACCAGAACCTGGAATACCAGATCGGC
TACATCTGCAGCGGCGTCTTCGGCGACAATCCTCGGCCAAATGAC
AAGACCGGCAGCTGTGGCCCTGTGTCCAGCAACGGAGCTAATGGC
GTGAAGGGCTTTAGCTTCAAGTACGGCAATGGCGTGTGGATCGGC
AGAACAAAGAGCACCTCCAGCAGGAAGGGGTTTGAGATGATCTGG
GACCCCAATGGTTGGACGGGCACCGATAACAAGTTCAGCATCAAA
CAGGACATCGTCGGAATCAACGAGTGGTCCGGCTACAGCGGCAGC
TTTGTGCAGCACCCCGAGCTGACAGGCCTGGACTGCATCCGGCCT
TGCTTCTGGGTGGAACTGATCCGTGGGAGACCTGAGGAAAACACC
ATTTGGACATCCGGCAGCAGCATCAGTTTCTGCGGCGTGAACAGC
GACACCGTGGGATGGAGCTGGCCCGATGGCGCCGAGCTGCCCTTC
ACCATCGACAAATAATAAA.
In some embodiments, an mRNA N2 NA vaccine has
the nucleic acid sequence
(SEQ ID NO: 51)
ATGAACCCCAATCAGAAGATCATCACCATCGGCTCTGTGTCCCTG
ACCATCAGCACCATCTGTTTTTTCATGCAGATCGCCATCCTGATC
ACAACCGTGACCCTGCACTTCAAGCAATACGAATTCAACAGCCCT
CCTAACAACCAGGTGATGCTGTGCGAGCCCACCATCATCGAGCGG
AACATCACCGAGATCGTCTACCTGACCAATACCACCATCGAGAAG
GAGATCTGTCCCAAGCCCGCCGAGTACCGGAACTGGTCCAAGCCT
CAGTGCGGCATCACCGGCTTCGCCCCCTTCTCCAAGGACAACTCT
ATCAGACTGTCCGCCGGAGGAGACATCTGGGTGACAAGGGAACCC
TATGTGAGCTGCGACCCCGACAAGTGCTACCAGTTCGCCCTGGGC
CAGGGCACCACCCTGAACAACGTGCATTCAAACGACACCGTGAGA
GACCGGACCCCTTACCGGACCCTGCTGATGAACGAGCTGGGCGTC
CCCTTCCACCTGGGCACAAAGCAGGTGTGCATCGCTTGGAGCAGC
AGCAGCTGCCACGATGGCAAGGCCTGGCTGCACGTGTGTATAACA
GGCGACGACAAGAACGCCACCGCCAGCTTCATCTACAACGGCCGG
CTGGTGGACAGCGTGGTGTCCTGGTCCAAAGATATCCTGCGGACA
CAGGAGAGCGAGTGCGTGTGCATCAATGGAACATGCACCGTGGTG
ATGACCGACGGCAGTGCTAGCGGAAAGGCCGACACAAGGATCCTG
TTCATCGAGGAGGGCAAAATCGTGCACACGAGCACACTCAGCGGT
TCTGCTCAGCACGTGGAGGAGTGTAGCTGCTATCCCAGATACCCC
GGCGTCCGGTGCGTGTGCAGAGATAACTGGAAAGGCAGCAACCGG
CCCATCGTGGACATCAACATCAAGGACCACAGCATCGTTAGCAGC
TACGTGTGTAGCGGCCTGGTGGGCGATACACCTAGAAAAAACGAC
AGCAGCTCCAGCAGCCACTGCCTGGACCCCAACAATGAAGAAGGC
GGCCACGGAGTGAAGGGATGGGCCTTCGACGACGGCAATGACGTG
TGGATGGGCCGGACCATCTCTGAGAAGAGCCGGTCCGGCTACGAG
ACATTCAAGGTGGTCGAGGGCTGGAGCAACCCCAAGTCCAAGCTG
CAGATCAACCGGCAGGTGATCGTTGACAGAGGCGATCGGAGCGGC
TACAGCGGCATCTTCAGCGTGGAAGGCAAGTCCTGCATCAATCGG
TGTTTCTACGTGGAGCTGATCCGGGGCAGAAAGGAGGAGACCGAG
GTGCTGTGGACCAGCAACTCCATTGTGGTGTTCTGCGGCACCAGC
GGCACCTACGGCACAGGAAGCTGGCCTGACGGCGCCGATCTGAAC
CTGATGCCCATCTAATAA.
In some embodiments, the mRNA Flu B NA-M2e vaccine
has the nucleic acid sequence
(SEQ ID NO: 52)
ATGGACGCCATGAAAAGAGGCCTGTGCTGCGTGCTGCTGCTCTGT
GGCGCCGTGTTCGTGTCCGCCTCTCAGGAGAGCCTGCTGACCGAG
GTTGAGACACCAATTCGGAACGAATGGGGATCTAGATCTAATGAC
AGCAGCGATGGCGGCTCCGGCGGCGGCAGCCTGCTGACCGAGGTG
GAAACCCCTACCAGAAGCGAGTGGGAGTCCAGATCCTCCGACTCT
AGCGACGCCGCTGCCCCCGGCGCCGCTTCTCTGCTGACAGAAGTG
GAAACCCCTACAAGAAACGAGTGGGAGAGCAGATCCAGCGACAGC
TCCGATGCCGCAGGAGGAGGTGCTAGCCTGCTGACAGAGGTGGAA
ACACCCACCCGGACCGGCTGGGAAAGCAACAGCAATGGCAGCAGC
GACGCTGCCCCTGGCGGAAGCGGCATCATCAACGAGACCGCCGAC
GACATCGTGTACAGACTGACCGTGATCATCGACGACAGATACGAG
AGCCTGAAGAACCTGATCACCCTGAGAGCCGACAGACTGGAGATG
ATCATCAACGACAACGTGAGCACCATCCTGGCCAGCGGCGGCAGC
GGCGGCCCCGAGTGGACCTACCCCCGCCTGTCCTGCCCCGGCTCC
ACCTTCCAGAAGGCCCTGCTGATCTCCCCCCACCGCTTCGGCGAG
ACCAAGGGCAACTCCGCCCCCCTGATCATCCGCGAGCCCTTCATC
GCCTGCGGCCCCAAGGAGTGCAAGCACTTCGCCCTGACCCACTAC
GCCGCCCAGCCCGGCGGCTACTACAACGGCACCCGCGAGGACCGC
AACAAGCTGCGCCACCTGATCTCCGTGAAGCTGGGCAAGATCCCC
ACCGTGGAGAACTCCATCTTCCACATGGCCGCCTGGTCCGGCTCC
GCCTGCCACGACGGCAAGGAGTGGACCTACATCGGCGTGGACGGC
CCCGACTCCAACGCCCTGCTGAAGATCAAGTACGGCGAGGCCTAC
ACCGACACCTACCACTCCTACGCCAAGAACATCCTGCGCACCCAG
GAGTCCGCCTGCAACTGCATCGGCGGCGACTGCTACCTGATGATC
ACCGACGGCCCCGCCTCCGGCGTGTCCGAGTGCCGCTTCCTGAAG
ATCCGCGAGGGCCGCATCATCAAGGAGATCTTCCCCACCGGCCGC
GTGAAGCACACCGAGGAGTGCACCTGCGGCTTCGCCTCCAACAAG
ACCATCGAGTGCGCCTGCCGCGACAACTCCTACACCGCCAAGCGC
CCCTTCGTGAAGCTGAACGTGGAGACCGACACCGCCGAGATCCGC
CTGATGTGCACCAAGACCTACCTGGACACCCCCCGCCCCAACGAC
GGCTCCATCACCGGCCCCTGCGAGTCCGACGGCGACAAGGGCTCC
GGCGGCATCAAGGGCGGCTTCGTGCACCAGAGAATGGCCTCCAAG
ATCGGCCGCTGGTACTCCCGCACCATGTCCAAGACCAAGAGAATG
GGCATGGGCCTGTACGTGAAGTACGACGGCGACCCCTGGACCGAC
TCCGAGGCCCTGGCCCTGTCCGGCGTGATGGTGTCCATGGAGGAG
CCCGGCTGGTACTCCTTCGGCTTCGAGATCAAGGACAAGAAGTGC
GACGTGCCCTGCATCGGCATCGAGATGGTGCACGACGGCGGCAAG
ACCACCTGGCACTCCGCCGCCACCGCCATCTACTGCCTGATGGGC
TCCGGCCAGCTGCTGTGGGACACCGTGACCGGCGTGAACATGACC
CTGTAATAA.
In some embodiments, the H1 full-length HA mRNA
has the nucleic acid sequence
(SEQ ID NO: 53)
ATGAAAGCCATCCTGGTGGTGCTCCTGTACACCTTCGCCACCGCT
AATGCCGACACCCTCTGCATCGGCTATCACGCCAACAACAGCACA
GATACAGTGGACACCGTGCTGGAAAAGAATGTTACAGTCACCCAC
AGCGTGAACCTGCTGGAAGACAAGCACAACGGCAAGCTCTGCAAA
CTGAGAGGAGTGGCCCCTCTGCACCTGGGCAAATGTAATATCGCC
GGATGGATCCTGGGCAACCCCGAGTGCGAGTCTCTGAGTACCGCT
TCTAGCTGGTCCTACATCGTGGAAACAAGCAGCTCCGATAACGGC
ACATGCTACCCCGGCGACTTCATCAACTACGAGGAACTGAGAGAA
CAACTGAGCAGCGTGTCCTCTTTCGAGCGGTTCGAGATCTTCCCT
AAGACCTCCAGCTGGCCTAACCACGAGAGCAACAAGGGAGTGACC
GCTGCTTGTCCTCACGCCGGCGCCAAGAGCTTCTACAAGAACCTG
ATCTGGCTGGTCAAGAAGGGCAACTCTTACCCTAAGCTGAGCCAG
TCCTACATCAACGACAAGGGCAAGGAGGTGCTGGTGCTGTGGGGC
ATCCACCACCCACCCACCACAGCCGACCAGCAGAGCCTGTACCAA
AACGCCGACGCCTATGTGTTCGTGGGCACCAGCAGATACAGCAAG
AAATTCAAGCCCGAGATCGCCATCCGGCCCAAGGTGCGGGACCAG
GAGGGAAGGATGAACTACTACTGGACACTGGTGGAACCCGGCGAT
AAGATCACCTTTGAGGCCACTGGGAATCTGGTGGTGCCCAGGTAC
GCCTTCACCATGGAACGGAACGCTGGCAGCGGCATTATCATCAGC
GACACCCCCGTGCATGACTGCAACACAACATGCCAGACACCCGAA
GGCGCAATCAACACCTCTCTGCCCTTTCAGAACATCCACCCCATC
ACAATCGGCAAGTGCCCCAAATACGTGAAGTCCACCAAGCTGCGG
CTGGCTACCGGCCTGAGGAACGTGCCCAGCATCCAGAGCCGGGGT
CTGTTCGGTGCCATCGCCGGCTTTATCGAGGGCGGCTGGACCGGC
ATGGTGGATGGATGGTACGGCTACCACCACCAGAACGAGCAGGGC
AGCGGCTACGCCGCCGATCTGAAGAGCACTCAGAACGCCATCGAC
AAGATCACCAACAAGGTGAACTCTGTGATCGAGAAGATGAACACA
CAGTTCACCGCCGTGGGAAAGGAGTTCAACCACCTGGAGAAGCGG
ATCGAAAATCTGAACAAGAAGGTGGACGACGGCTTCCTGGATATC
TGGACCTACAACGCCGAGCTGCTGGTCCTGCTCGAGAACGAGCGG
ACCCTGGACTACCACGATAGCAACGTGAAGAATCTGTACGAGAAA
GTGCGGAACCAGCTGAAGAACAACGCCAAGGAAATCGGGAACGGC
TGCTTCGAGTTCTACCATAAATGCGACAACACCTGCATGGAAAGC
GTGAAGAATGGCACCTACGACTACCCCAAGTACTCCGAGGAAGCC
AAGCTGAACCGGGAGAAGATCGACGGCGTGAAGCTGGAATCTACC
CGGATATATCAGATCCTGGCCATCTACAGCACCGTGGCCAGCAGC
GTGGTCCTGGTGGTGTCTCTGGGCGCCATTAGCTTTTGGATGTGC
AGCAACGGCAGCCTGCAGTGCCGGATCTGCATCTAATAA.

### Polynucleotides and Cells

Also disclosed are polynucleotides comprising nucleic acid sequences encoding the disclosed fusion proteins. For example, the nucleic acid sequences can be operably linked to expression control sequences. Thus, also disclosed are expression vectors for producing the disclosed fusion proteins as well as cells containing these polynucleotides and vectors for replicating the polynucleotides and vectors or to produce the disclose fusion proteins and/or VLPs. Therefore, the disclosed cell can also contain nucleic acid sequences encoding an M1 protein, including a vector comprising the nucleic acid sequences encoding an M1 protein.

The cell can be a prokaryotic or eukaryotic cell. For example, the cell can be a bacterium, an insect cell, a yeast cell, or a mammalian cell. The cell can be a human cell. Suitable vectors can be routinely selected based on the choice of cell used to produce the VLP. For example, where insect cells are used, suitable vectors include baculoviruses.

### Fusion Proteins

Fusion proteins, also known as chimeric proteins, are proteins created through the joining of two or more genes which originally coded for separate proteins. Translation of this fusion gene results in a single polypeptide with function properties derived from each of the original proteins. Recombinant fusion proteins can be created artificially by recombinant DNA technology for use in biological research or therapeutics.

The functionality of fusion proteins is made possible by the fact that many protein functional domains are modular. In other words, the linear portion of a polypeptide which corresponds to a given domain, such as a tyrosine kinase domain, may be removed from the rest of the protein without destroying its intrinsic enzymatic capability. Thus, any of the herein disclosed functional domains can be used to design a fusion protein.

A recombinant fusion protein is a protein created through genetic engineering of a fusion gene. This typically involves removing the stop codon from a cDNA sequence coding for the first protein, then appending the cDNA sequence of the second protein in frame through ligation or overlap extension PCR. That DNA sequence will then be expressed by a cell as a single protein. The protein can be engineered to include the full sequence of both original proteins, or only a portion of either.

If the two entities are proteins, often linker (or “spacer”) peptides are also added which make it more likely that the proteins fold independently and behave as expected. Especially in the case where the linkers enable protein purification, linkers in protein or peptide fusions are sometimes engineered with cleavage sites for proteases or chemical agents which enable the liberation of the two separate proteins.

### Virus Like Particles (VLPs)

The disclosed construct of heterologous M2e sequences may be expressed on the surface of a particle to mimic the natural conformation of M2 on influenza virions. For example, the disclosed fusion proteins may be incorporated into virus-like particles (VLPs) by including within the fusion protein a membrane anchor domain, such as a transmembrane domain and optional cytoplasmic domain of a viral envelope protein.

Non-replicating VLPs resemble infectious virus particles in structure and morphology, and contain immunologically relevant viral structural proteins. VLPs have been produced from both non-enveloped and enveloped viruses. Envelopes of VLPs are derived from the host cells similar to the way as enveloped viruses such as influenza A virus obtain their lipid envelopes from their host cells. Therefore, membrane-anchored proteins on the surfaces of enveloped viruses will be expressed in a native-like conformation if they are expressed in a membrane-anchored form.

Influenza VLPs involve lipid bilayers and host cell membrane proteins (Song, J. M., et al. J Proteome Res 2011 10:3450-3459). For example, Influenza VLPs containing the wild type M2 protein have been described (Song, J. M., et al. Proc Natl Acad Sci USA 2011 108:757-761; Song, J. M., et al. PLoS One 2011 6:e14538). Enveloped VLPs may be composed of influenza matrix 1 (M1) protein as a particle forming core. These VLPs are produced, for example, by coinfecting insect cells with one or more recombinant baculoviruses co-expressing M1 proteins and the disclosed fusion proteins, culturing the insect cells under physiological conditions, and purifying the VLPs from insect cell culture supernatants.

Influenza virus hemagglutinin (HA) and neuraminidase (NA) are large proteins that have the potential to mask smaller M2e proteins. Therefore, in some embodiments, HA and NA are not immobilized on the surface of the VLP.

mRNA vaccine platforms: The mRNA vaccine platform has many advantages over conventional flu vaccine approaches, which include rapid, scalable, sequence-independent production of mRNA vaccines, high flexibility to design new variant antigens, enabling easy combination of several antigen-encoding mRNAs into a single formulation, and immediate in vivo exposure of native-like antigens to the immune system. COVID-19 mRNA vaccines have been licensed earlier than other recombinant platforms. The technology of mRNA vaccine platform was found to be effective in expressing universal vaccine candidate protein antigens (5xM2e, chimeric fusion M2e-H3stalk proteins, consensus NA protein or chimeric M2e-NA fusion proteins). mRNA vaccines formulated in lipid nanoparticles (LNP) were immunogenic, inducing protective antibodies and protection against influenza viruses.

### Vaccine Compositions

Disclosed are vaccine compositions that comprise one or more of the fusion proteins described above. Although not required, the vaccine compositions optionally contain one or more immunostimulants. An immunostimulant refers to essentially any substance that enhances or potentiates an immune response (antibody or cell-mediated) to an exogenous antigen. One preferred type of immunostimulant is an adjuvant.

The disclosed vaccines can be used therapeutically in combination with a pharmaceutically acceptable carrier. By “pharmaceutically acceptable” is meant a material that is not biologically or otherwise undesirable, i.e., the material may be administered to a subject without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The materials may be in solution, suspension (for example, incorporated into microparticles, liposomes, or cells). Suitable carriers and their formulations are described in Remington: The Science and Practice of Pharmacy (22nd ed.) eds. Loyd V. Allen, Jr., et al., Pharmaceutical Press, 2012. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of vaccines to humans, including solutions such as sterile water, saline, and buffered solutions at physiological pH. Pharmaceutical compositions may include carriers, thickeners, diluents, buffers, preservatives, surface active agents and the like in addition to the vaccine. Pharmaceutical compositions may also include one or more active ingredients such as antimicrobial agents, antiinflammatory agents, anesthetics, and the like.

The disclosed vaccines are preferably formulated for delivery via intranasal, intramuscular, subcutaneous, transdermal or sublingual administration.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

### Combinations

The disclosed vaccine can be used to supplement existing human vaccines to improve cross protection. Therefore, the disclosed vaccine can further include (or be administered in combination with) a whole inactivated virus, split viral vaccine, live attenuated influenza vaccine, or another influenza virus-like particle (VLP) vaccine. For example, the disclosed vaccine can be combined with a trivalent inactivated vaccine (TIV) (e.g., containing killed A/H1N1, A/H3N2, and B), trivalent live attenuated influenza vaccine, trivalent split vaccines, or trivalent subunit influenza vaccines.

The disclosed vaccine can further include (or be administered in combination with) one or more of classes of antibiotics, steroids, analgesics, anti-inflammatory agents, anti-histaminic agents, or any combination thereof. Antibiotics include Aminoglycosides, Cephalosporins, Chloramphenicol, Clindamycin, Erythromycins, Fluoroquinolones, Macrolides, Azolides, Metronidazole, Penicillins, Tetracyclines, Trimethoprim-sulfamethoxazole, and Vancomycin. Suitable steroids include andranes, such as testosterone. Narcotic and non-narcotic analgesics include morphine, codeine, heroin, hydromorphone, levorphanol, meperidine, methadone, oxydone, propoxyphene, fentanyl, methadone, naloxone, buprenorphine, butorphanol, nalbuphine, and pentazocine. Anti-inflammatory agents include alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, decanoate, deflazacort, delatestryl, depo-testosterone, desonide, desoximetasone, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, mesterolone, methandrostenolone, methenolone, methenolone acetate, methylprednisolone suleptanate, momiflumate, nabumetone, nandrolone, naproxen, naproxen sodium, naproxol, nimazone, olsalazine sodium, orgotein, orpanoxin, oxandrolane, oxaprozin, oxyphenbutazone, oxymetholone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, stanozolol, sudoxicam, sulindac, suprofen, talmetacin, talniflumate, talosalate, tebufelone, tenidap, tenidap sodium, tenoxicam, tesicam, tesimide, testosterone, testosterone blends, tetrydamine, tiopinac, tixocortol pivalate, tolmetin, tolmetin sodium, triclonide, triflumidate, zidometacin, and zomepirac sodium. Anti-histaminic agents include ethanolamines (e.g., diphenhydrmine carbinoxamine), Ethylenediamine (e.g., tripelennamine pyrilamine), Alkylamine (e.g., chlorpheniramine, dexchlorpheniramine, brompheniramine, triprolidine), other anti-histamines like astemizole, loratadine, fexofenadine, bropheniramine, clemastine, acetaminophen, pseudoephedrine, triprolidine).

### Methods of Vaccinating a Subject

A method of vaccinating a subject for influenza A is disclosed that involves administering the disclosed cross-protective influenza vaccine to a subject in need thereof. The disclosed vaccine may be administered in a number of ways. For example, the disclosed vaccine can be administered intramuscularly, intranasally, or by microneedle in the skin. The compositions may be administered orally, intravenously, subcutaneously, transdermally (e.g., by microneedle), intraperitoneally, ophthalmically, vaginally, rectally, sublingually, or by inhalation.

Parenteral administration of the composition, if used, is generally characterized by injection. Injectables can be prepared in conventional forms, either as liquid solutions or suspensions, solid forms suitable for solution of suspension in liquid prior to injection, or as emulsions. A revised approach for parenteral administration involves use of a slow release or sustained release system such that a constant dosage is maintained.

The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. A typical dosage of the disclosed vaccine used alone might range from about 1 μg/kg to up to 100 mg/kg of body weight or more per vaccination, such as 10 μg/kg to 50 mg/kg, or 50 μg/kg to 10 mg/kg, depending on the factors mentioned above.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Thermostable Engineered H1 Hemagglutinin Stem with M2e Epitopes Provides Broad Cross-Protection Against Group 1 and 2 Influenza a Viruses

### Rational Design and Generation of Chimeric M2e H1Stem Fusion Construct

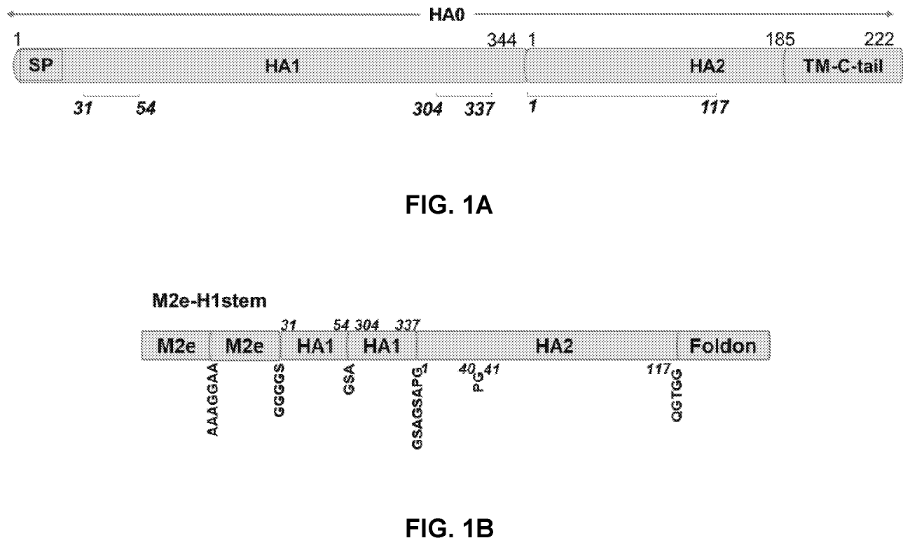

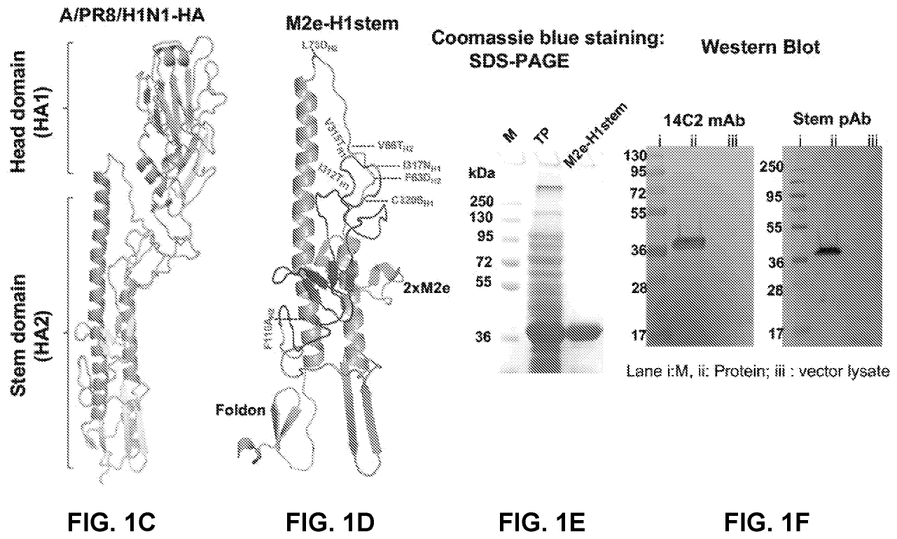

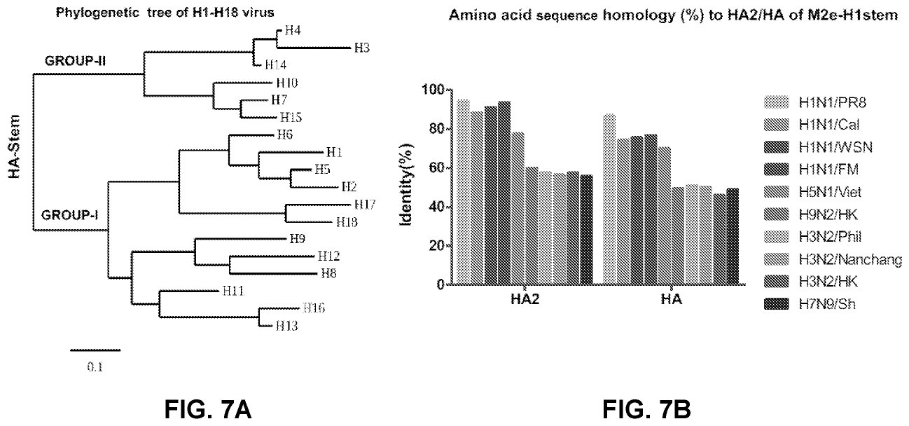

HA comprises HA1 globular, receptor-binding, variable head domain, and fusion inducing, comparatively conserved stem domain (FIGS. 1A and 1C). N- and C-terminal of the HA1 parts contribute to stabilizing the HA2 stem domain, providing the rationale for including HA1 parts in the M2e-H2stem construct. To overcome the low efficacy of stem-based vaccines in conferring cross-group protection due to HA stem sequence variations (FIGS. 7A-7B), we designed a chimeric M2e-stem fusion construct (FIGS. 1B and 1D). Two M2e domains were genetically linked to the N-terminal of the stem domain derived from A/PR8 HA (M2e-H1stem). The cysteine residues at position 17 and 19 in M2e were changed to serine to avoid unwanted cross links. To improve the immune response targeting stem and fusion domain epitopes and to minimize potential protein aggregation due to the exposure of hydrophobic residues on the surface of HA2, we deleted the C-terminal of the HA2 domain. The M2e-H1 stem construct contains 2xM2e, HA1 parts [aa 31-54, aa 304-337], and HA2 stem in α-helix [aa 1-117] (FIGS. 1B and 1D). Point mutations known to improve expression 8 were introduced in the hydrophobic patches in both HA1 (1312T, V315T, 1317N) and HA2 stem domains (F63D, 119 V66T, L73D, F110A). We also mutated cysteine residue into serine (C320S) to avoid intramolecular disulfide bond formation (FIG. 1D). Each domain is connected via flexible linkers to facilitate its domain-independent native-like conformation. Foldon was shown to be important for stabilizing the recombinant proteins and proteolytic resistance8. The β-rich trimeric foldon 23 is linked to the C-terminus of M2e-H1stem. Codon-optimized M2e-H1stem gene was synthesized and cloned into pCold II vector to induce protein expression at a low temperature in Escherichia coli (E. Coli). The M2e-H1stem protein was expressed at high levels (>50% of cell lysates in SDS-PAGE Coomassie Blue stain) in Rosetta (DE3) pLysS cells and affinity-purified (FIG. 1E); confirmed by Western blot using M2e-specific monoclonal antibody (mAb; 14C2) and stem-specific polyclonal antibody (pAb) (FIG. 1F).

### M2e-H1Stem Protein Displays Cross-Reactive Antigenicity

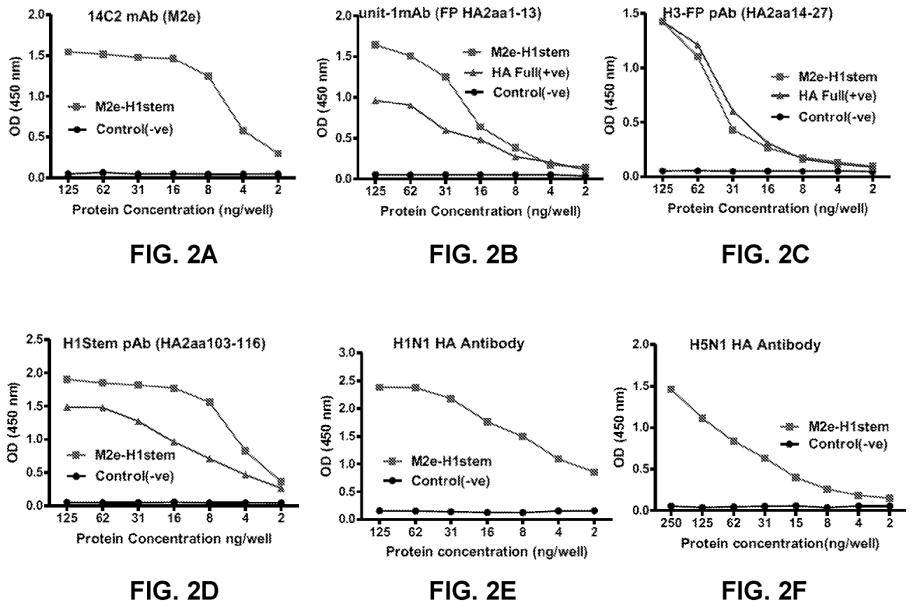

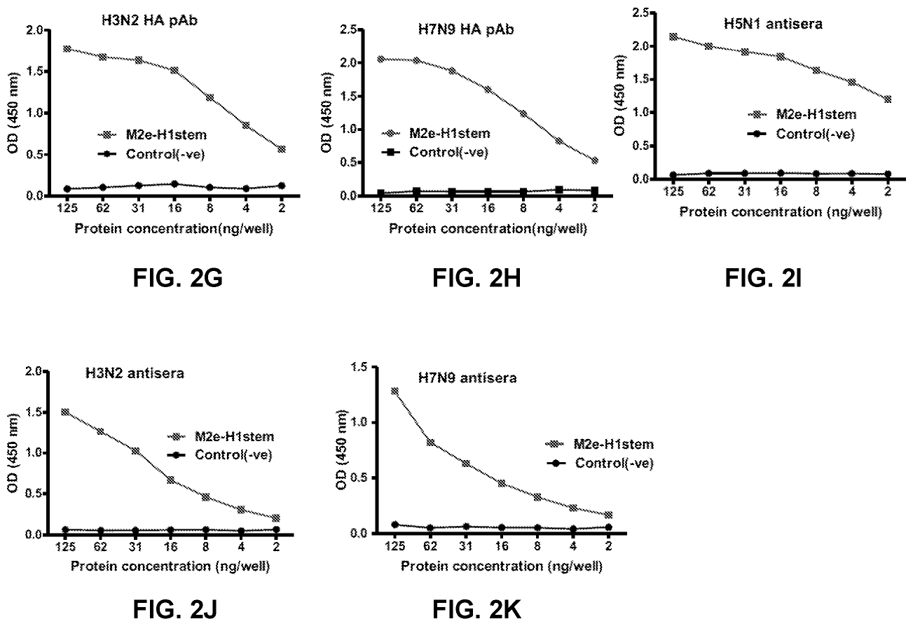

The antigenic epitope properties of M2e-H1stem protein with M2e specific, HA2 stem-specific, and virus-specific antibodies was investigated (FIGS. 2A-2K). M2e-H1stem was found to be reactive with M2e mAb (14C2) and pAbs raised against highly conserved HA2 aa1-13 fusion peptide, HA2 aa14-27, and HA2 aa103-116 (FIGS. 2A-2D). HA2 stem-specific pAbs also recognized a full-length HA, supporting the specific binding of these pAbs to native stem epitopes (FIGS. 2B-2D). In addition, M2e-H1stem was cross recognized by pAbs induced by different HA proteins derived from diverse subtypes, including H1N1, H5N1, H3N2, and H7N9 (FIGS. 2E-2H). Notably, M2e-H1stem was antigenically cross-reactive and recognized by antisera from infection with different subtypes of influenza viruses, such as H5N1, H3N2, and H7N9 at significant levels (FIGS. 2I-2K), suggesting the presence of native-like epitopes. Taken together, these results suggest that M2e-H1stem protein exposes native-like, conserved epitopes recognized by different pAbs against group 1 and 2 HA subtypes of influenza A viruses.

### Antisera of M2e-H1Stem Vaccination Recoqnize M2e, Stem and Cross-Qroup Viruses

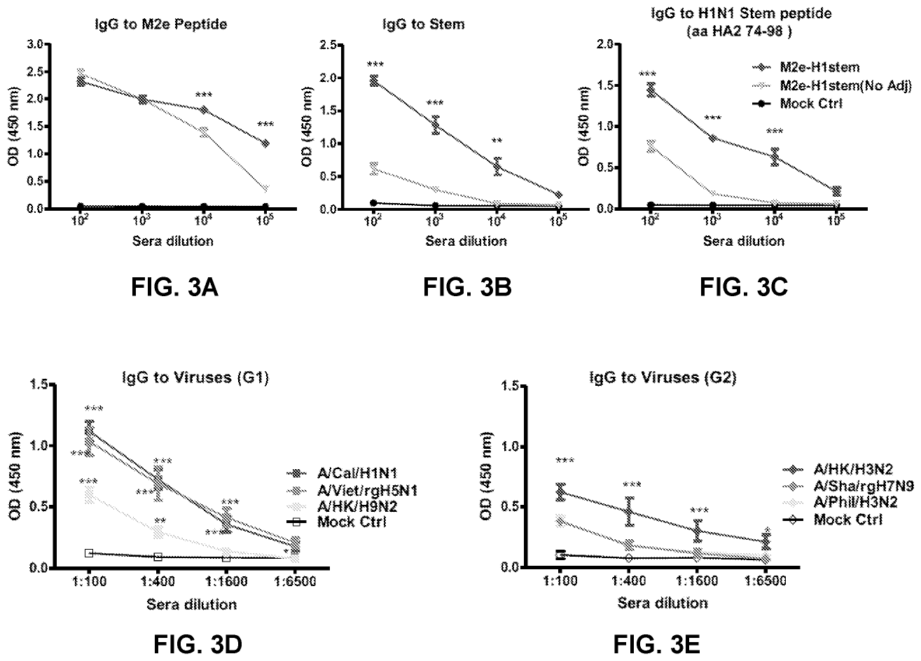

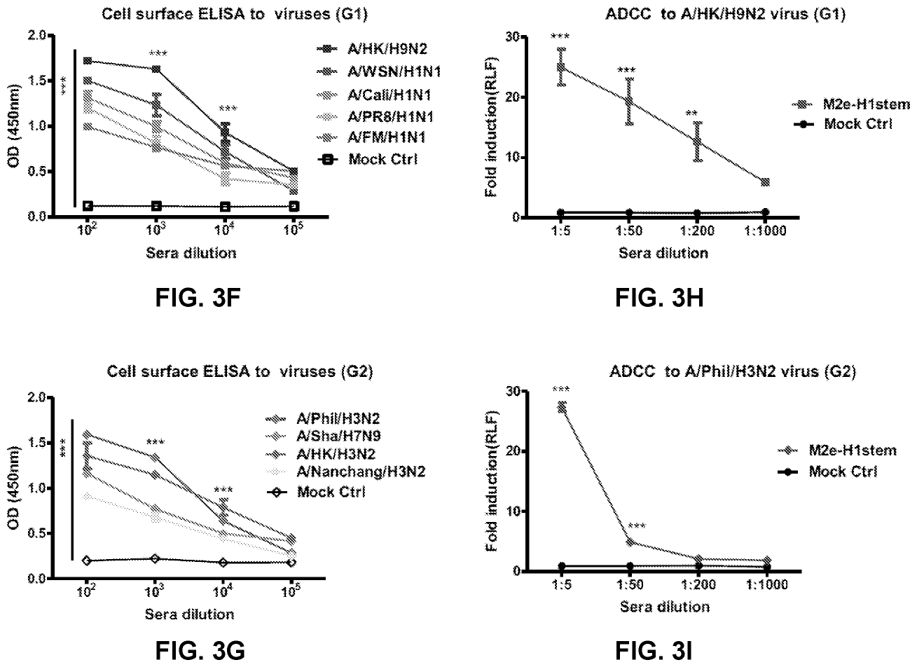

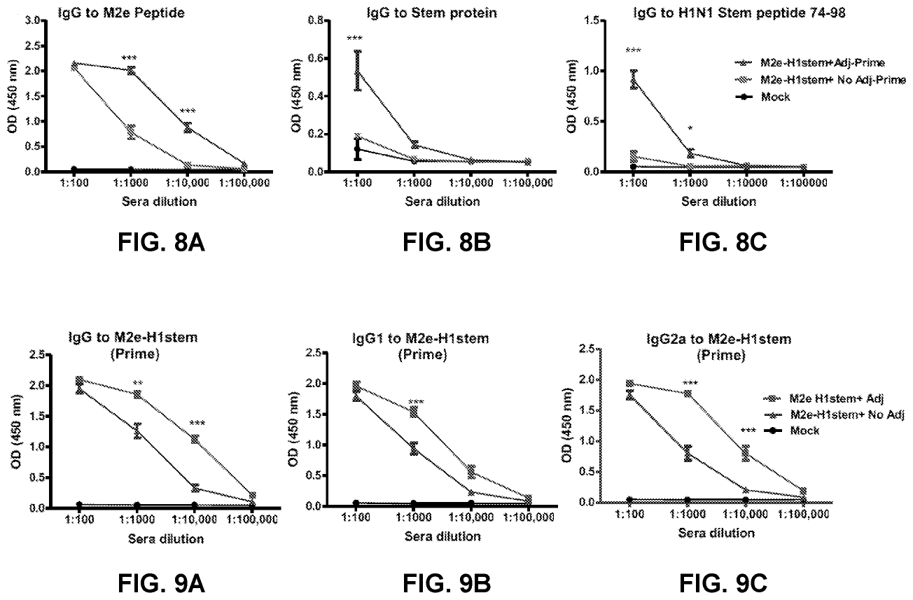

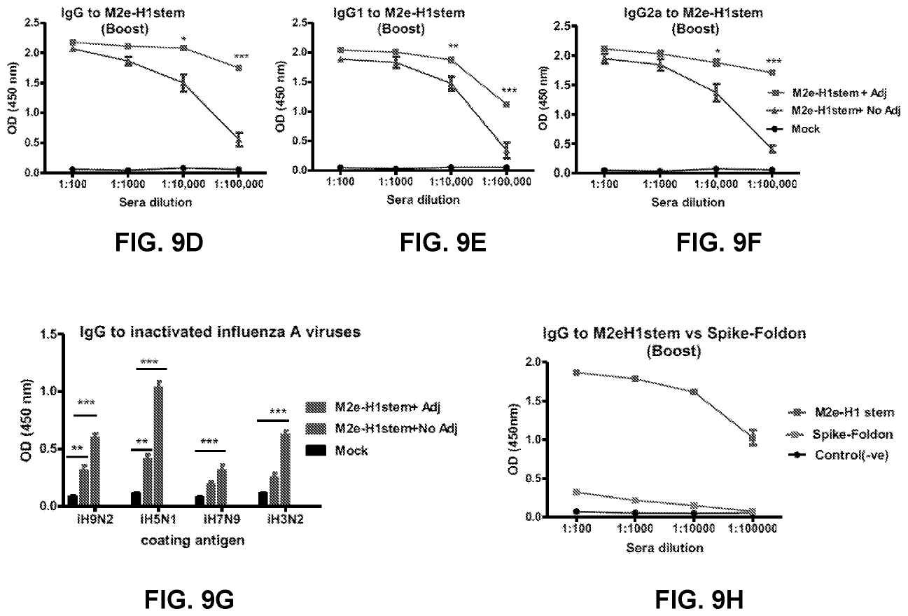

Liposome adjuvant AS01 (QS-21+MPL) is licensed for use in herpes zoster vaccination 24,25. Therefore, we determined whether the inclusion of AS01-like adjuvant (QS-21+MPL) would enhance vaccine antigen specific IgG antibody responses at two weeks after intramuscular (IM) vaccination of BALB/c mice with M2e-H1stem protein (20 μg). After prime, even in the absence of adjuvant, M2e-specific IgG Abs were induced at substantial levels, whereas stem peptide (HA2 aa 74-98) and stem protein-specific IgG levels were very low (FIGS. 8A-8C). With adjuvanted prime vaccination, IgG Abs specific for M2e and stem protein, as well as stem peptide were induced at significantly higher levels than non-adjuvanted counterpart (FIGS. 8A-8C). After boost dose, the levels of IgG Abs specific for M2e and HA2 stem protein as well as HA2 stem peptide (HA2 aa74-98) were significantly increased in the adjuvanted M2e-H1stem group, compared to unadjuvanted vaccination (FIGS. 3A-3C). Adjuvanted M2e-H1stem prime or prime-boost vaccination also induced IgG, IgG1, and IgG2a isotype Abs specific for M2e-H1 stem vaccine antigen at significantly higher levels than the unadjuvanted vaccination (FIGS. 9A-9F). The level of IgG antibodies binding to the foldon linked spike protein of SARS-CoV-2 was close to the background, suggesting that adjuvanted M2e-H1stem vaccination induced IgG responses specific for M2e-H1stem but not for foldon domain (FIG. 9H). Consistently, when viruses were used as a coating antigen, the adjuvanted M2e-H1stem-vaccinated group showed significantly higher levels of IgG Abs recognizing group 1 (rgA/Viet/H5N1, rgA/HK/H9N2) and group 2 (A/HK/H3N2, rgA/Shanghai/H7N9) viruses than those from the unadjuvanted vaccine group (FIG. 9G). IgG Ab responses specific for the group 1 (H1N1, H5N1, H9N2) viruses were higher than those against the group 2 (H3N2, H7N9) viruses in antisera from adjuvanted M2e-H1stem vaccination (FIGS. 3D-3E).

### Antisera of M2e-H1Stem Recoqnize Cell Surface-Exposed Viral Antiqens and Exhibit ADCC

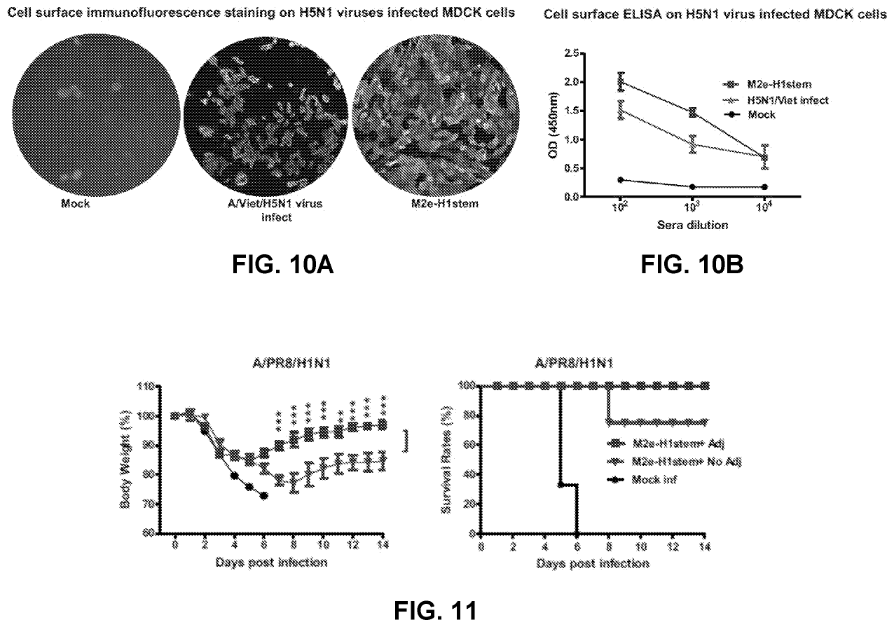

Immune sera of adjuvanted M2e-H1stem vaccination were highly reactive to the MDCK cell surface viral antigens after infection with group 1 [H1N1 (A/WSN, A/Cal, A/PR8, A/FM), A/HK/H9N2, A/Viet/H5N1] and group 2 viruses [H3N2 (A/Phil, A/HK, A/Nanchang), A/Sha/H7N9](FIGS. 3F-3G and 10A-10B). Consistently, antibody-dependent cell mediated cytotoxicity (ADCC) surrogate assay in influenza A virus-infected MDCK cells showed strong reporter signals in group 1 (FIG. 3H) and group 2 (FIG. 3I) virus-infected cells, suggesting that M2e-H1stem immune sera function via ADCC. Altogether, these results indicate that adjuvanted M2e-H1stem vaccination effectively induced antibodies recognizing M2e, stem, group 1 and 2 viruses as well as engaging in Fc-mediated ADCC, contributing to cross protection.

### M2e-H1Stem Provides Cross-Group Protection Against Diverse Group 1 and 2 Viruses

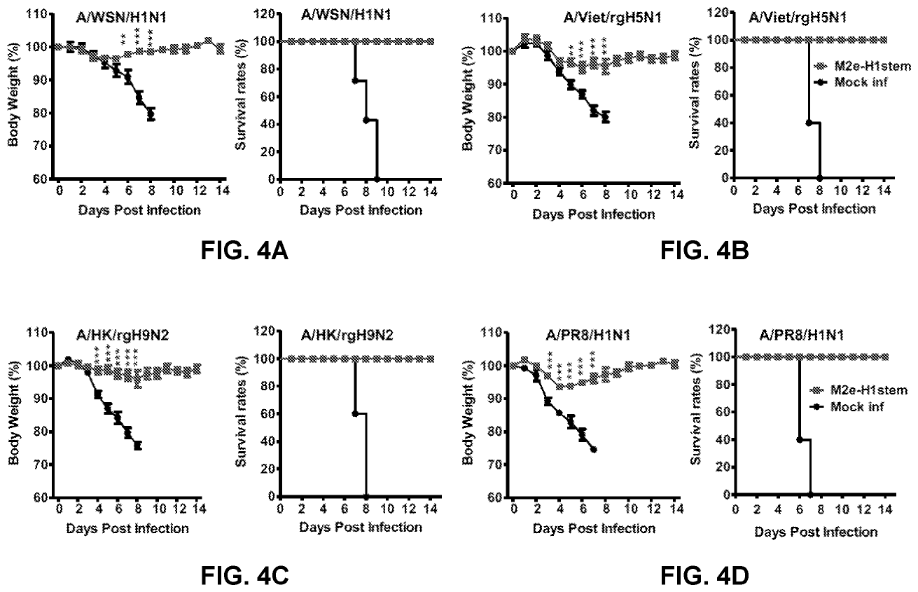

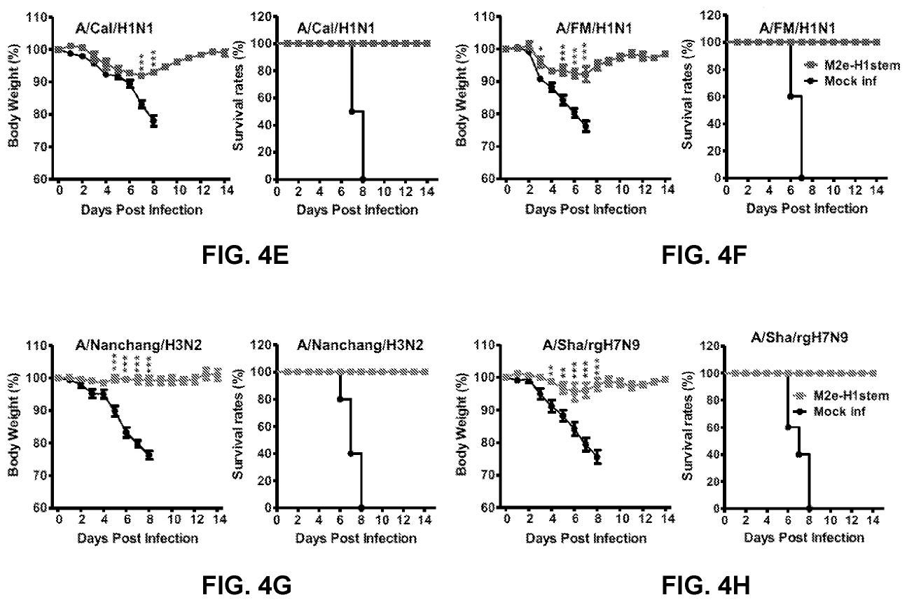

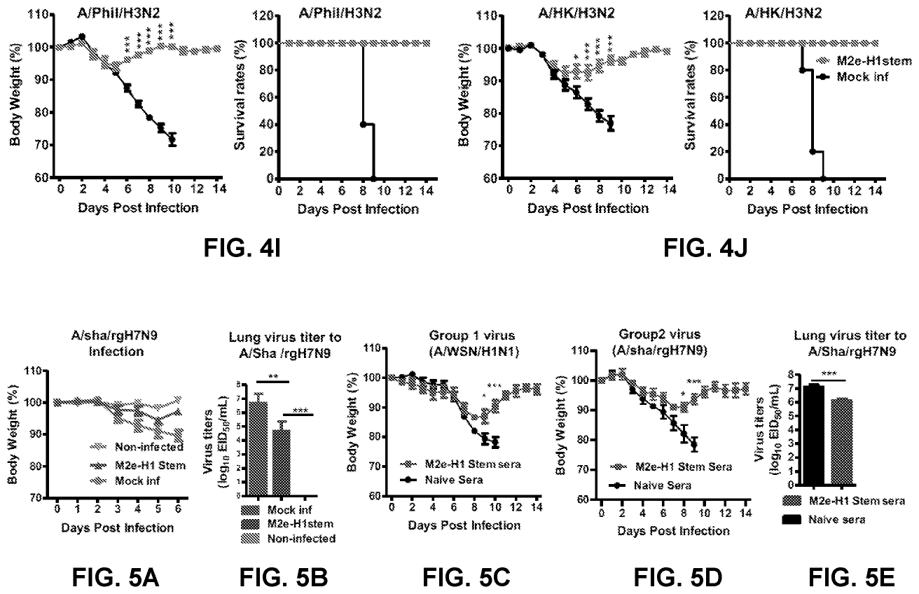

The inclusion of adjuvant (QS-21+MPL) was found to significantly enhance the efficacy of M2e-H1stem vaccination by preventing severe weight loss and promoting recovery after lethal challenge with A/PR8/H1N1 virus (FIG. 11), we focused adjuvanted vaccine on testing the breadth of cross-protection. Groups of mice with M2e-H1stem prime-boost vaccination were challenged with a lethal dose of antigenically different group 1 viruses (FIGS. 4A-4F). Effective protection against A/WSN/1933 H1N1 (FIG. 4A), rgA/Viet/2004 H5N1 (FIG. 4B), and rgA/HK/1999 H9N2 (FIG. 4C) was observed in vaccinated mice with minimum weight loss (˜2-5%); mock control mice did not survive. Moderate weight loss (6-8%) was observed in M2e-H1stem vaccinated mice after lethal challenge with pathogenic H1N1 viruses, including A/PR8/1934 (FIG. 4D), A/Cal/2009 (FIG. 4E), and A/FM/1947 (FIG. 4F). Moreover, we determined whether M2e-H1stem vaccination could provide cross protection against group 2 viruses. Remarkably, the groups of mice vaccinated with M2e-H1stem were well protected against lethal challenges with A/Nanchang/1995 H3N2 (FIG. 4G) and rgA/Shanghai/2013 rgH7N9 (FIG. 4H), as evidenced by no and minimum (˜5%) weight loss, respectively. When challenged with A/Phil/1982 H3N2 (FIG. 4I) and A/HK/1968 H3N2 (FIG. 4J) virus, M2e-H1stem vaccinated mice showed low to moderate (˜7%) weight loss and recovered. Mock control mice experienced severe weight loss and died of each virus infection (FIGS. 4A-4J). These results suggest that M2e-H1stem vaccination conferred broad cross protection against antigenically different group 1 and 2 influenza A viruses.

### M2e-H1Stem Vaccination Promotes Lung-Viral Clearance, Protective Humoral and Cellular Immunity

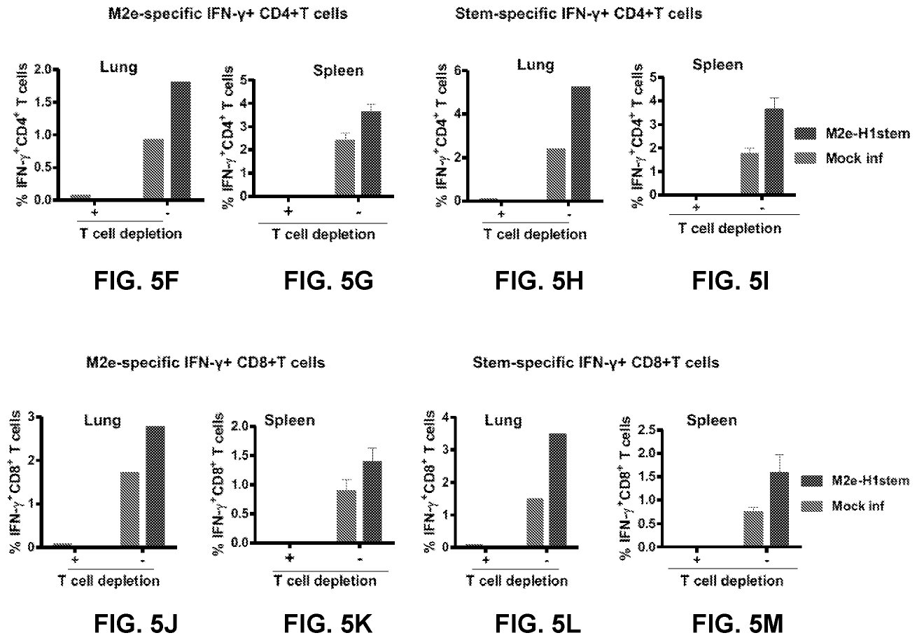

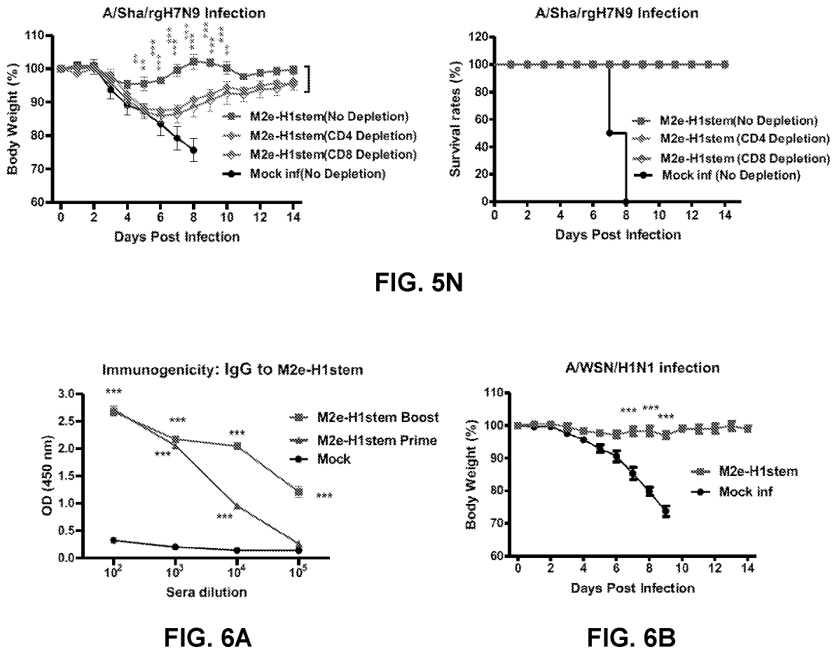

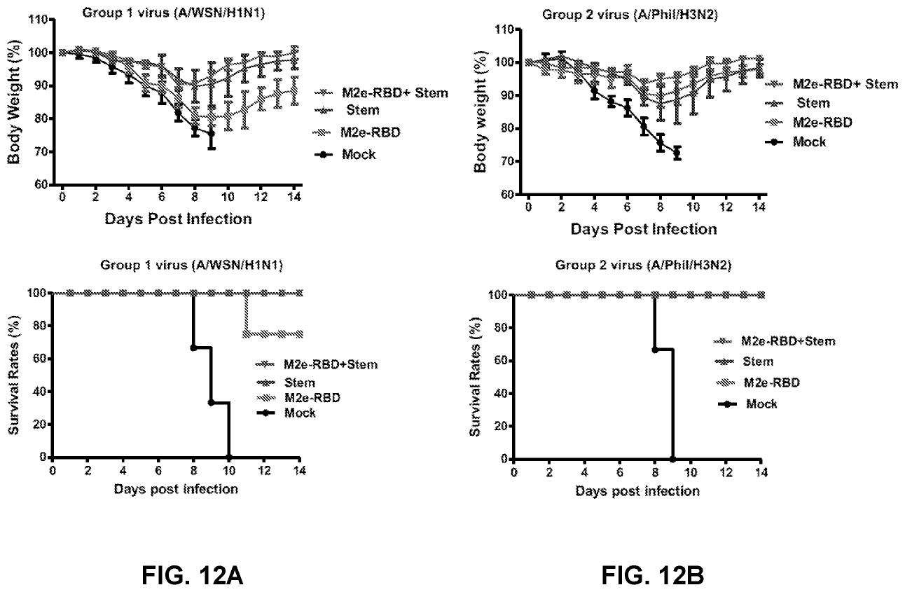

To further assess the effectiveness of cross-group protection, we determined lung viral titers as well as humoral and cellular immune responses. Compared to the unvaccinated mock control group after infection, M2e-H1stem vaccination led to rapid recovery of weight loss (FIG. 5A) and effective lung viral clearance by 1000-fold lower upon rgH7N9 infection (FIG. 5B). A modified passive transfer assay was reported as an effective and sensitive method to determine the roles and contribution of antisera in conferring protection in naïve mice, which we demonstrated in previous studies. To determine the roles of antiserum humoral immune responses in conferring cross-protection, naïve mice were intranasally infected with a mixture of virus and immune sera collected from M2e-H1stem-vaccinated mice (FIGS. 5C-5E). Naïve sera did not protect against rgH7N9 or A/WSN (H1N1) virus, as evidenced by severe weight loss and 0% survival. In contrast, M2e-H1stem immune sera conferred protection in naïve mice with moderate weight loss (˜10% and 13%, respectively) (FIGS. 5C-5D). M2e-H1stem immune sera lowered lung viral titers by 10-fold at day 5 post infection of rgH7N9 (FIG. 5E). To further determine the contribution and roles of M2e and HA stem antibodies in protection, naïve mice were intranasally infected with a mixture of group 1 virus (A/WSN/H1N1) or group 2 virus (A/Phil/H3N2) with antisera of M2e or group 1 HA2 stem or a mixture of M2e and group 1 HA2 stem (FIGS. 12A-12B). The group 1 HA2 stem group and the combination group of M2e-RBD and HA2 stem sera showed better protection (˜10% weight loss) against A/WSN/H1N1 compared to M2e alone antisera (˜20% weight loss). Importantly, the combination group of M2e and group 1 HA2 stem antisera more effectively prevented weight loss (˜6%) against group 2 virus A/Phil/H3N2, suggesting significantly improved cross protection, compared to the M2e or group 1 HA2 stem alone antiserum group displaying approximately 13% weight loss (FIGS. 12A-12B).

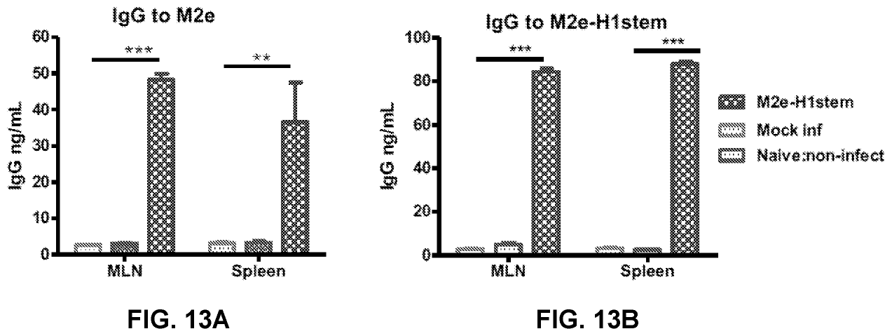

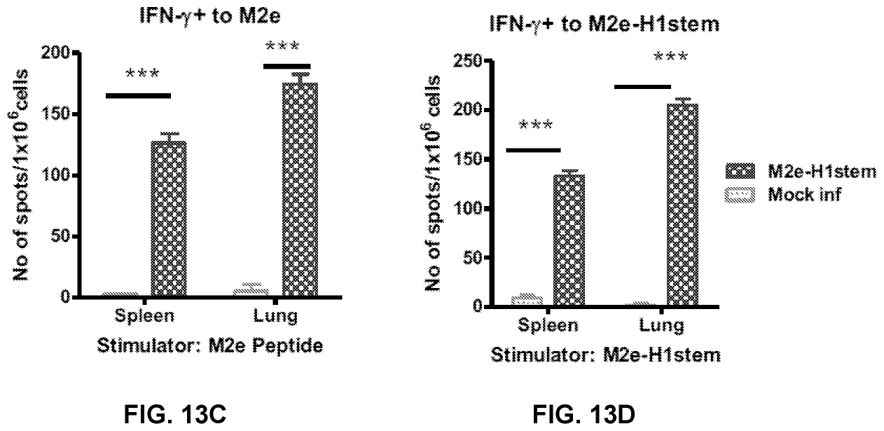

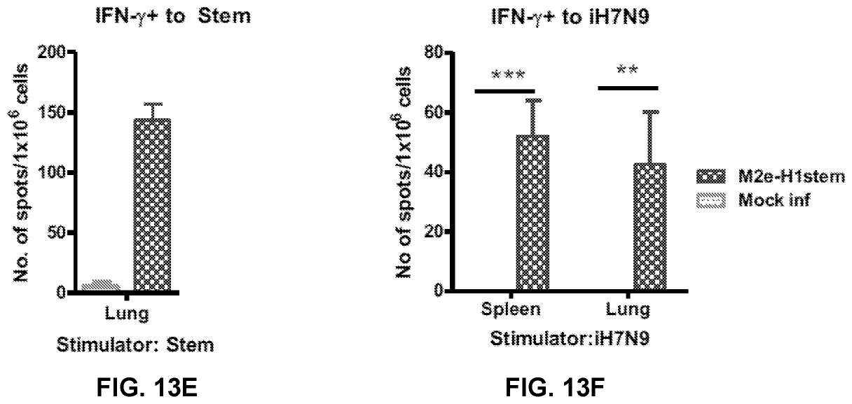

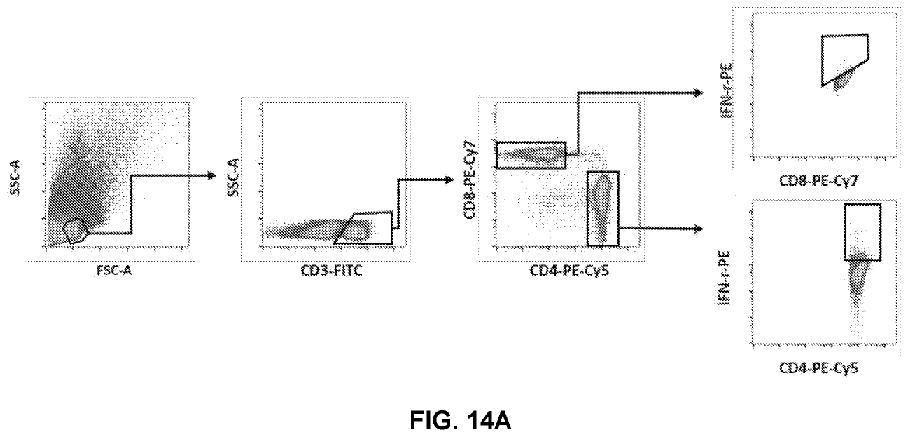

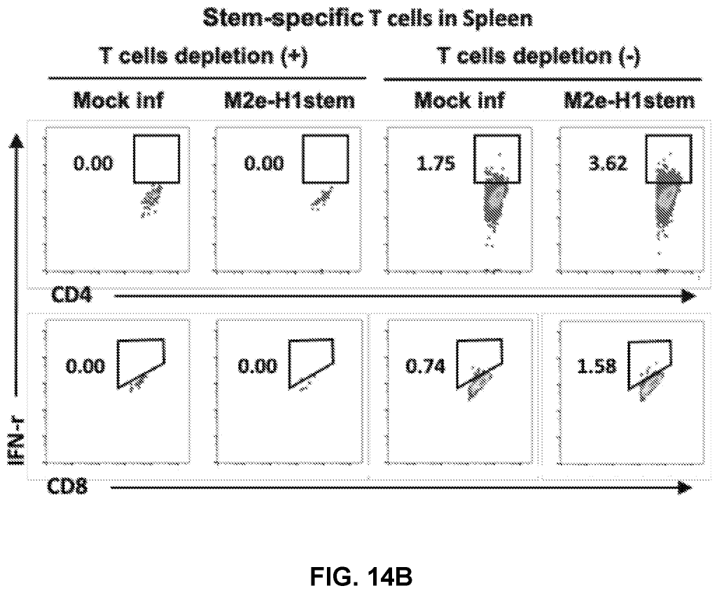

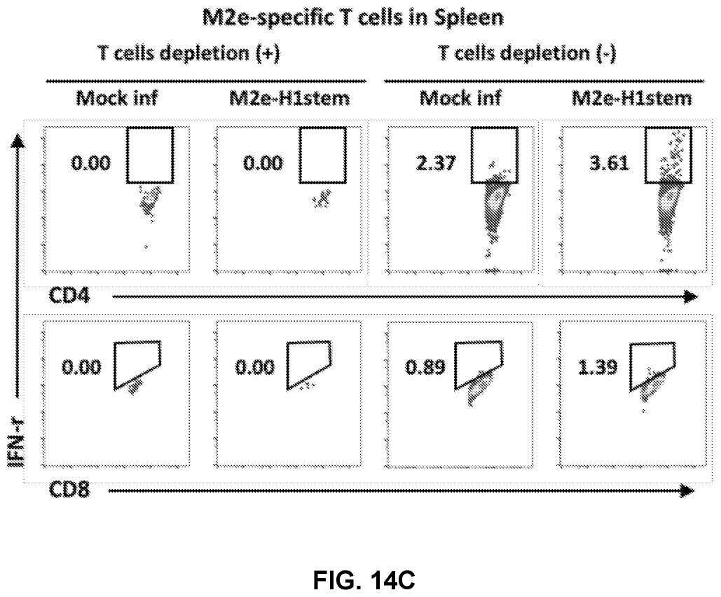

In addition, the mediastinal lymph nodes (MLN) and spleens collected from the M2e-H1stem vaccination group were effective in secreting M2e-specific IgG and chimeric M2e H1stem-specific IgG Abs after in vitro culturing, compared to the unvaccinated control group (FIGS. 13A-13B). These data suggest the presence of B cells (memory-like) rapidly responding to the induction of IgG antibodies specific for M2e and HA2 stem antigens upon antigenically different virus exposure. Furthermore, M2e-H1stem group induced significantly high levels of IFN-γ+ secreting T cells in spleen and lung after in vitro stimulation with M2e, M2e-H1stem, stem, and inactivated rgA/Sha/H7N9 virus (FIGS. 13C-13F); these data implicated that M2e-H1stem vaccination induced the generation of T cells rapidly responding to secret IFN-γ+ upon challenge. Consistently, flow cytometry of intracellular cytokine staining showed that M2e or stem-specific IFN-γ+ CD4+ T and IFN-γ+ CD8+ T cells were increased in the lung and spleen cells in the adjuvanted M2e-H1stem group compared to the unvaccinated control (FIGS. 5F-5M and 14A-14C). To further investigate whether T cell immunity would contribute to protection, each of CD4+ and CD8+ T cells was depleted from the mice immunized with M2e-H1stem prior to rgA/Sha/H7N9 challenge (FIG. 5N). Significant weight loss (˜13%) and delayed recovery were observed in M2e-H1stem vaccinated mice after CD4+ or CD8+ T cell depletion compared to the non-depleted M2e-H1stem vaccinated mice displaying minimum weight loss (3-4%) (FIG. 5N). Taken together, these data demonstrate that humoral and cellular immunity induced by M2e-H1stem vaccination have contributed to cross protection against different subtypes of influenza A viruses.

### M2e-H1Stem Retains Thermostable Universal Vaccine Features

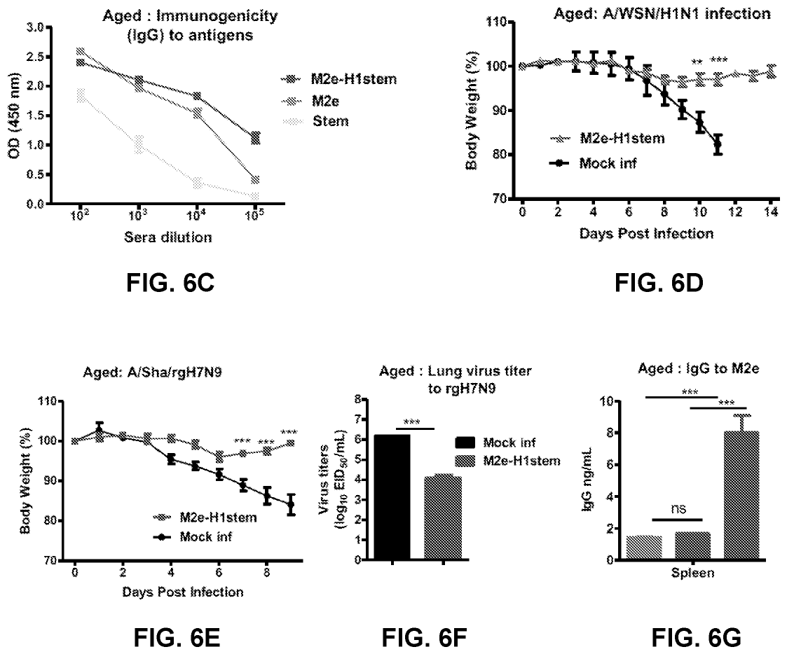

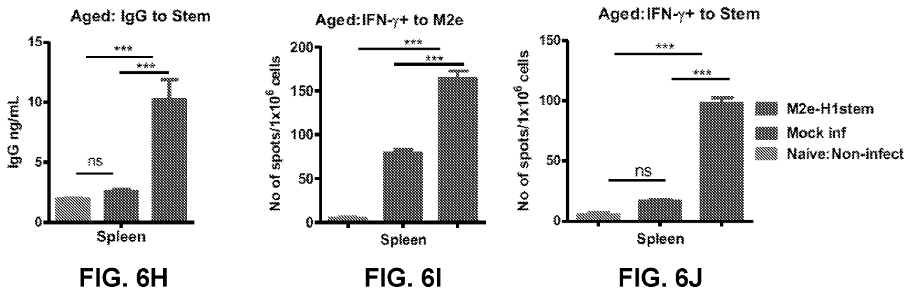

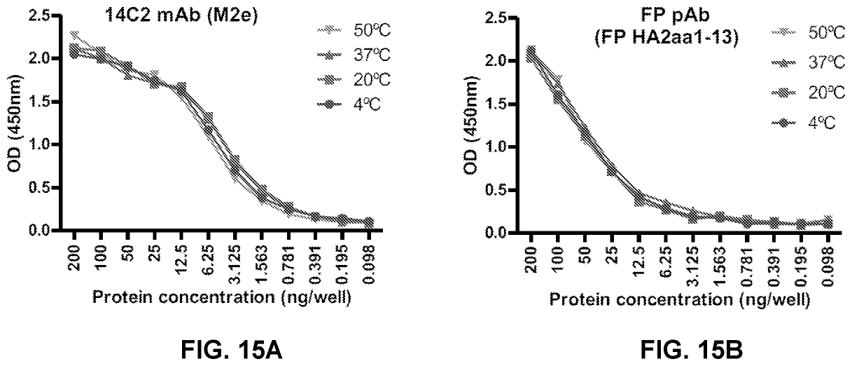

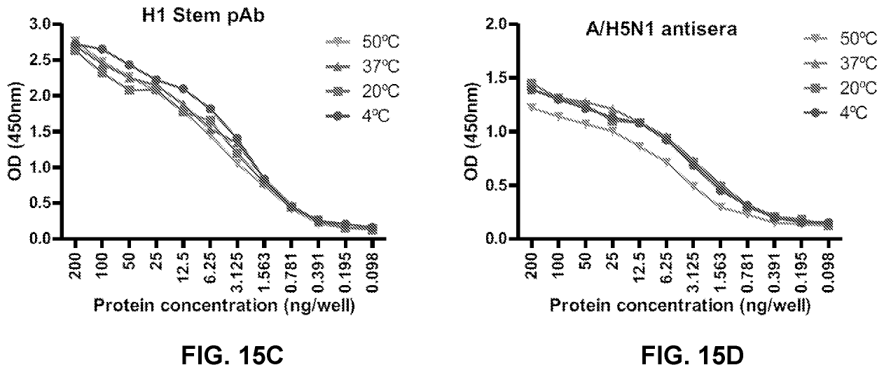

The thermostability of M2e-H1stem protein was determined after storage for 10 days at different temperatures (4, 20, 37, 50° C.). There was no significant difference of M2e-H1stem in retaining antigenic epitopes, based on the reactivity against mAb 14C2 specific for M2e, pAbs specific for fusion peptide and stem, and antisera of rgH5N1 virus infection after 10 days' storage at 50° C. (FIGS. 15A-15D. Adjuvanted vaccination of mice with M2e-H1stem protein pre-stored for 10 days at 50° C., induced comparable IgG Abs specific for M2e-H1stem (FIG. 6A) and protection against lethal challenge with A/WSN H1N1 virus (FIG. 6B), retaining M2e-H1stem vaccine immunogenicity and efficacy even after storage at 50° C. temperature.

### M2e-H1Stem Vaccine Provides Comparable Cross-Protection in Aged Mice

The efficacy of adjuvanted M2e-H1stem vaccination was further evaluated in aged mice. Prime-boost vaccination of 14 months old mice with M2e-H1stem induced high levels of serum IgG Abs specific for M2e, stem, and M2e-H1stem antigens (FIG. 6C). The M2e-H1stem vaccinated aged mice were protected against lethal challenge with A/WSN H1N1 virus, as evidenced by 100% survival rates and minimum weight loss (˜3.5%, FIG. 6D). Remarkably, Adjuvanted M2e-H1stem vaccination of aged mice provided cross-group protection against lethal challenge with group 2 virus (A/Sha/H7N9) by conferring 100% protection with minimal weight loss (˜4%, FIG. 6E). Additionally, M2e-H1stem vaccination led to significantly lowering lung viral titers by 100-fold in aged mice after challenge with rgH7N9 virus (FIG. 6F) than mock control. In addition, M2e-H1stem vaccination in aged mice induced the generation of B cells, which rapidly differentiated to plasma cells secreting IgG Abs, and IFN-γ+-secreting T cells in splenocytes upon in vitro stimulation with M2e peptide or stem protein (FIGS. 6G-6J). These results suggest that M2e-H1 stem vaccination might be effective in providing cross-group protection in elderly populations.

### DISCUSSION

Vaccination inducing antibodies against the conserved HA stem was considered as a promising strategy for cross protection against influenza virus infection. However, substantial challenges exist in developing stem-based universal vaccines, including difficulty in preparing stem immunogens, their nature of being immune-subdominant epitopes, and low efficacy of cross protection. In this study, to overcome the limited breadth and efficacy of stem-based vaccines, we uniquely designed a chimeric M2e-H1stem construct, which was successfully expressed in E. coli. The M2e-H1stem antigen was found to be highly reactive to antisera of different subtype HA protein immunization and group 1 and group 2 live virus infection, implicating the presentation of native-like epitopes. M2e-H1stem was immunogenic, inducing IgG antibodies specific for M2e and stem domains as well as group 1 and 2 HA viruses and virus-infected cell surface antigens. Vaccination of young adult and aged mice with adjuvanted M2e-H1stem protein induced broad cross protection against both group 1 and 2 HA subtype viruses. Therefore, our chimeric M2e-H1stem represents a potential strategy of developing a surpassed universal influenza A vaccine conferring cross-group protection in aged populations.

The HA stem domain has high sequence conservation within the same subtype, but moderate variations among the different subtypes and substantial differences across the other group HA viruses (FIGS. 7A-7B). Headless H1 and H3 HA full-length stem protein vaccines, which were structurally stabilized with trimeric GCN4 or ferritin nanoparticles expressed in mammalian cells, were reported to provide protection against homosubtypic virus in mice, ferrets, or non-human primates, but significantly less effective in conferring protection against different subtype viruses even within the same group. These and other studies suggest limitations on developing a stem only universal vaccine. Our previous study reported enhanced cross protection by inducing immunity to both M2e and stem upon vaccination of mice with double-layered nanoparticles composed of 3 separate proteins expressed in insect cells, where H1 and H3 stem proteins layered onto the M2e cores were physically mixed 21. A chemical cross linker was used to conjugate M2e particles and full-length H1 stem-GCN4 trimer protein with top helix C deleted, conferring HA group specific but not cross group virus protection.

Multi step preparations of nanoparticles likely raise concerns on scale-up vaccine manufacturing and chemical modifications of the potential epitopes. Here, we successfully designed a genetic fusion chimeric M2e-H1stem protein construct to be expressed in E. coli with the retention of M2e and stem epitopes, overcoming low efficacy of cross-group protection by stem vaccine.

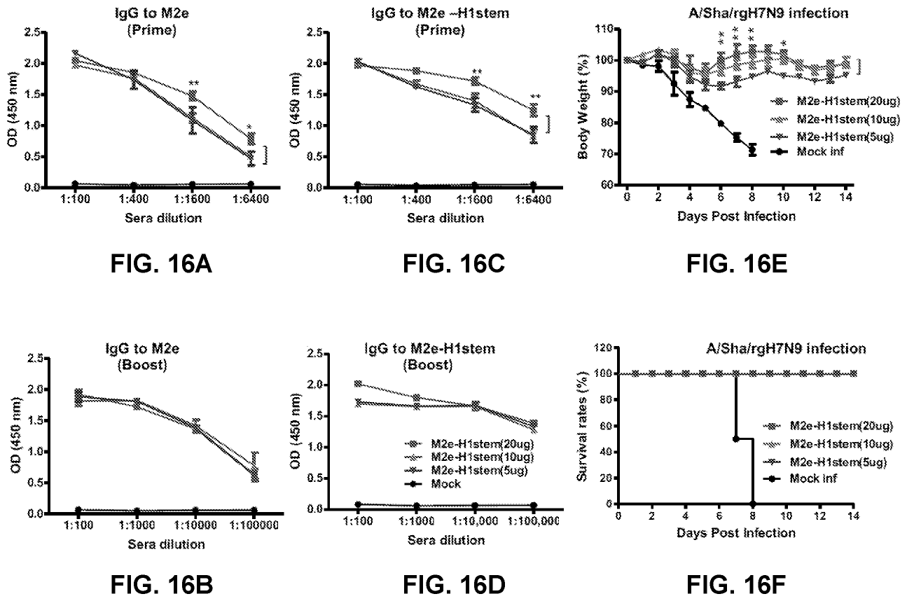

Previous studies identified monoclonal antibodies that recognize the epitopes located in the HA2 stem helix A and C domains as well as HA1 N- and C-terminal parts, which were structurally designed to stabilize the stem structure with point mutations. An earlier study reported near full-length stem (HA2 aa1-172) stabilized with HA1 (aa1-41, aa290-325) domain, which conferred low efficacy protection against homologous virus after CpG adjuvanted vaccination in mice. Prior mini-stem construct 8 lacks the highly conserved HA2 fusion peptide domain (aa1-40), which was shown to be a universal antigenic target inducing antibodies that recognize and cross-neutralize multiple subtypes of influenza A viruses. In contrast, M2e-H1stem construct contains HA2 fusion domain (aa1-40), major stem helix (aa41-117), and M2e tandem repeats. M2e-H1stem was immunogenic and highly reactive to antibodies for M2e and fusion peptide epitopes, inducing M2e and stem-specific antibodies recognizing viral particles and antigens expressed on cell surfaces. There were no significant differences in the levels of IgG Abs among the different vaccine doses (5 μg, 10 μg, 20 μg) (FIGS. 16A-16D). The 20 μg and 10 μg vaccine groups showed slight weight loss (˜5%) whereas the 5 μg vaccine group displayed 8% weight loss (FIGS. 16E-16F). M2e- but not stem-specific antibodies were induced after prime vaccination of M2e-H1stem, even without adjuvant, suggesting that M2e was more immunogenic than stem within the M2e-H1stem protein. Notably, addition of adjuvant (QS-21+MPL) in M2e-H1stem vaccination enhanced immune responses to M2e, stem, and viruses as well as protective efficacy. Previous studies reported the use of adjuvants, such as oil-in-water emulsion or CpG in stem protein (20 μg) vaccination. A different strategy to enhance stem-specific Abs was employed by multiple and sequential vaccinations with DNA, protein, and inactivated viruses with chimeric HAs containing the non-seasonal globular head and seasonal stem domains. While these prior approaches of stem target vaccines protected animals from homologous and heterologous virus within the same group, our study represents significant advancement where M2e-H1stem vaccination provided broad cross-group protection against antigenically different, cross-group subtype viruses.

Several mechanisms contribute to enhancing cross protection by inducing immunity to both M2e and stem, in the absence of receptor blocking neutralizing antibodies. Binding of stem antibodies interferes with subsequent membrane fusion, eventually preventing the infection. Passive transfer of M2e-specific or stem-specific antisera was shown to provide protection in mice. Antibody binding to the fusion domain prevents cleavage of the HA precursor (HA0) into HA1 and HA2. Stem antibodies can bind to HA0 on the infected cells, interfering with viral release. Natural killer (NK) cells can kill virus-infected cells via interaction between Fc receptors (FcR) on NK cells and Fc of antibodies bound to the M2e and HA stem.

Additionally, macrophages or neutrophils engulf M2e and stem IgG-bound influenza viral particles or virus-infected cells via interaction of FcR and Fc. We found that M2e-H1stem antisera recognized viral antigens expressed on the surface of MDCK cells infected with group 1 or 2 viruses and enabled the activation of Jurkat cells endogenously expressing a transcription factor involved in the signaling events of ADCC, suggesting a protective mechanism. Antisera of M2e-H1stem protected naïve mice from both group 1 (H1N1) and 2 (rgH7N9) viruses, further supporting cross protective roles of humoral immune responses. Mice immunized with adjuvanted tandem repeat M2e protein vaccine (no stem) could not prevent severe weight loss, in line with low efficacy of M2e alone immunity. Consistently, M2e-H1stem vaccination induced high levels of IFN-γ+ secreting CD4+ and CD8+ T cells in spleen and lung. Either CD4+ or CD8+ T cell depletion in M2e-H1stem vaccinated mice led to lower protection efficacy, implicating an important role of T cells in bestowing protection by M2e-H1stem vaccination.

It has been shown that the pigs that were vaccinated with inactivated virus vaccine (H1N2) induced antibodies binding to the HA2 domain but displayed enhanced pneumonia and disease after heterologous pandemic virus H1N1 infection. Promoting viral infection and disease might be due to the induction of antibodies binding close to the fusion peptide in the absence of neutralizing antibodies or the use of a swine animal model prone to this possibility. Vaccination inducing broader humoral and cellular immunity to M2e and stem domains or supplementing cross protective immunogens to seasonal vaccination might provide a strategy of attenuating or preventing respiratory disease against heterologous virus infection. Nonetheless, a possibility of disease enhancement by anti-HA2 stem antibodies requires cautious awareness in developing universal vaccines.

In summary, our chimeric M2e-H1stem protein immunogen was unique in presenting multi cross protective epitopes and inducing IgG antibodies specific for M2e, fusion peptide, stem, and cross-group viral antigens on the virion particles and the surfaces of virus-infected cells. Adult and aged mice vaccinated with adjuvanted M2e-H1stem could provide a broad range of cross-group protection against lethal challenge with group 1 and 2 viruses. Mechanisms, such as cellular and humoral immunity, including ADCC, to both M2e and stem might be contributing to broad cross-group protection. Conjugation of M2e and stem domain as a single antigen would have some benefits of enabling the induction of M2e and HA2 stem specific immune responses and broadening cross protection. Importantly, E. coli expressed M2e-H1stem protein was thermostable, enabling rapid scale-up during a pandemic outbreak even in low resource countries. Further studies in more relevant animal models such as ferrets are warranted.

### Materials and Methods

Chimeric M2e-H1stem construct, expression, and protein purification. Influenza A virus [A/PR8/1934 (H1N1)] HA gene sequence was obtained from GenBank (NC_002017) and used for the design of H1 stem protein construct. The HA2 region of group 1 HA was multiply aligned using Clustal Omega, and amino acid residues (aa) 31-54 and 304-337 of HA1 as well as 1-117 of HA2 region were selected as a vaccine target based on the conserved region of the HA stem and stabilizing domains, without altering the structure of the HA. The hydrophobic residues were modified by introducing polar aa mutations. Tandem 2xM2e repeat was genetically connected to the N-terminal of the HA1 via flexible linkers. The C terminus of chimeric M2e-H1stem construct was connected to the β-rich trimer-stabilizing foldon. The M2e and foldon structures were derived from the PDB ID codes 3BKD and 4NCB, respectively. The 3D structure of HA was predicted using SWISS-model and visualized in PyMOL.

The nucleotide sequence of the M2e-H1stem construct was codon-optimized for expression in Escherichia coli (E. Coli) and synthesized by GenScript (USA). The synthesized gene was ligated into the pCold II cold expression vector (Takara Bio. Inc) containing N-terminal 6× His-tagged, and subsequently transformed into E. coli (DH5-α) and Rosetta (DE3) pLysS cells (Novagen, USA). The expression of M2e-H1stem was induced in transformed DE3 bacteria by 1 mM isopropyl-β-D-1-thiogalactopyranoside and cultured at 16° Celsius for 14 hours. To obtain higher yields of protein preparations, we harvested and solubilized E. coli cell pellets containing both soluble and insoluble forms in 8M urea lysis buffer (20 mM HEPES, pH 8.0, 300 mM NaCl, 2 mM CHAPS, 8 M urea, 10 mM imidazole). After sonication, cleared lysates were applied to His tag affinity Ni-NTA beads. The bound M2e-H1stem protein was eluted with lysis buffer containing 250 mM imidazole and refolded by step dialysis in 20 mM HEPES, pH 8.0, 200 mM NaCl, 5% glycerol, and 1 mM DTT with a gradual decrease in urea.

The final refolded M2e-H1stem protein was further dialyzed against PBS, quantified, and stored at −80° C. until further use. The purified M2e-H1stem protein was separated by 12% sodium dodecyl sulfate polyacrylamide gel electrophoresis (SDS-PAGE) and further evaluated by western blot using M2e-specific 14C2 mAb and H1-stem specific rabbit pAbs. The M2e and stem epitopes in the purified M2e-H1stem protein were determined by standard ELISA using M2e and stem specific antibodies and live virus antisera. The level of endotoxin in M2e-H1stem vaccine formulation was within a low range (0.5 units/20 μg/mL) as tested by chromogenic LAL Endotoxin Assay Kit (GenScript), which is below the allowable level (20 endotoxin units/mL) for recombinant protein subunit vaccines.

Antibodies and protein. M2e specific mAb (14C2) was purchased from Santa Cruz Biotechnology (USA). Unit-1 mAb was kindly provided by Xuguang Li (University of Ottawa). The following rabbit polyclonal antibodies (pAbs) specific for stem were generated by GenScript (USA) via hyper immunizations with HA2 stem peptide epitope-conjugates and purification by using the peptide-linked affinity column: anti-H3-FP specific for HA2aa14-27, anti-fusion peptide (HA2aa1-13), and anti-H1stem (HA2aa103-116). Goat polyclonal antibodies (pAbs) specific for recombinant HA were acquired from BEI resources (ATCC/NIH): anti-H1 HA pAbs (NR 15696); anti-H5 HA pAbs (NR 2705); anti-H3 HA pAbs (NR-48597,); anti-H7 HA pAbs (NR-48597). Preparation of the stem protein (without M2e) was previously described 49 and used as an ELISA coating antigen for measuring HA stem-specific IgG antibodies.

Immunization and virus challenges. Mouse experiments were approved by Georgia State University Institutional Animal Care and Use Committee (IACUC A21004). BALB/c female-mice (6 to 8 weeks old, Jackson Laboratories) were intramuscularly immunized in a prime-boost schedule with a three-week interval in the hind legs. M2e-H1stem protein (5, 10, 20 μg) vaccine in 100 μL PBS (50 μL in each leg) was used for prime with adjuvants [10 μg QS-21 (Desert King International) plus 1 μg monophosphoryl lipid A (MPL, Sigma Aldrich)] and boost vaccination with adjuvants (5 μg QS-21+0.5 μg MPL). Immunized mice were anesthetized with isoflurane prior to blood collection, and approximately 100 μL of blood samples were collected through retro-orbital sinus at two weeks after prime or prime and boost immunization. For aged mice, 14-month old BALB/c mice were intramuscularly immunized with adjuvanted M2e-H1stem vaccine (20 μg). Blood samples were collected after two weeks of prime and boost immunization. Eight weeks after boost, mice were challenged intranasally with a lethal dose of influenza A virus in 50 μL PBS. Weight loss >20% was considered as the IACUC endpoint. Group 1 influenza A viruses were as follows: A/Puerto Rico/8/1934/H1N1 (A/PR8/H1N1), A/California/04/2009/H1N1 (A/Cal/H1N1), A/WSN/1933/H1N1 (A/WSN/H1N1), A/Hong Kong/1073/1999 H9N2 (A/HK/H9N2), mouse-adapted A/Fort Monmouth/1/1947 (A/FM/H1N1), and reassortant A/Vietnam/1203/2004/H5N1 with A/PR8 backbone (A/Viet/H5N1). Group 2 viruses used include A/Philippine/2/1982/H3N2 (A/Phil/H3N2), A/Hong Kong/I/1968/H3N2 (A/HK/H3N2), reassortants A/Shanghai/11/2013/H7N9 with A/PR8 backbone (A/Sha/H7N9), and A/Nanchang/933/1995/H3N2 with A/PR8 backbone (A/Nanchang/H3N2).

Enzyme-linked immunosorbent assay. M2e-H1stem protein vaccine antigenicity using antibodies specific for known epitopes, IgG antibody responses in sera and in vitro cultures were determined by standard ELISA as previously described. ELISA coating antigens included M2e peptide (100 ng/well), M2e-H1stem or group 1 stem protein (50 ng/well) prepared as previously described, foldon-linked spike protein (BEI NR-52396, 50 ng/well) of severe acute respiratory syndrome coronavirus 2 (SARS-CoV-2), H1 stem peptides (100 ng/well), or inactivated viruses (200 ng/well). In brief, we performed a serial dilution of pAbs to find an optimal concentration for M2e-H1stem protein vaccine antigenicity assay. Then we determined the antigenicity of the M2e-H1stem protein vaccine to determine the antigenic affinity and exposure to the epitope specific pAbs by testing different protein coating concentrations. IgG antibody levels were determined with serial dilutions of immune sera from vaccinated mice on a specific antigen coated-ELISA plate.

In vitro detection of IgG antibody. Mediastinal lymph node (MLN) and spleen cells were isolated from young adult (6-week-old) or aged (14 months-old) BALB/c mice at 6 days post infection. The cells were cultured on the plates pre-coated with antigens (M2e peptide, M2e-H3 stalk, or stalk protein) for 5 days at 37° C. The quantitation of antigen-specific IgG production in ng/mL after in vitro culture was determined by ELISA using mouse standard IgG antibody (Cat no 1010-01, Southern Biotech) as previously described.

Determination of lung viral titers. The lung tissues collected at day 5, 6, 9 post infection were homogenized, serially diluted (10-fold) lung extracts (200 μL) were injected into 10-day-old embryonated chicken eggs. The viral titers were presented in median embryo infectious dose (EID₅₀) after incubation for 3 days by hemagglutination assay in allantoic fluids as described.

Interferon-γ ELISpot and flow cytometry. IFN-γ+ secreting cells were evaluated on the 96-well ELISpot plates pre-coated with IFN-γ-capture antibody. ELISpot plates were seeded with splenocytes (5×10⁵ cells) or lung cells (2×10⁵ cells) and incubated with the stimulators: M2e peptide (5 μg/mL), M2e-H1stem protein (2 μg/mL), stem protein (2 μg/mL), or inactivated A/Sha/H7N9 influenza virus (4 μg/mL). IFN-γ-secreting cell spots were visualized with color-developing 3,3′-diaminobenzidine substrates and counted as described. In vitro cultures of the isolated lung and spleen cells were stimulated with 5 μg/mL of M2e peptide and/or stem protein with Brefeldin A (20 μg/mL). After 5 hours' culture, the cells were stained with mouse anti-CD3 (clone 17A2, BD), anti-CD4 (clone 553051, BD) and anti-CD8 (clone 25-0081-82, eBiosciences), followed by fixation and permeabilization using a Cytofix/Cytoperm kit (BD Biosciences) and then, staining of intracellular cytokine IFN-γ using IFN-γ mAb (anti-mouse IFN-γ, clone XMG1.2, BD). Table 1 contains the source and working dilutions of antibodies used for flow cytometry and intracellular cytokine staining listing. Lymphocytes were gated to exclude dead cell-like events in flow cytometry experiments (FIGS. 14A-14C). The IFN-γ+ T-cells were analyzed by Becton-Dickinson LSR-II/Fortessa flow cytometer (BD) and FlowJo software (FlowJo V10, Tree Star, Inc.) as described.

In vivo protection of immune sera and in vivo T-cell depletion. Sera were heat-inactivated, diluted (4-fold), and mixed with a lethal dose of A/Sha/H7N9, A/Phil/H3N2 or A/WSN/H1N1 virus. Body weight and survival rates were monitored after intranasal inoculation of naive mice with the mixture of sera and virus. Antisera were obtained from the groups of mice that were boost vaccinated with adjuvanted M2e-H1stem. T-cell depletion in mice was performed with anti-CD4 (GK1.5) and anti-CD8 (53.6.7) mAbs (BioXCell) as previously described 53. The levels of CD4+ and CD8+ T-cells were determined by flow cytometry.

Cell surface ELISA and ADCC assay. The MDCK cells were seeded on 96-well cell culture plates (3×10⁴ cells) and then infected with virus. Day 1 post-infection, cells were fixed with 4% paraformaldehyde and incubated with diluted sera, followed by standard ELISA to determine IgG antibodies bound to the viral antigens on the MDCK cell surface. The ADCC Reporter Kit (cat no:M1215, Promega Life Sciences, USA) was used to measure the activation of Jurkat cells mimicking natural killer cells as a surrogate indicator for ADCC by serum antibodies bound to the virus-infected MDCK cells (3×10⁴ cells). After incubation with Jurkat (7×10⁴) effector cells for 6 hours, luminescence was read to calculate fold increases using a Cytation 5 imaging reader (BioTek).

Statistical analyses. Data analyses were performed using Prism software (GraphPad Software Inc). The statistical significance was determined by either one- or two-way ANOVA followed by Tukey's multiple comparison or Bonferroni post-test. All the data were represented as the mean±the standard errors of the mean (SEM). P values less than 0.05 (p<0.05) were considered statistically significant.

### Example 2: A Chimeric Thermostable M2e and H3 Stalk-Based Universal Influenza a Virus Vaccine

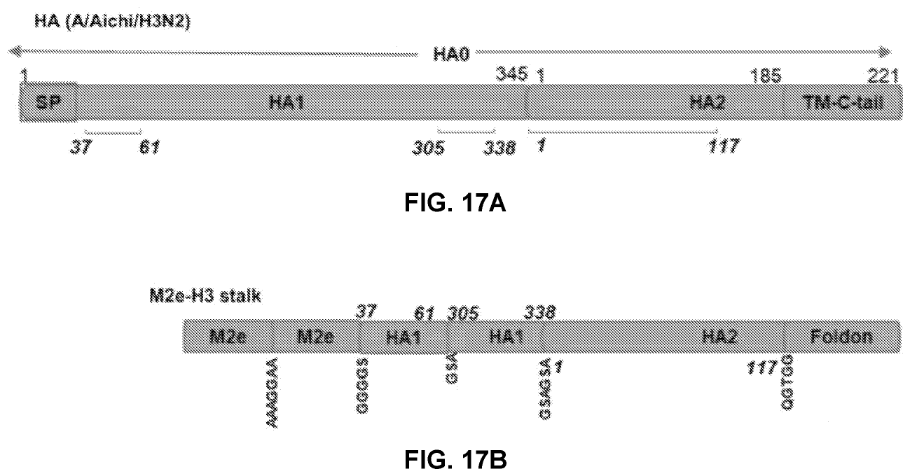

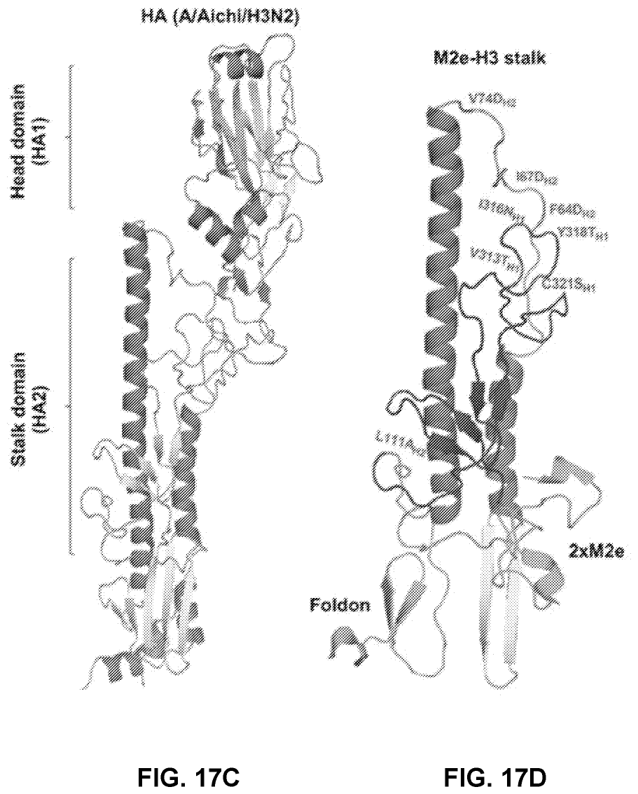

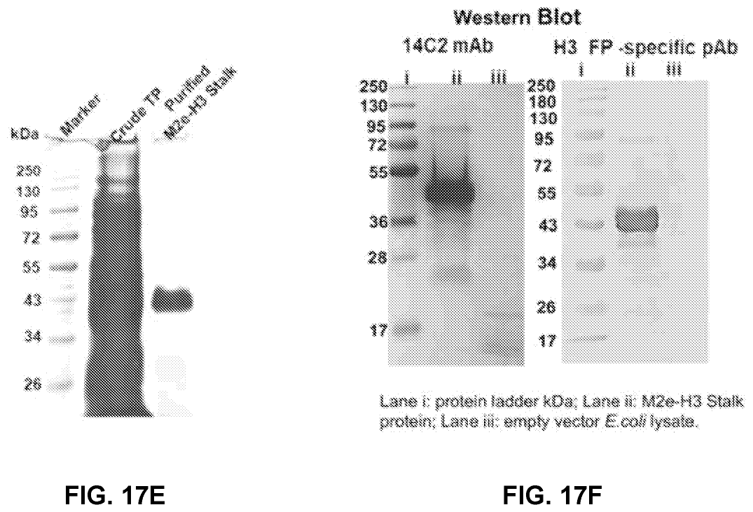

Rational design and development of chimeric M2e-H3 stalk universal vaccine construct Structural conformation of HA2 stalk domain was previously modeled to be stabilized with the N- and C-terminal HAI parts. To extend and enhance the breadth of cross protection, a genetic fusion of M2e epitopes and H3 stalk was constructed (FIGS. 17A-17D). The H3 shortened stalk domain contains HAI parts [aa37-61, aa305-338 of H3 HA from A/Aichi], and HA2 stalk in α-helix conformation [aal-117, FIGS. 17B-17D]. Tandem 2× repeat of M2e (23 aa) epitope domains was genetically fused to the H3 stalk N-terminus (M2e-H3 stalk) from A/Aichi/H3N2 influenza A virus.

The N-terminal half of the HA2 stalk domain is enriched with broadly neutralizing B cell epitopes as previously identified. Therefore, the C terminal hydrophobic stalk part was excluded in the M2e-H3 stalk construct and replaced with the p rich trimeric nature of the foldon sequence to enhance the stability and proper folding of the protein (FIG. 17D). Point mutations shown in FIG. 17D were introduced in the hydrophobic patches in the HAI (V313T_{H1}, 1316N_{H1}, and Y318T_{H1}) and HA2 stalk domains (F64D_{H2}, 167D_{H2}, V74D_{H2}, LI111A_{H2}). These point mutations were previously described to attenuate strong hydrophobic interactions and to avoid protein aggregations in neutral pH conformation, potentially improving the protein preparation in a soluble form. In addition, cysteine residue on 321 position was replaced by serine residue (C321S) to prevent non-specific intermolecular disulfide formation. A previous study demonstrated that the foldon trimer stabilizing domain was required for helical trimer formation and thermal stabilization, and for enabling resistance to proteolysis. To facilitate protein purification, 6xHis tag was fused to the N-terminus of the M2e-H3 stalk domain. Flexible linkers were used to connect independent domains and to facilitate the display of native-like conformation.

A codon-optimized gene encoding M2e-H3 stalk protein was synthesized and cloned into pCold II, a high expression vector in E. Coli. Chimeric M2e-H3 stalk proteins were expressed in E. Coli cells. Cell lysates containing M2e-H3 stalk proteins were dissolved in 8 M urea and fractions collected through the Ni-affinity His trap column were refolded into soluble M2e-H3 stalk protein with high purity (FIG. 17E). Chimeric M2e-H3 stalk proteins were further confirmed by western blot with M2e specific mAb 14C2 and fusion epitope specific polyclonal antibody (pAb, FIG. 17F).

### Chimeric M2e-H3 Stalk Protein Displays Cross Reactive Antigenicity and Thermostability

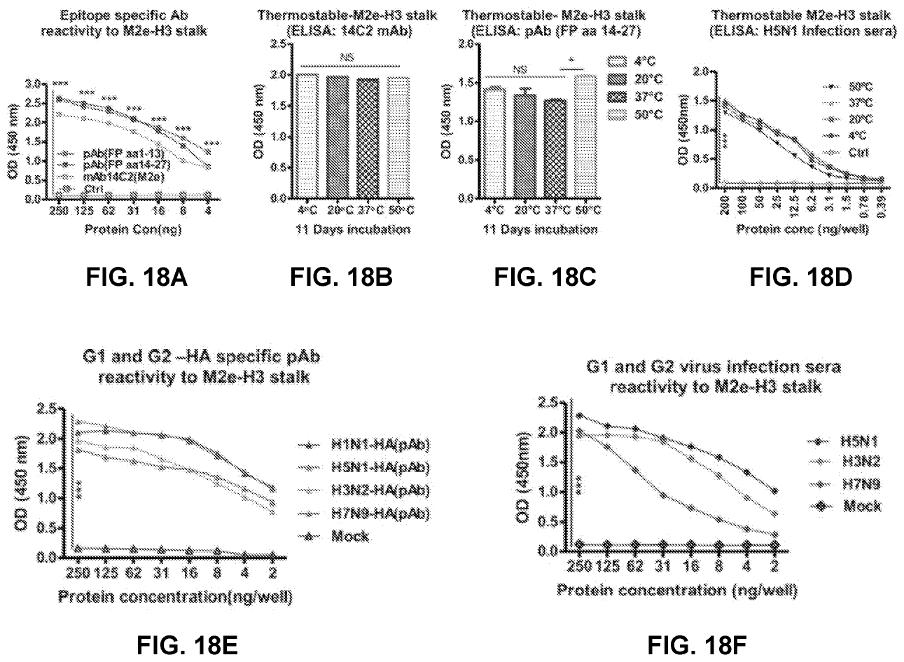

Epitope integrity and thermostability of chimeric M2e-H3 stalk protein were examined. M2e-H3 stalk proteins were highly reactive with M2e specific mAb 14C2 as well as rabbit polyclonal antibodies specific for highly conserved HA2 aal-13 fusion peptide, and HA2 aa14-27 peptide (FIG. 18A). M2e and fusion epitope antigenicity was retained even after incubation for 11 days at low (4° C.) to high temperature (50° C.) storage (FIGS. 18B-18C). The antigen was also reactive to antisera from mice recovered from H5N1 virus infection (FIG. 18D), indicating high thermostability of native-like epitope integrity. M2e-H3 stalk protein displayed strong antigenic reactivities for pAbs against HA proteins derived from diverse subtypes, including RINI (A/California/2009), H5NI (A/Vietnam/2004), H3N2 (A/Swine/2011), and H7N9 (A/Shanghai/2013) (FIG. 18E). More importantly, antisera from infection with different subtype influenza A viruses (H5NI, H3N2, H7N9) exhibited high reactivities to the M2e-H3 stalk protein antigen (FIG. 18F). Overall, these results suggest that M2e-H3 stalk protein exposes diverse native-like conserved epitopes which are recognized by 14C2 mAb, different subtypes HA pAbs and antisera from virus infection.

### Adjuvanted M2e-H3 Stalk Protein Vaccination Induces IgG Antibodies Recognizing M2e, Stalk, and Diverse Subtype Viruses

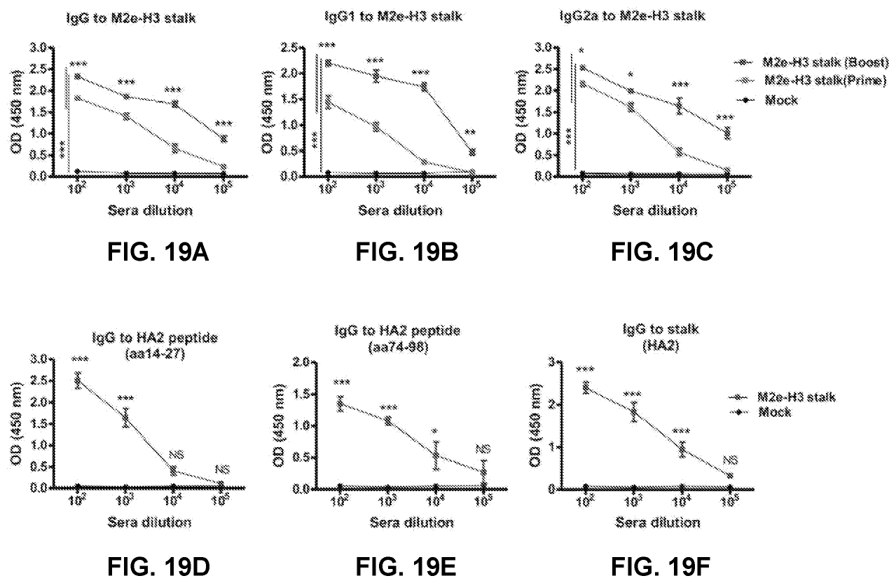

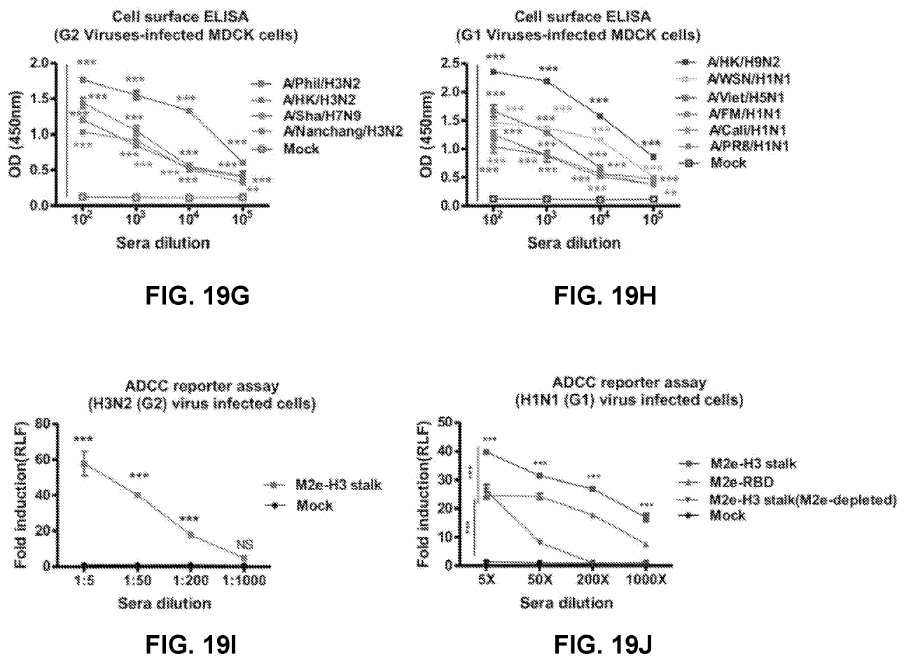

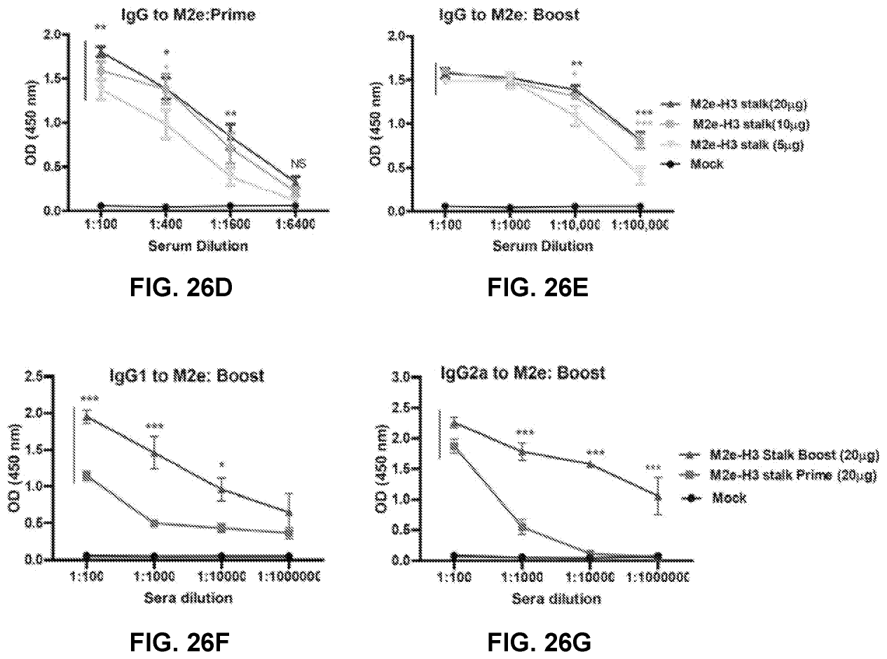

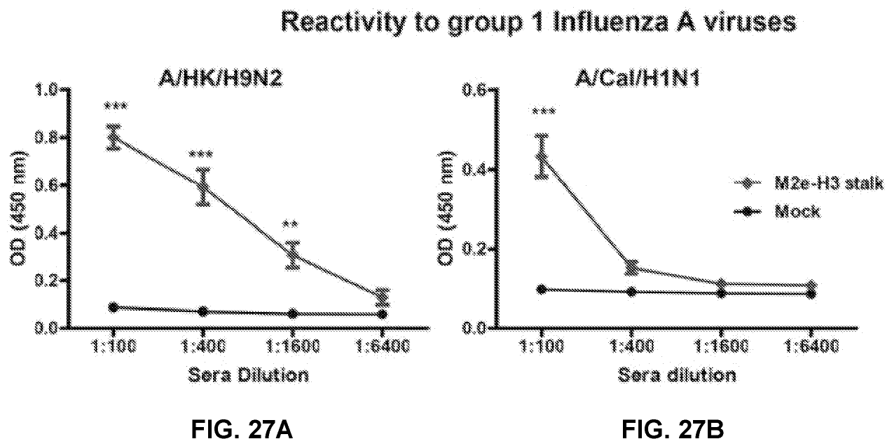

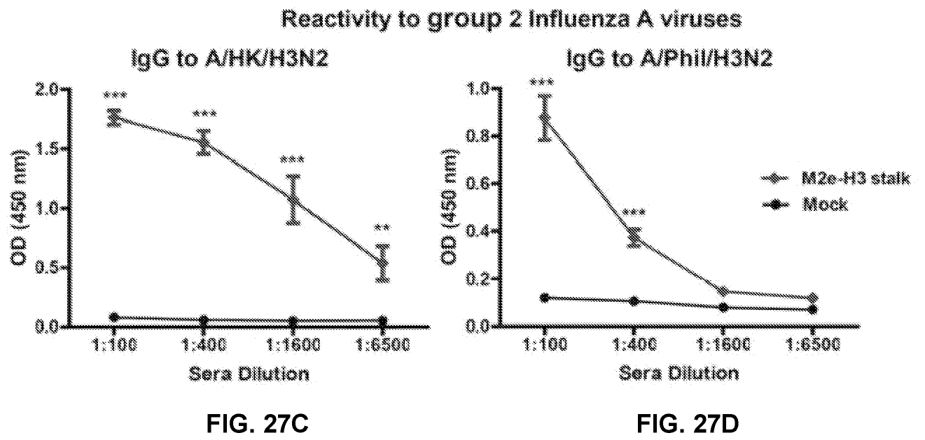

Protein subunit vaccines in general require adjuvanted formulations to enhance the immune responses and protective efficacy. We tested the effects of ASOI-like adjuvant (QS-21+MPL), major ingredients in ASOI liposome adjuvant licensed for use in herpes Zoster vaccination 24·25. Adjuvant (QS-21+MPL) included in M2e-H3 stalk protein prime boost IM vaccination of mice with a 3-week interval exhibited significant impacts on enhancing IgG 1 and IgG2a antibodies specific for M2e and M2e-H3 stalk antigens (FIGS. 26A-26G). The impact on eliciting immune response with vaccine dosage and adjuvants was evaluated with different vaccine antigen amounts with or without adjuvants. The adjuvanted M2e-H3 stalk (20 μg) induced significantly higher IgG, IgGI, and IgG2 specific M2e-H3 stalk antibodies than the unadjuvanted group (FIGS. 26A-26C). Four-fold less (5 μg) vaccine dose with adjuvanted M2e-H3 stalk induced higher levels of IgG responses than those with 20 μg of vaccination without adjuvant. Therefore, we focused on testing immune responses and efficacy after vaccination with adjuvanted (QS-21+MPL) M2e-H3 stalk vaccination of mice. At 2 weeks after M2e-H3 stalk protein (20 μg) prime vaccination, substantial levels of IgG, IgG 1, and IgG2a specific to M2e-H3 stalk protein antigens were induced after IM prime dose (FIGS. 19A-19C). After boost, the levels of IgG, IgG1, and IgG2a antibodies specific to vaccine antigens or M2e epitope, were increased by approximately 10 folds (FIGS. 19A-19C and 26D-26G). Boost immune sera showed substantial levels of IgG antibodies binding to HA2 fusion peptides (aa 14-27), HA2 stalk epitope (HA2aa 74-98), one of the essential epitopes on stalk domain, and stalk protein (FIGS. 19D-19F). Also, M2e-H3 stalk boost sera displayed higher levels of IgG antibodies against inactivated group 2 viruses (A/HK/1968/H3N2 and A/Phil/H3N2) than those inactivated group 1 viruses (rgA/HK/1999/H9N2 and A/California/2009/H1N1) (FIGS. 27A-27D).

Furthermore, M2e-H3 stalk antisera strongly recognized both group 1 (G1) and group 2 (G2) viral antigens expressed on the surface of MDCK cells infected with a broad range of influenza A viruses (FIGS. 19G-19H). Antisera of adjuvanted M2e-H3 stalk vaccination were highly reactive to the cell surface viral antigens after infection with group 2 influenza A viruses, including A/Phil H3N2, A/HK H3N2, rgA/Shanghai H7N9, and A/Nanchang/1995 H3N2 (FIG. 19G). Interestingly, antisera of M2e-H3 stalk vaccination were also highly reactive to the cell surface viral antigens after infection with group 1 influenza viruses (A/Cal H1N1, rgA/HK H9N2, A/WSN/19 33 H1N1, AIPR8 H1N1, A/FM 1N11, and A/Viet/H5NI) (FIG. 19H).

To investigate whether the M2e-H3 stalk vaccination would engage in Fe-mediated activation of effector Jurkat cells, supporting a possible role of antibody-dependent cellular cytotoxicity (ADCC) in providing cross-protection, we performed an ADCC assay. The ADCC surrogate reporter assay showed an elevated reporter signal with M2e-H3 stalk antisera on both group 2 A/Phil H3N2 (FIG. 19I) and group 1 (A/WSN/H1N1 in FIG. 19I, A/HK/H9N2 in FIG. 33) influenza A virus-infected MDCK cells. In addition, M2e antibody-depleted M2e-H3 stalk antisera lost a significant fraction of their ADCC activity against A/WSN/H1N1 whereas M2e-RBD vaccine sera as a control of M2e alone antisera retained a substantial level of ADCC activity against A/WSN/H1N1 and showed less reduction in ADCC activity, as compared to the M2e-H3 stalk vaccine antisera (FIG. 19I). These new ADCC experimental data suggest that M2e specific IgG antisera might have played a significant role in exhibiting elevated levels of ADCC activity compared to the stalk antisera only when tested against A/WSN/H1N1 group 1 virus, and that the combination of M2e with H3 stalk antisera resulted in increasing the ADCC activity by over 20 folds. These results suggest that M2e-H3 stalk antisera has ADCC reporter assay activity against group 1 and group 2 influenza A viruses and that adjuvanted M2e-H3 stalk vaccination effectively induced antibodies recognizing M2e, stalk, and group I and 2 virus antigens on virions and on the surfaces of infected cells.

### M2e-H3 Stalk Protein Vaccine Provides Broad and Effective Cross Protection Against Group 2 Viruses

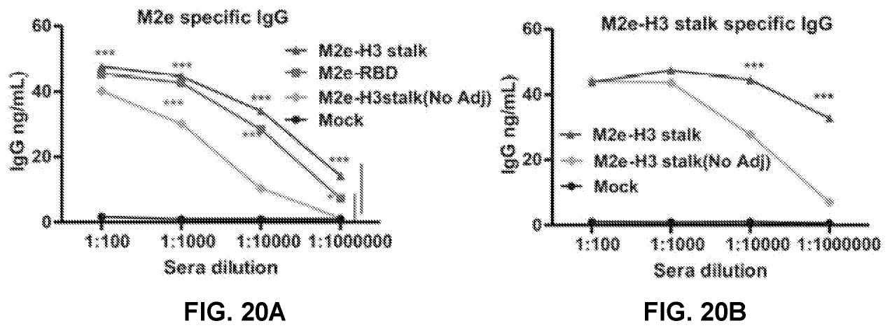

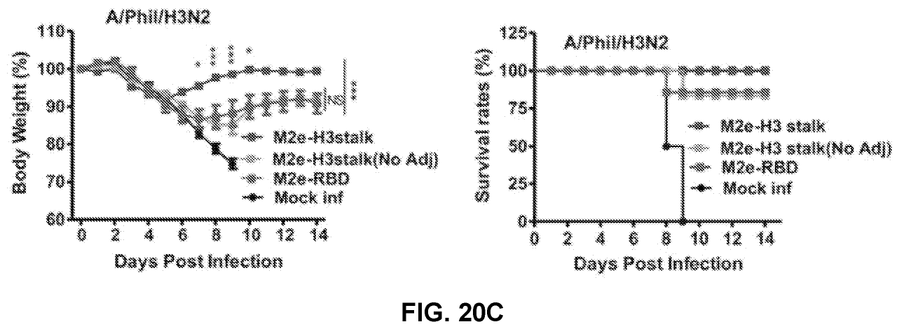

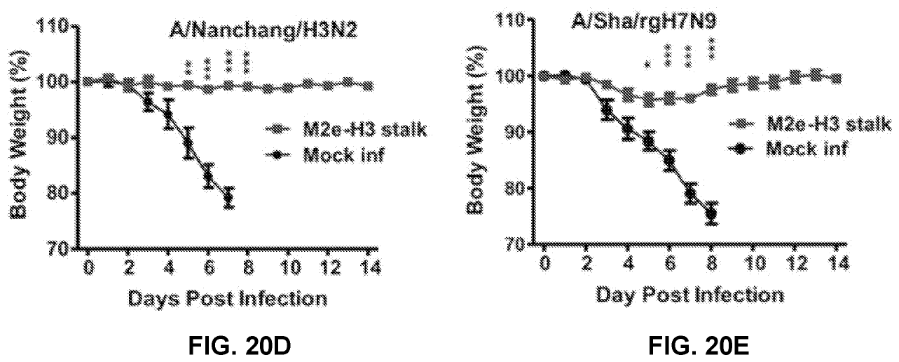

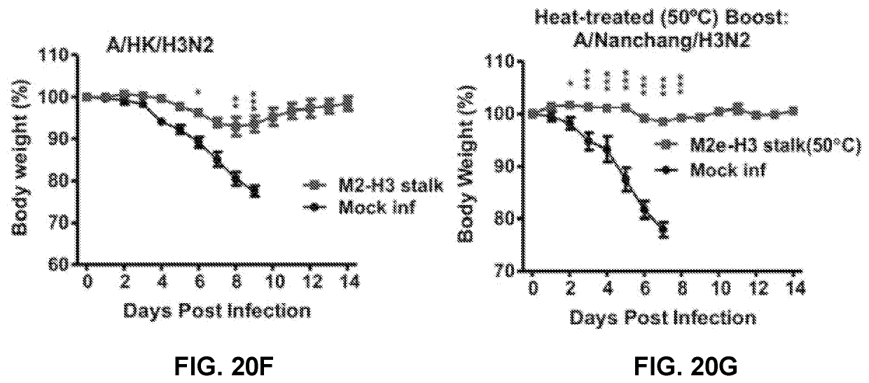

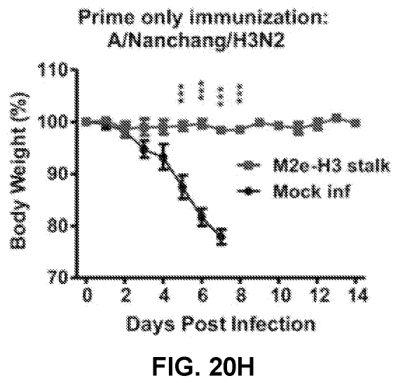

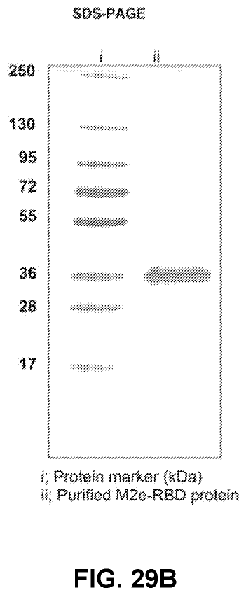

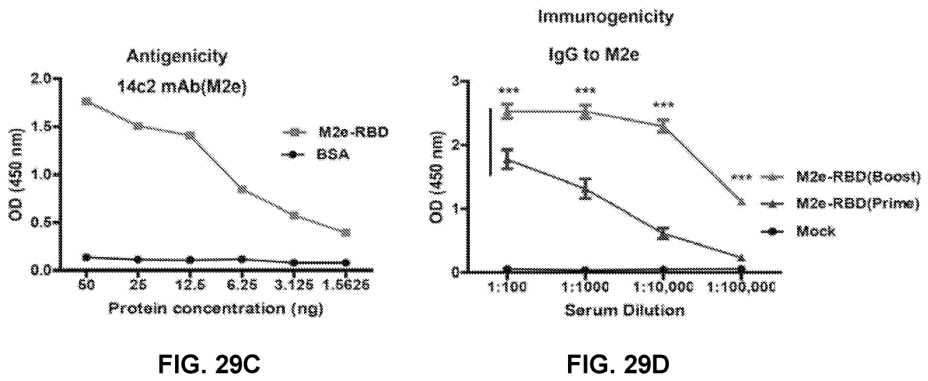

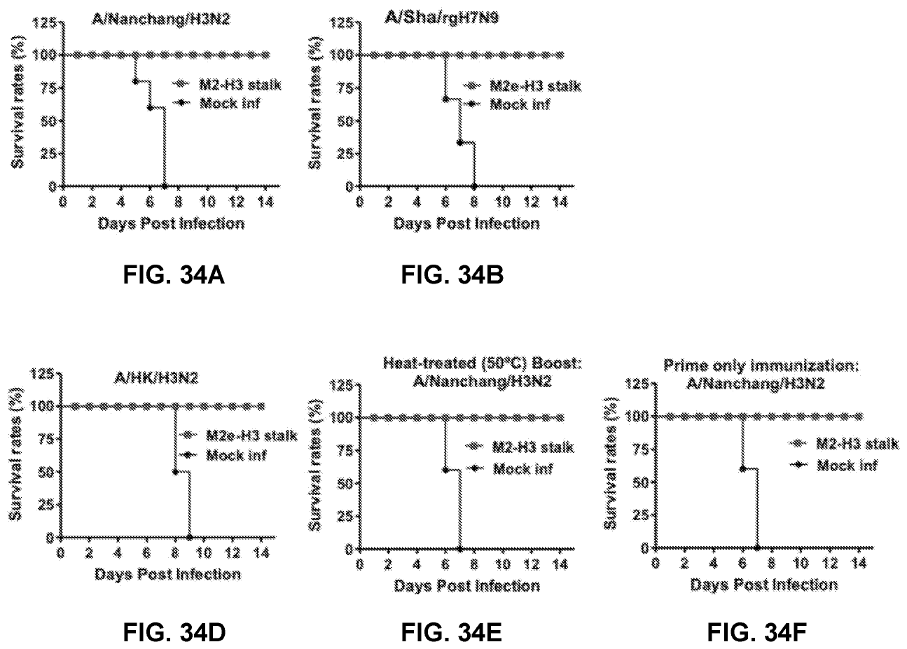

Unadjuvanted M2e-H3 stalk protein (20 μg) vaccination resulted in weight loss up to 20% and survival rates 80% after challenge with A/Phil (H3N2) virus whereas unvaccinated mice did not survive A/Phil virus infection (FIG. 20A). The adjuvanted M2e-H3 stalk group exhibited significant higher efficacy of protection against lethal A/Phil vims infection, as evidenced by less weight loss (−8.5%) and 100% survival rates (FIG. 20A), supporting significant effects of adjuvant (QS-21+MPL) on enhancing the protection. To compare with the protection efficacy by M2e alone, we designed and purified another chimera protein (M2e-RBD) containing N terminal M2e fused with receptor-binding domain (RBD) of SARS-COV-2 and stabilized with a foldon trimer (FIGS. 29A-29D). M2e-RBD protein was reactive with M2e specific mAb (14C2, FIG. 29C) and the adjuvanted M2e-RBD group induced high levels of M2e-specific antibodies (FIG. 29D). The M2e specific and M2e-H3 stalk specific IgG levels in the adjuvanted vaccine groups were significantly higher than unadjuvanted M2e-H3 stalk (FIGS. 20A-20B). However, the adjuvanted M2e-RBD group showed partial protection against A/Phil/H3N2 virus lethal challenge (FIG. 20C). Therefore, we extended the efficacy testing of adjuvanted M2e-H3 stalk vaccination to additional group 2 viruses. The adjuvanted M2e-H3 stalk group provided significantly enhanced protection against H3N2 (A/Nanchang/1995, FIG. 20D) and rgH7N9 (A/Shanghai/2013, FIG. 20E), as shown by minimum weight loss {<4%) and 100% survival rates (FIGS. 34A-34B). A moderate level of weight loss (˜9%) was observed in the adjuvanted M2e-H3 stalk group after challenge with A/HK/1968 H3N2 virus (FIGS. 20F and 34C). These results indicate that adjuvanted M2e-H3 stalk protein vaccination provides effective cross protection against the antigenically different group 2 viruses.

Next, to evaluate the thermostability of the vaccine antigen, the M2e-H3 stalk was stored at high temperature (50° C.) for eleven days prior to vaccination. The vaccinated mice with M2e-H3 stalk after storage at 50° C. showed 100% protection against A/Nanchang/H3N2 virus challenge (FIGS. 20G and 34D). A single dose of M2e-H3 stalk (20 μg) vaccination showed 100% protection against the A/Nanchang/H3N2 virus, preventing weight loss, while the mock group did not survive (FIGS. 20H and 34E). These results revealed that M2e-H3 stalk protein is thermostable, and prime-only vaccination can provide protection against the group 2 influenza A viruses.

### M2e-H3 Stalk Protein Vaccination Prevents Severe Weight Loss and Confers Cross Protection Against Group 1 Viruses

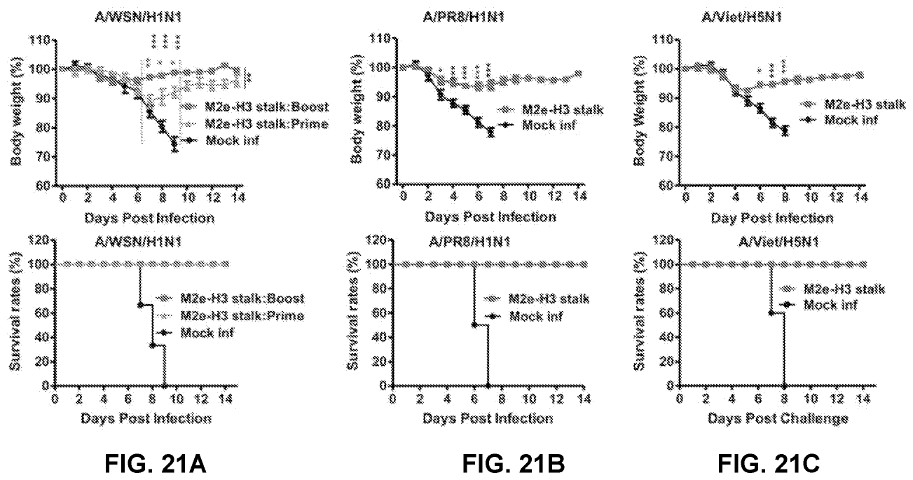

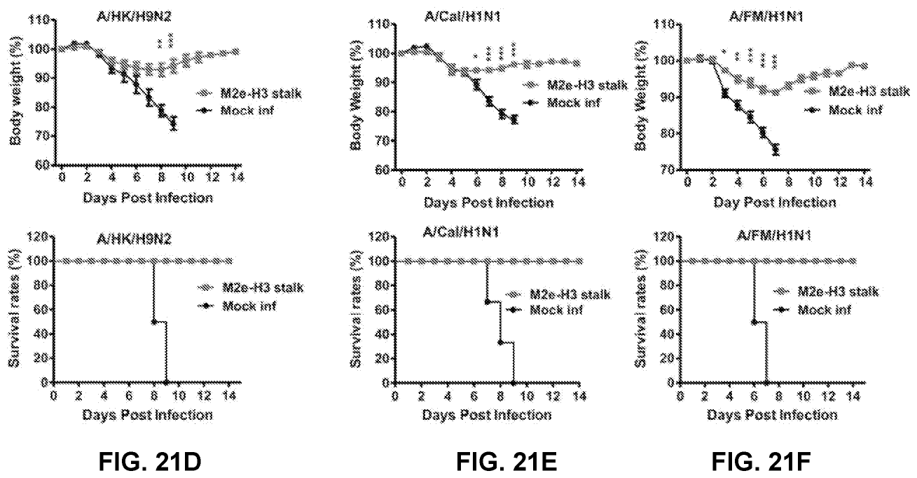

HA2 stalk immunity is known to be group specific and ineffective in inducing cross group protection. Here we tested whether adjuvanted M2e-H3 (group 2) stalk protein vaccination would induce protection against group 1 viruses after 4 to 8 weeks post boost dose. The M2e-H3 stalk primed group showed complete protection against group 1 virus (A/WSN), although the mice displayed moderate weight loss (˜10%) whereas minimum weight loss (<4%) was observed in the M2e-H3 stalk boosted group (FIG. 21A). Prevention of severe weight loss as well as 100% survival rates were observed with adjuvanted M2e-H3 stalk protein vaccination after lethal challenge with H1N1 viruses (A/WSN/1933, A/PR8), H5NI virus (rgA/Vietnam/2004), and H9N2 virus (rgA/HK/1997) (FIGS. 21A-21D). Cross-group protection against other additional HINI strains such as A/California/2009 and A/FM/1947 was observed with the outcomes of a low-level weight loss (5-8%) and 100% survival rates after adjuvanted M2e-H3 stalk protein vaccination of mice (FIGS. 21E-21F). These results support that adjuvanted M2e-H3 stalk protein vaccination can provide cross-group protection and prevent severe weight loss.

### M2e-H3 Stalk Protein Vaccination Induces Effective Lung Viral Clearance and Protective Humoral and Cellular Immune Responses

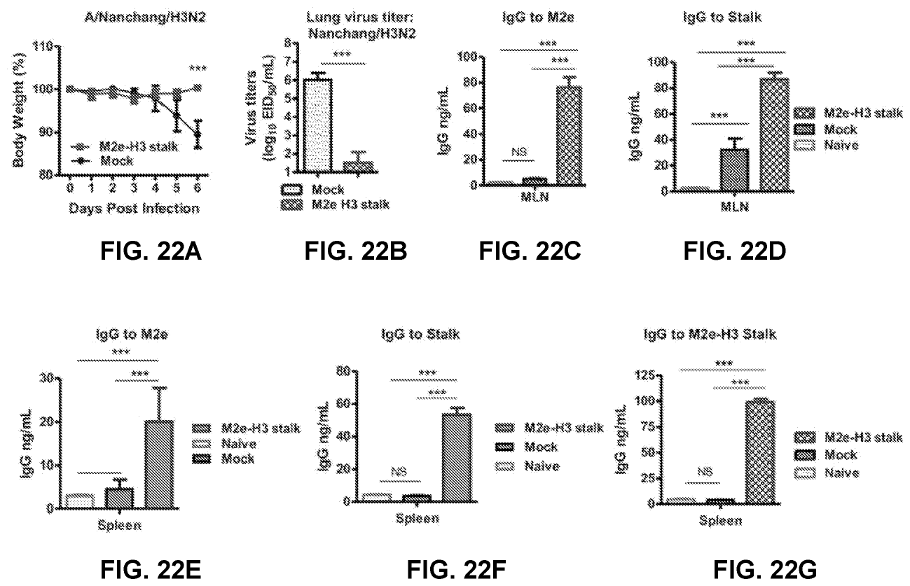

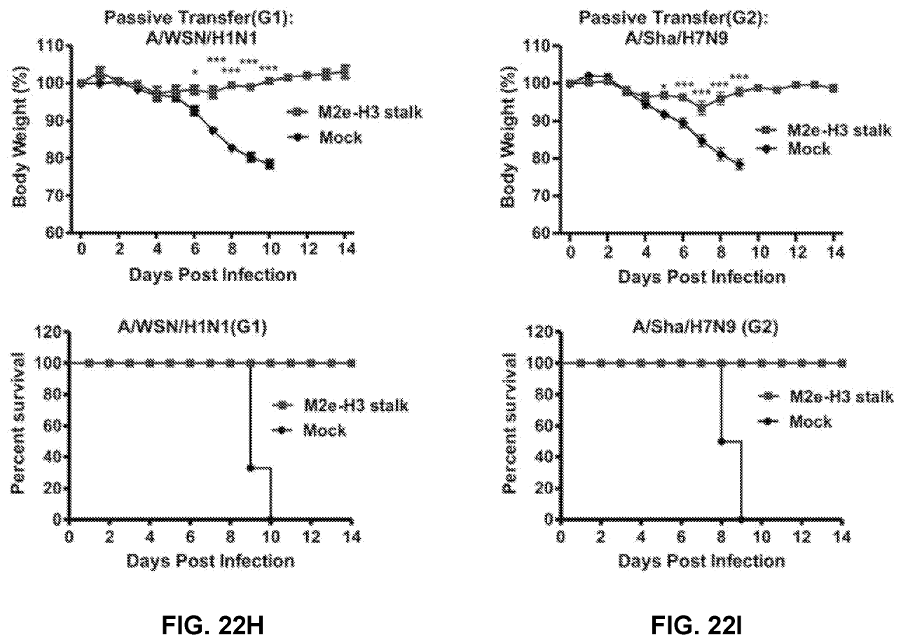

For better assessment of protective efficacy, we analyzed lung viral titers, humoral and cellular immune responses. M2e-H3 stalk vaccination significantly reduced lung viral titers by a magnitude of over 4 log 10 day 6 post-challenge with A/Nanchang/1995(H3N2) compared to mock group (FIG. 22B). M2e and stalk specific IgG secreting cell responses were determined in the culture supernatants of MLN and spleens collected at day 6 after challenge with rgA/Nanchang/1995 (H3N2) (FIGS. 22C-22G). M2e specific IgG antibodies were secreted in both MLN and spleen cell cultures only from M2e-H3 stalk vaccinated mice (FIGS. 22C and 22E). Also, significantly higher levels of stalk-specific IgG antibodies were produced in both MLN and spleen cell in vitro cultures from M2e-H3 stalk vaccinated mice (FIGS. 22D and 22F).

To evaluate the role of humoral immune responses in providing cross-protection, naive mice were intranasally infected with a mixture of lethal dose virus and boost antisera of adjuvanted M2e-H3 stalk vaccination (FIGS. 22H-22I). Consistent with cross group protection, the naive mice inoculated with a mixture of group 1 virus (A/WSN/H1N1) and M2e-H3 stalk boost sera showed complete protection without severe weight loss. In contrast, the control mice given this mixture of naive sera and virus did not survive (FIG. 22H). Also, the naive mice infected with lethal group 2 virus (rgA/H7N9) and M2-H3 stalk antisera were protected, despite a moderate weight loss (<10%), whereas mock sera with rgA/H7N9 virus failed to provide any protection (FIG. 22D).

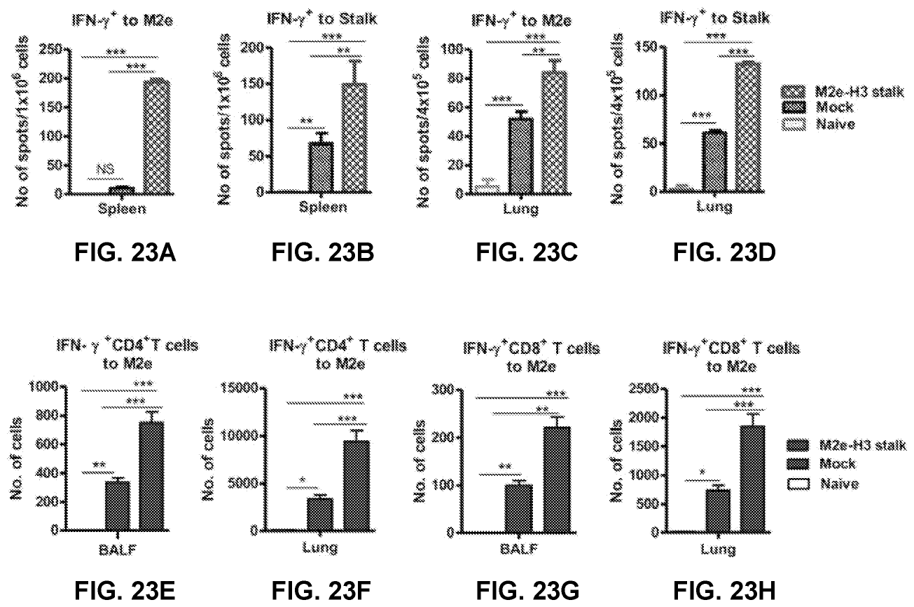

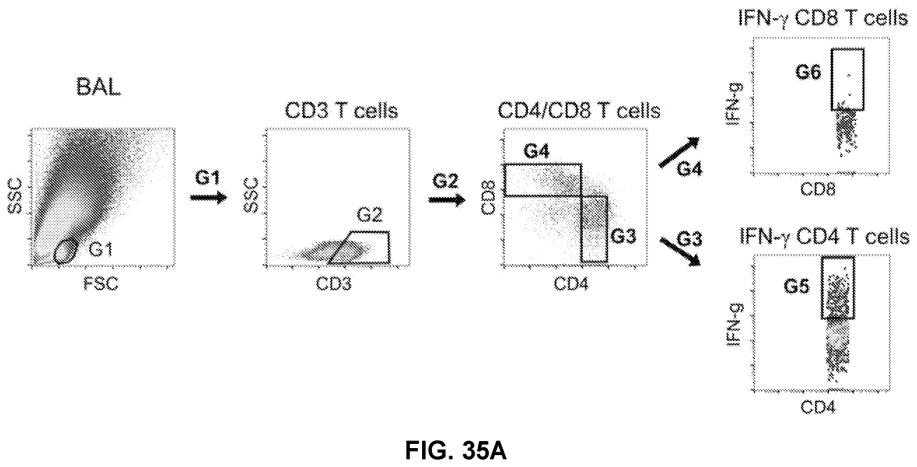

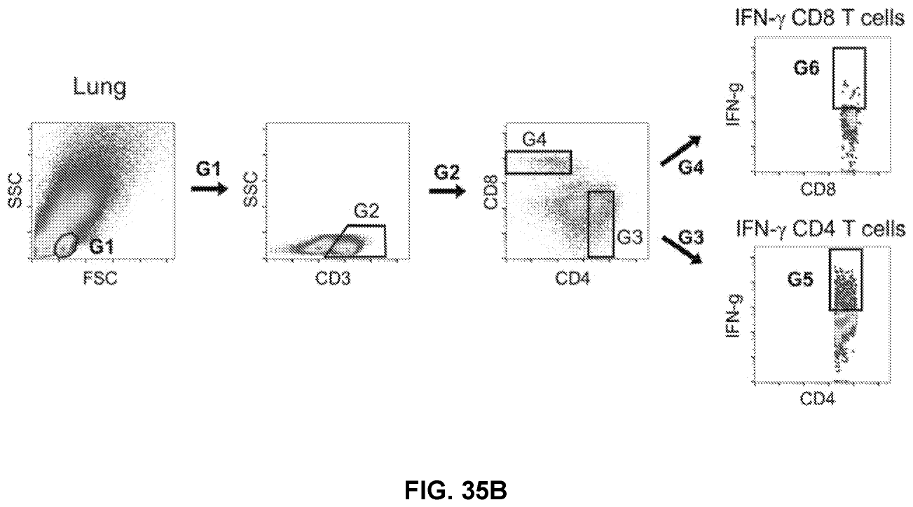

T cell immune responses were assessed by ELI spot assay and flow cytometry analysis (FIGS. 23A-23H). M2e-stimulated IFN-γ+ secreting splenocyte cell spots were observed only in M2e-H3 stalk vaccinated mice at significant levels (FIGS. 23A and 23C). Stalk-stimulated IFN-γ+ secreting splenocyte cell spots were induced at higher levels by adjuvanted M2e-H3 stalk vaccination than those in naive mice as determined day 6 post infection (FIGS. 23B and 23D). Intracellular cytokine staining and flow cytometry analysis indicated significantly enhanced levels of M2e-specific IFN-γ+ CD4+ T cells and IFN-γ+ CD8+ T cells in the airway bronchoalveolar lavage fluid (BALF) and lung samples in the adjuvanted M2e-H3 stalk group compared to those in naive mice collected day 6 post infection (FIGS. 23E-23H and 35A-35B). These data suggest that adjuvanted M2e-H3 stalk vaccination induces enhanced lung viral clearance and humoral and cellular immune responses.

In addition, the contribution of T cell immunity to cross-protection was determined in boost-vaccinated M2e-H3 stalk by depleting CD4+ and CD8+ cells before lethal challenge with rgA/Shanghai (H7N9) virus (FIG. 23I). Depleting CD4 and CD8 T cells in M2e-H3 stalk vaccinated mice resulted in significantly more weight loss (˜12%) than the T cell-nondepleted M2e-H3 stalk vaccinated mice which displayed minimum weight loss (˜6%) and quickly recovered, supporting the contribution of T cells to enhancing cross-protection.

### M2e-H3 Stalk Vaccine Provides Effective Protection Against Both Group 1 and 2 Viruses in Old-Aged Mice

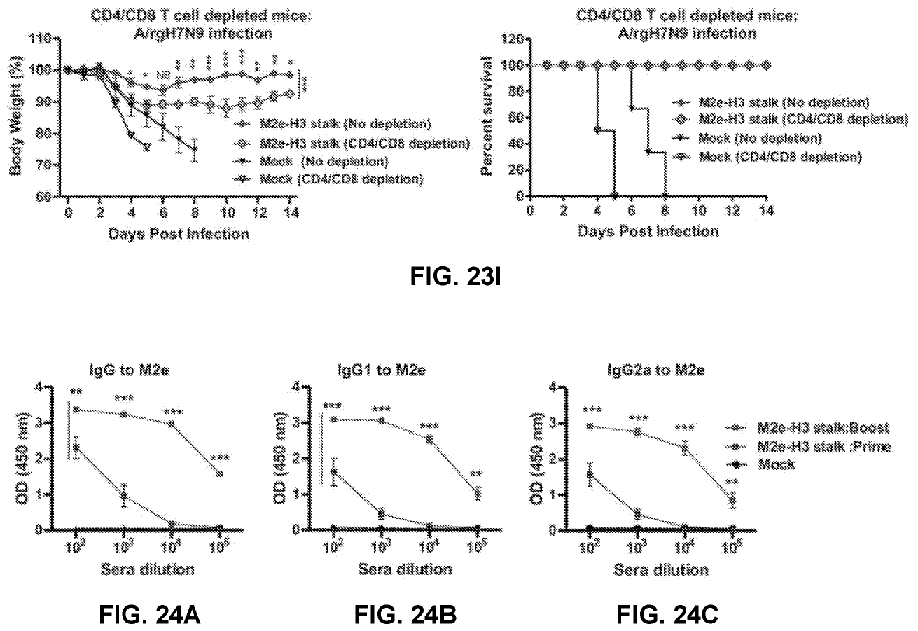

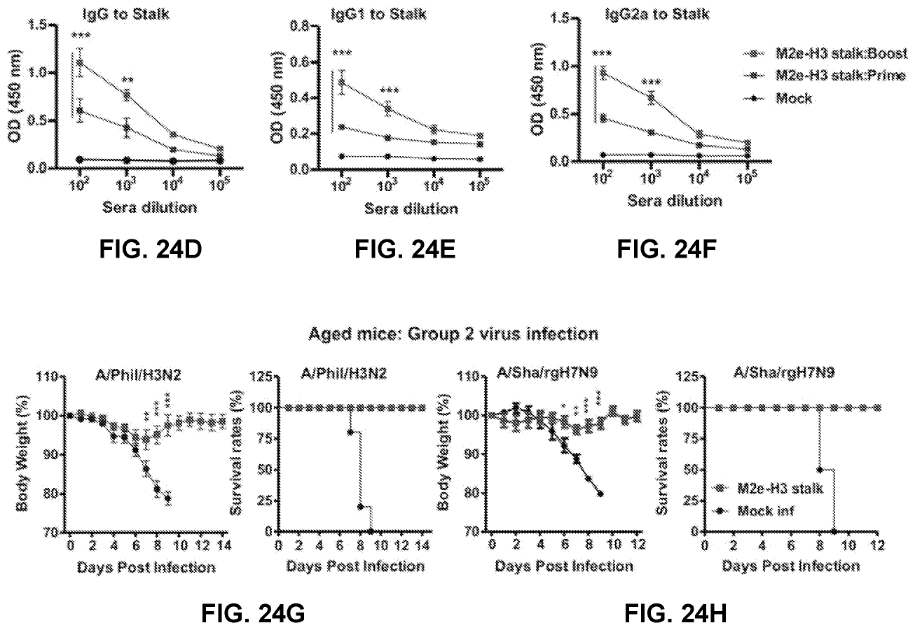

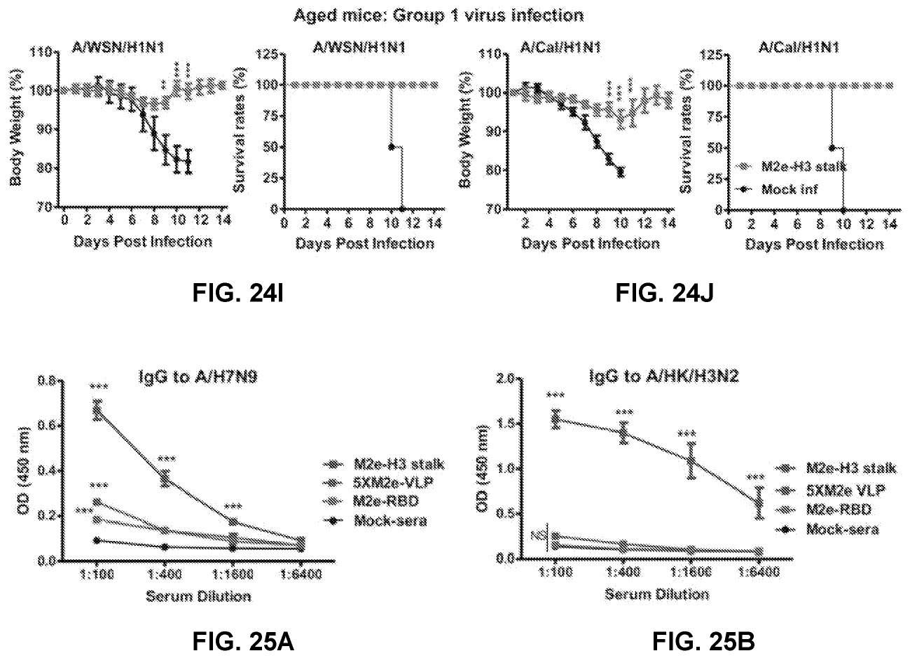

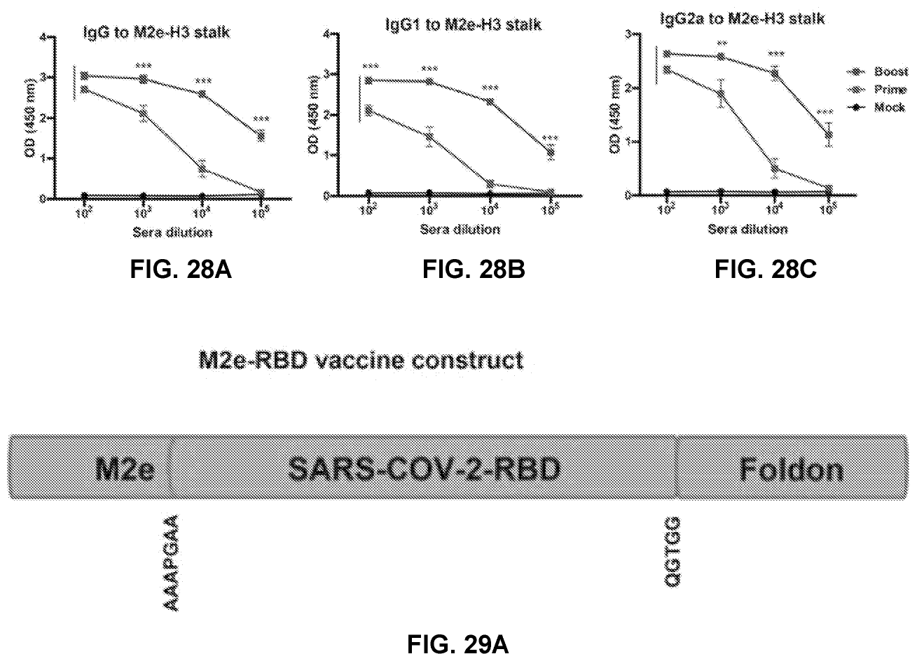

Groups of old aged (16 months old) mice were prime boost vaccinated with adjuvanted M2e-H3 stalk protein (20 μg) at a 3-week interval. High levels of M2e (FIGS. 24A-24C) and specific IgG antibodies were induced by adjuvanted M2e-H3 stalk vaccination of aged mice (FIGS. 24D-24F). Similarly, the levels of IgG specific for M2e-H3 stalk were significantly increased after boost in aged mice (FIGS. 28A-28C). The vaccinated aged mice were protected against NPhil (H3N2, group 2) virus challenge at 8 weeks after boost and prevented weight loss to minimum (<5%) compared to the mock control group displaying severe weight loss (>20%) with partial 50% survival rates (FIG. 24G). Consistent, high efficacy of protection against a lethal dose of rgA/Shanghai/2013 (H7N9, group 2) was observed with minimum weight loss (<5%) in the vaccinated aged mice (FIG. 24H). Similarly, adjuvanted M2e-H3 stalk vaccination of aged mice provided protection against H1N1 group 1 viruses (A/California/2009, A/WSN/1933), preventing severe weight loss (<7%) whereas the mock control mice did not survive H1N1 virus infection (FIGS. 23I-23J). These results suggest that M2e-H3 stalk vaccination induces effective protection against both group 1 and 2 viruses in old-aged mice.

M2e-H3 Stalk Protein Vaccine Dosage Effects on Inducing IgG Responses and Protection Efficacy with M2e Only Vaccines

We tested different dosage effects of M2e-H3 stalk protein (5 μg, 10 μg, 20 μg) on inducing IgG responses and protection after prime boost adjuvanted vaccination. There were no significant differences in the levels of IgG Abs specific for M2e and stalk antigens between the 10 μg and 20 μg vaccine dose groups whereas the 5 μg dose group induced lower levels of M2e and M2e-H3 stalk binding antibodies than those in the higher dose groups (FIGS. 26A-26E).

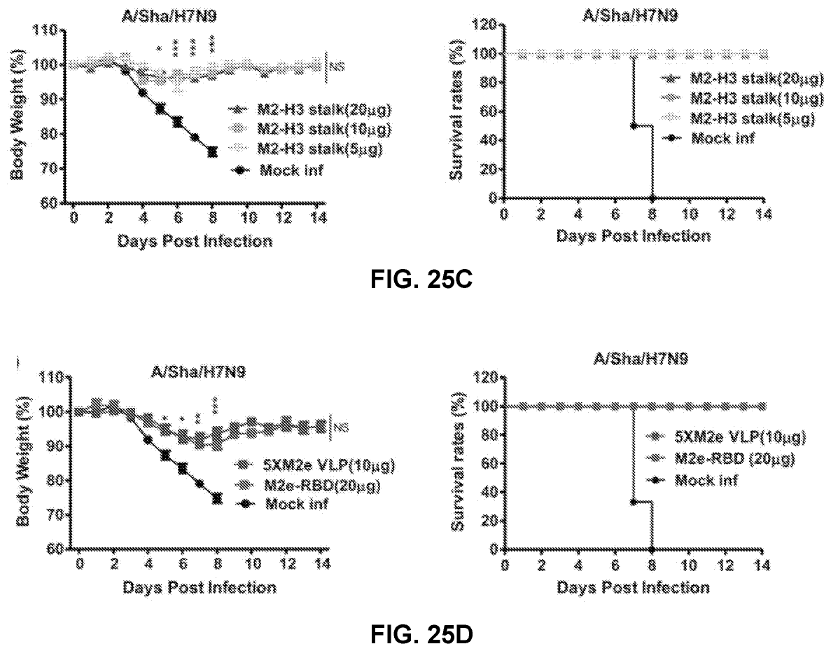

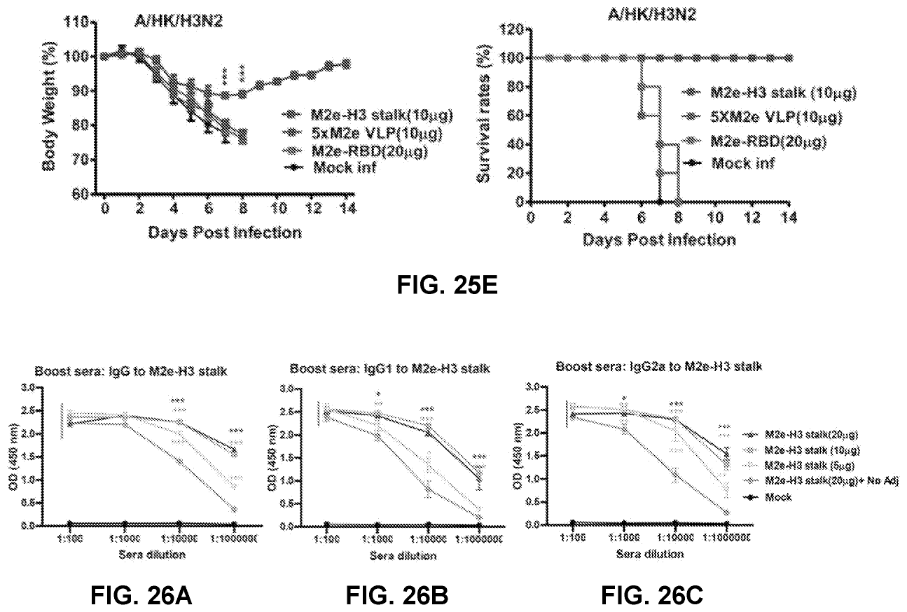

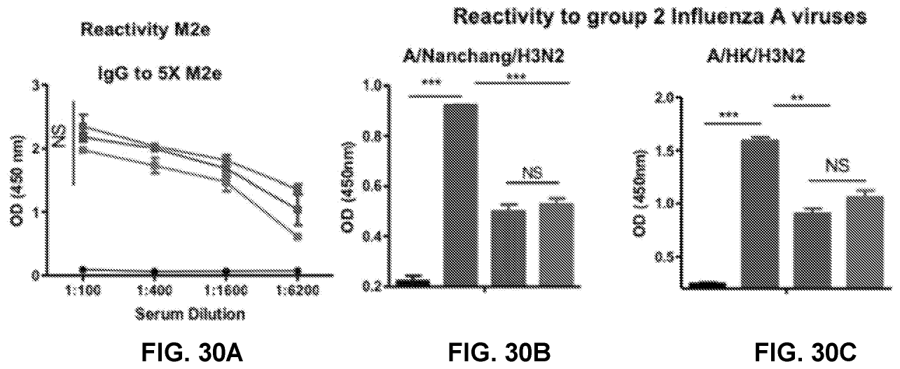

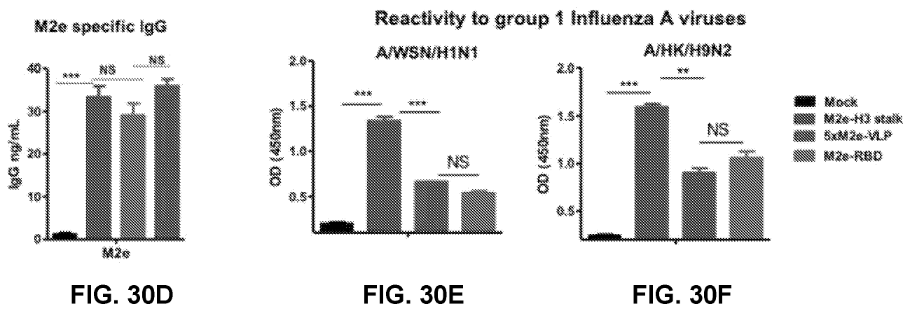

We also compared M2-H3 stalk vaccine immunogenicity and efficacy with M2e only vaccines (M2e-RBD, 5xM2e VLP). High levels of IgG antibodies specific for M2e were induced by vaccination with M2e-H3 stalk, M2e-RBD, or 5xM2e virus-like particle (VLP) without significant differences among the groups (FIG. 30A). The group of M2e-H3 stalk but not M2e only vaccination induced IgG antibodies highly reactive to inactivated group 2 viruses such as A/H7N9 and AIHK/H3N2 (FIGS. 25A-25B). In addition, M2e-H3 stalk vaccination induced significantly higher levels of IgG antibodies binding to cell-expressed viral antigens after infection of MDCK cells with group 2 viruses (NNanchang/H3N2, NHK/H3N2) and group 1 viruses (A/WSN/H1N1, A/H9N2) than those by M2e alone vaccination (FIGS. 30B-30E).

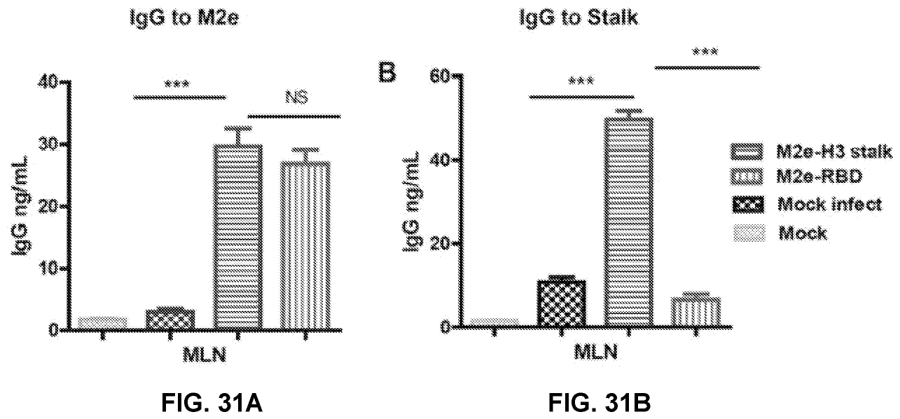

The groups of mice vaccinated with different doses (5 μg, 10 μg, 20 μg) of M2c-H3 stalk were similarly well protected against rgA/Shanghai/H7N9 virus, preventing weight loss (FIG. 25C). Meanwhile, the groups of mice vaccinated with 5xM2e VLP (10 μg) or M2-RBD (20 μg) displayed a moderate level (˜10%) weight loss after rgA/Shanghai/H7N9 virus challenge (FIG. 25D). The mock group did not survive after vims infection. After challenge with a high lethal dose of A/HK/H3N2 virus (10×LD₅₀), all mice in the 5xM2e VLP group died of infection (FIG. 25E). The M2e-H3 stalk group was completely protected against A/HK/H3N2 virus challenge, displaying only a moderate level (10%) weight loss. The mediastinal lymph nodes (MLN) collected from either the M2e-H3 stalk or M2e-RBD vaccination group, after challenging with A/Nanchang/H3N2 virus, were highly effective in inducing rapid plasma cell responses secreting M2e specific IgG antibodies (FIG. 31A). As expected, only the M2e-H3 stalk group showed IgG antibodies specific for stalk domain at significantly higher levels in culture supernatants of MLN (FIG. 31B). These results support that chimeric M2e-H3 stalk vaccine can be more effective in inducing cross protection than M2e alone based vaccine construct.

### DISCUSSION

Group 1 stalk only vaccines were ineffective in inducing cross protection against heterosubtypic and group 2 viruses due to sequence variations. In particular, the vaccine effectiveness against H3N2 over the past decade was in a low range of approximately 33% and significantly low down to 6% during the 2014-2015 season. New H3N2 variants with drifting mutations emerged with increased virulence. The outbreak of group 2 H7N9 subtype virus represented one of potential pandemics. Thus, it is of high priority to develop an effective group 2 or cross group vaccine. Here we presented a unique design and successful expression of M2e-H3 stalk protein construct in E. coli, which was thermostable and antigenically exposing conserved M2e, fusion peptide, and native-like stalk epitopes recognized by antisera of live group I and 2 vims infections. Vaccination of mice using M2e-H3 stalk protein with adjuvant (QS-2 I+MPL) induced IgG antibodies specific for M2e, HA stalk, and group 2 viruses and, to a lesser degree, group 1 viruses. Mice with adjuvanted M2e-H3 stalk vaccination were broadly protected against both group I and 2 viruses, supporting further development as a promising universal influenza A vaccine candidate.

Earlier studies reported isolation and characterization of human monoclonal antibodies (mAbs: FI6v3, CR9114, CT149) that could broadly neutralize group 1 and 2 viruses. Footprint and X-ray crystallography studies revealed the contact area between broadly neutralizing mAbs and HA conserved domains and residues. The group 1 HA stem mAbs (C 179, CR6261, F10) were shown to contact the HA2 stem N-terminal fusion peptide region and helix A domain proximal to the membrane as well as the N- and C-terminal regions of HA 1, with some differences among the mAbs. The contact sites of group 2 HA stem mAbs (CR8020, CR8043) and cross group rnAbs (FI6v3, CR9114, CT149) were mapped to be HA2 multi-domains including the fusion peptide C-terminal region, an outmost edge of the P-sheet and helix A (FIG. 17D). Although the contact sites were overlapping with those of group 1 rnAbs, the group 2 and cross group rnAbs appeared to have some differences such as a larger area of contact spanning the fusion peptides, the viral membrane proximal outer P-strands preceding helix A (FIG. 17D). These epitope mapping data of broadly neutralizing rnAbs have provided a rational design of stalk stabilized immunogens. Corbett et al. (2019) reported group 2 headless H3 stalk-stabilized (H3ssF) and H7 stalk-stabilized (H7ssF) ferritin nanoparticle immunogens constructed through helix stabilization (HA2 aa60-92 replaced with a G-rich loop), Bloop optimization, and multiple point mutations, and expressed in mammalian cells. Vaccination of mice with adjuvanted H3ssF and H7ssF immunogens induced subtype virus specific neutralizing activities and moderate efficacy of protection against lethal challenge. However, H3ssF and H7ssF immunogens did not induce heterologous cross neutralizing Abs and cross protection remains unknown, suggesting a limitation of stem only vaccination. In an attempt to overcome limited cross protection, we designed a chimeric M2e-H3 stalk construct expressed in E. coli. The M2e-H3 stalk contains M2e repeat, HA1 stem-interacting fragments, HA2 aa 1-117 stem domain composed of fusion peptide with membrane proximal P-strands, helix A, loop B with point mutations, and helix C, which covers most epitopes known for broadly neutralizing stalk rnAbs (FIG. 17D). Antigenicity data of M2e-H3 stalk suggest the presentation of native-like conserved epitopes to be exposed for recognition by Abs specific for both group 1 and 2 HA and antisera from infection with live viruses in addition to M2e and stalk.

Full-length H3 stalk (aa 1-172) proteins with point mutations to retain prefusion-like conformation and headless HA1 fragments expressed in E. coli provided low efficacy of homologous and partial heterologous H3N2 protection in mice. In a follow up study, E. coli expressed, reduced sizes of H3 stalk helix domains (aa 44-113) with similar mutations and HA I fragments conferred only partial survival protection (40-50%) against homologous H3N2 virus despite the induction of cross-reactive IgG Abs 35. Sutton et al. (2017) demonstrated that the group 2 headless H3 and H7 stalk immunogens with several residues extended (aa 37-115) and stabilizing point mutations, expressed in E. coli, induced homologous H3N2 protection with minimum weight loss but low efficacy of cross protection against heterologous H3N2 or heterosubtypic H7N9 virus as shown by severe weight loss and partial survival rates.

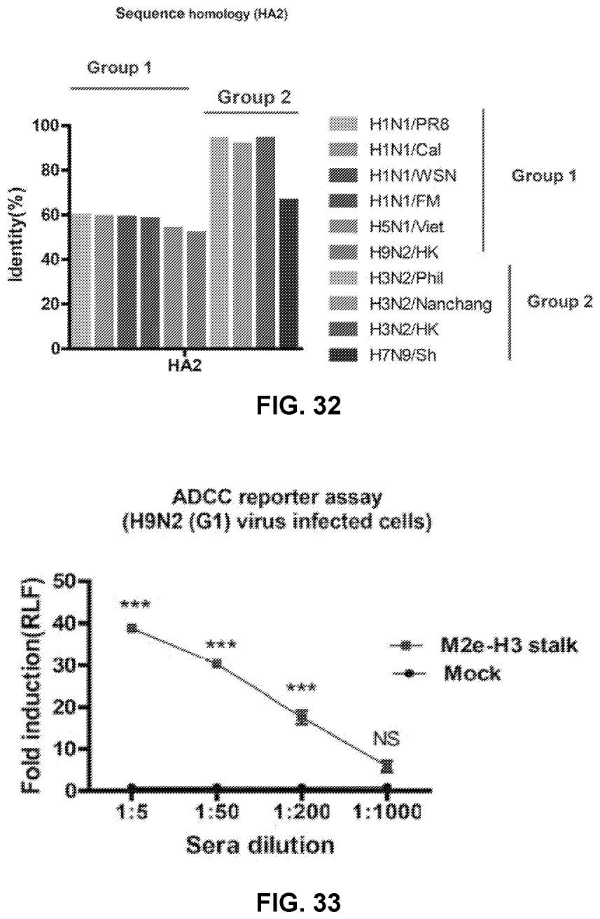

Stabilization of the group 2 HA headless stalk proteins required additional modifications as compared to the group 1 HA stalk. Here we demonstrate that M2e-H3 stalk protein vaccination induced broad cross-protection against both group 1 (H1N1, rgH5NI, rgH9N2) and group 2 (H3N2, rgH7N9) influenza A viruses, which supports that M2e-H3 stalk protein can be developed as a potential universal vaccine candidate. As expected, H3N2 virus specific IgG responses were induced at higher levels by M2e-H3 stalk vaccination than those for intragroup heterosubtypic (rgH7N9) or cross-group viruses (H1N1, rgH9N2). The HA stalk sequence homology is as high as over 94% among the same H3N2 subtype viruses but reduced to below 70% among different subtypes within the group 2, and further down to 60% homology with among the group 1 viruses (FIG. 32). Despite the low homology of stalk sequences, M2e-H3 stalk provided cross protection against heterosubtypic rgH7N9 and group 1 viruses such as H1N1 (A/WSN, NPR8, A/FM, A/Cal/2009), rgH5N1, and rgH9N2. The efficacy of cross protection by M2e-H3 stalk was significantly improved as evidenced by preventing weight loss in mice under lethal challenge. Particularly, it is the first time to report that M2e-H3 stalk provided cross protection against both group 1 and 2 viruses in aged mice without apparent weight loss under lethal challenge. Since the mortality rates of seasonal influenza viruses are relatively low, a condition of lethal dose challenge is considered appropriate to assess the efficacy of cross protection. In contrast, previous studies reported severe weight loss after heterologous challenge in mice with adjuvanted headless-stalk vaccination. Nonetheless, it is not possible to compare the cross protective efficacy with other studies, due to the differences in vaccine doses, adjuvants used, challenge virus and doses, and the number of vaccinations.

### Methods

Rational molecular design and synthesis of M2e-H3 stalk vaccine construct. HA gene sequence of influenza A virus [A/Aichi/12/1968(H3N2)] was obtained from GenBank (ID: M55059) and used to design the H3 stalk vaccine construct. The conserved domains of HA were identified by multiple alignment of influenza A virus sequences. The amino acid (aa) residues of the HA1 (aa37-61 and 305-338) and HA2 (aa 1-117) domains were included as a vaccine target based on the major conserved region of the HA stalk and stabilizing domain. Point mutations were introduced on the hydrophobic aa residues of the targeted fragments by replacing with polar and hydrophilic residues without affecting the structure of the HA stem. Cysteine 321 was replaced with serine (C321 S) on the HA 1 region. The conserved M2e sequence (SLLTEVETPIRNEWGSRSNDSSD, SEQ ID NO:5) repeat was introduced in the N terminal and the foldon sequence was connected to the C terminal of the selected H3 stalk domain. The structure of M2e and foldon was derived from the protein data bank (PDB) ID codes 4N8C and IRFO, respectively. The 3D structure of HA was predicted using the SWISS model and visualized in PyMol. The newly designed vaccine construct was named as M2e-H3 stalk. The nucleotide sequence of the M2e-H3 stalk construct was codon-optimized for expression in Escherichia coli (E. coli) and synthesized by Genscript (USA). In addition, to develop an M2e alone based vaccine, receptor-binding domain (RBD) of SARS-Co V-2 was fused with the M2e epitope and β-rich trimeric nature of foldon sequence on the N and C terminal with soluble linker sequences, respectively. The vaccine construct was codon-optimized for E. coli; synthesized and named as M2e-RBD.

### Example 3: Universal Protection Against Influenza Viruses by Multi-Subtype Neuraminidase and M2 Ectodomain Virus-Like Particle

### Results

Preparation of m-cNA-M2e VLP as a Single Entity Universal Vaccine Candidate

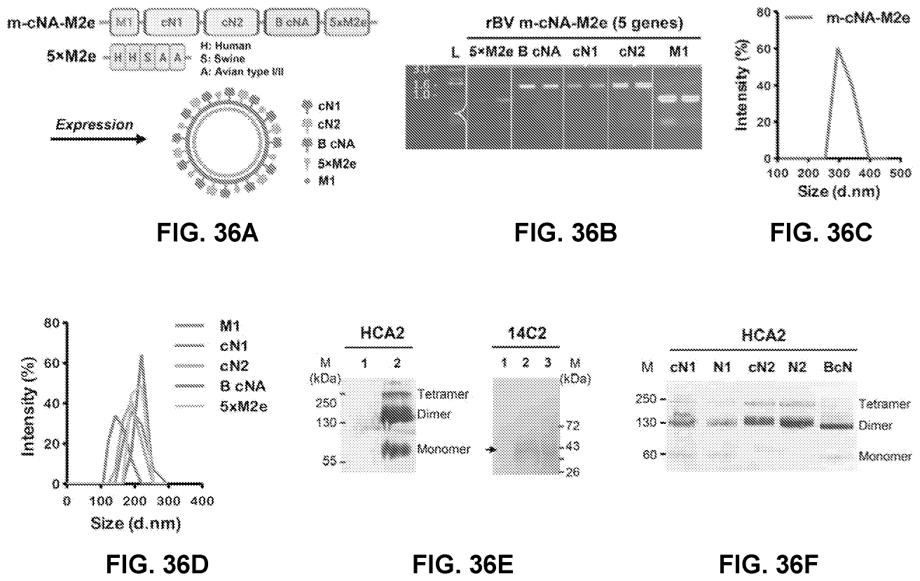

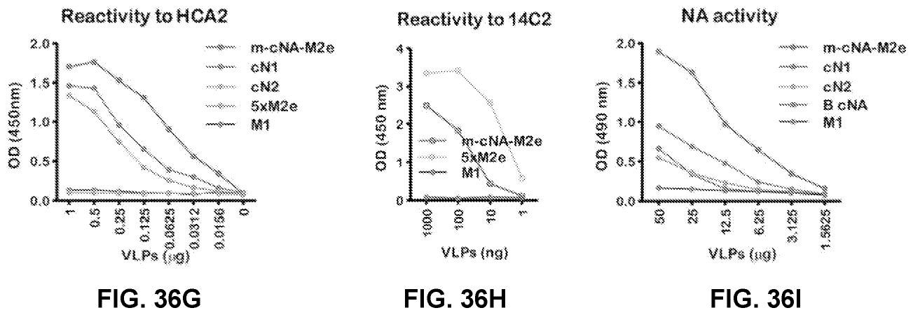

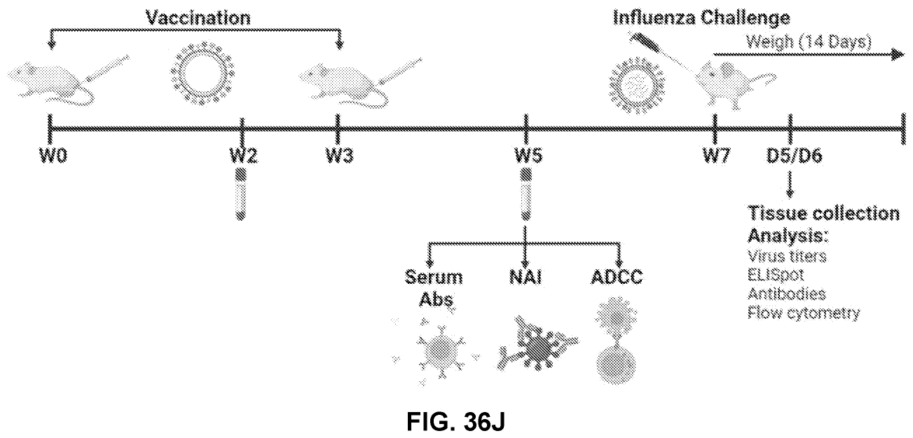

Consensus N1 NA (cN1), N2 NA (cN2), and influenza B NA (B cNA) sequences were generated from human isolates (2010-2019) available at NCBI, by using the sequence alignment program. The highly conserved tandem repeat 5xM2e VLP has been described in prior studies. The rBV vector (pFastBac1) expressing 5 genes (M1-cN1-cN2-B cNA-5xM2e) under each polyhedrin promoter was confirmed for each gene by PCR (FIGS. 36A and 36B). The particle sizes of multi-component VLPs produced in insect cells were found to be in a range between 250 to 400 nm with an average 325 nm in diameter (FIG. 36C), which was slightly bigger than those (averages 142-220 nm) of mono subtype (cN1, cN2, or B cNA) consensus NA VLPs (FIG. 36D). The western blot data indicated that NA proteins were presented in heterogeneous multimeric forms on m-cNA-M2e VLP, which includes dimers in majority, monomers, and tetramers (FIG. 36E left). Mono subtype cN2 and B cNA VLP preparations showed dimeric forms in majority (FIG. 1F). The m-cNA-M2e construct containing cN1, cN2, and B cNA as well as 5xM2e on the same VLP as a single entity showed high reactivity to monoclonal antibody (mAb) HCA-2 specific for pan-NA222-230 (FIG. 1 G) and M2e-specific 14C2 mAb (FIG. 36H). The monomeric cN1 and cN2 VLP preparations also displayed moderate reactivity to HCA-2 mAb (FIG. 36G). In addition, m-cNA-M2e VLP vaccine retained high functional activity of NA (FIG. 36I) which was also observed in mono subtype cN1, cN2, and B cNA VLP preparations (FIG. 36I). Taken together, we successfully developed m-cNA-M2e VLP containing consensus multi subtype NA (cN1, cN2, and B cNA) and 5xM2e proteins as a single entity universal vaccine candidate.

m-cNA-M2e VLP Vaccination Induces IgG Antibodies Specific for M2e and NA as Well as Broad NA Inhibition Activity Against to Influenza a and B Viruses

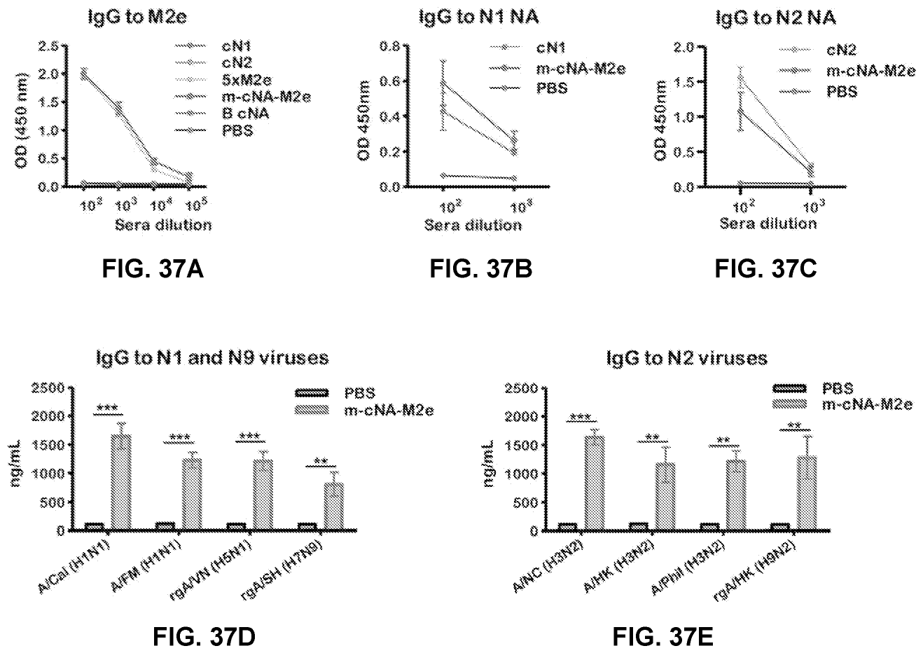

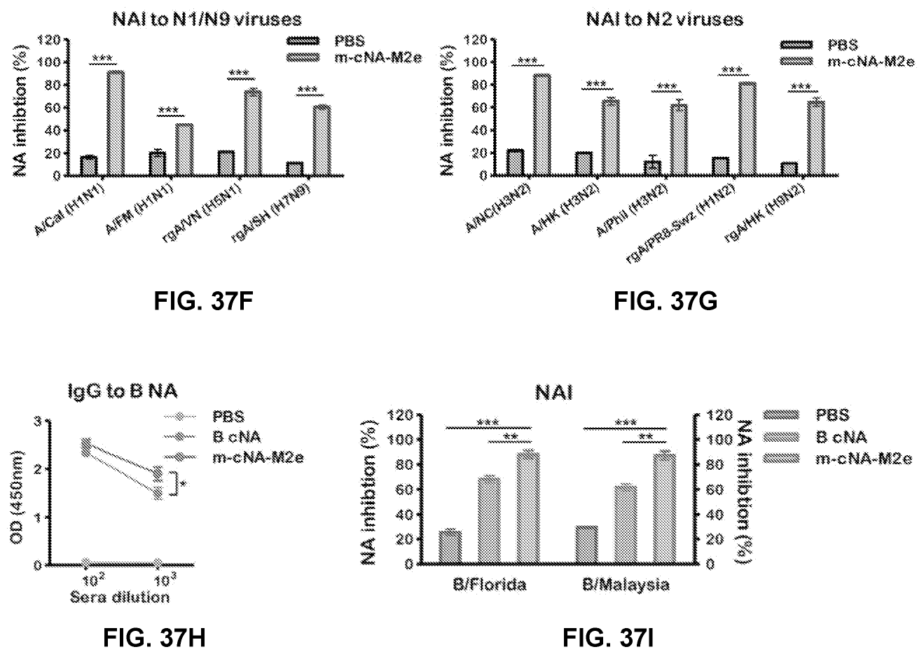

Mice (n=10 per group) were intramuscularly (IM) prime boost immunized with indicated VLP vaccines at a 3-week interval (FIG. 36J). Antisera of m-cNA-M2e VLP boost showed high levels of IgG antibodies for M2e, which are comparable to 5xM2e VLP, but not from the cN1 and cN2 VLP (FIG. 37A). The m-cNA-M2e VLP group also induced IgG antibodies specific for N1 and N2 NA proteins at comparable levels to mono cN1 and cN2 VLP (FIGS. 37B and 37C). The m-cNA-M2e VLP group induced high levels of IgG antibodies specific for inactivated H1 N1 (A/Cal) and H3N2 (A/NC) viruses, moderate levels for H1 N1 (A/FM), H5N1 (rgA/VN) (FIG. 37D), H3N2 (A/HK, A/Phil), and H9N2 (rgA/HK) (FIG. 37E), and for H7N9 (rgA/SH) viruses (FIG. 37D).

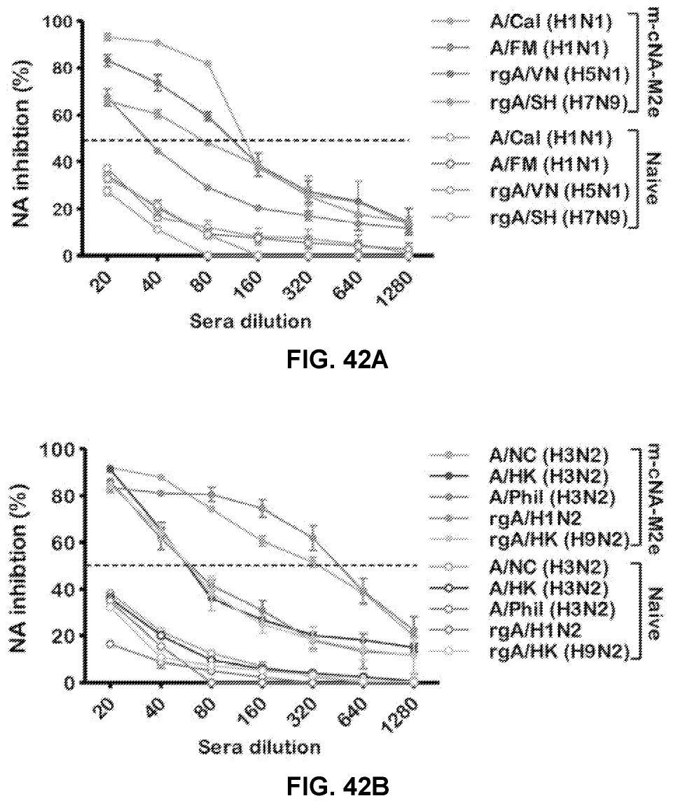

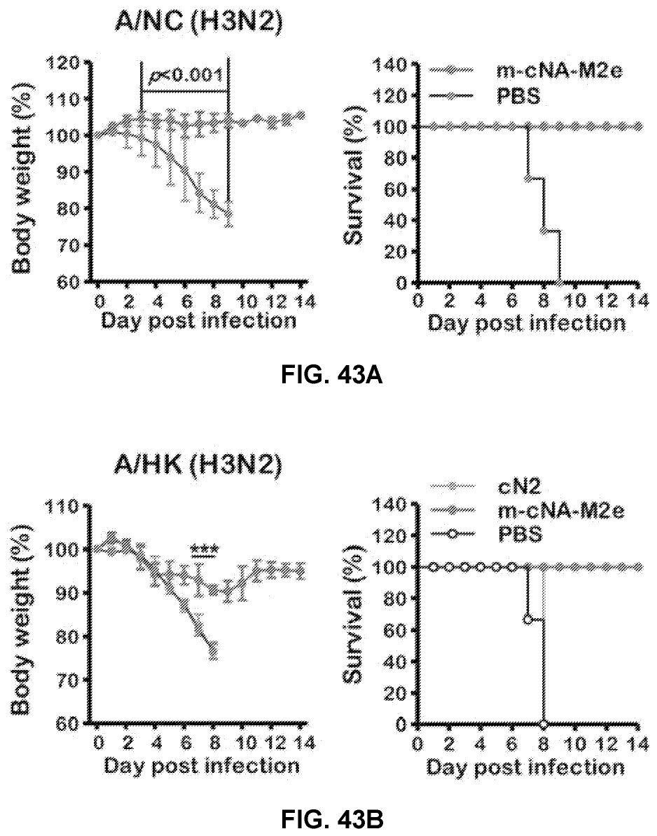

NA inhibition (NAI) titers likely correlate with protection. m-cNA-M2e VLP immune sera exhibited high levels of NAI titers against A/Cal H1N1, rgA/VN H5N1, A/NC H3N2, and rgA/H1 N2 (FIGS. 37F and 37G, 42A). NAI titers against H7N9 (rgA/SH) (FIG. 37F, 42A), H3N2 (NHK, NPhil), and H9N2 (NHK) (FIG. 37G, 42B) were at a moderate level. Next, investigated was whether m-cNA-M2e VLP containing consensus B-NA provides cross lineage protection against influenza B viruses. The IgG antibodies specific for influenza B NA protein (FIG. 37H) and NA inhibition activity to influenza B viruses (FIG. 37I) were induced at higher levels in boost immune sera of m-cNA-M2e VLP group compared to those in B cNA VLP and mock control groups. These results indicate that m-cNA-M2e VLP vaccination induces M2e and NA specific IgG antibodies as well as NA inhibition activity against a broad range of influenza A and B viruses.

Single m-cNA-M2e VLP is Superior to Mono VLPs in Inducing Broad Cross Protection Against Influenza a Viruses with N1, N2 or N9 NA as Well as B Viruses

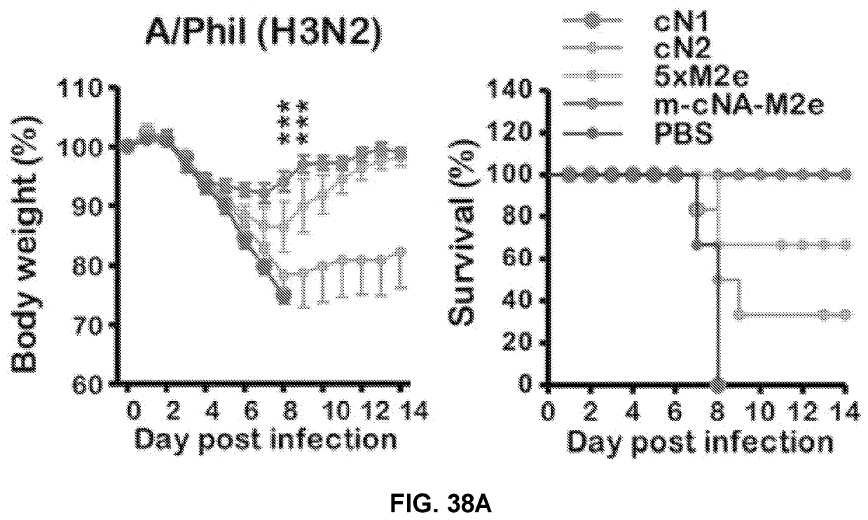

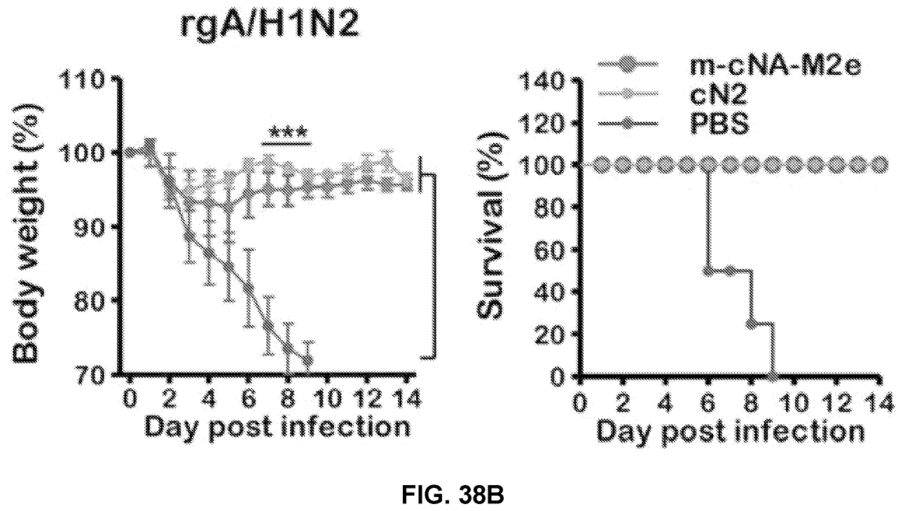

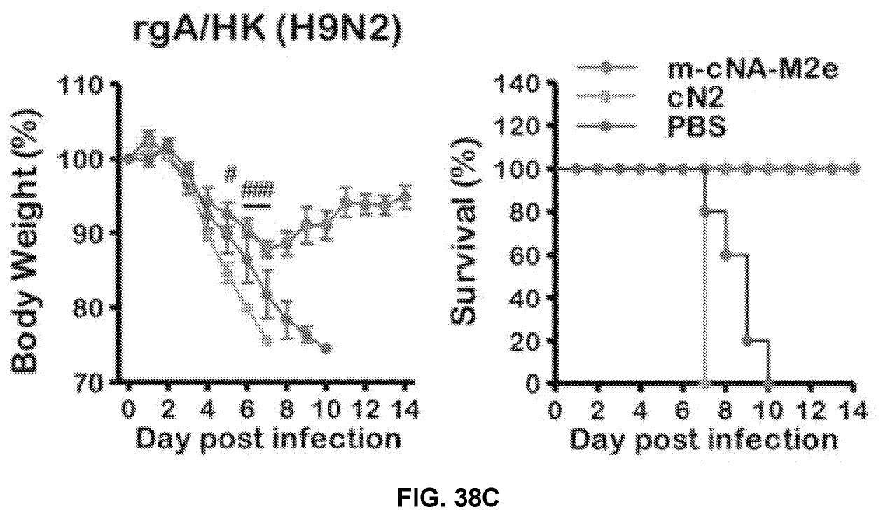

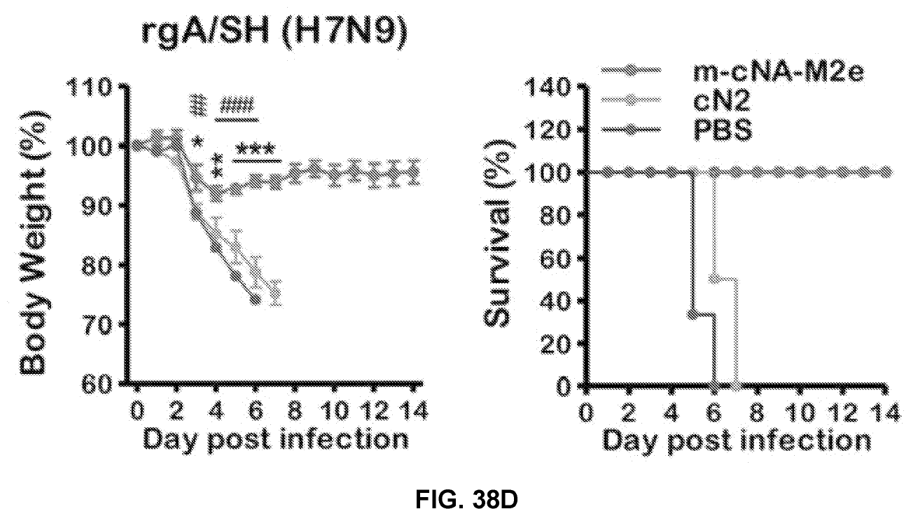

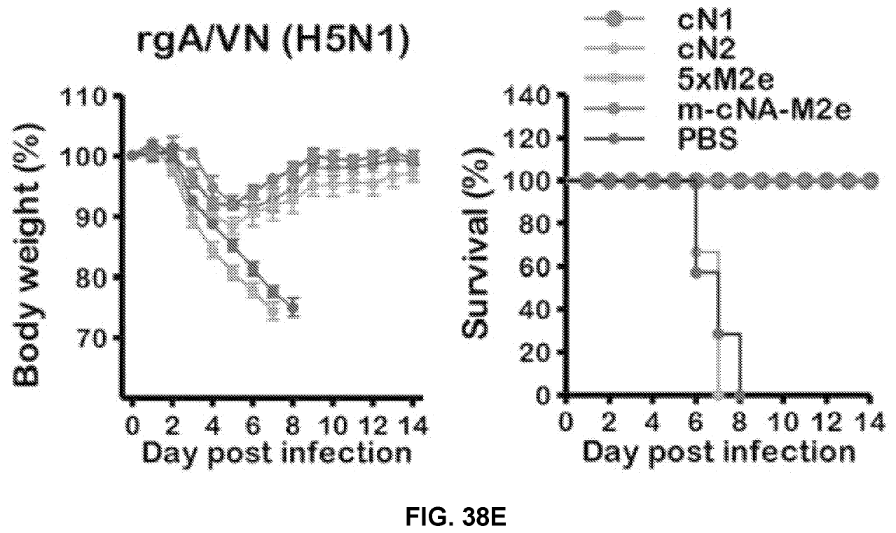

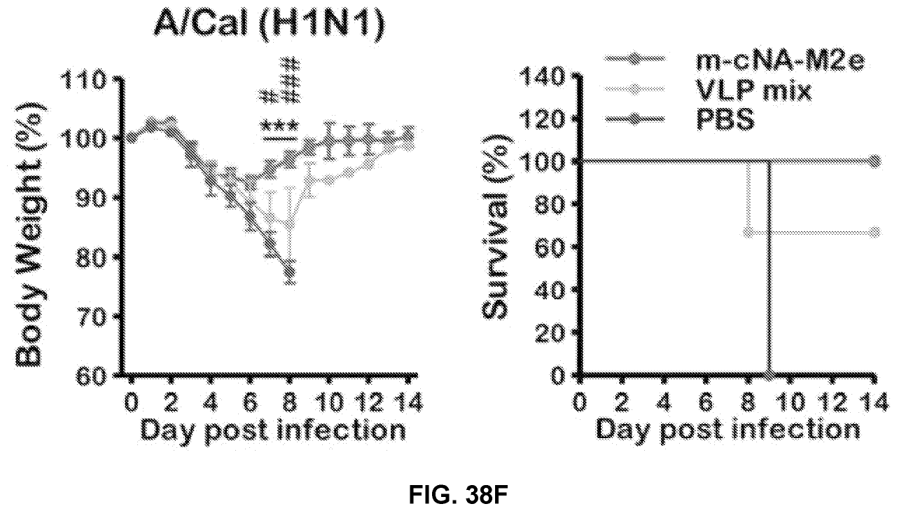

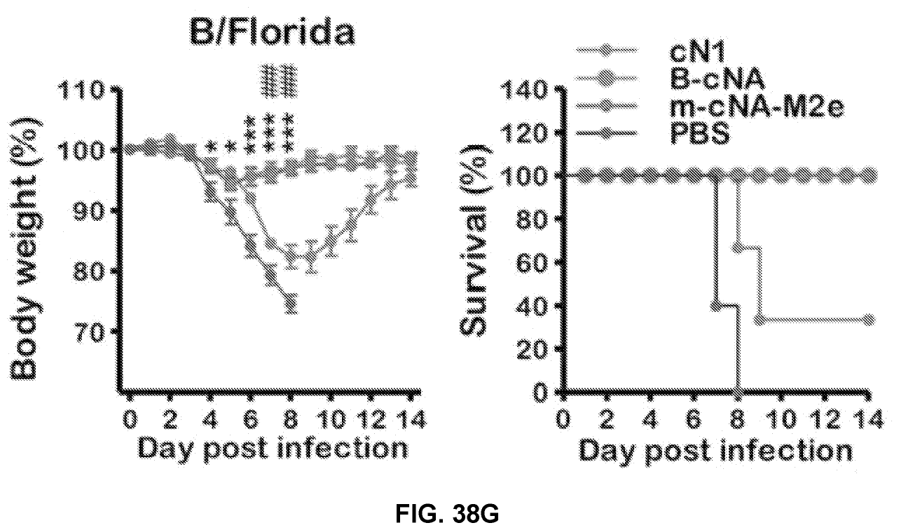

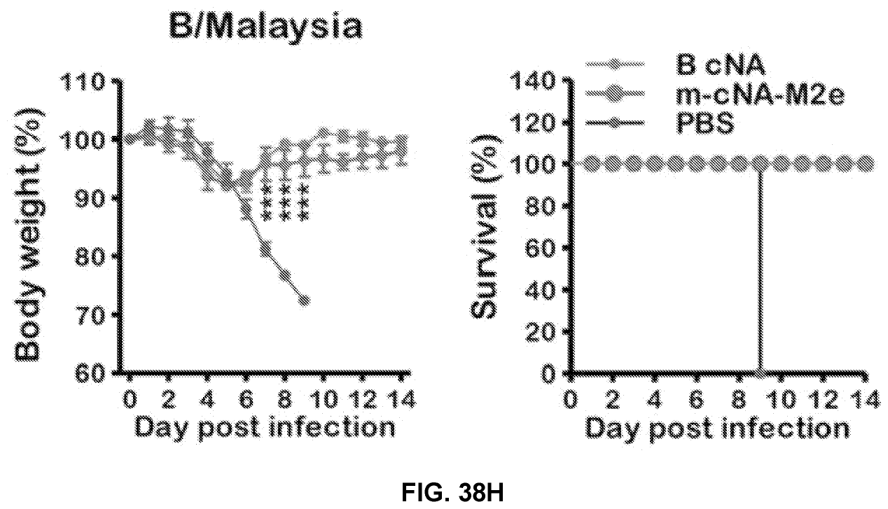

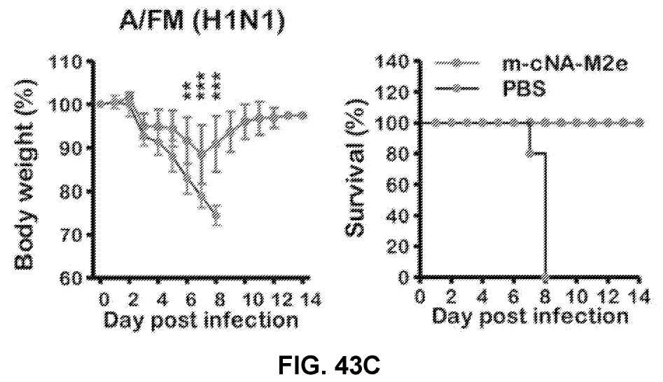

The efficacy of m-cNA-M2e VLP, cN1 VLP, cN2 VLP, and 5xM2e VLP was compared after challenge with A/Phil/1982 H3N2 virus at 4 weeks after boost. The cN1 VLP and mock control mice did not survive (FIG. 38A). The cN2 VLP group displayed severe weight loss over 20% and survival rates 30%. Substantial weight loss (˜15%) and partial protection (60%) were observed in the 5xM2e VLP group (FIG. 38A). The m-cNA-M2e VLP group induced most effective protection, with moderate (10%) weight loss (FIG. 38A). Furthermore, the m-cNA-M2e VLP group did not display any weight loss against lethal challenge with A/NC/1995 H3N2 virus whereas the mock control group showed no protection (FIG. 43A). More effective protection against A/HK/1968 H3N2 virus was observed with m-cNA-M2e VLP than cN2 VLP displaying no protection (FIG. 43B). Both the cN2 VLP and m-cNA-M2e VLP groups were well protected against rgA/H1 N2 virus with homologous NA, derived from A/Switzerland/2013 (H3N2) (FIG. 38B). Also, m-cNA-M2e VLP vaccination induced protection against lethal challenges with rgA/HK/1999 H9N2 (FIG. 38C) and rgA/SH/2013 H7N9 (FIG. 38D) viruses, despite moderate (8-11%) weight loss.

Next, we compared the efficacy of m-cNA-M2e VLP, cN1 VLP, cN2 VLP, and 5xM2e VLP after challenge with rgANN H5N1 virus (FIG. 38E). The m-cNA-M2e VLP and cN1 VLP groups showed a similar pattern of weight loss (˜9%) at days 4 to 6 post challenge whereas 5xM2e VLP exhibited moderate (˜12%) weight loss (FIG. 38E). As expected, cN2 VLP was not protected against NA heterosubtypic rgANN/2004 H5N 1 (FIG. 38E). Furthermore, we evaluated whether m-cNA-M2e VLP would have potential advantages over a simple mixture of each mono VLP (FIG. 38F). The m-cNA-M2e VLP group showed less weight loss than the VLP mix group (˜10% versus 15%) after challenge with NCal/2009 H1 N1 virus at 4 weeks post boost (FIG. 38F). Similarly, the m-cNA-M2e VLP group displayed efficient protection against NFM H1 N1 virus with a moderate level of weight loss (˜10%) whereas mock control group did not survive (FIG. 43C).

Furthermore, the mice vaccinated with m-cNA-M2e VLP were equally protected against Yamagata lineage B/Florida/4/2006 virus, exhibiting minimum weight loss (˜5%) against lethal challenge (FIG. 38G). Comparable to protection by B cNA VLP, the m-cNA-M2e VLP group displayed low to moderate weight loss (˜6-7%) with 100% survival rates after lethal challenge with Victoria lineage B/Malaysia/2056/2004 virus (FIG. 38H). Taken together, these data suggest that m-cNA-M2e VLP vaccination induced broad cross protection against influenza A containing N1, N2, N9 and both lineage influenza B viruses in mice, providing further evidence for m-cNA-M2e VLP as a universal vaccine candidate.

Vaccination of Mice with m-cNA-M2e VLP Elicits CD4+ and

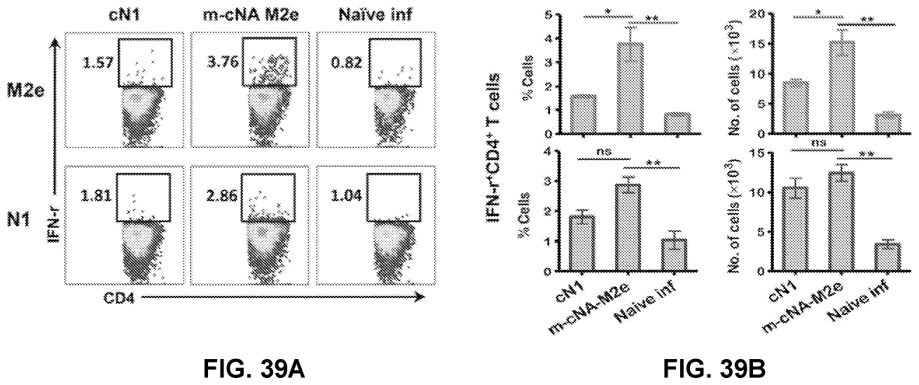

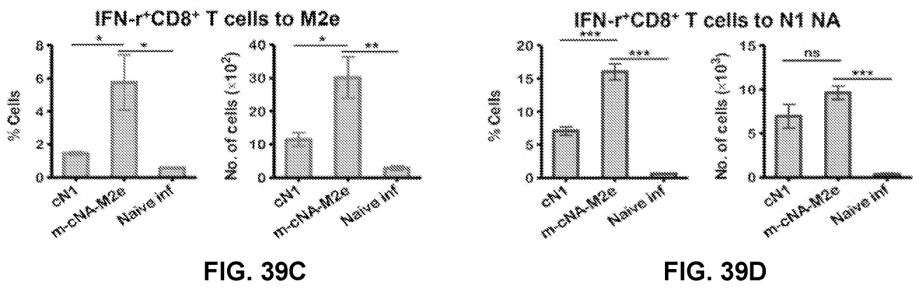

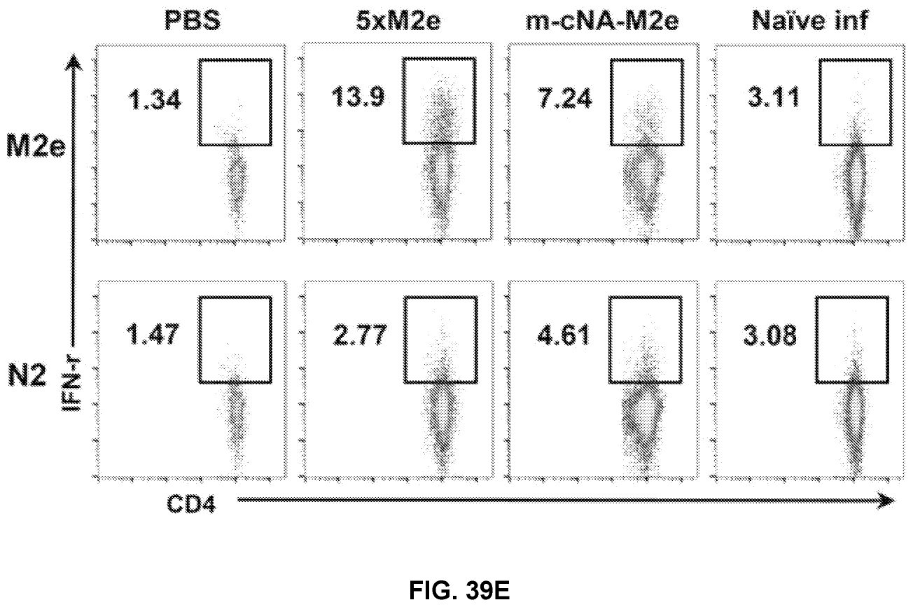

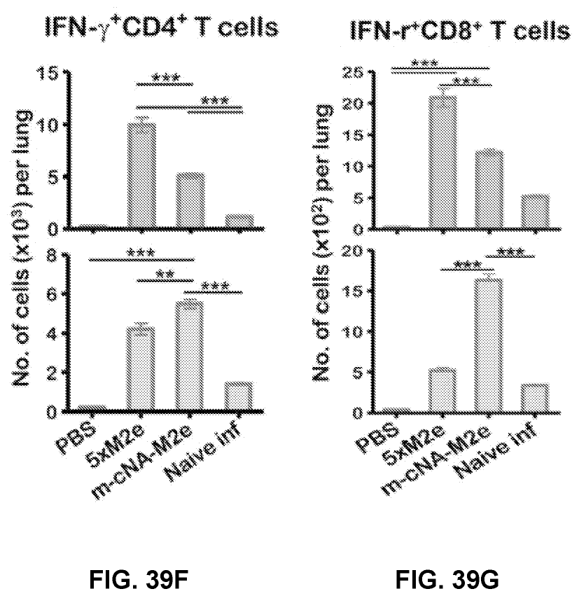

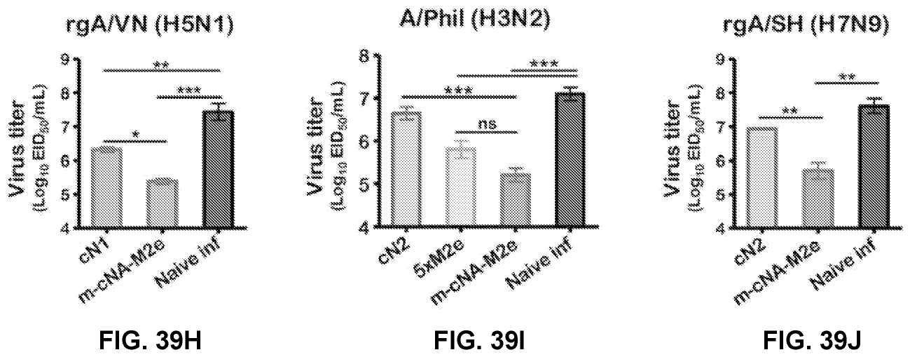

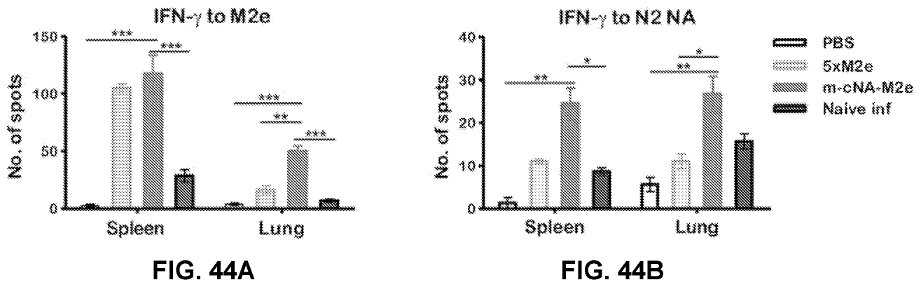

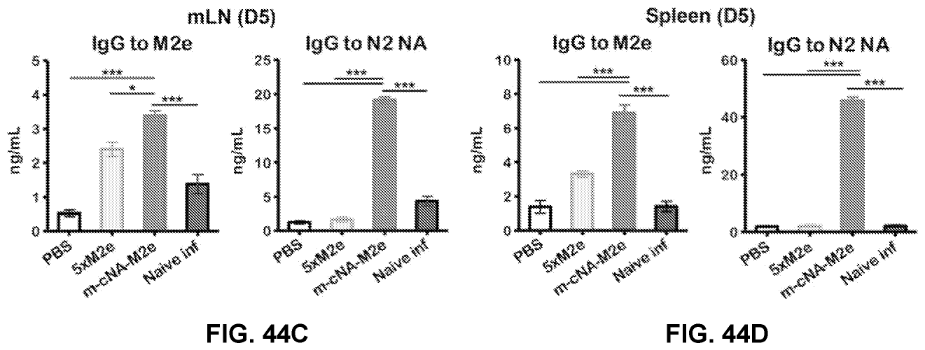

CDS+ T cell responses and antibody responses specific for M2e and N1 or N2 NA Cellular immunity plays an essential role in limiting infection and eventually clearing the virus from the body. Flow cytometry data showed significantly higher levels of IFN-γ+CD4 and IFN-γ+CD8 T cells upon M2e stimulation of lung cells from m-cNA-M2e VLP vaccination compared to cN1 VLP day 6 post rgA/VN H5N1 challenge (FIGS. 39A-39C). Also, N1 NA stimulated IFN-γ+CD8 T cells were induced at higher levels after m-cNA-M2e VLP vaccination than those after cN1 VLP vaccination (FIG. 39D) whereas the levels of NA stimulated IFN-γ+CD4 T cells were significantly different between the two groups (FIGS. 39A and 39B). Following A/Phil H3N2 challenge, the m-cNA-M2e VLP group induced significantly high levels of IFN-γ secreting splenocytes and lung cells after in vitro stimulation with M2e or N2 peptides by ELISpot assay (FIGS. 44A and 39B). Consistently, flow cytometry of intracellular cytokine staining showed that M2e or N2-specific CD4+ T (FIGS. 39E and 39F) and CD8+ T cells (FIG. 39G), which secrete IFN-γ, were substantially increased in the lung of m-cNA-M2e VLP group compared to the naive infection group.

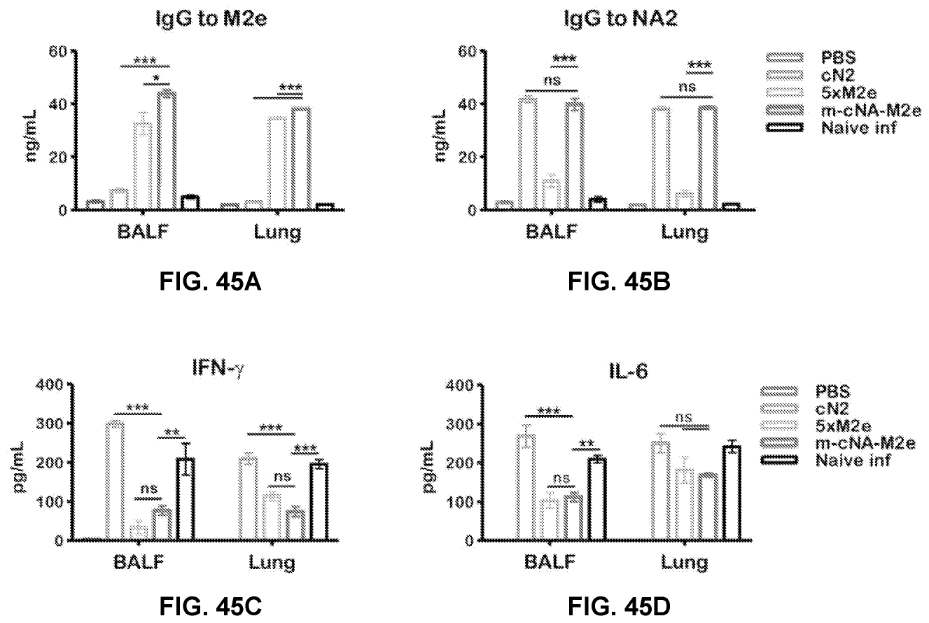

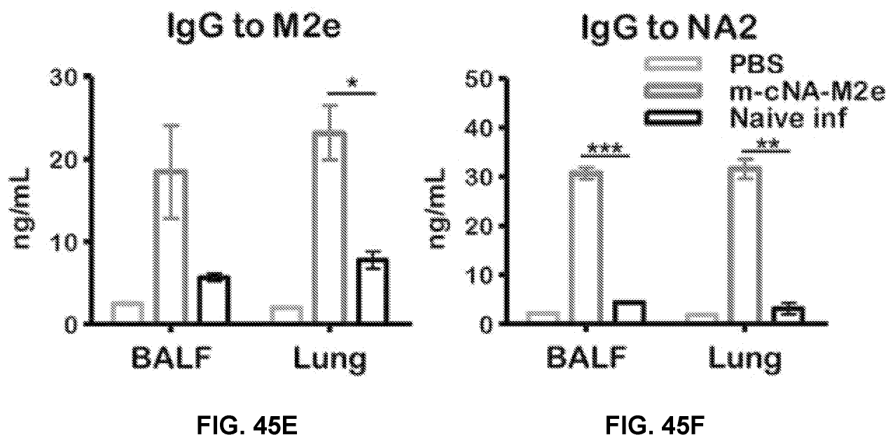

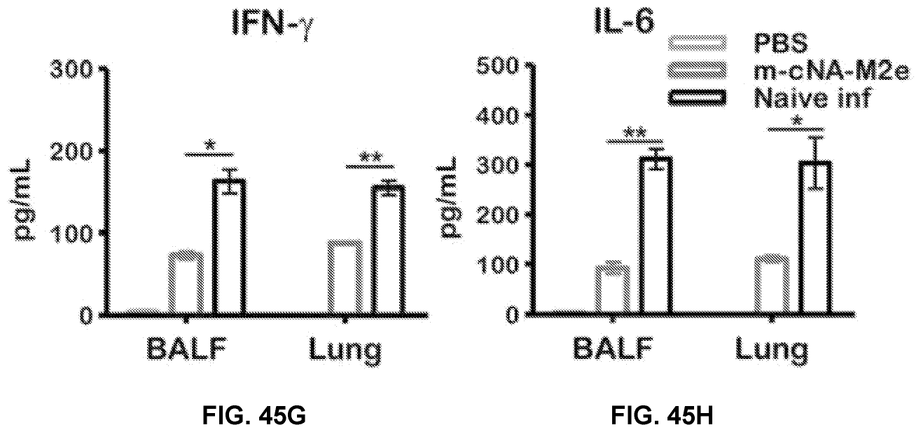

In vitro IgG producing cell responses induced by immunization with m-cNA-M2e VLP versus mono VLP after A/Phil H3N2 virus challenge was next analyzed. IgG antibodies specific for M2e peptide and N2 NA protein were produced at significantly high levels in culture supernatants of mediastinal lymph node (mLN) (FIG. 44C) and spleen cells (FIG. 44D) from the m-cNA-M2e VLP immunized mice, collected day 6 post challenge. In addition, m-cNA-M2e VLP vaccination induced notably higher levels of IgG specific for M2e in bronchoalveolar lavage fluids (BALF) and lung lysates than cN2, 5xM2e, and naive infection (FIG. 45A), while IgG antibodies specific for NA protein (A/Brisbane H3N2) were found to be comparable with the cN2 VLP (FIG. 45B).

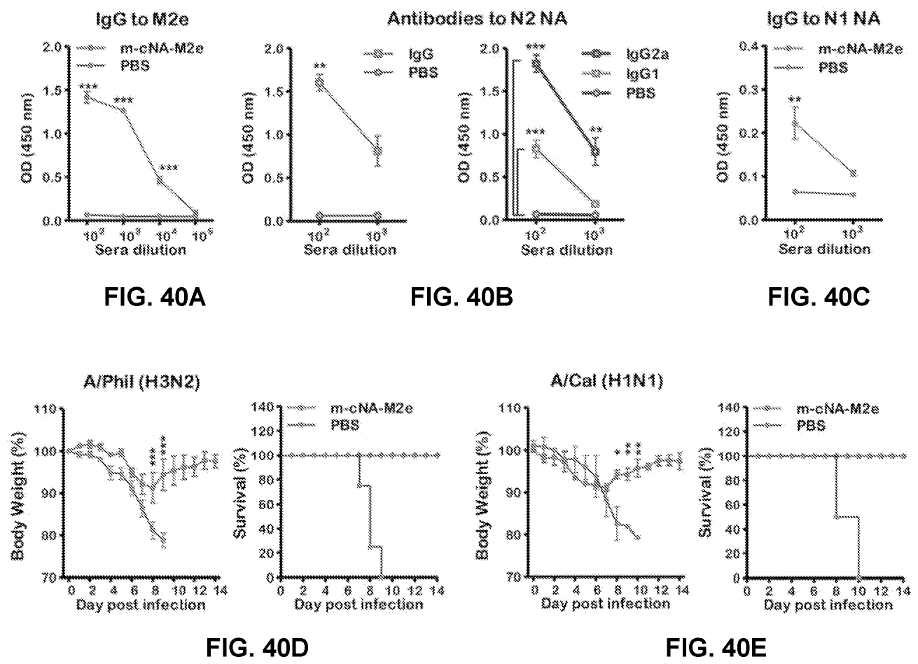

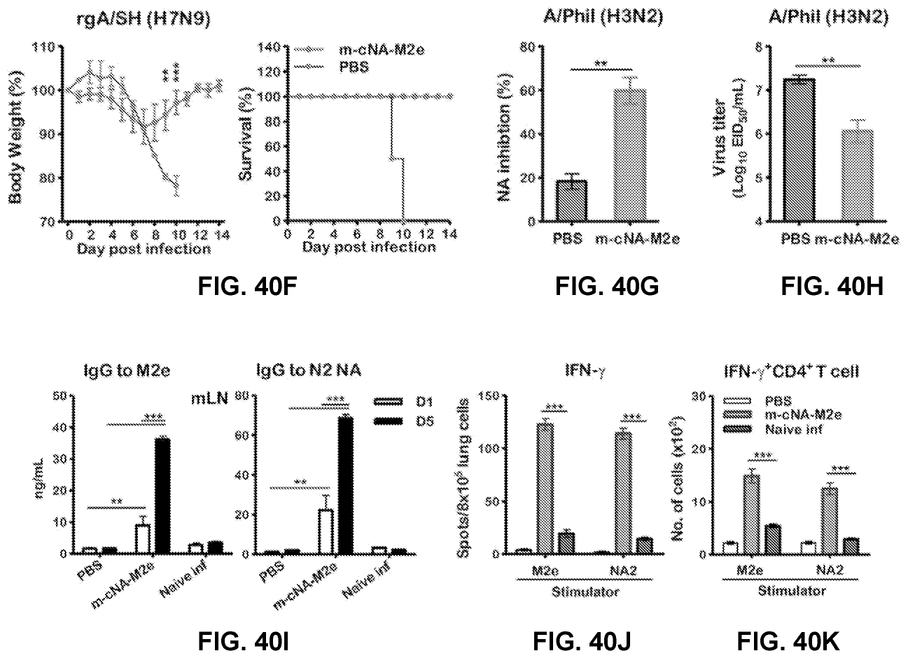

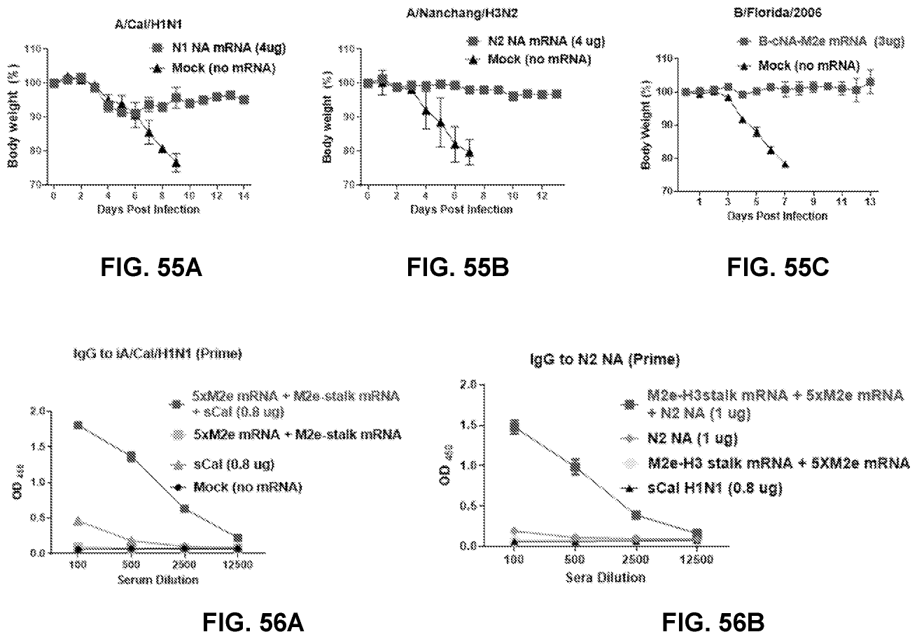

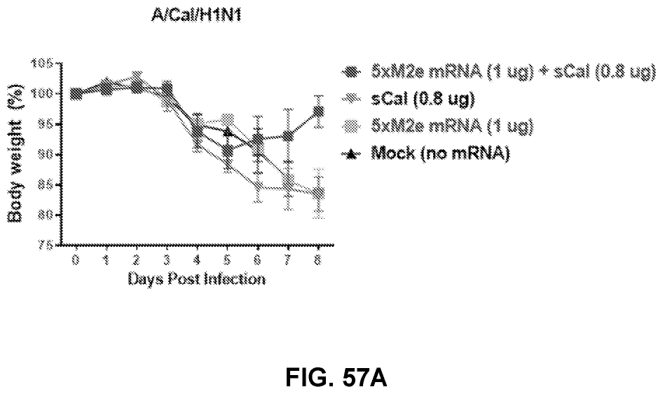

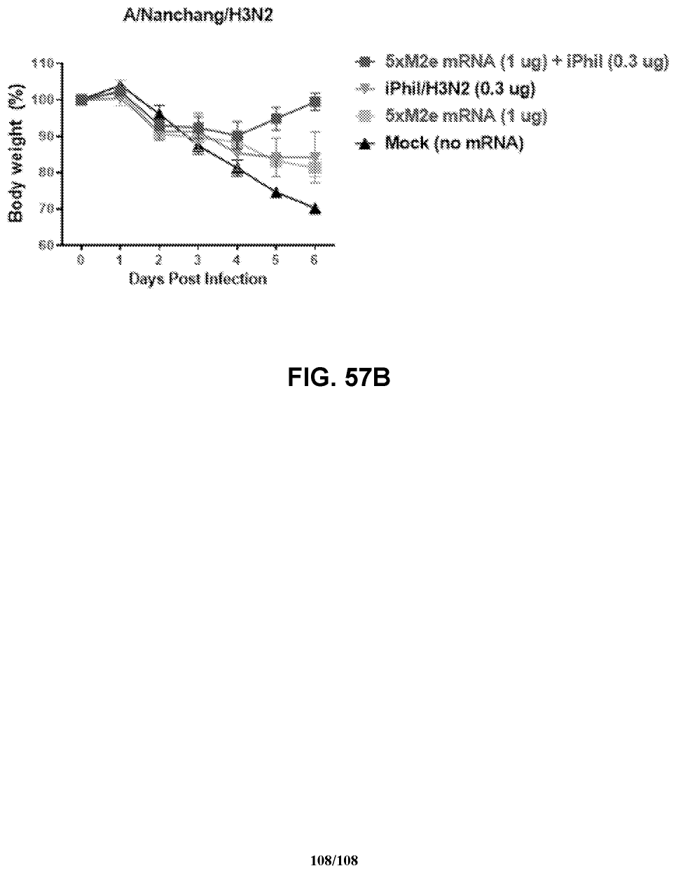

To assess pulmonary immunopathology, inflammatory cytokines (IFN-γ and IL-6) were further measured in BALF and lung lysates. As expected, the m-cNA-M2e VLP and 5xM2e VLP groups showed significantly lower levels of inflammatory cytokines in BALF and lungs of infected mice than the cN2 or naive infection group (FIGS. 55C and 40D). Consistently, the m-cNA-M2e VLP group showed significant lower lung viral titers at day 6 post infection with rgANN H5N1 (FIG. 39H) compared to mock or cN1 VLP, and with A/Phil H3N2 and rgA/SH H7N9 compared to cN2 VLP (FIGS. 391 and 39J). Taken together, these data suggest that m-cNA-M2e VLP vaccination effectively induced cellular and humoral immunity, contributing to cross protection against different subtypes of influenza A viruses.

Aged Mice Vaccinated with m-cNA-M2e VLP Effectively Induce Crossprotection Against Influenza a Viruses

Aged mice vaccinated with m-cNA-M2e VLP effectively induce cross protection against influenza A viruses Aged mice vaccinated with m-cNA-M2e VLP induced M2e peptide or N2 NA protein-specific IgG in boost immune sera (FIGS. 40A and 40B), BALF, and lung lysates (FIGS. 45E, 40F), which were comparable to those in young adult mice (FIGS. 37A and 37C, 45A and 40B), as well as IgG1, and IgG2a antibodies specific for N2 NA protein (FIG. 40B). IgG antibodies specific for N1 NA were induced at lower levels (FIG. 40C), which is consistent with those in young mice (FIG. 37B).

Notably, the aged mice vaccinated with m-cNA-M2e VLP were well protected against lethal challenge with A/Phil H3N2 (FIG. 5D), A/Cal H1 N1 (FIG. 40E), and rgA/SH H7N9 (FIG. 40F) viruses, showing minimum weight loss (8-10%) with 100% survival rates. In addition, appreciably increased NA inhibition activity (60%) (FIG. 5G) and reduced lung viral loads (FIG. 40H) were observed in the m-cNA-M2e VLP group compared to the mock control group.

Humeral and cellular immunity induced by m-cNA-M2e VLP vaccination was further determined in aged mice. M2e or NA-specific IgG antibody production in mLN (FIG. 40I), IFN-γ secreting cells (FIG. 40J), and IFN-γ+co4+ T cells (FIG. 40K) in lungs were observed at higher levels in aged mice with m-cNA-M2e VLP vaccination compared to mock control at day 6 after A/Phil infection. Consistent with young adult mice (FIGS. 45C and 40D), m-cNA-M2e VLP-immunized aged mice showed significantly lower levels of inflammatory IFN-γ and IL-6 cytokines after A/Phil challenge (FIGS. 45G and 40H). Altogether, these data suggest that vaccination of aged mice with m-cNA-M2e VLP provides cross protection against different influenza A viruses by inducing humoral and cellular immune responses comparable to those in young adult mice.

Antibody-Dependent Effector Function and Cellular Immunity 2s2 Induced by m-cNA-M2e VLP Vaccination Contributes to Protection

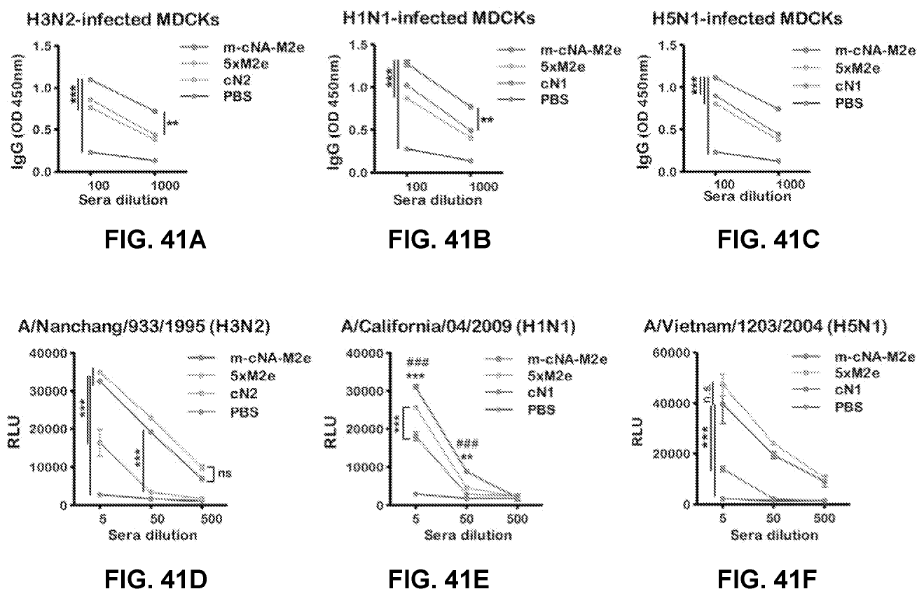

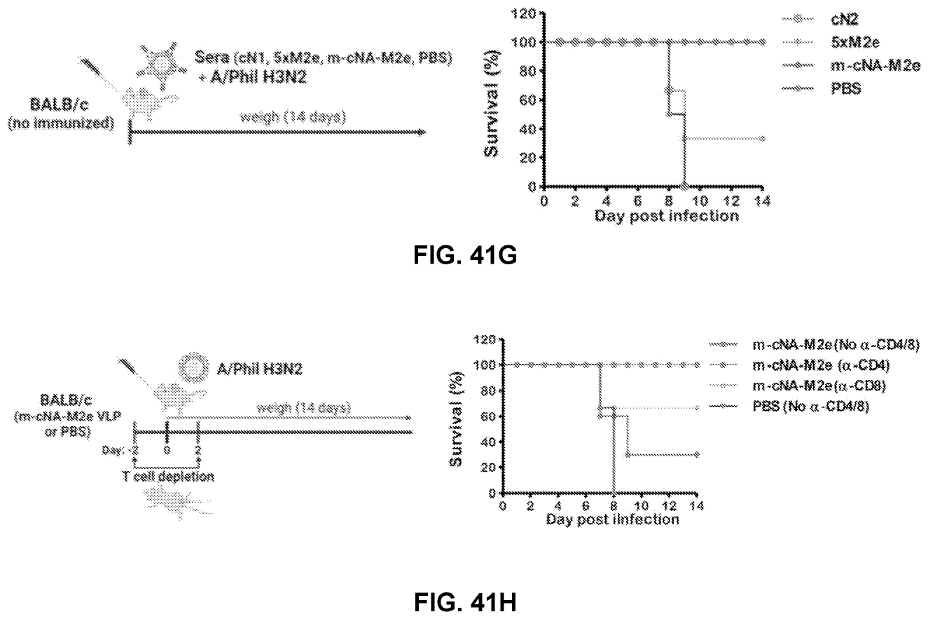

The levels of antibodies binding to the viral antigens was determined on MOCK cells infected with different influenza viruses (FIGS. 41A-41C). Higher levels of binding IgG were observed in immune sera from the m-cNA-M2e VLP group than those from the mono VLP group (cN1, cN2, and SxM2e) consequent to infection with A/NC H3N2, A/Cal H1N1, rgA/VN HSN1 viruses (FIGS. 41A-41C). In line with these results, ADCC assay in MDCKs which were infected with H3N2 (A/NC), H1N1 (A/Cal), and H5N1 (rgA/VN) viruses, showed stronger induction of the reporter signal of Jurkat cell activation upon treatment with immune sera from m-cNA-M2e or 5xM2e VLP vaccination (FIGS. 41D-41F). Immune sera of mono VLP (cN1, cN2, and 5xM2e) vaccination triggered mild induction of the reporter signal, indicating moderate levels of ADCC activity.

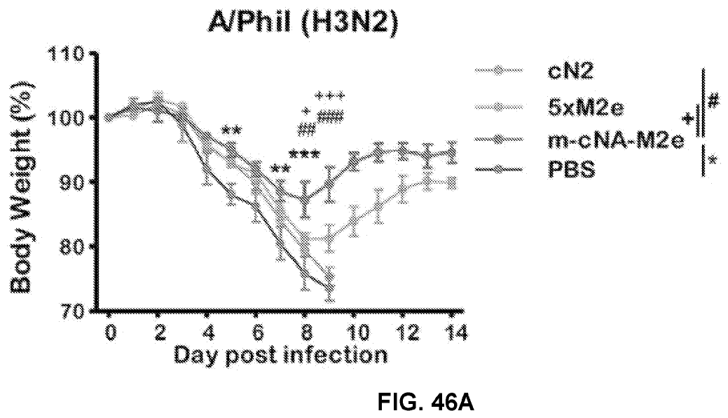

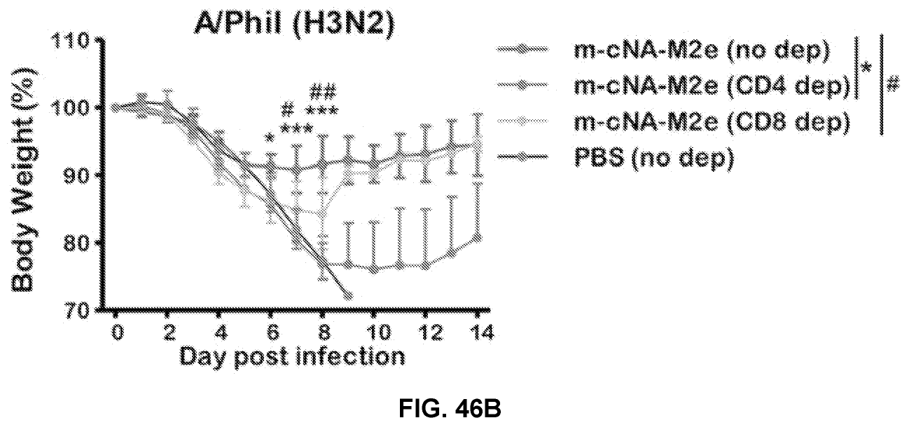

To determine the effects of humeral responses in immune sera on cross protection, narve BALB/c mice were intranasally inoculated with a mixture of A/Phil H3N2 virus and immune sera collected from m-cNA-M2e VLP- or 5xM2e VLP-immunized mice, or naive sera (PBS). Either cN2 VLP or narve sera (PBS) did not provide protection against A/Phil H3N2 virus as evidenced by severe weight loss (>25%, S6A FIG.) and 0% survival rates (FIG. 6G) in na″ive mice. In contrast, m-cNA-M2e VLP immune sera conferred protection in 271 naive mice with moderate weight loss (−12%, FIG. 46A) and 100% survival rates (FIG. 41G), meanwhile 5xM2e VLP immune sera provided partial protection to naive mice with more severe weight loss (˜19%) and 30% survival rates (FIG. 41G, 46A).

To investigate whether T cell immunity would contribute to protection of m-cNA-M2e VLP, CD4 Tor CD8 T cells were depleted from the mice immunized with m-cNA-M2e VLP prior to A/Phil H3N2 virus challenge (FIG. 41H, 46B). Severe weight loss (>20%, FIG. 46B) and low survival rates (FIG. 41H) were observed in the m-cNA-M2e VLP vaccinated mice after CD4 T cell depletion whereas CD8 T cell depletion led to moderate weight loss (˜15%) and partial protection compared to the untreated m-cNA-M2e VLP group (FIG. 41H, 46B). Altogether, these results indicate that CD4 T cells play more protective roles than CD8 T cells in providing protection in the m-cNA-M2e VLP vaccinated mice; furthermore, humeral immune responses, including ADCC and NAI activity antibodies, contribute to broad cross protection in m-cNA-M2e VLP vaccinated mice.

### DISCUSSION

NA contents in seasonal vaccines are low, variable, and not standardized. In addition, due to competition of intravironic HA and NA antigens within the same particle, strain-specific HA immunity is dominant over NA in both T- and B-cell responses. Physical separation of HA and NA immunization was shown to induce IgG responses to HA and NA, avoiding competition of intravironic antigens. The differential contribution of NA immunity to homologous and heterologous protection within the same NA subtype viruses was well documented with adjuvanted recombinant NA protein (10 μg) vaccination in mice. N1 NA (A/PR8, H1 N1) could provide complete protection against homologous virus from morbidity and mortality, but lower efficacy of cross protection against heterologous H1N1 (2009 pandemic) and avian H5N1 viruses as evident by severe weight loss. Reduced heterologous protection was similarly observed with recombinant N2 NA protein vaccination in mice, consistent with NA antigenic drifts, and heterosubtypic NA protection was not induced by monomeric NA vaccination, which is consistent in other studies reporting lower heterologous cross protection by NA-immunity after intramuscular vaccination. Prior studies demonstrated broader cross-protection against heterosubtypic viruses by 5xM2e VLP vaccine, although M2e immunity alone has a limitation of providing low efficacy. To further overcome NA antigenic drifts and we investigated new approaches to improve the breadth and efficacy of NA plus M2e immunity, consensus NA sequences were designed from the isolates after 2010 and implemented in the full-length NA constructs. The VLP platform expressed in insect cells has a unique feature to incorporate multiple NA proteins (cN1, cN2, and B cNA) in a membrane anchoring form, mimicking viral surface glycoproteins, and covering both seasonal influenza A and B viruses. Additionally, 5xM2e tandem repeat was incorporated into the same multi-NA VLP format using a multi-gene expressing baculovirus vector. Monomeric N1 NA VLP vaccines were shown to induce protective immunity against homologous virus in ferrets [1 O] and protection against homologous and heterologous viruses in mice. Influenza HA (2009 H1 N1, H5N1, H7N9) VLP vaccines produced in insect cells were safe and efficacious in clinical trials, suggesting VLP as a promising vaccine platform delivering multi-NA and M2e immunogens.

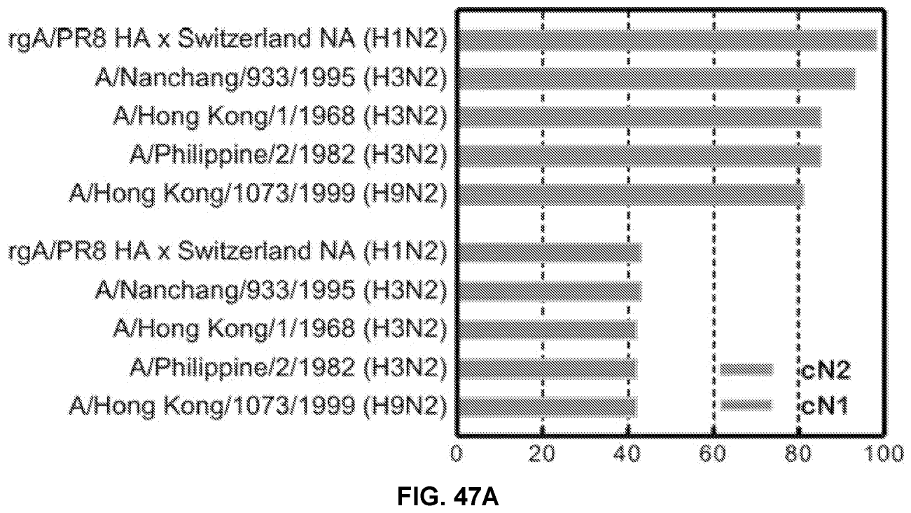

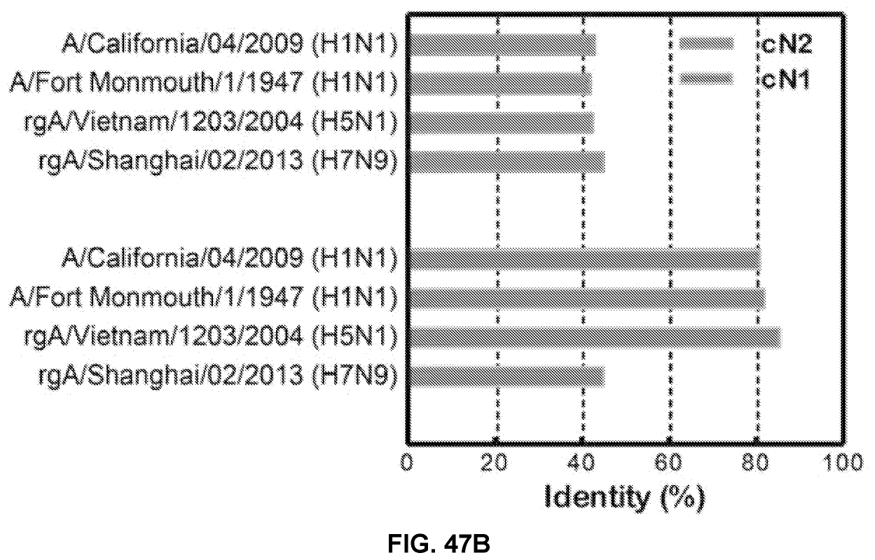

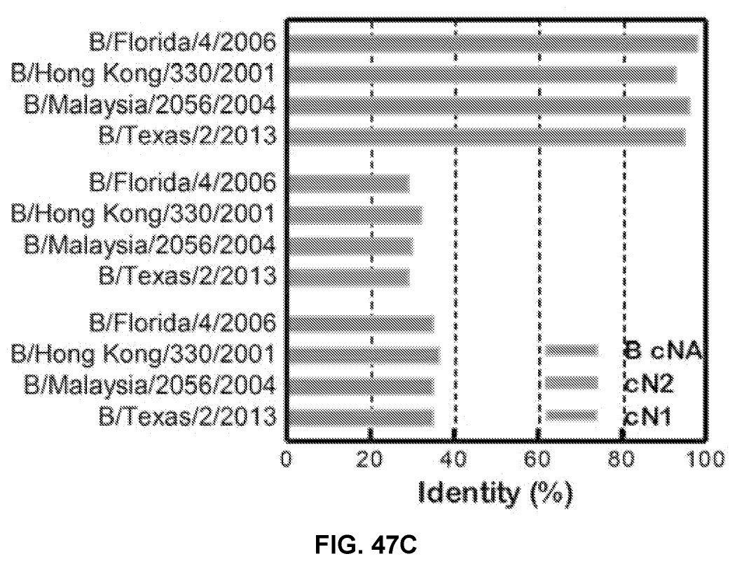

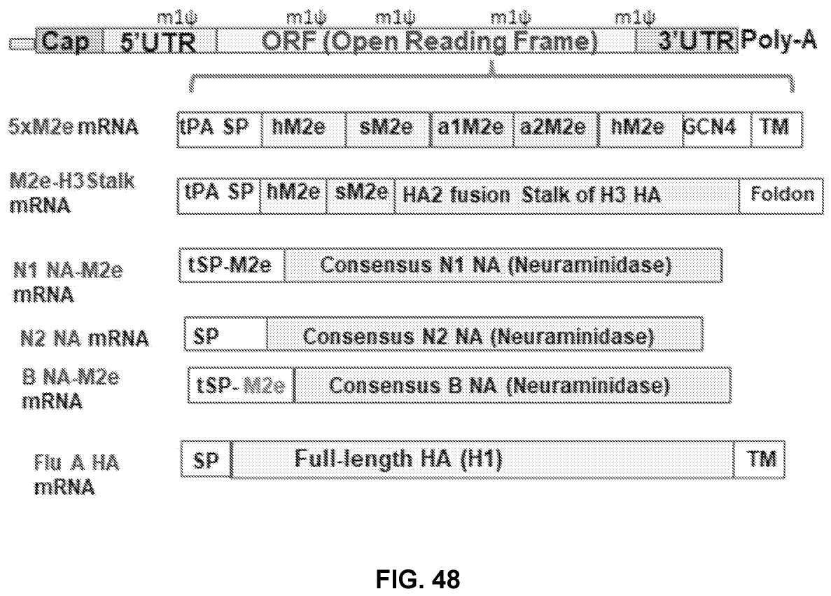

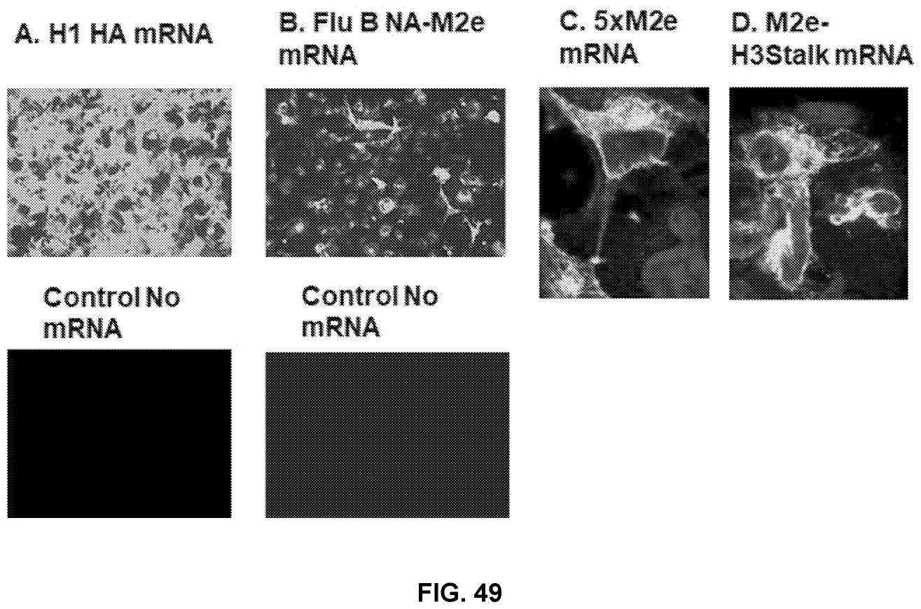

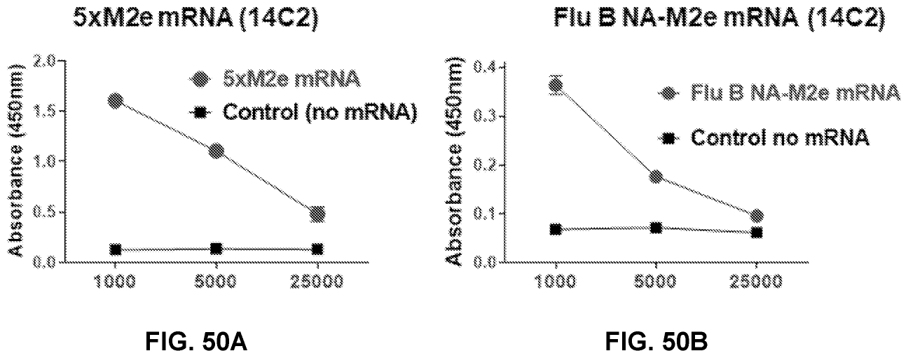

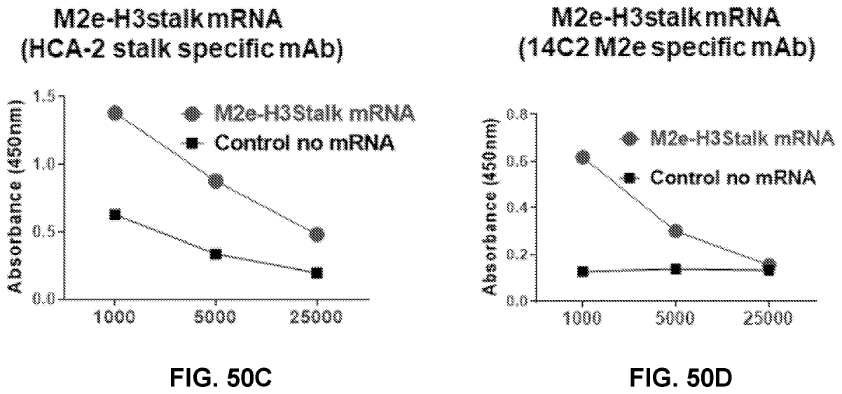

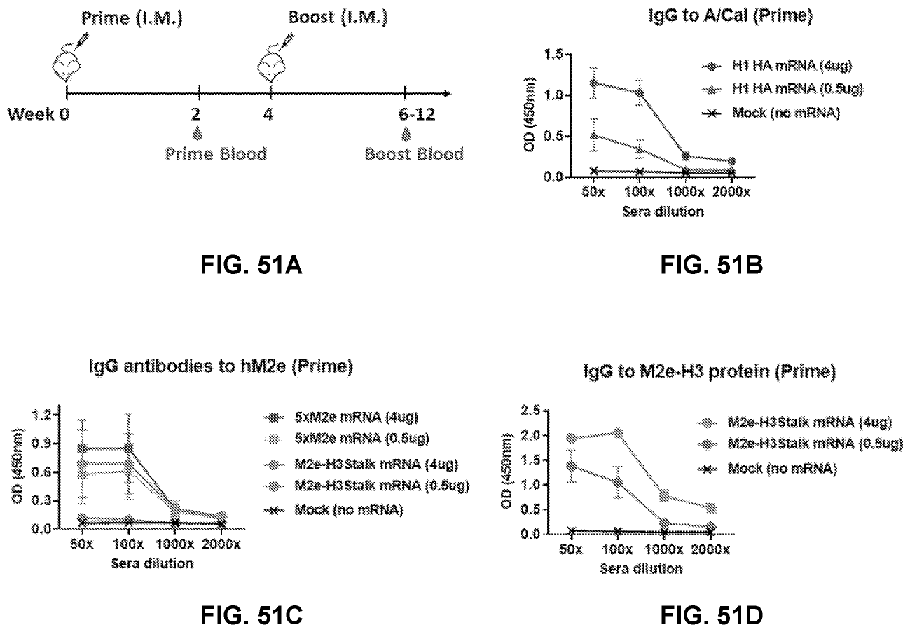

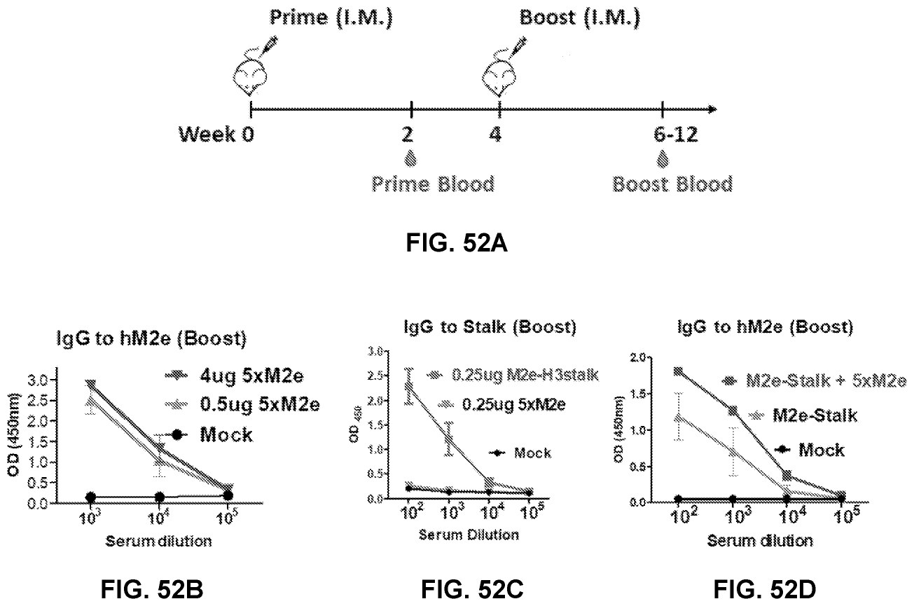

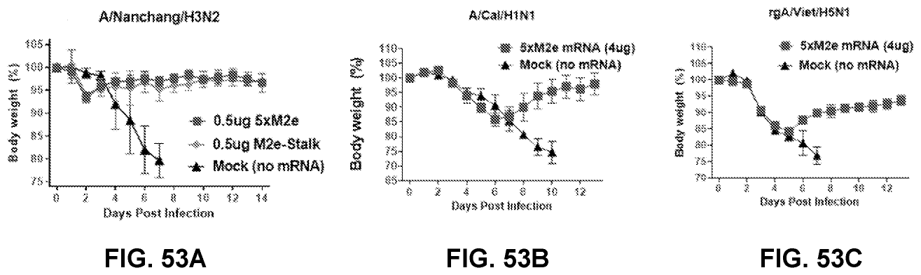

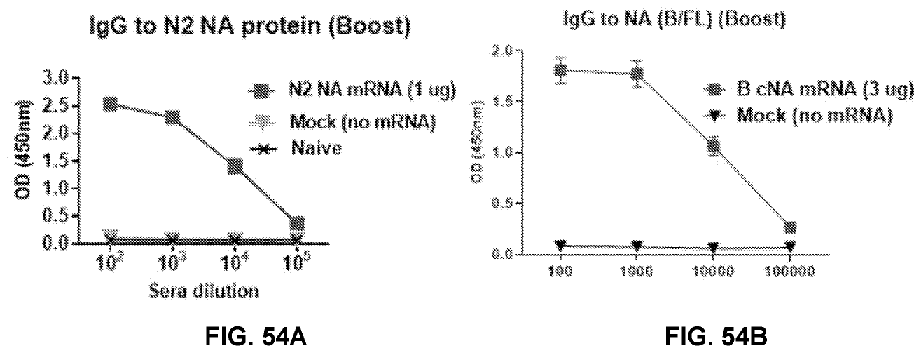

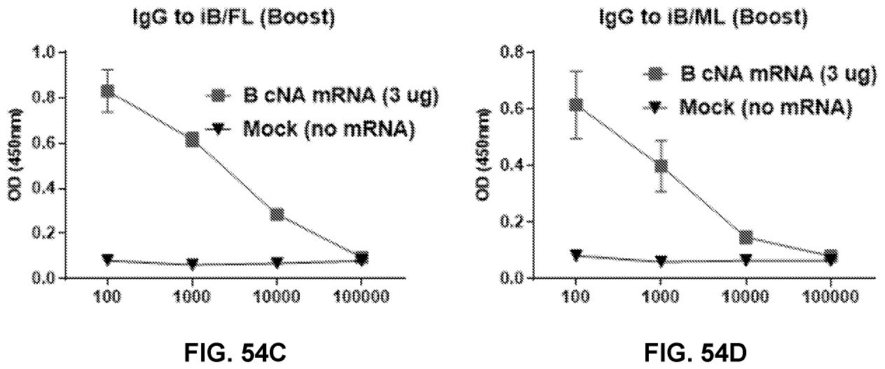

m-cNA-M2e VLP vaccination induced NAI activities, a known correlate of NA immunity, against a broad range of viruses including heterologous and heterosubtypic H1 N1, H5N1, H3N2, H1 N2, H7N9, and H9N2. Challenge viruses were heterologous since multi cNA-M2e VLP vaccine contains multi-NA proteins with artificial consensus sequences. m-cNA-M2e VLP vaccination protected mice against A/Nanchang/1995 and rgA/H1 N2 (N2 of A/Switzerland/2013) viruses containing NA that has high homology (93% and 98%) with cN2 (FIG. 47A). Even for N2 viruses with phylogenetically more distant 81% to 85% homology with cN2, m-cNA-M2e VLP vaccination could confer broad protection against cross-subtype N2-expressing viruses (H3N2 and H9N2) despite moderate weight loss. In contrast, monomeric cN2 VLP conferred protection against rgH1 N2 virus (98% homology) but did not provide protection against drifted H3N2 (A/HK/1968 and A/Phil/1982) and H9N2 (rgA/HK/1999 H9N2) viruses, suggesting the contribution of 5xM2e in m-cNA-M2e VLP to inducing protection against NA drifted viruses. Significant protection against H1 N1, rgA/VN H5N1, and rgA/SH H7N9 viruses was induced by vaccination with m-cNA-M2e VLP but not with monomeric cN2 VLP vaccination, consistent with the impact of m-cNA-M2e immunity on broadening the cross protection. The monomeric cN1 VLP was similarly effective in inducing protection against rgA/VN H5N 1 virus with 85% NA homology (FIG. 47B). In particular, the contribution of 5xM2e appears to be significant as shown by 5xM2e VLP-induced protection against rgA/VN H5N1 virus at a comparable level. It should be noted that in aged mice, m-cNA-M2e VLP was immunogenic in inducing humeral and cellular responses to M2e and NA, and provided 100% cross protection against H1N1, H3N2, and rgH7N9 viruses, preventing severe weight loss.

The NA genetic diversity appears to be limited in influenza B viruses [29). Consistently, adjuvanted recombinant NA vaccination of influenza B virus was also reported to provide cross-lineage protection. Influenza B virus consensus NA (B cNA) shows high homology (96%) with both lineages of B viruses (FIG. 47C). Prominent protection against weight loss and mortality was observed in mice vaccinated with m-cNA-M2e VLP or monomeric B cNA VLP after lethal dose challenge with Victoria or Yamagata lineage viruses, correlating with broadly cross-reactive NAI activities. For the first time, this study provides evidence that a single entity of m-cNA-M2e VLP could be developed as a universal vaccine protecting both lineage influenza B viruses and antigenically distinct influenza A viruses.

In summary, this data supports that m-cNA-M2e VLP has the capacity to induce immunity to M2e and multi-subtype NA of influenza A and B viruses, and broad cross protection against morbidity and mortality under lethal challenges in mice. Developing m-cNA-M2e VLP as a new universal vaccine candidate, this study provides a supportive proof-of-concept approach to overcome a limitation of NA antigenic drifts in broadening cross protection and extend cross immunity to both influenza A N1 and N2 major subtypes, heterologous and heterosubtypic viruses, and influenza B NA, in addition to M2e. In an outbreak of HA variants or pandemic, immunity to broad NA and universal M2e epitopes is expected to provide protection against severe disease and mortality. Nonetheless, protective immunity by m-cNA-M2e VLP is not sterilizing, permitting a certain level of viral replications in the lung due to the non-neutralizing nature of NA and M2e immunity. Permissive protection was reported to provide immunologic benefits of effectively protecting future pandemics. An alternative will be to supplement seasonal HA-based vaccines with m-cNA-M2e VLP as reported with purified recombinant NA or 5xM2e VLP or to test the efficacy of m-cNA-M2e VLP under pre-existing immunity, mimicking the general human population. Overall, this study warrants further testing of m-cNA-M2e VLP as a universal vaccine candidate such as in relevant ferret animals.

### Materials and Methods

### Ethics Statement

Mouse studies were approved by Georgia State University (GSU) Institutional Animal Care and Use Committee (IACUC, A21004) and carried out with the Guide for the Care and Use of Laboratory Animals of the NIH. Young and aged BALB/c mice were purchased from Jackson Laboratory and Taconic respectively, were housed in the animal facility at GSU.

Influenza genes and recombinant baculovirus (rBV) constructs Consensus NA (cN1 NA, cN2 NA, influenza B cNA) sequences (FIG. 41) were obtained from aligning the NA sequences of the human isolates (2010-2019) available at NCBI by using the sequence alignment program (UGENE software). NA and 5xM2e genes were codon-optimized (FIG. 41) for high-level expression in Sf9 insect cells and, synthesized (Gen-script, Piscataway, NJ). The 5xM2e construct consists of M2e from, human, swine, and type 1/11 avian influenza A viruses as previously detailed. A, plasmid DNA (pFastBac1) was engineered to express 3 full-length NA genes (cN1 NA, cN2 NA, influenza B cNA), 5xM2e, and M1 genes in tandem under each transcriptional, polyhedrin promoter (FIG. 36A) as previously described. The 5 genes to be expressed were introduced into the single pFastBac1 transfer vector and confirmed for correct insertions. A baculovirus expressing 5 genes (m-cNA-M2e, FIG. 36A) was generated by using the Bae-to-Bae expression system, and plaques were purified and amplified, and high titer stocks were prepared and confirmed using gene specific PCR (FIG. 36B). To prepare cNA or 5xM2e VLP, bacmid DNA containing each consensus NA or 5xM2e gene were isolated from DH10Bac E. coli after cloning into pFastBac and used to transfect Sf9 cells to generate mono-expressing baculovirus as described.

Expression and Characterization of Consensus Multi-Subtype, m-cNA-M2e VLPs

Influenza VLP vaccines were produced as previously described. Sf9 cells maintained in suspension cultures of SF900II-SFM serum free medium were infected with baculovirus expressing consensus monomeric NA and 5xM2e, or multi 5 genes (cN1, cN2, B-cNA, 5xM2e, M1). VLPs were harvested from the culture supernatants containing released VLPs by low-speed centrifugation (2,000×g) to remove cell debris, then purified by ultracentrifugation (100,000×g) and resuspended in phosphate buffered saline (PBS). The protein concentration of VLPs were quantified by a protein assay kit (Bio-rad, Irvine, CA) and characterized by ELISA and western blot using M2e mAb 14C2, pan NA HCA-2 mAb. SOS-PAGE was performed using 4-12% gradient polyacrylamide gels (Invitrogen). Nanoparticle size distribution of VLPs was determined by dynamic light scattering (DLS) with a Malvern Zetasizer Nano ZS (Malvern Instruments, Westborough, MA). The functional activity for NA expressed on the surface of VLPs was determined by enzyme linked lectin assay (ELLA) as described.

### Immunization and Challenge of Mice

Female young (6- to 8-week-old) and aged (14-month-old) BALB/c mice were IM immunized twice, at a 3-week interval, with 100 μl (50 μl in left and in right leg) of VLPs; 10 μg of m-cNA-M2e VLP, 3 μg of cN1 VLP, 3 μg or 10 μg of cN2 VLP, 3 μg of B cNA VLP, 3 μg of 5xM2e VLP. Use of a higher dose (10 μg) for m-cNA-M2e VLP than that (3 μg) of monomeric VLP was based on the observation that m-cNA-M2e VLP showed 10-fold lower reactivity than monomeric 5xM2e VLP (FIGS. 1 G and H). PBS was used as a mock control. Bloods were collected at 2 weeks after prime and boost immunization. After boost, mice were challenged intranasally with a lethal dose of influenza A and B viruses in 50 μl PBS. Body weight changes and survival rates were monitored daily for 14 days.

The influenza viruses used for challenge were as follows; A/California/04/2009 H1N1 (A/Cal H1 N1), mouse adapted A/Fort Monmouth/1/1947 H1N1 (A/FM H1N1), rgA/VN H5N1 containing H5 HA with the polybasic cleavage site deleted and N1 NA derived from A/Vietnam/1203/2004 and the backbone genes from A/Puerto Rico/8/1937 (A/PR8 H1N1). The rgA/H1 N2 virus contains N2 NA derived from A/Switzerland/2013 H3N2 and the remaining seven genes from A/PR8 (38), A/Philippine/2/1982 H3N2 (A/Phil), rgA/NC H3N2 with H3 HA and N2 NA from A/Nanchang/933/1995 and A/PR8 backbone, A/Hong Kong/1/1968 H3N2 (A/HK H3N2), A/Hong Kong/1073/99 H9N2 (A/HK H9N2), rgA/SH H7N9 containing H7 HA and N9 NA from A/Shanghai/02/2013 and A/PR8 backbone. For influenza B virus challenges, we used Victoria lineage B/Malaysia/2056/2004 (B/ML) and Yamagata lineage B/Florida/4/2006 (B/FL). All viruses were propagated in embryonated chicken eggs, and 50% egg-infectious doses (EID₅₀) were determined using 0.5% chicken red blood cells.

### Enzyme-Linked Immunosorbent Assay (ELISA)

ELISA virus antigens include N1 NA (A/Cal, H1 N1, BEi, NR-19234), N2 NA (A/Brisbane/10/2007 H3N2, BEi, NR-43784), influenza B NA proteins (B/Florida/4/2006, BEi, NR-19236), human M2e (hM2e, SLLTEVETPIRNEWGSRSNDSSD, SEQ ID NO:5) peptide, and inactivated influenza A viruses. Serially diluted immune sera were applied onto 96-well microtiter plates pre-coated with antigens at 200 ng/ml of N1, N2 NA, influenza B/FL NA proteins, 4 μg/ml of hM2e peptide and inactivated influenza viruses. For assays of IgG antibody-secreting mediastinal lymph node (mLN) and spleen cell responses, the cells isolated were in vitro cultured for 1 (01) or 5 days (05) on the plate precoated with influenza virus antigens. The combined levels of IgG antibodies secreted into the culture supernatants and captured on the plate were analyzed. The IgG and IgG isotypes were determined using horseradish peroxidase (HRP)-conjugated goat anti-mouse IgG, IgG1, IgG2a secondary antibodies (SouthernBiotech, Birmingham, AL) and tetramethylbenzidine (TMB) (eBiosciences, San Diego, CA), and antibody levels are presented as optical density (OD) at 450 nm (BioTeck ELISA plate reader) or concentrations as calculated using standard IgG (Southern Biotech) as previously described.

### Enzyme-Linked Lectin Assay (ELLA)

NA inhibition (NAI) activity of immune sera against influenza virus was determined by ELLA using a fetuin-based procedure as described. Briefly, virus and immune sera were added to 96-well plates coated with 25 μg/mL of fetuin (Sigma-Aldrich) and then the plates were incubated at 37° C. for 20 h. After incubation with 1 μg/mL of HRP-labeled peanut lectin, NAI activity was measured by using TMB substrate (eBiosciences) to develop colorimetric reaction. The inhibition percentage was calculated using the formula: 100×(OD_{virus only control}−OD_{test sample})/OD_{virus only control}.

### Lung Viral Titration

Lung viral titers were determined in embryonated chicken eggs. Ten-fold serial dilutions of lung lysates were injected into 10-day embryonated chicken eggs and then incubated for 3 days. Virus titers were determined by hemagglutination assay of the allantoic fluids. The titers of EIDso were determined according to the Reed and Muench method.

### Elispot Assay

IFN-γ secreting cells were evaluated in the lung and spleen samples by enzyme-linked immunospot (ELISpot) analysis as described previously. Stimulating antigens used for ELISpot assay were M2e peptide and NA peptide pools (A/Brisbane/10/2007 H3N2). Lung (5×10⁵ cells/well) and spleen cells (10⁶ cells/well) were cultured on 96-well ELI Spot plates precoated with anti-mouse IFN-γ capture antibody (BD Biosciences, San Diego, CA) in the presence of 5 μg/ml peptides or virus antigens. The spots were developed with biotinylated anti-mouse IFN-γ antibody and alkaline phosphatase-labeled streptavidin (BO Pharmingen), visualized with a 3,3′-diaminobenzidine substrate, and counted by an ELISPOT reader (BioSys, Miami, FL).

### Flow Cytometry

Single cell suspensions for flow cytometry analysis were prepared on 44/67% Percell gradient after homogenizing the lung tissues collected day 6 or 5 post challenge as described. Then, the cells were in vitro stimulated with 5 μg/ml of M2e peptide or NA [A/Brisbane/10/2007 H3N2 (BEi, NR-19251) and ANietnam/1203/2004 H5N1 (BEi, NR-19258) peptide pools in presence of Brefeldin A (20 μg/ml) for 5 h at 37° C. The lymphocytes were stained with anti-mouse CD3 (clone 17A2, BO, San Diego, CA), CD4 (clone 553051, BO), COB (clone 25-0081-82, eBiosciences, San Diego, CA), and IFN-γ (clone XMG1.2, BO) mAb. Intracellular cytokine staining of lymphocytes was followed by using BD Cytofix/Cytoperm™ Plus kit. Cytokine-expressing cells were acquired on a Becton-Dickinson LSR-11/Fortessa flow cytometer and analyzed by Flowjo software (Tree Star, Inc., Ashland, OR).

### In Vivo Protection Test of Immune Sera

Sera (25 μl) after heat-inactivation were mixed with 25 μl of 4×LD₅₀ A/Phil and incubated at room temperature for 30 min as described. A mixture of A/Phil H3N2 virus and sera was intranasally administered to naive BALB/c mice, and body weight changes and survival rates were monitored daily for 14 days.

### Antibody-Dependent Cellular Cytotoxicity (ADCC) Assay

ADCC activity of immune sera was performed according to the manufacturer's protocol (Promega). Briefly, Madin-Darby Canine Kidney cells (MDCKs, ATCC) maintained in Dulbecco's Modified Eagle Medium media (DMEM) supplemented with 10% heat inactivated fetal bovine serum were seeded in sterile white 96 well plates. The MDCKs on the 96-well plates were infected with 100xTCID₅₀ of influenza A viruses a day prior to assay. Immune sera diluted in assay buffer and effector Jurkat cells expressing mouse FcγRIV (Promega) were added to virus-infected MOCK target cells and then incubated for 6 h. Luminescence was read on a Cytation 5 imaging reader (BioTek) after 5 min incubation with 75 μL of Bio-Glo luciferase assay substrate (Promega).

### In Vivo Depletion of T Cells

To deplete CD4 or CDS T cells, BALB/c mice immunized with m-cNA-M2e VLP were injected intraperitoneally (i.p.) with 200 μg of anti-CD4 mAb (clone GK1.5, BioXCell) or 150 μg of anti-CDS mAb (clone 53.6.7, BioXCell) on day −2 and +2 before/after challenge as previously described (44). The depletion of CD4 and CD8 T cells was confirmed by flow cytometry of blood samples.

### Statistical Analysis

Data are represented as mean±standard errors of the mean (SEM). The statistical significance was performed by one-way ANOVA with Tukey's multiple comparison post-test and by two-way ANOVA with Bonferroni posttests. P values of less than 0.05 (p<0.05) were considered statically significant. Data were analyzed using a Prism software (GraphPad Software, Inc., San Diego, CA).

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure.

### Example 4: Rational Design of HA mRNA and Universal Flu mRNA Vaccine Constructs

(1) Full-length HA mRNA constructs were generated to produce full-length HA mRNA constructs: H1 HA mRNA (from A/South Africa/2013 H1N1) with 97% homology to A/California/2009. This H1 HA mRNA vaccine was constructed to test the immunogenicity and efficacy of HA-based conventional flu vaccine but in an mRNA vaccine platform. (2) 5xM2e mRNA: Tandem repeat 5xM2e contains M2e derived from human (2×, hM2e), swine (sM2e), and avian (2×, a1M2e, a2M2e) flu H5N1 and H7N9 viruses plus signal peptide (SP, tissue plasminogen activator tPA), a tetramer stabilizing domain (GCN4), and transmembrane (TM) domain derived from HA. 5xM2e was significantly more effective in conferring broader cross-protection than monomeric wild type (WT) full-length M2 {Kim, 2013 #4478; Kim, 2013 #4466}. Similarly, we constructed 5xM2e mRNA. (3) M2e-stalk mRNA constructs: For stable expression and cross-group protection, chimeric M2e-stalk genetic fusion constructs with stabilizing mutations were molecularly designed {Subbiah, 2022 #5715; Subbiah, 2022 #5740}. M2e-stalk mRNA construct contains SP (tPA), 2xM2e (hM2e-sM2e likely contributes to stabilizing HA stalk domain), HA1 parts [aa 18-41, aa 290-323 of H1 HA; aa 21-46, aa 290-323 of H3 HA] and consensus HA2-stalk parts [aa 1-117 from H1 or H3] as described {Subbiah, 2022 #5715; Subbiah, 2022 #5740}. Also, to improve expression, point mutations were introduced in the hydrophobic patches, in HA1 (V296T, I299N, Y301T, C304S) and HA2 stalk (F64D, I67D, V74D, L111A).

### Example 5: Codon Optimization, Production, and Expression of Nucleoside-Modified mRNA Vaccines

Genes for mRNA were codon-optimized by using a GenSmart™ Codon Optimization Tool (GenScript) and then further optimized by increasing G/C contents and minimizing Uridine usage, which maximizes mRNA stability and expression {Vaidyanathan, 2018 #332; Tai, 2020 #157; Freyn, 2020 #169; Thess, 2015 #436}. The 5′-untranslated region (UTR) and 3′ UTR in mRNA in vitro production were modified from the sequence reported {Nance, 2021 #452}. We utilized custom mRNA production service (TriLink or GenScript) with CleanCap1 capping strategy and poly-A addition {Vaidyanathan, 2018 #332} to expedite the progress. All mRNA vaccines were produced or will be produced to contain N1-methylpseudouridine (m1Ψ) instead of ∪ nucleosides to avoid inflammatory side effects of mRNA vaccines while enhancing mRNA translation efficiency, functional half-life, and adaptive immune responses to mRNA vaccination {Nance, 2021 #452; Weiner, 2013 #546; Mauger, 2019 #441; Kariko, 2008 #286}. Expression of the mRNA constructs was confirmed in HEK293T cells after transfection and examination under fluorescent microscope, by probing with antigen specific monoclonal (mAbs) or polyclonal sera as primary Abs and by ELISA.

### Example 6: HA mRNA-LNP and Universal Flu mRNA-LNP Vaccines are Immunogenic and Effectively Induce IgG Responses at a Low Dose. LNP Encapsulation of mRNA

Optimized LNP mixtures (GenVoy-ILM, Precision Nanosystems) contain ionizable cationic lipid, DSPC (1,2-distearoyl-sn-glycero-3-phosphocholine), helper PEG-lipids, and cholesterol. GenVoy-ILM is based on FDA-approved clinical LNP containing the MC3 ionizable cationic lipid and can be used as a representative formulation for proof-of-concept studies. We prepared mRNA-LNPs by using NanoAssemblr Benchtop Instrument (Precision Nanosystems), which enables rapid fluidic mixing and encapsulating mRNA into lipid LNPs. mRNA-LNP retains high stability, translation efficiency, and immunogenicity {Tai, 2020 #157; Fang, 2022 #540}.

### Example 7: IM Immunization of Mice with mRNA-LNP Vaccines

Mice (BALB/c, n=5) were prime or prime boost intramuscularly (IM) immunized with low (0.5 μg) or high (4 or 5 μg) dose of mRNA-LNP vaccine. Boost vaccination with H1 HA mRNA-LNP (0.5 μg, 4 μg) increased IgG Ab levels specific for A/California/2009 (A/Cal H1 N1) up to 10⁵ titers. M2e and HA stalk previously known to be low-immunogenic and sub-immunodominant, thus requiring high dose protein (10 μg or more) with adjuvants {Lee, 2015 #4449; Subbiah, 2022 #5715; Subbiah, 2022 #5740; Kim, 2013 #4466}. Newly developed our 5xM2e mRNA-LNP and M2e-Stalk mRNA vaccination effectively induced M2e and stalk specific IgG Abs even with 0.5 μg mRNA dose. We observed no significant differences in IgG levels between low (0.5 μg) and high (4 or 5 μg) dose mRNA-LNP vaccine groups.

### Example 8: Adjuvant Effects of Flu mRNA Vaccines on Enhancing Immune Responses

Dual roles of mRNA-LNP vaccine were discovered. IgG Abs specific for A/Cal H1N1 and A/Switzerland (A/SW, H3N2) were induced at higher levels (˜100 folds) when IM co-immunized with H1 HA mRNA-LNP (1 μg)+inactivated split sCal+sSW vaccine (sCal: A/Cal, 0.8 μg+sSW: A/SW, 0.8 μg), than either mRNA or split only. Similarly, IgG Abs to A/Cal were induced at 20 folds higher levels when IM co-immunized with 5xM2e mRNA (1 μg)+sCal (0.8 μg) than sCal alone. Also, the levels of IgG Abs to hM2e were higher in the 5xM2e mRNA-LNP (1 μg)+sCal (0.8 μg) co-immunized group than 5xM2e mRNA-LNP alone. These data support the hypothesis that multivalent HA mRNA or universal mRNA (M2e, Stalk) supplemented with inactivated flu vaccines will enhance the magnitudes of IgG Abs to vaccine antigens.

### Example 9

These data showed that a low (0.5 μg) dose H1 HA (A/South Africa/2013) mRNA-LNP could induce protection against lethal dose of A/Cal/09 (H1N1) by preventing body weight (BW) loss. Our prior studies {Kim, 2013 #4478; Kim, 2013 #4466}{Subbiah, 2022 #5715; Subbiah, 2022 #5740}reported that 5xM2e VLP (10 μg), adjuvanted M2e-H1stalk or M2e-H3stalk protein (10 μg) could provide broader protection against different subtype viruses: H1N1 (A/PR8/34, A/WSN/33, A/Cal/09, A/FM/47), H3N2 (A/Phil/82, A/Nanchang/95, A/HK/68), A/Viet/04 rgH5N1, rgH7N9/13. M2e-H3stalk mRNA-LNP or 5xM2e mRNA-LNP vaccine with prime boost dose of 0.5 μg mRNA could induce protection against A/Nanchang/95 H3N2 virus in mice. These data support the scientific premise and feasibility of mRNA-based universal influenza vaccines.

21-month(M)-old mice (n=5, BALB/c) were prime boost immunized with H1 HA mRNA-LNP (1 μg) mixed with sCal (0.8 μg) or sCal vaccine alone. The H1 HA mRNA-LNP+sCal group induced significantly higher levels of IgG Abs specific for A/Cal virus and no BW loss after lethal challenge with A/Cal virus compared to the sCal group. These data suggest that co-immunization with flu mRNA-LNP and split vaccines will provide enhanced immunogenicity and efficacy in aged mouse models.

Co-immunization with H1 HA (0.5 μg) mRNA-LNP+5xM2e (0.5 μg) mRNA-LNP enhanced protection against heterosubtypic rgH7N9 virus, preventing severe weight loss (˜6%) compared to H1 HA mRNA only immune mice (>20% weight loss) possibly by M2e immunity. Under pre-existing immunity by H3N2 (A/Phil/82)-H1N1 (A/Cal/09) virus sequential infections, 5xM2e (0.5 μg) mRNA prime dose enhanced M2e specific IgG Abs. These data support that a strategy of supplementing multi HA mRNA with multi universal mRNA will overcome the limitation of strain-specific HA alone vaccines even under pre-existing immunity.

1. A cross-protective influenza vaccine comprising a virus-like particle (VLP) displaying on its surface consensus N1 neuraminidase (cN1); consensus N2 neuraminidase (cN2); consensus influenza B neuraminidase (B-cNA); and a tandem repeat of two influenza virus matrix protein 2 extracellular (M2e) domains derived from a human influenza A subtype (hM2e), one M2e domain derived from a swine influenza A subtype (sM2e), one avian M2e domain derived an avian type I influenza A subtype (a1M2e), and one avian M2e domain derived an avian type II influenza A subtype (a2M2e).

2. The vaccine of claim 1, wherein the cN1 has the amino acid sequence SEQ ID NO:1, the cN2 has the amino acid sequence SEQ ID NO:2, and the B-cNA has the amino acid sequence SEQ ID NO:3.

3. The vaccine of claim 1, wherein the hM2e domain comprises the amino acid sequence SEQ ID NO:4 or an amino acid sequence having at least 90% sequence identity to SEQ ID NO:4, the sM2e domain comprises the amino acid sequence SEQ ID NO:6 or an amino acid sequence having at least 90% sequence identity to SEQ ID NO:6, the a1M2e domain comprises the amino acid sequence SEQ ID NO:8 or an amino acid sequence having at least 90% sequence identity to SEQ ID NO:8, and the a2M2e domain comprises the amino acid sequence SEQ ID NO:12 or an amino acid sequence having at least 90% sequence identity to SEQ ID NO:12.

4. A recombinant expression vector encoding the vaccine of claim 1, having the formula:
M1-cN1-cN2-B-cNA-5xM2e-X₂-X₃,
M1-cN2-cN1-B-cNA-5xM2e-X₂-X₃,
M1-B-cNA-cN1-cN2-5xM2e-X₂-X₃,
M1-B-cNA-cN2-cN1-5xM2e-X₂-X₃,
M1-cN1-B-cNA-cN2-5xM2e-X₂-X₃, or
M1-cN2-B-cNA-cN1-5xM2e-X₂-X₃,
wherein “M1” consists of a gene encoding an influenza matrix protein 1,
wherein “cN1” consists of a gene encoding the consensus N1 neuraminidase,
wherein “cN2” consists of a gene encoding the consensus N2 neuraminidase,
wherein “B-cNA” consists of a gene encoding the consensus influenza B neuraminidase,
wherein “5xM2e” consists of a gene encoding the tandem repeat of two hM2e domains, one sM2e domains, one a1M2e domain and one a2M2e domain in any order, and
wherein “-” consists of a nucleic acid linker.

5. The vaccine of claim 4, wherein the expression vector construct has the formula:
M1-cN1-cN2-B-cNA-hM2e-hM2e-sM2e-a1M2e-a3M2e-X₂-X₃.

6. The vaccine of claim 1, wherein the vaccine is produced by coinfecting insect cells with one or more recombinant baculoviruses expressing the fusion protein, culturing the insect cells under physiological conditions, and purifying the VLPs from insect cell culture supernatants, or is expressed by multivalent mRNA molecules encoding each vaccine protein.

7. The vaccine of claim 1, further comprising an influenza virus-like particle (VLP) vaccine, mRNA vaccine, a whole inactivated virus, split viral vaccine, or live attenuated influenza vaccine.

8. The vaccine of claim 1, formulated for delivery via intranasal, intramuscular, subcutaneous, transdermal or sublingual administration.

9. A method of vaccinating a subject for influenza A comprising administering the cross-protective influenza vaccine of claim 1 to a subject in need thereof by intranasal, intramuscular, subcutaneous, transdermal, or sublingual administration.

10. The method of claim 9, further comprising administering to the subject a composition comprising an influenza virus-like particle (VLP) vaccine, mRNA vaccine, a whole inactivated virus, split viral vaccine, or live attenuated influenza vaccine.

11. The method of claim 10, wherein the cross-protective influenza vaccine and the influenza virus-like particle (VLP) vaccine, the m RNA vaccine, the whole inactivated virus, the split viral vaccine, or the live attenuated influenza vaccine are in the same composition.

12. The method of claim 10, wherein the composition comprising influenza virus-like particle (VLP) vaccine, a whole inactivated virus, split viral vaccine, or live attenuated influenza vaccine is administered before or after the cross-protective influenza vaccine.

13. The method of claim 10, wherein the cross-protective influenza vaccine is administered prior to influenza seasonal vaccination or after influenza seasonal vaccination.

14. The method of claim 13, wherein the period between cross-protective influenza vaccine and seasonal vaccination administration is one day to 10 years.

15. A cross-protective influenza vaccine comprising a fusion protein comprising two influenza virus matrix protein 2 extracellular (M2e) domains, amino acids 37-61 of an influenza virus hemagglutinin (HA) H3 stalk head domain (HA1), amino acids 305-338 of the HA1, amino acids 1-117 of an HA H3 stalk stem domain (HA2), and a trimeric foldon domain.

16-25. (canceled)

26. A cross-protective influenza vaccine comprising a fusion protein comprising two influenza virus matrix protein 2 extracellular (M2e) domains, amino acids 31-54 of an influenza virus hemagglutinin (HA) H1 stalk head domain (HA1), amino acids 304-337 of the HA1, amino acids 1-117 of an HA H3 stalk stem domain (HA2), and a trimeric foldon domain.

27-33. (canceled)

34. A cross-protective influenza vaccine comprising an mRNA that comprises a nucleic acid sequence encoding a signal peptide, two human influenza A subtypes (hM2e), one M2e domain derived from a swine influenza A subtype (sM2e), one avian M2e domain derived an avian type I influenza A subtype (a1M2e), one avian M2e domain derived an avian type II influenza A subtype (a2M2e), a tetramer stabilizing domain, and a transmembrane domain derived from HA.

35. (canceled)

36. A cross-protective influenza vaccine comprising an mRNA that comprises a nucleic acid sequence encoding a signal peptide, one human influenza A subtypes (hM2e), one M2e domain derived from a swine influenza A subtype (sM2e), amino acids 18-41 and 290-323 of H1 HA, amino acids 21-46 and 290-323 of H3 HA, a consensus HA2-stalk comprising amino acids 1-117 from H1 or H3, and a trimeric foldon domain.

37-39. (canceled)

40. A method of vaccinating a subject for influenza A comprising administering to the subject the vaccine of claim 1.

41. The method of claim 40, further comprising administering to the subject a composition comprising an influenza virus-like particle (VLP) vaccine, mRNA vaccine, a whole inactivated virus, split viral vaccine, or live attenuated influenza vaccine.


SEQUENCE LISTING (55 sequences)

SEQ ID NO: 1 - protein, 468 aa, unidentified
MNPNQKIITIGSVCMTIGMANLILQIGNIISIWVSHSIQIGNQSQIETCNQSVITYENNTWVNQTYVNISNTNFAAGQSWSVKLAGNSSLCPVSGWAIYSKDNSVRIGSKGDVFVIREPFISCSPLECRTFFLTQGALLNDKHSNGTIKDRSPHRTLMSCPIGEAPSPYNSRFESVAWSASACHDGTSWLTIGISGPDSGAVAVLKYNGIITDTIKSWRNNILRTQESECACVNGSCFTIMTDGPSDGQASYKIFKMEKGKIVKSVEMDAPNYHYEECSCYPDSSEITCVCRDNWHGSNRPWVSFNQNLEYQIGYICSGVFGDNPRPNDKTGSCGPVSSNGANGVKGFSFKYGNGVWIGRTKSTSSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 2 - protein, 465 aa, unidentified
MNPNQKIITIGSVSLTISTICFFMQIAILITTVTLHFKQYEFNSPPNNQVMLCEPTIIERNITEIVYLTNTTIEKEICPKPAEYRNWSKPQCGITGFAPFSKDNSIRLSAGGDIWVTREPYVSCDPDKCYQFALGQGTTLNNVHSNDTVRDRTPYRTLLMNELGVPFHLGTKQVCIAWSSSSCHDGKAWLHVCITGDDKNATASFIYNGRLVDSWSWSKDILRTQESECVCINGTCTWMTDGSASGKADTKILFIEEGKIVHTSKLSGSAQHVEECSCYPRYPGVRCVCRDNWKGSNRPIVDINIKDHSIVSSYVCSGLVGDTPRKNDSSSSSHCLDPNNEEGGHGVKGWAFDDGNDVWMGRTISEKSRSGYETFKWEGWSNPKSKLQINRQVIVDRGDRSGYSGIFSVEGKSCINRCFYVELIRGRKEETEVLWTSNSIWFCGTSGTYGTGSWPDGADLNLMPI

SEQ ID NO: 3 - protein, 466 aa, unidentified
MLPSTIQTLTLFLTSGGVLLSLYVSASLSYLLYSDILLKFSRTEITAPIMPLDCANASNVQAVNRSATKGVTPLLPEPEWTYPRLSCPGSTFQKALLISPHRFGETKGNSAPLIIREPFIACGPKECKHFALTHYAAQPGGYYNGTREDRNKLRHLISVKLGKIPTVENSIFHMAAWSGSACHDGKEWTYIGVDGPDSNALLKIKYGEAYTDTYHSYAKNILRTQESACNCIGGDCYLMITDGPASGVSECRFLKIREGRIIKEIFPTGRVKHTEECTCGFASNKTIECACRDNSYTAKRPFVKLNVETDTAEIRLMCTKTYLDTPRPNDGSITGPCESDGDKGSGGIKGGFVHQRMASKIGRWYSRTMSKTKRMGMGLYVKYDGDPWTDSEALALSGVMVSMEEPGWYSFGFEIKDKKCDVPCIGIEMVHDGGKTTWHSAATAIYCLMGSGQLLWDTVTGVNMTL

SEQ ID NO: 4 - protein, 11 aa, unidentified
PIRNEWGSRSN

SEQ ID NO: 5 - protein, 23 aa, unidentified
SLLTEVETPIRNEWGSRSNDSSD

SEQ ID NO: 6 - protein, 11 aa, unidentified
PTRSEWESRSS

SEQ ID NO: 7 - protein, 23 aa, unidentified
SLLTEVETPTRSEWESRSSDSSD

SEQ ID NO: 8 - protein, 23 aa, unidentified
SLLTEVETPTRNEWESRSSDSSD

SEQ ID NO: 9 - protein, 22 aa, unidentified
SLLTEVEPTRNGWECRCSDSSD

SEQ ID NO: 10 - protein, 23 aa, unidentified
SLLTEVETPTRHEWECRCSDSSD

SEQ ID NO: 11 - protein, 23 aa, synthetic construct
SLLTEVETPTRKEWECRCSDSSD

SEQ ID NO: 12 - protein, 11 aa, unidentified
LTRNGWGCRCS

SEQ ID NO: 13 - protein, 23 aa, unidentified
SLLTEVETLTRNGWGCRCSDSSD

SEQ ID NO: 14 - protein, 272 aa, unidentified
MKFLVNVALVFMWYISYIYADPINMTTSINNNLQRVRELAVQSANSAAAPGAAVDGTSLLTEVETPIRNEWGSRSNDSSDAAAGGAASLLTEVETPIRNEWGSRSNDSSDAAAPGAASLLTEVETPTRSEWESRSSDSSDAAAGGAASLLTEVETPTRNEWESRSSDSSDAAAPGAASLLTEVETLTRNGWGCRCSDSSDGGLKQIEDKLEEILSKLYHIENELARIKKLLGELEILAIYSTVASSLVLLVSLGAISFWMCSNGSLQCRICI

SEQ ID NO: 15 - protein, 240 aa, unidentified
MDAMKRGLCCVLLLCGAVFVSASQESLLTEVETPIRNEWGSRSNDSSDAAAGGASLLTEVETPTRSEWESRSSDSSDAAAPGAASLLTEVETPTRNEWESRSSDSSDAAAGGASLLTEVETPTRTGWESNSNGSSDAAAPGAASLLTEVETPIRNEWGSRSNDSSDAAAGGGQIEDKLEEILSKLYHIENELARIKKLLGEYQILAIYSTVASSLVLLVSLGAISFWMCSNGSLQCRICI

SEQ ID NO: 16 - protein, 301 aa, synthetic construct
MDAMKRGLCCVLLLCGAVFVSASQESLLTEVETPIRNEWGSRSNDSSDAAAGGAASLLTEVETPTRSEWESRSSDSSDGGGGSPNGTLVKTITDDQIEVTNATELVQSSGSAPNDKPFQNTNKNTTGASPKYVKQNTLKLATGMRNGSAGSAGLFGAIAGFIENGWEGMIDGWYGFRHQNSEGTGQAADLKSTQAAIDQINGKLNRVIEKTNEKDHQDEKEFSEDEGRIQDLEKYVEDTKIDLWSYNAELLVALENQHTIDATDSEMNKQGTGGGYIPEAPRDGQAYVRKDGEWVLLSTFL

SEQ ID NO: 17 - protein, 519 aa, synthetic construct
MDAMKRGLCCVLLLCGAVFVSASQESLLTEVETPIRNEWGSRSNDSSDGGSGGGSLLTEVETPTRSEWESRSSDSSDAAAPGAASHSIQIGNQSQIETCNQSVITYENNTWVNQTYVNISNTNFAAGQSVVSVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 18 - protein, 469 aa, unidentified
MNPNQKIITIGSVSLTISTICFFMQIAILITTVTLHFKQYEFNSPPNNQVMLCEPTIIERNITEIVYLTNTTIEKEICPKPAEYRNWSKPQCGITGFAPFSKDNSIRLSAGGDIWVTREPYVSCDPDKCYQFALGQGTTLNNVHSNDTVRDRTPYRTLLMNELGVPFHLGTKQVCIAWSSSSCHDGKAWLHVCITGDDKNATASFIYNGRLVDSVVSWSKDILRTQESECVCINGTCTVVMTDGSASGKADTKILFIEEGKIVHTSTLSGSAQHVEECSCYPRYPGVRCVCRDNWKGSNRPIVDINIKDHSIVSSYVCSGLVGDTPRKNDSSSSSHCLDPNNEEGGHGVKGWAFDDGNDVWMGRTISEKSRSGYETFKVVEGWSNPKSKLQINRQVIVDRGDRSGYSGIFSVEGKSCINRCFYVELIRGRKEETEVLWTSNSIVVFCGTSGTYGTGSWPDGADLNLMPI

SEQ ID NO: 19 - protein, 586 aa, unidentified
MDAMKRGLCCVLLLCGAVFVSASQESLLTEVETPIRNEWGSRSNDSSDGGSGGGSLLTEVETPTRSEWESRSSDSSDAAAPGAASLLTEVETPTRNEWESRSSDSSDAAGGGASLLTEVETPTRTGWESNSNGSSDAAPGGSGIINETADDIVYRLTVIIDDRYESLKNLITLRADRLEMIINDNVSTILASGGSGGPEWTYPRLSCPGSTFQKALLISPHRFGETKGNSAPLIIREPFIACGPKECKHFALTHYAAQPGGYYNGTREDRNKLRHLISVKLGKIPTVENSIFHMAAWSGSACHDGKEWTYIGVDGPDSNALLKIKYGEAYTDTYHSYAKNILRTQESACNCIGGDCYLMITDGPASGVSECRFLKIREGRIIKEIFPTGRVKHTEECTCGFASNKTIECACRDNSYTAKRPFVKLNVETDTAEIRLMCTKTYLDTPRPNDGSITGPCESDGDKGSGGIKGGFVHQRMASKIGRWYSRTMSKTKRMGMGLYVKYDGDPWTDSEALALSGVMVSMEEPGWYSFGFEIKDKKCDVPCIGIEMVHDGGKTTWHSAATAIYCLMGSGQLLWDTVTGVNMTL

SEQ ID NO: 20 - protein, 27 aa, unidentified
MKFLVNVALVFMVVYISYIYADPINMT

SEQ ID NO: 21 - protein, 20 aa, unidentified
MVSAIVLYVLLAAAAHSAFA

SEQ ID NO: 22 - protein, 25 aa, unidentified
MDAMKRGLCCVLLLCGAVFVSASQE

SEQ ID NO: 23 - protein, 33 aa, unidentified
GGLKQIEDKLEEILSKLYHIENELARIKKLLGE

SEQ ID NO: 24 - protein, 37 aa, unidentified
QMDRVVKEMRRQLEMIDKLTTREIEQVELLKRIYDKL

SEQ ID NO: 25 - protein, 37 aa, unidentified
ILAIYSTVASSLVLLVSLGAISFWMCSNGSLQCRICI

SEQ ID NO: 26 - protein, 273 aa, unidentified
TKCQTPLGAINSSLPYQNIHPVTIGECPKYVRSAKLRMVTGLRNNPSIQSRGLFGAIAGFIEGGWTGMIDGWYGYHHQNEQGSGYAADQKSTQNAINGITNKVNTVIEKMNIQFTAVGKEFNKLEKRMENLNKKVDDGFLDIWTYNAELLVLLENERTLDFHDSNVKNLYEKVKSQLKNNAKEIGNGCFEFYHKCDNECMESVRNGTYDYPKYSEESKLNREKVDGVKLESMGIYQILAIYSTVASSLVLLVSLGAISFWMCSNGSLQCRICI

SEQ ID NO: 27 - protein, 119 aa, unidentified
GLFGAIAGFIEGGWTGMIDGWYGYHHQNEQGSGYAADQKSPGTQNAINGITNKVNTVIEKMNIQDTATGKEFNKDEKRMENLNKKVDDGFLDIWTYNAELLVLLENERTLDAHDSNVKN

SEQ ID NO: 28 - protein, 566 aa, unidentified
MKAILVVLLYTFATANADTLCIGYHANNSTDTVDTVLEKNVTVTHSVNLLEDKHNGKLCKLRGVAPLHLGKCNIAGWILGNPECESLSTASSWSYIVETSSSDNGTCYPGDFINYEELREQLSSVSSFERFEIFPKTSSWPNHESNKGVTAACPHAGAKSFYKNLIWLVKKGNSYPKLSQSYINDKGKEVLVLWGIHHPPTTADQQSLYQNADAYVFVGTSRYSKKFKPEIAIRPKVRDQEGRMNYYWTLVEPGDKITFEATGNLVVPRYAFTMERNAGSGIIISDTPVHDCNTTCQTPEGAINTSLPFQNIHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVKLESTRIYQILAIYSTVASSVVLVVSLGAISFWMCSNGSLQCRICI

SEQ ID NO: 29 - protein, 528 aa, unidentified
MKTIIALSYIFCLPLGQDLPGNDNSTATLCLGHHAVPNGTLVKTITDDQIEVTNATELVQSSSTGKICNNPHRILDGIDCTLIDALLGDPHCDVFQNETWDLFVERSKAFSNCYPYDVPDYASLRSLVASSGTLEFITEGFTWTGVTQNGGSNACKRGPGSGFFSRLNWLTKSGSTYPVLNVTMPNNDNFDKLYIWGIHHPSTNQEQTSLYVQASGRVTVSTRRSQQTIIPNIGSRPWVRGLSSRISIYWTIVKPGDVLVINSNGNLIAPRGYFKMRTGKSSIMRSDAPIDTCISECITPNGSIPNDKPFQNVNKITYGACPKYVKQNTLKLATGMRNVPEKQTRGLFGAIAGFIENGWEGMIDGWYGFRHQNSEGTGQAADLKSTQAAIDQINGKLNRVIEKTNEKFHQIEKEFSEVEGRIQDLEKYVEDTKIDLWSYNAELLVALENQHTIDLTDSEMNKLFEKTRRQLRENAEEMGNGCFKIYHKCDNACIESIRNGTYDHDVYRDEALNNRFQIKGVELKSGYK

SEQ ID NO: 30 - protein, 26 aa, unidentified
PNGTLVKTITDDQIEVTNATELVQSS

SEQ ID NO: 31 - protein, 34 aa, unidentified
PNDKPFQNVNKITYGACPKYVKQNTLKLATGMRN

SEQ ID NO: 32 - protein, 117 aa, unidentified
GLFGAIAGFIENGWEGMIDGWYGFRHQNSEGTGQAADLKSTQAAIDQINGKLNRVIEKTNEKFHQIEKEFSEVEGRIQDLEKYVEDTKIDLWSYNAELLVALENQHTIDLTDSEMNK

SEQ ID NO: 33 - protein, 182 aa, unidentified
GLFGAIAGFIEGGWTGMIDGWYGYHHQNEQGSGYAADQKSTQNAINGITNKVNSVIEKMNIQFTAVGKEFNKLEKRMENLNKKVDDGFLDIWTYNAELLVLLENERTLDFHDSNVKNLYEKVKSQLKNNAKEIGNGCFEFYHKCDNECMESVRNGTYDYPKYSEESKLNREKVDGVKLESMG

SEQ ID NO: 34 - protein, 24 aa, unidentified
DTVDTVLEKNVTVTHSVNLLEDSH

SEQ ID NO: 35 - protein, 34 aa, unidentified
NSSLPYQNTHPTTNGESPKYVRSAKLRMVTGLRN

SEQ ID NO: 36 - protein, 119 aa, unidentified; identical to SEQ ID NO: 27

SEQ ID NO: 37 - protein, 305 aa, unidentified
MDAMKRGLCCVLLLCGAVFVSASQESLLTEVETPIRNEWGSRSNDSSDAAAGGAASLLTEVETPTRSEWESRSSDSSDGGGGSDTVDTVLEKNVTVTHSVNLLEDSHGSANSSLPYQNTHPTTNGESPKYVRSAKLRMVTGLRNGSAGSAPGGLFGAIAGFIEGGWTGMIDGWYGYHHQNEQGSGYAADQKSPGTQNAINGITNKVNTVIEKMNIQDTATGKEFNKDEKRMENLNKKVDDGFLDIWTYNAELLVLLENERTLDAHDSNVKNQGTGGGYIPEAPRDGQAYVRKDGEWVLLSTFLKL

SEQ ID NO: 38 - protein, 27 aa, unidentified
GYIPEAPRDGQAYVRKDGEWVLLSTFL

SEQ ID NO: 39 - protein, 7 aa, unidentified
AAAGGAA

SEQ ID NO: 40 - protein, 5 aa, unidentified
GGGGS

SEQ ID NO: 41 - protein, 6 aa, unidentified
GSAGSA

SEQ ID NO: 42 - protein, 5 aa, unidentified
QGTGG

SEQ ID NO: 43 - protein, 24 aa, unidentified
MSLLTEVETPIRNEWGSRSNDSSD

SEQ ID NO: 44 - protein, 14 aa, unidentified
GLFGAIAGFIEGGW

SEQ ID NO: 45 - protein, 14 aa, unidentified
WEGMVDGWYGFRHQ

SEQ ID NO: 46 - protein, 14 aa, unidentified
ENERTLDYHDSNVK

SEQ ID NO: 47 - protein, 11 aa, unidentified
  SITE 4: note = Xaa is Asn, His, or Lys
  SITE 5: note = Xaa is Glu or Gly
PTRXXWESRSS

SEQ ID NO: 48 - DNA, 726 bp, unidentified
  source 1..726: mol_type = other DNA
atggacgccatgaaaagaggcctgtgctgcgtgctgctgctctgtggcgccgtgttcgtgtccgcctctcaggagagcctgctgaccgaggttgagacaccaattcggaacgaatggggatctagatctaatgacagcagcgatgccgcagctggaggagccagcctgctgaccgaggtggaaacccctaccagaagcgagtgggagtccagatcctccgactctagcgacgctgcagcccccggcgccgcttctctgctgacagaagtggaaacccctacaagaaacgagtgggagagcagatccagcgacagctccgatgcagccgctggaggtgccagcctgctgacagaggtggaaacacccacccggaccggctgggaaagcaacagcaatggcagcagcgacgctgccgctcctggcgctgccagcctgctgaccgaggtggaaacccctatcagaaacgagtggggcagccggagcaacgacagctctgatgctgccgctggaggtggccagatcgaggacaagctggaagagatcctgagcaagctgtaccacatcgagaacgaactggccagaatcaagaagctgctgggcgagtaccagattctggctatctacagcaccgtggcctcttccctggtgctgctggtcagcctcggcgccatcagcttctggatgtgcagcaacggctccctgcaatgtagaatctgcatctaataa

SEQ ID NO: 49 - DNA, 909 bp, unidentified
  source 1..909: mol_type = other DNA
atggacgccatgaagagaggactgtgctgcgtgctgctgctgtgtggagctgtgttcgtgtccgcctcccaggagtccctgctgaccgaggtggaaacccctatccggaacgagtggggcagcagaagcaacgacagctctgatgcagctgctggaggagcagccagcctgctgacagaggtcgagacacccacccggagcgaatgggagagcagaagcagcgacagcagcgatggcggcggcggaagccctaatggcacactggtgaaaaccatcaccgacgaccaaatcgaggtgaccaatgccacagaactggtgcagagcagcggcagcgctcctaacgacaagcctttccagaacaccaacaagaacacaaccggcgcctccccaaagtacgtgaagcagaataccctgaaactggccaccggcatgagaaatggctcagctggctctgccggactgttcggcgccatcgccggcttcatcgagaacggatgggaaggcatgatcgacggctggtacggctttagacaccagaacagcgagggcacaggccaggccgccgacctcaagagcacccaggctgccatcgaccagatcaacggcaagctgaaccgggtgatcgagaagaccaacgagaaagaccaccaagacgaaaaggaattcagcgaagatgagggcagaatccaggacctggaaaagtacgtggaagataccaagatcgacctgtggtcctacaacgccgagctgctggtcgccctggaaaaccagcacaccatcgacgccaccgatagcgagatgaacaagcagggaacaggcggcggctacatccccgaggcccctagagatggccaggcctacgtgagaaaggacggcgagtgggtgctgctcagcaccttcctgtaataa

SEQ ID NO: 50 - DNA, 1414 bp, unidentified
  source 1..1414: mol_type = other DNA
atgaaccccaaccagaagatcatcaccatcggcagcgtgtgtatgacaatcggaatggccaacctcatcctgcagatcggcaacatcatcagcatctgggtcagccactctatccagatcggcaaccagtcccagattgagacatgcaaccaaagcgtgatcacctacgagaacaacacctgggtcaaccagacctacgtgaacatcagcaataccaacttcgccgctggccagagcgtggtgtccgtgaagctggccggcaactctagcctgtgccccgtgagcggctgggccatctactctaaggacaacagcgtgcggatcggctccaagggagatgtgttcgtgatcagggaacccttcatcagctgttctcccctggaatgcaggaccttcttcctgacccagggcgctctgctgaacgacaagcattccaatggcacaatcaaggataggtctccatatcggaccctgatgtcctgtcccatcggcgaggtgccatctccctacaactccaggttcgagtctgtggcatggagcgcctccgcctgccacgacggaatcaactggctgaccatcggcatcagcggccctgacagcggagccgtggctgtgctgaagtacaacggaatcatcacagataccatcaagagctggcggaacaacatcctgcggacacaggagagcgagtgcgcctgtgtgaacggctcctgttttaccatcatgaccgacggccctagcgatggacaggccagctacaagatcttcagaatcgaaaagggcaagatcgtgaagagcgtggaaatgaaggcccctaactaccactacgaggaatgcagctgctaccccgacagcagcgagatcacatgcgtgtgccgggacaactggcacggctccaatagaccctgggtcagcttcaaccagaacctggaataccagatcggctacatctgcagcggcgtcttcggcgacaatcctcggccaaatgacaagaccggcagctgtggccctgtgtccagcaacggagctaatggcgtgaagggctttagcttcaagtacggcaatggcgtgtggatcggcagaacaaagagcacctccagcaggaaggggtttgagatgatctgggaccccaatggttggacgggcaccgataacaagttcagcatcaaacaggacatcgtcggaatcaacgagtggtccggctacagcggcagctttgtgcagcaccccgagctgacaggcctggactgcatccggccttgcttctgggtggaactgatccgtgggagacctgaggaaaacaccatttggacatccggcagcagcatcagtttctgcggcgtgaacagcgacaccgtgggatggagctggcccgatggcgccgagctgcccttcaccatcgacaaataataaa

SEQ ID NO: 51 - DNA, 1413 bp, unidentified
  source 1..1413: mol_type = other DNA
atgaaccccaatcagaagatcatcaccatcggctctgtgtccctgaccatcagcaccatctgttttttcatgcagatcgccatcctgatcacaaccgtgaccctgcacttcaagcaatacgaattcaacagccctcctaacaaccaggtgatgctgtgcgagcccaccatcatcgagcggaacatcaccgagatcgtctacctgaccaataccaccatcgagaaggagatctgtcccaagcccgccgagtaccggaactggtccaagcctcagtgcggcatcaccggcttcgcccccttctccaaggacaactctatcagactgtccgccggaggagacatctgggtgacaagggaaccctatgtgagctgcgaccccgacaagtgctaccagttcgccctgggccagggcaccaccctgaacaacgtgcattcaaacgacaccgtgagagaccggaccccttaccggaccctgctgatgaacgagctgggcgtccccttccacctgggcacaaagcaggtgtgcatcgcttggagcagcagcagctgccacgatggcaaggcctggctgcacgtgtgtataacaggcgacgacaagaacgccaccgccagcttcatctacaacggccggctggtggacagcgtggtgtcctggtccaaagatatcctgcggacacaggagagcgagtgcgtgtgcatcaatggaacatgcaccgtggtgatgaccgacggcagtgctagcggaaaggccgacacaaggatcctgttcatcgaggagggcaaaatcgtgcacacgagcacactcagcggttctgctcagcacgtggaggagtgtagctgctatcccagataccccggcgtccggtgcgtgtgcagagataactggaaaggcagcaaccggcccatcgtggacatcaacatcaaggaccacagcatcgttagcagctacgtgtgtagcggcctggtgggcgatacacctagaaaaaacgacagcagctccagcagccactgcctggaccccaacaatgaagaaggcggccacggagtgaagggatgggccttcgacgacggcaatgacgtgtggatgggccggaccatctctgagaagagccggtccggctacgagacattcaaggtggtcgagggctggagcaaccccaagtccaagctgcagatcaaccggcaggtgatcgttgacagaggcgatcggagcggctacagcggcatcttcagcgtggaaggcaagtcctgcatcaatcggtgtttctacgtggagctgatccggggcagaaaggaggagaccgaggtgctgtggaccagcaactccattgtggtgttctgcggcaccagcggcacctacggcacaggaagctggcctgacggcgccgatctgaacctgatgcccatctaataa

SEQ ID NO: 52 - DNA, 1764 bp, unidentified
  source 1..1764: mol_type = other DNA
atggacgccatgaaaagaggcctgtgctgcgtgctgctgctctgtggcgccgtgttcgtgtccgcctctcaggagagcctgctgaccgaggttgagacaccaattcggaacgaatggggatctagatctaatgacagcagcgatggcggctccggcggcggcagcctgctgaccgaggtggaaacccctaccagaagcgagtgggagtccagatcctccgactctagcgacgccgctgcccccggcgccgcttctctgctgacagaagtggaaacccctacaagaaacgagtgggagagcagatccagcgacagctccgatgccgcaggaggaggtgctagcctgctgacagaggtggaaacacccacccggaccggctgggaaagcaacagcaatggcagcagcgacgctgcccctggcggaagcggcatcatcaacgagaccgccgacgacatcgtgtacagactgaccgtgatcatcgacgacagatacgagagcctgaagaacctgatcaccctgagagccgacagactggagatgatcatcaacgacaacgtgagcaccatcctggccagcggcggcagcggcggccccgagtggacctacccccgcctgtcctgccccggctccaccttccagaaggccctgctgatctccccccaccgcttcggcgagaccaagggcaactccgcccccctgatcatccgcgagcccttcatcgcctgcggccccaaggagtgcaagcacttcgccctgacccactacgccgcccagcccggcggctactacaacggcacccgcgaggaccgcaacaagctgcgccacctgatctccgtgaagctgggcaagatccccaccgtggagaactccatcttccacatggccgcctggtccggctccgcctgccacgacggcaaggagtggacctacatcggcgtggacggccccgactccaacgccctgctgaagatcaagtacggcgaggcctacaccgacacctaccactcctacgccaagaacatcctgcgcacccaggagtccgcctgcaactgcatcggcggcgactgctacctgatgatcaccgacggccccgcctccggcgtgtccgagtgccgcttcctgaagatccgcgagggccgcatcatcaaggagatcttccccaccggccgcgtgaagcacaccgaggagtgcacctgcggcttcgcctccaacaagaccatcgagtgcgcctgccgcgacaactcctacaccgccaagcgccccttcgtgaagctgaacgtggagaccgacaccgccgagatccgcctgatgtgcaccaagacctacctggacaccccccgccccaacgacggctccatcaccggcccctgcgagtccgacggcgacaagggctccggcggcatcaagggcggcttcgtgcaccagagaatggcctccaagatcggccgctggtactcccgcaccatgtccaagaccaagagaatgggcatgggcctgtacgtgaagtacgacggcgacccctggaccgactccgaggccctggccctgtccggcgtgatggtgtccatggaggagcccggctggtactccttcggcttcgagatcaaggacaagaagtgcgacgtgccctgcatcggcatcgagatggtgcacgacggcggcaagaccacctggcactccgccgccaccgccatctactgcctgatgggctccggccagctgctgtgggacaccgtgaccggcgtgaacatgaccctgtaataa

SEQ ID NO: 53 - DNA, 1704 bp, unidentified
  source 1..1704: mol_type = other DNA
atgaaagccatcctggtggtgctcctgtacaccttcgccaccgctaatgccgacaccctctgcatcggctatcacgccaacaacagcacagatacagtggacaccgtgctggaaaagaatgttacagtcacccacagcgtgaacctgctggaagacaagcacaacggcaagctctgcaaactgagaggagtggcccctctgcacctgggcaaatgtaatatcgccggatggatcctgggcaaccccgagtgcgagtctctgagtaccgcttctagctggtcctacatcgtggaaacaagcagctccgataacggcacatgctaccccggcgacttcatcaactacgaggaactgagagaacaactgagcagcgtgtcctctttcgagcggttcgagatcttccctaagacctccagctggcctaaccacgagagcaacaagggagtgaccgctgcttgtcctcacgccggcgccaagagcttctacaagaacctgatctggctggtcaagaagggcaactcttaccctaagctgagccagtcctacatcaacgacaagggcaaggaggtgctggtgctgtggggcatccaccacccacccaccacagccgaccagcagagcctgtaccaaaacgccgacgcctatgtgttcgtgggcaccagcagatacagcaagaaattcaagcccgagatcgccatccggcccaaggtgcgggaccaggagggaaggatgaactactactggacactggtggaacccggcgataagatcacctttgaggccactgggaatctggtggtgcccaggtacgccttcaccatggaacggaacgctggcagcggcattatcatcagcgacacccccgtgcatgactgcaacacaacatgccagacacccgaaggcgcaatcaacacctctctgccctttcagaacatccaccccatcacaatcggcaagtgccccaaatacgtgaagtccaccaagctgcggctggctaccggcctgaggaacgtgcccagcatccagagccggggtctgttcggtgccatcgccggctttatcgagggcggctggaccggcatggtggatggatggtacggctaccaccaccagaacgagcagggcagcggctacgccgccgatctgaagagcactcagaacgccatcgacaagatcaccaacaaggtgaactctgtgatcgagaagatgaacacacagttcaccgccgtgggaaaggagttcaaccacctggagaagcggatcgaaaatctgaacaagaaggtggacgacggcttcctggatatctggacctacaacgccgagctgctggtcctgctcgagaacgagcggaccctggactaccacgatagcaacgtgaagaatctgtacgagaaagtgcggaaccagctgaagaacaacgccaaggaaatcgggaacggctgcttcgagttctaccataaatgcgacaacacctgcatggaaagcgtgaagaatggcacctacgactaccccaagtactccgaggaagccaagctgaaccgggagaagatcgacggcgtgaagctggaatctacccggatatatcagatcctggccatctacagcaccgtggccagcagcgtggtcctggtggtgtctctgggcgccattagcttttggatgtgcagcaacggcagcctgcagtgccggatctgcatctaataa

SEQ ID NO: 54 - protein, 301 aa, unidentified; identical to SEQ ID NO: 16

SEQ ID NO: 55 - protein, 301 aa, unidentified; identical to SEQ ID NO: 16